(19) United States
(12) Patent Application Publication
(10) Pub. No.: US 2019/0023745 A1
(43) Pub. Date: Jan. 24, 2019

(54) METHODS AND COMPOSITIONS FOR ZIKA VIRUS VACCINES

(71) Applicant: The University of North Carolina at Chapel Hill, Chapel Hill, NC (US)

(72) Inventors: Ralph Baric, Haw River, NC (US); Jessica A. Plante, Texas City, TX (US); Jesica Swanstrom, Morrisville, NC (US); Matthew Begley, Durham, NC (US)

(21) Appl. No.: 16/040,267

(22) Filed: Jul. 19, 2018

Related U.S. Application Data
(60) Provisional application No. 62/534,570, filed on Jul. 19, 2017.

(51) Int. Cl. C07K 14/005; C07K 16/10; A61P 31/14

(52) U.S. Cl. CPC C07K 14/005; C07K 16/10; A61P 31/14; A61K 38/00; C12N 2770/24171; C12N 2770/24134; C12N 2770/24122

(57) ABSTRACT

The present invention methods and compositions for treating a Zika virus infection in a subject, comprising administering to the subject an effective amount of an antibody against Zika virus and/or an epitope that induces an immune response to Zika virus.

### PRIORITY STATEMENT

This application claims the benefit, under 35 U.S.C. § 119(e) of U.S. Provisional Application Ser. No. 62/534,570, filed Jul. 19, 2017, the entire contents of which are incorporated by reference herein.

### STATEMENT OF GOVERNMENT SUPPORT

This invention was made with government support under Grant Nos. AI100625, AI107731 and AI007151 awarded by the National Institutes of Health. The United States government has certain rights in the invention.

### STATEMENT REGARDING ELECTRONIC FILING OF A SEQUENCE LISTING

A Sequence Listing in ASCII text format, submitted under 37 C.F.R. § 1.821, entitled 5470-801_ST25.txt, 721,944 bytes in size, generated on Jul. 19, 2018 and filed via EFS-Web, is provided in lieu of a paper copy. This Sequence Listing is incorporated by reference into the specification for its disclosures.

### FIELD OF THE INVENTION

The present invention is directed to flavivirus vaccines that neutralize Zika virus infection.

### BACKGROUND OF THE INVENTION

Zika virus (ZIKV) is a mosquito-borne flavivirus responsible for thousands of cases of severe fetal malformations and neurological disease since its introduction to Brazil in 2013. Antibodies to flaviviruses can be protective, resulting in lifelong immunity to re-infection by homologous virus. However, cross-reactive antibodies can complicate flavivirus diagnostics and promote more severe disease, as noted after serial dengue virus (DENV) infections. The endemic circulation of DENV in South America and elsewhere raises concerns that preexisting flavivirus immunity may modulate ZIKV disease and transmission potential.

Dengue virus (DENV) is the causative agent of dengue fever and dengue hemorrhagic fever. DENV and its mosquito vectors are widely distributed in tropical and subtropical regions and the disease is endemic in over 100 countries. There are no approved vaccines for dengue.

Dengue virus induced antibody responses are mainly targeted against the envelope (E) protein. Many non-neutralizing antibodies are cross-reactive between the 4 different DENV serotypes (DENV-1-4) and recognize specific epitopes on E that do not attribute to the protection against DENV infections. Highly potent neutralizing antibodies are often targeted against epitopes that require higher order quaternary protein structures that are assembled and displayed on intact virions only. Between serotypes, the neutralizing epitopes differ in structure, complexity and location. These serotype specific neutralizing antibodies render protection against subsequent virus infections of the same serotype.

The present invention overcomes previous shortcomings in the art by providing compositions and methods directed to antibodies and epitopes for use in diagnostics and vaccines directed to Zika virus.

### BRIEF DESCRIPTION OF THE DRAWINGS

FIG. 1. Neutralization and binding of DENV and ZIKV by monoclonal antibodies. (Panel A) mAbs elicited by DENV were evaluated for their ability to neutralize and bind DENV-1, DENV-2, DENV-3, DENV-4, ZIKV H/PF/2013, and ZIKV PRVABC59. (Panel B) The ability of the EDE1 mAbs to neutralize ZIKV H/PF/2013 and ZIKV PRVABD59 was confirmed in both U937+DC-SIGN and Vero cells. (Panel C) Binding of the neutralizing EDE1 mAbs and the non-neutralizing EDE2 B7 mAb to DENV-1, DENV-2, DENV-3, DENV-4, and ZIKV H/PF/2013 was assessed via ELISA. Bars for neutralization data (Panels A and B) represent the mean of two replicates with upper and lower 95% confidence intervals. The dashed line indicates the limit of detection for the assay. Non-neutralizing antibodies were assigned a value of twice the limit of detection for visualization. Bars for binding data (Panel C) represent the mean of two replicates with standard deviations.

FIG. 2. EDE1 C10 protects ZIKV-susceptible mice from infection. Five-week-old type I/II interferon receptor knockout mice on a C57BL/6 backbone received either EDE1 C10 (n=5) or mock (n=5) treatment and were challenged with 10² FFU of ZIKV H/PF/2013. A mock cohort (n=2) was also included. (Panel A) Survival and (Panel B) weight loss was monitored and differences between the mock-treated and EDE1 C10-treated cohorts.

FIG. 3. Neutralization of DENV and ZIKV by DENV primary sera. Geometric mean titers of (Panel A) DENV-1 primary sera, (Panel B) DENV-2 primary sera, (Panel C) DENV-3 primary sera, and (Panel D) and DENV-4 primary sera. Points represent individual sera, and horizontal lines represent the geometric mean titers of all sera with upper and lower 95% confidence intervals. The dashed line indicates the limit of detection for the assay. Non-neutralizing sera were assigned a value of one-half of the limit of detection for visualization and calculation of the geometric means and confidence intervals.

FIG. 4. Neutralization of DENV and ZIKV by DENV secondary sera. Geometric mean titers of DENV secondary sera. Points represent individual sera, and horizontal lines represent the geometric mean titers of all sera with upper and lower 95% confidence intervals. The dashed line indicates the limit of detection for the assay. Non-neutralizing sera were assigned a value of one-half of the limit of detection for visualization and calculation of the geometric means and confidence intervals.

FIG. 5. Neutralization curves of DENV primary sera with ZIKV H/PF/2013. Neutralization curves for primary (Panel A) DENV-1, (Panel B) DENV-2, (Panel C) DENV-3, and (Panel D) DENV-4 sera against ZIKV H/PF/2013 in a U937+DC-SIGN assay.

FIG. 6. Neutralization curves of DENV secondary sera with ZIKV H/PF/2013. Neutralization curves for all DENV secondary sera against ZIKV H/PF/2013 in a U937+DC-SIGN assay.

FIG. 7. CryoEM micrographs of the uncomplexed ZIKV control and the Fab C10-ZIKV complex samples at various pH levels. The deformed particles and aggregates are indicated with gray and black arrows, respectively. The right upper corner inset shows a quarter of a 2D class average of the round particles. The E protein layer is indicated with an arrow, the outer and inner leaflets of the bilayer lipid membrane with arrows. In the pH5.0 uncomplexed ZIKV control, the E protein layer is missing in the 2D class average. Bottom right inset in the pH5.0 uncomplexed ZIKV control is a median filtered (5×5 pixel) image that showed particles with hair-like protrusions, which are likely the E proteins flopping on the virus surface. Scale bar is 500 Å.

FIGS. 8a-8c. Fourier shell correlation plots for the cryoEM maps of the ZIKV-C10 complexes.

### SUMMARY OF THE INVENTION

In one aspect, the present invention provides a method of treating a Zika virus infection in a subject, comprising administering to the subject an effective amount of an EDE1 antibody and/or an epitope that binds an EDE1 antibody.

In a further aspect, the present invention provides a method of protecting a subject from the effects of Zika virus infection, comprising administering to the subject an effective amount of an EDE1 antibody and/or an epitope that binds an EDE1 antibody.

In an additional aspect, the present invention provides a method of protecting a fetus from the effects of Zika virus infection, comprising administering to the mother of the fetus an effective amount of an EDE1 antibody and/or an epitope that binds an EDE1 antibody.

Furthermore, the present invention provides a method of reducing the complications caused by Zika virus infection in a subject, comprising administering to the subject an effective amount of an EDE1 antibody and/or an epitope that binds an EDE1 antibody.

Also provided herein is a method of neutralizing Zika virus, comprising contacting the Zika virus with an EDE1 antibody.

Additionally, the present invention provides a method of treating a disorder caused by Zika virus infection in a subject, comprising administering to the subject an effective amount of an EDE1 antibody and/or an epitope that binds an EDE1 antibody.

In a further aspect, the present invention provides a method of producing an immune response to a Zika virus in a subject, comprising administering to the subject an effective amount of an epitope that binds an EDE1 antibody.

In another aspect, the present invention provides a method of preventing a Zika virus infection in a subject, comprising administering to the subject an effective amount of an EDE1 antibody and/or an epitope that binds an EDE1 antibody.

Further provided herein is a method of identifying the presence of a Zika virus neutralizing epitope in a chimeric Zika virus vaccine, comprising contacting the chimeric vaccine with an EDE1 antibody and detecting binding of the EDE1 antibody to the chimeric vaccine, thereby identifying the presence of a Zika virus neutralizing epitope in the chimeric Zika virus vaccine.

In an additional aspect, the present invention provides a method of identifying an immunogenic composition that induces a neutralizing antibody to a Zika virus in a subject, the method comprising a) contacting a biological sample from a subject that has been administered the immunogenic composition with an epitope that binds an EDE1 antibody; b) determining if the biological sample comprises an antibody that binds the epitope that binds an EDE1 antibody; and c) identifying the immunogenic composition as inducing a neutralizing antibody to a Zika virus in the subject if the biological sample comprises an antibody that binds to the epitope that binds an EDE1 antibody.

As a further aspect, the present invention provides a method of identifying an immunogenic composition that induces a neutralizing antibody to a Zika virus in a subject, the method comprising: a) administering an immunogenic composition comprising a Zika virus antigen to the subject in an amount effective to induce antibodies against the Zika virus antigen; b) contacting a biological sample from the subject with an epitope that binds an EDE1 antibody; c) determining if the biological sample comprises an antibody that binds epitope that binds an EDE1 antibody; and d) identifying the immunogenic composition as inducing a neutralizing antibody to a Zika virus in the subject if the biological sample comprises an antibody that binds to the epitope that binds an EDE1 antibody.

The present invention also provides a method of identifying a neutralizing antibody to a Zika virus, comprising: a) contacting an antibody with an epitope that binds an EDE1 antibody under conditions whereby formation of an antibody/antigen complex can occur; and b) detecting formation of an antibody/antigen complex, thereby identifying a neutralizing antibody to a Zika virus.

Further provided herein is a method of identifying a neutralizing antibody to a Zika virus in a biological sample from a subject, comprising: a) contacting the biological sample comprising an antibody, or suspected of comprising an antibody, with an epitope that binds an EDE1 antibody under conditions whereby formation of an antibody/antigen complex can occur; and c) detecting formation of an antibody/antigen complex, thereby identifying a neutralizing antibody to a Zika virus in the biological sample.

In further aspects, the present invention provides a chimeric dengue virus envelope protein comprising a dengue virus backbone from DENV1 (GenBank Accession No. U88535.1), DENV2 (GenBank Accession No. GU289914.1), DENV3 (GenBank Accession No. JQ411814.1) or DENV4 (GenBank Accession No. KJ160504.1) in which a set of amino acid residues are substituted to introduce an EDE1 epitope that induces a neutralizing antibody response to Zika virus.

Additionally provided herein is a chimeric flavivirus E glycoprotein comprising amino acid residue substitutions that introduce a heterologous epitope that induces an antibody response to Zika virus, selected from the group consisting of: a) Zika virus backbone with DENV1 “5J7” epitope; b) Zika virus backbone with DENV2 “5J7” epitope; c) Zika virus backbone with DENV3 “5J7” epitope; d) Zika virus backbone with DENV4 “5J7” epitope; e) a DENV1 backbone with “5J7” Zika epitope; f) a DENV2 backbone with “5J7” Zika epitope; g) a DENV3 backbone with “5J7” Zika epitope; h) a DENV4 backbone with “5J7” Zika epitope; i) a DENV1 backbone with EDIII from Zika; j) a DENV2 backbone with EDIII from Zika; k) a DENV3 backbone with EDIII from Zika; 1) a DENV4 backbone with EDIII from Zika; m) a DENV1 backbone with “Z20” Zika epitope; n) a DENV2 backbone with “Z20” Zika epitope; o) a DENV3 backbone with “Z20” Zika epitope; p) a DENV4 backbone with “Z20” Zika epitope; q) a DENV1 backbone with “Z3L1” Zika epitope; r) a DENV2 backbone with “Z3L1” Zika epitope; s) a DENV3 backbone with “Z3L1” Zika epitope; t) a DENV4 backbone with “Z3L1” Zika epitope; u) a DENV1 backbone with “Z20” and “Z3L1” Zika epitopes; v) a DENV2 backbone with “Z20” and “Z3L1” Zika epitopes; w) a DENV3 backbone with “Z20” and “Z3L1” Zika epitopes; and x) a DENV4 backbone with “Z20” and “Z3L1” Zika epitopes.

The present invention additionally provides a chimeric flavivirus E glycoprotein comprising substitutions in the amino acid sequence that ablate an epitope that induces an immune response to Zika virus, selected from the group consisting of: a) a Zika virus backbone with “Z20” epitope ablation by DENV1 (SEQ ID NO:37); b) a Zika virus backbone with “Z20” epitope ablation by DENV2 (SEQ ID NO:38); c) a Zika virus backbone with “Z20” epitope ablation by DENV3 (SEQ ID NO:39); d) a Zika virus backbone with “Z20” epitope ablation by DENV4 (SEQ ID NO:40); e) a Zika virus backbone with “Z3L1” epitope ablation by DENV1 (SEQ ID NO:41); f) a Zika virus backbone with “Z3L1” epitope ablation by DENV2(SEQ ID NO:42); g) a Zika virus backbone with “Z3L1” epitope ablation by DENV3 (SEQ ID NO:43); h) a Zika virus backbone with “Z3L1” epitope ablation by DENV4 (SEQ ID NO:44); i) a Zika virus backbone with “Z20” and “Z3L1” epitope ablation by DENV1 (SEQ ID NO:45); j) a Zika virus backbone with “Z20” and “Z3L1” epitope ablation by DENV2 (SEQ ID NO:46); k) a Zika virus backbone with “Z20” and “Z3L1” epitope ablation by DENV3 (SEQ ID NO:47); 1) a Zika virus backbone with “Z20” and “Z3L1” epitope ablation by DENV4 (SEQ ID NO:48); m) Zika virus backbone with EDE1 epitope ablation by tamana bat virus (SEQ ID NO:52); n) Zika virus backbone with core ablation by tamana bat virus (SEQ ID NO:50); o) Zika virus backbone with EDE1 epitope and core ablation by tamana bat virus (SEQ ID NO:51); p) DENV1 backbone with EDE1 epitope ablation by tamana bat virus (SEQ ID NO:56); q) DENV1 backbone with core ablation by tamana bat virus (SEQ ID NO:54); r) DENV1 backbone with EDE1 epitope and core ablation by tamana bat virus (SEQ ID NO:55); s) DENV2 backbone with EDE1 epitope ablation by tamana bat virus (SEQ ID NO:59); t) DENV2 backbone with core ablation by tamana bat virus (SEQ ID NO:57); u) DENV2 backbone with EDE1 epitope and core ablation by tamana bat virus (SEQ ID NO:58); v) DENV3 backbone with EDE1 epitope ablation by tamana bat virus (SEQ ID NO:62); w) DENV3 backbone with core ablation by tamana bat virus (SEQ ID NO:60); x) DENV3 backbone with EDE1 epitope and core ablation by tamana bat virus (SEQ ID NO:61); y) DENV4 backbone with EDE1 epitope ablation by tamana bat virus (SEQ ID NO:65); z) DENV4 backbone with core ablation by tamana bat virus (SEQ ID NO:63); and aa) DENV4 backbone with EDE1 epitope and core ablation by tamana bat virus (SEQ ID NO:64).

### DETAILED DESCRIPTION OF THE INVENTION

The present invention is based on the unexpected discovery of antibodies that neutralize Zika virus and epitopes useful in eliciting an immune response to Zika virus. Thus, in one embodiment, the present invention provides a method of treating a Zika virus infection in a subject, comprising administering to the subject an effective amount of an EDE1 antibody and/or an epitope that binds an EDE1 antibody.

Also included is a method of protecting a subject from the effects of Zika virus infection, comprising administering to the subject an effective amount of an EDE1 antibody and/or an epitope that binds an EDE1 antibody.

Further included is a method of protecting a fetus from the effects of Zika virus infection, comprising administering to the mother of the fetus an effective amount of an EDE1 antibody and/or an epitope that binds an EDE1 antibody.

A method is also provided of reducing the complications caused by Zika virus infection in a subject, comprising administering to the subject an effective amount of an EDE1 antibody and/or an epitope that binds an EDE1 antibody.

In addition, the present invention provides a method of neutralizing Zika virus, comprising contacting the Zika virus with an EDE1 antibody.

Also provided herein is a method of treating a disorder caused by Zika virus infection in a subject, comprising administering to the subject an effective amount of an EDE1 antibody and/or an epitope that binds an EDE1 antibody.

Furthermore, the present invention provides a method of producing an immune response to a Zika virus in a subject, comprising administering to the subject an effective amount of an epitope that binds an EDE1 antibody.

Additionally, the present invention provides a method of preventing a Zika virus infection in a subject, comprising administering to the subject an effective amount of an EDE1 antibody and/or an epitope that binds an EDE1 antibody.

An EDE1 antibody of this invention can be a monoclonal antibody, a polyclonal antibody, a humanized antibody, an antibody fragment, a short chain variable fragment (scFv) or any other type of antibody now known or later identified. The EDE1 antibody can also be an “EDE1-like” antibody, which is an antibody that binds all or a portion of the residues identified in a EDE1 epitope to mediate binding and/or neutralization. Nonlimiting examples of EDE1 antibodies of this invention include C8 and C10. The production of C8 and C10 antibodies is described herein.

The present invention also provides compositions, including a chimeric dengue virus envelope protein comprising a dengue virus backbone from DENV1 (e.g., GenBank Accession No. U88535.1), DENV2 (e.g., GenBank Accession No. GU289914.1), DENV3 (e.g., GenBank Accession No. JQ411814.1) or DENV4 (e.g., GenBank Accession No. KJ160504.1) in which a set of amino acid residues are substituted to introduce an EDE1 epitope that induces a neutralizing antibody response and/or other immune response to Zika virus.

In some embodiments, the dengue virus envelope protein can be DENV1 and the amino acid substitutions include one or more of the following: L46V, K47T, V55E, N67D, T68M, T69A, T70S, T81Y, V83D, E84K, S138R, 1140M, Q148H, Q150D, G159R, T176G, D177G, A180S, S225D, K247R, E249T, Q271E, T272M, S273D, T278R, T293R, E309T, Q323E, K325Q, E362T, K363S, and/or P364K in any combination, to introduce a Zika C10 epitope into DENV1.

In some embodiments, the dengue virus envelope protein can be DENV1 and the amino acid substitutions include one or more of the following: N67D, T68M, T69A, T70S, V83D, E84K, S138R, Q14811, 11158N, K247R, E249T, S273D, T278R, E311I, and/or Q323E, in any combination, to introduce a Zika C8 epitope into DENV1.

In some embodiments, the dengue virus envelope protein can be DENV2 and the amino acid substitutions include one or more of the following: I46V, K47T, Q52N, T55E, N67D, T68M, T69A, T70S, E71D, S81Y, N83D, E84K, I113L, T138R, V140M, E148H, A150D, G159R, T176G, T180S, K247R, D249T, Q271E, S273D, S274G, L278R, Q293R, V309T, R323E, D362T, and/or P364K in any combination, to introduce a Zika C10 epitope into DENV2.

In some embodiments, the dengue virus envelope protein can be DENV2 and the amino acid substitutions include one or more of the following: N67D, T68M, T69A, T70S, E71D, N83D, E84K, I113L, E148H, H158N, K247R, D249T, S273D, S274G, V309T, E311I, and/or R323E, in any combination, to introduce a Zika C8 epitope into DENV2.

In some embodiments, the dengue virus envelope protein can be DENV3 and the amino acid substitutions include one or more of the following: Q46V, K47T, Q52N, T55E, N67D, I68M, T69A, T70S, V81Y, P83D, E84K, T138R, 1140M, Q148H, Q150D, G159R, P174G, E175G, T178S, T223D, K245R, E247T, Q269E, N270M, S271D, S276R, E291R, K307T, K321E, E323Q, E360T, E361S, and/or P362K, in any combination, to introduce a Zika C10 epitope into DENV3.

In some embodiments, the dengue virus envelope protein can be DENV3 and the amino acid substitutions include one or more of the following: N67D, I68M, T69A, T70S, P83D, E84K, Q148H, Q158N, K245R, E247T, S271D, K307T, E3091, and/or K321E, in any combination, to introduce a Zika C8 epitope into DENV3.

In some embodiments, the dengue virus envelope protein can be DENV4 and the amino acid substitutions include one or more of the following: N67D, I68M, T69A, T70S, A71D, T72S, K83D, E84K, V113L, T148H, H158N, D249T, G273D, D274G, D309T, E311E, and/or K323E, in any combination, to introduce a Zika C10 epitope into DENV4.

In some embodiments, the dengue virus envelope protein can be DENV4 and the amino acid substitutions include one or more of the following: T46V, K47T, E52N, L55E, N67D, I68M, T69A, T70S, A71D, T72S, K83D, E84K, V113L, T138R, V140M, T148H, A150D, G159R, P176G, D177G, E180S, D249T, D271E, S272M, G273D, D274G, H278R, D309T, R323E, K325Q, N362T, and/or V364K, in any combination, to introduce a Zika C8 epitope into DENV4.

The present invention additionally provides other chimeric flavivirus E glycoproteins comprising amino acid residue substitutions that introduce a heterologous epitope that induces an antibody response and/or other immune response to Zika virus. Nonlimiting examples of such chimeric flavivirus E glycoproteins include a) Zika virus backbone with DENV1 “5J7” epitope (e.g., SEQ ID NO:13); b) Zika virus backbone with DENV2 “5J7” epitope (e.g., SEQ ID NO:14); c) Zika virus backbone with DENV3 “5J7” epitope (e.g., SEQ ID NO:15); d) Zika virus backbone with DENV4 “5J7” epitope (e.g., SEQ ID NO:16); e) a DENV1 backbone with “5J7” Zika epitope (e.g., SEQ ID NO:17); f) a DENV2 backbone with “5J7” Zika epitope (e.g., SEQ ID NO:18); g) a DENV3 backbone with “5J7” Zika epitope (e.g., SEQ ID NO:19); h) a DENV4 backbone with “5J7” Zika epitope (e.g., SEQ ID NO:20); i) a DENV1 backbone with EDIII from Zika (comprising residues 1-299 of DENV1 followed by residues 306-404 of Zika virus) (e.g., SEQ ID NO:21); j) a DENV2 backbone with EDIII from Zika; comprising residues 1-299 of DENV2 followed by residues 306-404 of Zika virus) (e.g., SEQ ID NO:22); k) a DENV3 backbone with EDIII from Zika; (comprising residues 1-297 of DENV3 followed by residues 306-404 of Zika virus) (e.g., SEQ ID NO:23); 1) a DENV4 backbone with EDIII from Zika (comprising residues 1-299 of DENV4 followed by residues 306-404 of Zika virus) (e.g., SEQ ID NO:24); m) a DENV1 backbone with “Z20” Zika epitope (e.g., SEQ ID NO:25); n) a DENV2 backbone with “Z20” Zika epitope (e.g., SEQ ID NO:26); o) a DENV3 backbone with “Z20” Zika epitope (e.g., SEQ ID NO:27); p) a DENV4 backbone with “Z20” Zika epitope (e.g., SEQ ID NO:28); q) a DENV1 backbone with “Z3L1” Zika epitope (e.g., SEQ ID NO:29); r) a DENV2 backbone with “Z3L1” Zika epitope (e.g., SEQ ID NO:30); s) a DENV3 backbone with “Z3L1” Zika epitope (e.g., SEQ ID NO:31); t) a DENV4 backbone with “Z3L1” Zika epitope (e.g., SEQ ID NO:32); u) a DENV1 backbone with “Z20” and “Z3L1” Zika epitopes (e.g., SEQ ID NO:33); v) a DENV2 backbone with “Z20” and “Z3L1” Zika epitopes (e.g., SEQ ID NO:34); w) a DENV3 backbone with “Z20” and “Z3L1” Zika epitopes (e.g., SEQ ID NO:35); x) a DENV4 backbone with “Z20” and “Z3L1” Zika epitopes (e.g., SEQ ID NO:36); y) a Zika virus backbone with a DENV1 C10 epitope (SEQ ID NO:66); z) a Zika virus backbone with a DENV2 C10 epitope (SEQ ID NO:67); aa) a Zika virus backbone with a DENV3 C10 epitope (SEQ ID NO:68); bb) a Zika virus backbone with a DENV4 C10 epitope (e.g., SEQ ID NO:69); cc) a Zika virus backbone with a DENV1 C10 epitope (non-neurotropic) (e.g., SEQ ID NO:70); dd) a Zika virus backbone with a DENV2 C10 epitope (non-neurotropic (e.g., SEQ ID NO:71); ee) a Zika virus backbone with a DENV3 C10 epitope (non-neurotropic) (e.g., SEQ ID NO:72); ff) a Zika virus backbone with a DENV4 C10 epitope (non-neurotropic) (e.g., SEQ ID NO:73); gg) a Zika virus backbone with a DENV1 C8 epitope (e.g., SEQ ID NO:74); hh) a Zika virus backbone with a DENV2 C8 epitope (e.g., SEQ ID NO:75); ii) a Zika virus backbone with a DENV3 C8 epitope (e.g., SEQ ID NO:76); jj) a Zika virus backbone with a DENV4 C8 epitope (e.g., SEQ ID NO:77); kk) a DENV1 backbone with a ZIKV C10 epitope (e.g., SEQ ID NO:78); 11) a DENV2 backbone with a ZIKV C10 epitope (e.g., SEQ ID NO:79); mm) a DENV3 backbone with a ZIKV C10 epitope (SEQ ID NO:80); nn) a DENV4 backbone with a ZIKV C10 epitope (e.g., SEQ ID NO:81); oo) a DENV1 backbone with a ZIKV C10 epitope (neurotropic) (e.g., SEQ ID NO:82); pp) a DENV2 backbone with a ZIKV C10 epitope (neurotropic) (e.g., SEQ ID NO:83); qq) a DENV3 backbone with a ZIKV C10 epitope (neurotropic) (e.g., SEQ ID NO:84); rr) a DENV4 backbone with a ZIKV C10 epitope (neurotropic) (e.g., SEQ ID NO:85); ss) a DENV1 backbone with a ZIKV C8 epitope (e.g., SEQ ID NO:86); tt) a DENV2 backbone with a ZIKV C8 epitope (e.g., SEQ ID NO:87); uu) a DENV3 backbone with a ZIKV C8 epitope (e.g., SEQ ID NO:88); and vv) a DENV4 backbone with a ZIKV C8 epitope (e.g., SEQ ID NO:89).

Chimeric flavivirus E glycoproteins of this invention can be made by introducing substituted amino acid residues that make up a heterologous epitope into a flavivirus E glycoprotein. The amino acid residues to be substituted to produce a chimeric flavivirus E glycoprotein of this invention are identified in Tables 10-35 herein.

A “heterologous epitope” as used herein can be an epitope that is introduced into an amino acid sequence by substituting amino acid residues and is an epitope that was not previously present in the amino acid sequence in which the substitutions have been made. The introduction of a heterologous epitope allows for the production of multivalent compositions to use for example to induce an immune response. Nonlimiting examples include the chimeric flavivirus E glycoproteins described herein. As a particular example, by introducing the C10 epitope from Zika virus into a DENV1 backbone amino acid sequence by substitution of residues, the resulting chimeric polypeptide can be used to induce an immune response to both Zika virus and DENV1. In some embodiments, the chimeric polypeptide can be a flavivirus E glycoprotein or active fragment or portion thereof. An active fragment or portion thereof is the fragment or portion that is sufficient to induce an immune response.

The chimeric polypeptides and/or other molecules that comprise an epitope of this invention (e.g., a synthetic backbone comprising amino acid residues that make up a C8 or a C10 epitope) can be used in a variety of diagnostic and therapeutic methods. Thus, the present invention provides a method of treating a Zika virus infection in a subject, comprising administering to the subject an effective amount of the chimeric flavivirus E glycoprotein and/or polypeptide that comprises an epitope that induces a neutralizing antibody response and/or other immune response to Zika virus.

Furthermore, the present invention provides a method of protecting a subject from the effects of Zika virus infection, comprising administering to the subject an effective amount of the chimeric flavivirus E glycoprotein and/or polypeptide that comprises an epitope that induces a neutralizing antibody response and/or other immune response to Zika virus.

The present invention additionally provides a method of protecting a fetus from the effects of Zika virus infection, comprising administering to the mother of the fetus an effective amount of the chimeric flavivirus E glycoprotein and/or polypeptide that comprises an epitope that induces a neutralizing antibody response and/or other immune response to Zika virus.

Also provided herein is a method of reducing the complications caused by Zika virus infection in a subject, comprising administering to the subject an effective amount of the chimeric flavivirus E glycoprotein and/or polypeptide that comprises an epitope that induces a neutralizing antibody response and/or other immune response to Zika virus.

A method is also provided of treating a disorder caused by Zika virus infection in a subject, comprising administering to the subject an effective amount of the chimeric flavivirus E glycoprotein and/or polypeptide that comprises an epitope that induces a neutralizing antibody response and/or other immune response to Zika virus.

Further provided herein is a method of producing an immune response to a Zika virus in a subject, comprising administering to the subject an effective amount of the chimeric flavivirus E glycoprotein and/or polypeptide that comprises an epitope that induces a neutralizing antibody response and/or other immune response to Zika virus.

In addition, the present invention provides a method of preventing a Zika virus infection in a subject, comprising administering to the subject an effective amount of the chimeric flavivirus E glycoprotein and/or polypeptide that comprises an epitope that induces a neutralizing antibody response and/or other immune response to Zika virus.

In some embodiments, the chimeric flavivirus E glyprotein can be present in a virus particle, a virus like particle (VLP), and/or a composition for administration as a protein vaccine or therapeutic. In some embodiments, the chimeric flavivirus E glycoprotein can be encoded in nucleic acid that can be naked nucleic acid, nucleic acid in a virus particle, and/or nucleic acid in a composition for administration as a nucleic acid vaccine or therapeutic.

In further embodiments of this invention, a flavivirus E glycoprotein is provided that comprises substitutions in the amino acid sequence that ablate an epitope that induces an immune response to Zika virus. Such “null mutants” can be used for various diagnostic and prognostic purposes. For example, a null mutant of this invention can be included in an assay to measure the fraction of a neutralizing response from a natural infection or a vaccination that targets the quaternary conformation epitopes described herein. The null mutants of this invention can also be used in assays to identify the presence and/or strength of an immune response to a vaccination and such information can be used, for example, to monitor immune status, guide decisions on further immunization and the like, as would be known in the art. In some embodiments, the null mutants of this invention can be used to track the proportion of an immune response directed to EDE and further classify antibodies as more EDE1-like or EDE2-like, e.g., by comparing antibody neutralization and binding results of these null mutants to their wild type parental strains. Such information is useful in determining how prevalent EDE-like antibodies are in naturally occurring individuals and in designing DENV and Zika vaccines that preferentially elicit EDE-like antibodies.

Non-limiting examples of such “null” polypeptides (null mutants) include a) a Zika virus backbone with “Z20” epitope ablation by DENV1; b) a Zika virus backbone with “Z20” epitope ablation by DENV2; c) a Zika virus backbone with “Z20” epitope ablation by DENV3; d) a Zika virus backbone with “Z20” epitope ablation by DENV4; e) y) a Zika virus backbone with “Z3L1” epitope ablation by DENV1; f) a Zika virus backbone with “Z3L1” epitope ablation by DENV2; g) a Zika virus backbone with “Z3L1” epitope ablation by DENV3; h) a Zika virus backbone with “Z3L1” epitope ablation by DENV4; i) a Zika virus backbone with “Z20” and “Z3L1” epitope ablation by DENV1; j) a Zika virus backbone with “Z20” and “Z3L1” epitope ablation by DENV2; k) a Zika virus backbone with “Z20” and “Z3L1” epitope ablation by DENV3; 1) a Zika virus backbone with “Z20” and “Z3L1” epitope ablation by DENV4; m) Zika virus backbone with EDE1 epitope ablation (e.g., by substitutions from the amino acid sequence of tamana bat virus); n) Zika virus backbone with core ablation (e.g., by substitutions from the amino acid sequence of tamana bat virus); o) Zika virus backbone with EDE1 epitope and core ablation (e.g., by substitutions from the amino acid sequence of tamana bat virus)p) DENV1 backbone with EDE1 epitope ablation (e.g., by substitutions from the amino acid sequence of tamana bat virus); q) DENV1 backbone with core ablation (e.g., by substitutions from the amino acid sequence of tamana bat virus); r) DENV1 backbone with EDE1 epitope and core ablation (e.g., by substitutions from the amino acid sequence of tamana bat virus); s) DENV2 backbone with EDE1 epitope ablation (e.g., by substitutions from the amino acid sequence of tamana bat virus); t) DENV2 backbone with core ablation (e.g., by substitutions from the amino acid sequence of tamana bat virus); u) DENV2 backbone with EDE1 epitope and core ablation (e.g., by substitutions from the amino acid sequence of tamana bat virus); v) p) DENV3 backbone with EDE1 epitope ablation (e.g., by substitutions from the amino acid sequence of tamana bat virus); w) DENV3 backbone with core ablation (e.g., by substitutions from the amino acid sequence of tamana bat virus); x) DENV3 backbone with EDE1 epitope and core ablation (e.g., by substitutions from the amino acid sequence of tamana bat virus); y) DENV4 backbone with EDE1 epitope ablation (e.g., by substitutions from the amino acid sequence of tamana bat virus); z) DENV4 backbone with core ablation (e.g., by substitutions from the amino acid sequence of tamana bat virus); and aa) DENV4 backbone with EDE1 epitope and core ablation (e.g., by substitutions from the amino acid sequence of tamana bat virus). Nonlimiting examples of the residues that can be substituted to produce the various null mutant flavivirus E glycoproteins of this invention are provided in Tables 5-9.

Zika virus is a flavivirus. A nonlimiting example of a Zika virus of this invention includes ZIKV-FP2013 (GenBank Accession No. KJ776791.2), also described as ZIKV H/PF/2013. Another example is ZIKV PRVABC59 (GenBank Accession No. KU01215.1).

Tamana bat virus (TABV) is a flavivirus that has no known vector and has limited cross-reactivity for antibodies elicited by other flaviviruses. A nonlimiting example of a Tamana bat virus of this invention has the amino acid sequence as provided under GenBank Accession No. NC_003996.

There are four serotypes of dengue virus (DENV1, DENV2, DENV3 and DENV4). Within each serotype there are a number of different strains or genotypes. The dengue virus antigens and epitopes of the invention can be derived from any dengue virus, including all serotypes, strains and genotypes, now known or later identified. Nonlimiting examples of dengue viruses of this invention include DENV1 (GenBank Accession No. U88535.1), DENV2 (GenBank Accession No. GU289914.1), DENV3 (GenBank Accession No. JQ411814.1), and DENV4 (GenBank Accession No. KJ160504.1).

In some embodiments of the invention, the dengue virus is UNC1017 strain (DENV-1), West Pacific 74 strain (DENV-1), S16803 strain (DEN2), UNC2005 strain (DENV-2), UNC3001 strain (DENV-3), UNC3043 (DENV-3 strain 059.AP-2 from Philippines, 1984), UNC3009 strain (DENV-3, D2863, Sri Lanka 1989), UNC3066 (DEN3, strain 1342 from Puerto Rico 1977), CH53489 strain (DENV-3), UNC4019 strain (DENV-4), or TVP-360 (DENV-4).

Nonlimiting examples of other flaviviruses that can be used in this invention include yellow fever virus (YFV) (e.g., GenBank® Database Accession No. JX503529) Japanese encephalitis virus (JEV) (e.g., GenBank® Database Accession No. U14163), West Nile virus (WNV) (e.g., GenBank® Database Accession No. DQ211652), tick-borne encephalitis virus (TBEV) (e.g., GenBank® Database Accession No. P14336) and any other flavivirus now known or later identified. The amino acid sequence of any of these flaviviruses (which is known) can be used a backbone sequence into which amino acid substitutions can be made to introduce an epitope of this invention to produce a chimeric polypeptide of this invention.

The present invention provides polypeptides (e.g., a chimeric flavivirus E glycoprotein) into which one or more amino acid residues have been substituted in order to introduce a heterologous epitope that induces an immune response to a different flavivirus (I.e., different from the backbone polypeptide). A given amino acid residue in a backbone sequence can be substituted with any amino acid residue (nonlimiting examples of amino acid residues are shown in Table 3).

Alternatively, the amino acid can be a modified amino acid residue (nonlimiting examples are shown in Table 4) and/or can be an amino acid that is modified by post-translation modification (e.g., acetylation, amidation, formylation, hydroxylation, methylation, phosphorylation or sulfatation).

The term “flavivirus E ectodomain” refers to all of the amino acid sequence that is outside the virus and does not include portions of the protein that are embedded in the membrane or inside the virus. In the case of dengue virus E ectodomains, there are some helical segments that are outside the virus and loosely associated with the viral membrane, which are typically not included in the soluble protein referred to as the ectodomain. The E ectodomain typically comprises about 400 amino acids, typically numbered as 1 to 400 in the amino acid sequence of the flavivirus E protein.

The present invention further provides a synthetic backbone comprising an epitope of this invention. As nonlimiting examples, the synthetic backbone can comprise the residues identified in Tables 10-35, which show the amino acid residues in each of the respective amino acid sequences of DENV1, DENV2, DENV3, DENV4 and Zika virus E glycoprotein that comprise an epitope of this invention. In some embodiments, the amino acid residues that make up an epitope of this invention are included in the synthetic backbone in a manner that allows the backbone to assume the appropriate conformation for the epitope to be functional. This approach can be taken to introduce C10 epitope, the C8 epitope, the Z20 epitope, the Z3L1 epitope or both the Z20 and Z3L1 epitopes into a synthetic backbone, in any combination. It would be well understood that the information provided in Tables 10-36 could also be used to construct chimeric E glycoproteins and chimeric flavivirus sequences wherein DENV1, DENV2, DENV3, DENV4 or Zika virus is the backbone sequence and substitutions are made to introduce a heterologous epitope into the backbone sequence. The present invention also provides various screening and diagnostic methods that can employ any of the antibodies, epitopes, polypeptides, virus particles, VLPs and/or compositions of this invention. For example, the present invention provides a method of identifying the presence of a Zika virus neutralizing epitope in a chimeric Zika virus vaccine, comprising contacting the chimeric vaccine with an EDE1 antibody and detecting binding of the EDE1 antibody to the chimeric vaccine, thereby identifying the presence of a Zika virus neutralizing epitope in the chimeric Zika virus vaccine.

Also provided herein is a method of identifying an immunogenic composition that induces a neutralizing antibody to a Zika virus in a subject, the method comprising: a) contacting a biological sample from a subject that has been administered the immunogenic composition with an epitope that binds an EDE1 antibody; b) determining if the biological sample comprises an antibody that binds the epitope that binds an EDE1 antibody; and c) identifying the immunogenic composition as inducing a neutralizing antibody to a Zika virus in the subject if the biological sample comprises an antibody that binds to the epitope that binds an EDE1 antibody.

A method is also provided herein of identifying an immunogenic composition that induces a neutralizing antibody to a Zika virus in a subject, the method comprising: a) administering an immunogenic composition comprising a Zika virus antigen to the subject in an amount effective to induce antibodies against the Zika virus antigen; b) contacting a biological sample from the subject with an epitope that binds an EDE1 antibody; c) determining if the biological sample comprises an antibody that binds epitope that binds an EDE1 antibody; and d) identifying the immunogenic composition as inducing a neutralizing antibody to a Zika virus in the subject if the biological sample comprises an antibody that binds to the epitope that binds an EDE1 antibody.

Further provided herein is a method of identifying a neutralizing antibody to a Zika virus, comprising: a) contacting an antibody with an epitope that binds an EDE1 antibody under conditions whereby formation of an antibody/antigen complex can occur; and c) detecting formation of an antibody/antigen complex, thereby identifying a neutralizing antibody to a Zika virus. Exemplary epitopes that bind an EDE1 antibody are described herein.

Additionally provided herein is a method of identifying a neutralizing antibody to a Zika virus in a biological sample from a subject, comprising: a) contacting the biological sample comprising an antibody, or suspected of comprising an antibody, with an epitope that binds an EDE1 antibody under conditions whereby formation of an antibody/aritigen complex can occur; and b) detecting formation of an antibody/antigen complex, thereby identifying a neutralizing antibody to a Zika virus in the biological sample.

A “sample” or biological sample” of this invention can be any sample that can contain an antibody or polypeptide or antigen as described herein. Nonlimiting examples of a biological sample include blood, serum, plasma, saliva, urine, tears, semen, fecal matter, joint fluid, sputum, lavage fluid, cerebrospinal fluid, mucous, cells, tissue, etc.

As used herein, “5J7” is the term to describe an epitope that is recognized by the monoclonal antibody designated 5J7. Also, “Z20” is the term to describe an epitope that is recognized by the monoclonal antibody designated Z20. Furthermore, “Z3L1” is the term to describe an epitope that is recognized by the monoclonal antibody designated Z3L1.

It is contemplated that the epitopes of this invention can be formed on a solid substrate. A solid substrate of this invention can be any solid surface to which the amino acid residues that make up the epitope can attach in an orientation that allows for formation of the epitope in a functional conformation, according to the methods described herein. In some embodiments, the solid substrate can be, but is not limited to a plate, resin, dish, slide, well, etc., as would be commonly used in an immunoassay or any other type of assay or reaction.

In some embodiments of this invention, the solid substrate can be any type of carrier that has a surface to which amino acid residues and/or polypeptides can attach in an orientation that allows for epitope formation according to the methods described herein. In some embodiments, the solid substrate can be a microparticle or nanoparticle.

Exemplary types of nanoparticles of this invention include but are not limited to, polymer nanoparticles such as PLGA-based, PLA-based, polysaccharide-based (dextran, cyclodextrin, chitosan, heparin), dendrimer, hydrogel; lipid-based nanoparticles such as lipid nanoparticles, lipid hybrid nanoparticles, liposomes, micelles; inorganics-based nanoparticles such as superparamagnetic iron oxide nanoparticles, metal nanoparticles, platin nanoparticles, calcium phosphate nanoparticles, quantum dots; carbon-based nanoparticles such as fullerenes, carbon nanotubes; and protein-based complexes with nanoscales.

Types of microparticles of this invention include but are not limited to particles with sizes at micrometer scale that are polymer microparticles including but not limited to, PLGA-based, PLA-based, polysaccharide-based (dextran, cyclodextrin, chitosan, heparin), dendrimer, hydrogel; lipid-based microparticles such as lipid microparticles, micelles; inorganics-based microparticles such as superparamagnetic iron oxide microparticles, platin microparticles and the like as are known in the art.

As used herein, the terms “nanoparticle” and “nanosphere” describe a polymeric particle or sphere in the nanometer size range. The term microparticle” or “microsphere” as used herein describes a particle or sphere in the micrometer size range. Both types of particles or spheres can be used as carriers of this invention.

A nanoparticle or nanosphere of this invention can have a diameter of 100 nm or less (e.g., in a range from about 1 nm to about 100 nm). In some embodiments, a particle with dimensions more than 100 nm can still be called a nanoparticle. Thus, an upper range for nanoparticles can be about 500 nm. A microparticle or microsphere of this invention can have a diameter of about 0.5 micrometers to about 100 micrometers.

In some embodiments of a nanoparticle or microparticle of this invention, the dimer or multiplicity of dimers is attached to the exterior surface using hydrophobic noncovalent interaction or covalent linkage based on amine/carboxylate chemistry, thiol/maleimide chemistry, and disulfide chemistry. For hydrophobic noncovalent interaction, unmodified monomer and/or dimer can be directly absorbed on the surface of particles. Alternatively, the monomer or dimer can be first chemically or enzymatically modified by conjugation with a fatty acid (i.e., lauric acid, myristic acid, palmitic acid, stearic acid, arachidic acid, oleic acid, etc.), whose long carbon chain allows for tight and strong hydrophobic interaction with or insertion into the surface of particles. For covalent linkage, the functional groups on the surface of particles are first derivatized or activated to introduce activated ester, activated disulfide, or maleimide, followed by reaction with the monomer and/or dimer of this invention.

In some embodiments, a particle of this invention can comprise a polymer that can be PLGA-based, PLA-based, and/or polysaccharide-based (dextran, cyclodextrin, chitosan, heparin etc.); a dendrimer; a hydrogel; a lipid base; a lipid hybrid base; a liposome; a micelle; an inorganic base such as, e.g., superparamagnetic iron oxide, metal, platin, calcium phosphate; a quantum dot; a carbon base, such as, e.g., a fullerene, a carbon nanotube; and a protein-based complex with nanoscales.

In certain embodiments, liposomes may also be employed with the monomers and/or dimers of this invention. The formation and use of liposomes is generally known to those of skill in the art, as summarized below.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via at least four different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one may operate at the same time.

In some embodiments of this invention, the solid substrate can be a nanocapsule. Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made.

In still further embodiments of the invention, the present invention provides peptide mimitopes (see, Meloen et al. (2000) J. Mol. Recognit. 13, 352-359) that mimic the individual and conformational epitopes of the E glycoproteins of the invention. Mimitopes may be identified using any technique known in the art, such as by surface stimulation, random peptide libraries or phage display libraries, using an antibody or antibodies to the individual and conformational epitopes of the E glycoproteins of the invention.

The invention further provides a nucleic acid molecule (e.g., isolated nucleic acid) encoding a polypeptide or peptide of this invention. Also provided are vectors (e.g., plasmids, viral vectors, etc.) encoding the nucleic acid molecules of the invention.

Also provided are cells comprising the polypeptides, peptides, nucleic acid molecules, vectors, virus particles and/or VLPs of this invention.

In additional embodiments, the present invention provides immunogenic compositions comprising the dimers, vectors, nucleic acid molecules, polypeptides and/or any of the compositions of the invention. In some embodiments, the immunogenic composition can be monovalent. In some embodiments, the immunogenic composition is multivalent (e.g., bivalent, trivalent, tetravalent) for dengue virus serotypes DENV-1, DENV-2, DENV-3 and/or DENV-4 in any combination.

The present invention further provides a method of producing an immune response to a flavivirus in a subject, comprising administering to the subject an effective amount of a chimeric polypeptide of this invention and/or any of the compositions of this invention, in any combination.

Furthermore, the present invention provides a method of treating a flavivirus infection in a subject, comprising administering to the subject an effective amount of a chimeric polypeptide of this invention and/or any of the compositions of this invention, in any combination.

Additionally provided herein is a method of preventing a flavivirus infection in a subject, comprising administering to the subject an effective amount of a chimeric polypeptide of this invention and/or any of the compositions of this invention, in any combination.

A method is also provided herein, of protecting a subject from the effects of flavivirus infection, comprising administering to the subject an effective amount of a chimeric polypeptide of this invention and/or any of the compositions of this invention, in any combination.

Further, it is contemplated that the present invention can advantageously be practiced to induce an immune response against one, two, three or all four of DENV-1, DENV-2, DENV-3 and DENV-4 serotypes, in addition to inducing an immune response to Zika virus. It is well-known in the art that effective and safe multivalent dengue vaccines have been a challenge to design because of the problem of interference among serotypes. For example, the immune response may be predominantly directed against only some of the target serotypes. Multiple vaccinations are then required to try to achieve a response against all serotypes; however, in the case of dengue virus, this approach can be dangerous because repeated administrations to a subject with pre-existing antibodies can lead to deleterious effects, such as dengue hemorrhagic fever.

In embodiments of the invention, an “immunogenically active fragment” of a flavivirus E protein ectodomain comprises, consists essentially of or consists of at least about 200, 275, 300, 325, 350, 375, 380, 390 or 395 amino acids, optionally contiguous amino acids, and/or less than about 400, 410, 420, 430, 440 450 or 451 amino acids, optionally contiguous amino acids, including any combination in between the foregoing as long as the lower limit is less than the upper limit, and the “immunogenically active fragment” induces an immune response (e.g., IgG and/or IgA that react with the native antigen), optionally a protective immune response, against flavivirus in a host and induces the production of antibodies that specifically bind to one or more quaternary flavivirus epitopes as described herein.

The term “epitope” as used herein means a specific combination of amino acid residues that, when present in the proper conformation, provide a reactive site for an immune response, e.g., involving an antibody (e.g., B cell epitope) and/or T cell receptor (e.g., T cell epitope).

Portions of a given polypeptide that include a B-cell epitope can be identified using any number of epitope mapping techniques that are known in the art. (See, e.g., Epitope Mapping Protocols in Methods in Molecular Biology, Vol. 66, Glenn E. Morris, Ed., 1996, Humana Press, Totowa, N.J.). For example, linear epitopes can be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715.

Similarly, conformational epitopes can be readily identified by determining spatial conformation of amino acids such as by, e.g., cryoelectron microscopy, x-ray crystallography and 2-dimensional nuclear magnetic resonance. Antigenic regions of proteins can also be identified using standard antigenicity and hydropathy plots, such as those calculated using, e.g., the Omiga version 1.0 software program available from the Oxford Molecular Group. This computer program employs the Hopp/Woods method (Hopp et al., Proc. Natl. Acad. Sci USA (1981) 78:3824-3828) for determining antigenicity profiles and the Kyte-Doolittle technique (Kyte et al., J. Mol. Biol. (1982) 157:105-132) for hydropathy plots.

Generally, T-cell epitopes that are involved in stimulating the cellular arm of a subject's immune system are short peptides of about 8-25 amino acids. A common way to identify T-cell epitopes is to use overlapping synthetic peptides and analyze pools of these peptides, or the individual ones, that are recognized by T cells from animals that are immune to the antigen of interest, using, for example, an enzyme-linked immunospot assay (ELISPOT). These overlapping peptides can also be used in other assays such as the stimulation of cytokine release or secretion, or evaluated by constructing major histocompatibility (MHC) tetramers containing the peptide. Such immunogenically active fragments can also be identified based on their ability to stimulate lymphocyte proliferation in response to stimulation by various fragments from the antigen of interest.

The present invention can be practiced for prophylactic, therapeutic and/or diagnostic purposes. In addition, the invention can be practiced to produce antibodies for any purpose, such as diagnostic or research purposes, or for passive immunization by transfer to another subject.

The present invention further provides a kit comprising one or more compositions of this invention. It would be well understood by one of ordinary skill in the art that the kit of this invention can comprise one or more containers and/or receptacles to hold the reagents (e.g., antibodies, antigens, nucleic acids) of the kit, along with appropriate buffers and/or diluents and/or other reagent and/or solutions and directions for using the kit, as would be well known in the art. Such kits can further comprise adjuvants and/or other immunostimulatory or immunomodulating agents, as are well known in the art.

The compositions and kits of the present invention can also include other medicinal agents, pharmaceutical agents, carriers, diluents, immunostimulatory cytokines, etc. Actual methods of preparing such dosage forms are known, or will be apparent, to those skilled in this art.

Administration to a subject can be by any route known in the art. As non-limiting examples, the route of administration can be by inhalation (e.g., oral and/or nasal inhalation), oral, buccal (e.g., sublingual), rectal, vaginal, topical (including administration to the airways), intraocular, transdermal, by parenteral (e.g., intramuscular [e.g., administration to skeletal muscle], intravenous, intra-arterial, intraperitoneal and the like), subcutaneous (including administration into the footpad), intradermal, intrapleural, intracerebral, and/or intrathecal routes.

The epitopes, polypeptides and other compositions of the invention can be delivered per se or by delivering a nucleic acid (e.g., DNA) that encodes the same.

Immunomodulatory compounds, such as immunomodulatory chemokines and cytokines (preferably, CTL inductive cytokines) can be administered concurrently to a subject.

Cytokines may be administered by any method known in the art. Exogenous cytokines may be administered to the subject, or alternatively, a nucleic acid encoding a cytokine may be delivered to the subject using a suitable vector, and the cytokine produced in vivo. In particular embodiments, a viral adjuvant expresses the cytokine.

In embodiments of the invention, multiple dosages (e.g., two, three or more) of a composition of the invention can be administered without detectable pathogenicity (e.g., Dengue Shock Syndrome/Dengue Hemorrhagic Fever).

In some embodiments of the invention, the multivalent vaccines of the invention do not result in immune interference, e.g., a balanced immune response is induced against all antigens presented. In some embodiments of the invention, the balanced response results in protective immunity against Zika virus, DENV-1, DENV-2, DENV-3 and/or DENV-4 in any combination.

In embodiments of the invention, the multivalent vaccine can be administered to a subject that has anti-dengue maternal antibodies present.

It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of dose (e.g., an amount of a fatty acid) and the like, is meant to encompass variations of ±20%, ±10%, ±5%, ±1%, ±0.5%, or even ±0.1% of the specified amount.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” when used in a claim of this invention is not intended to be interpreted to be equivalent to “comprising.”

As used herein, the term “nucleic acid” and “nucleic acid molecule” encompasses both RNA and DNA, including cDNA, genomic DNA, synthetic (e.g., chemically synthesized) DNA and chimeras of RNA and DNA. The nucleic acid may be double-stranded or single-stranded. The nucleic acid may be synthesized using nucleotide analogs or derivatives (e.g., inosine or phosphorothioate nucleotides). Such nucleotides can be used, for example, to prepare nucleic acids that have altered base-pairing abilities or increased resistance to nucleases.

The term “dengue virus E protein domain I and domain II hinge region” and similar terms would be understood in the art to include the three-dimensional interface between domain I and II in the dengue virus E glycoprotein and, optionally, the adjacent amino acid residues. In addition, those skilled in the art will appreciate that certain amino acid residues in the hinge region may facilitate proper folding and presentation of the epitope, even if they do not form part of the epitope per se. In representative embodiments, the dengue virus E protein domain I and domain II hinge region comprises, consists essentially of, or consists of amino acid positions 47-59, 124-133, 199-222 and/or 206-228 of the E protein of dengue virus serotype 3 (DENV-3; e.g., GenBank® Database Accession No. JQ411814) or the corresponding positions of the E protein of other dengue virus serotypes as described herein.

The term “at least a portion of a dengue virus E protein domain III” and similar terms refer to those portions of E protein domain III that form part of the epitope as well as those amino acid residues that facilitate proper folding and presentation of the epitope, even if they do not form part of the epitope per se. In representative embodiments, the dengue virus E protein domain III comprises, consists essentially of, or consists of amino acid positions 305-308, 323-325, 359-362 and/or 389-390 of the E protein of dengue virus serotype 3 or the corresponding positions of the E protein of other dengue virus serotypes as described herein.

As used herein, the term “polypeptide” encompasses both peptides and proteins (including fusion proteins), unless indicated otherwise.

A “fusion protein” is a polypeptide produced when two heterologous nucleotide sequences or fragments thereof coding for two (or more) different polypeptides not found fused together in nature are fused together in the correct translational reading frame.

A “recombinant” nucleic acid, polynucleotide or nucleotide sequence is one produced by genetic engineering techniques.

A “recombinant” polypeptide is produced from a recombinant nucleic acid, polypeptide or nucleotide sequence.

As used herein, an “isolated” polynucleotide (e.g., an “isolated nucleic acid” or an “isolated nucleotide sequence”) means a polynucleotide at least partially separated from at least some of the other components of the naturally occurring organism or virus, for example, the cell or viral structural components or other polypeptides or nucleic acids commonly found associated with the polynucleotide. Optionally, but not necessarily, the “isolated” polynucleotide is present at a greater concentration (i.e., is enriched) as compared with the starting material (e.g., at least about a two-fold, three-fold, four-fold, ten-fold, twenty-fold, fifty-fold, one-hundred-fold, five-hundred-fold, one thousand-fold, ten thousand-fold or greater concentration). In representative embodiments, the isolated polynucleotide is at least about 1%, 5%, 10%, 20%, 30%, 40%, 50%, 60%, 70%, 80%, 90%, 95% or more pure.

An “isolated” polypeptide means a polypeptide that is at least partially separated from at least some of the other components of the naturally occurring organism or virus, for example, the cell or viral structural components or other polypeptides or nucleic acids commonly found associated with the polypeptide. Optionally, but not necessarily, the “isolated” polypeptide is present at a greater concentration (i.e., is enriched) as compared with the starting material (e.g., at least about a two-fold, three-fold, four-fold, ten-fold, twenty-fold, fifty-fold, one-hundred-fold, five-hundred-fold, one thousand-fold, ten thousand-fold or greater concentration). In representative embodiments, the isolated polypeptide is at least about 1%, 5%, 10%, 20%, 30%, 40%, 50%, 60%, 70%, 80%, 90%, 95% or more pure.

Furthermore, an “isolated” cell is a cell that has been partially or completely separated from other components with which it is normally associated in nature. For example, an isolated cell can be a cell in culture medium and/or a cell in a pharmaceutically acceptable carrier.

“Antibody” as used herein refers to an intact immunoglobulin of any isotype, or a fragment thereof that can compete with the intact antibody for specific binding to the target antigen, and includes chimeric, humanized, caninized, equinized, felinized, fully human, fully canine, fully equine, fully feline, and bispecific antibodies. An intact antibody generally will comprise at least two full-length heavy chains and two full-length light chains, but in some instances may include fewer chains such as antibodies naturally occurring in camelids which may comprise only heavy chains Antibodies according to the invention may be derived solely from a single source, or may be “chimeric,” that is, different portions of the antibody may be derived from two different antibodies. For example, the CDR regions may be derived from a rat or murine source, while the framework regions of the V region are derived from a different animal source, such as a human. The antibodies or binding fragments of the invention may be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below.

“Light chain” as used herein includes a full-length light chain and fragments thereof having sufficient variable region sequence to confer binding specificity. A full-length light chain includes a variable region domain, V_{L}, and a constant region domain, C_{L}. The variable region domain of the light chain is at the amino-terminus of the polypeptide. Light chains according to the invention include kappa chains and lambda chains.

“Heavy chain” includes a full-length heavy chain and fragments thereof having sufficient variable region sequence to confer binding specificity. A full-length heavy chain includes a variable region domain, V_{H}, and three constant region domains, C_{H1}, C_{H2}, and C_{H3}. The V_{H} domain is at the amino-terminus of the polypeptide, and the C_{H} domains are at the carboxyl-terminus, with the C_{H3} being closest to the —COOH end. Heavy chains according to the invention may be of any isotype, including IgG (including IgG1, IgG2, IgG3 and IgG4 subtypes), IgA (including IgA₁ and IgA₂ subtypes), IgM and IgE.

“Immunologically functional fragment” (or simply “fragment”) of an immunoglobulin chain, as used herein, refers to a portion of an antibody light chain or heavy chain that lacks at least some of the amino acids present in a full-length chain but which is capable of binding specifically to an antigen. Such fragments are biologically active in that they bind specifically to the target antigen and can compete with intact antibodies for specific binding to a given epitope. In one aspect of the invention, such a fragment will retain at least one CDR present in the full-length light or heavy chain, and in some embodiments will comprise a single heavy chain and/or light chain or portion thereof. These biologically active fragments may be produced by recombinant DNA techniques, or may be produced by enzymatic or chemical cleavage of intact antibodies Immunologically functional immunoglobulin fragments of the invention include, but are not limited to, Fab, Fab′, F(ab′)₂, Fv, domain antibodies and single-chain antibodies, and may be derived from any mammalian source, including but not limited to human, mouse, rat, camelid or rabbit. It is contemplated further that a functional portion of the antibodies, for example, one or more CDRs, could be covalently bound to a second protein or to a small molecule to create a therapeutic agent directed to a particular target in the body, possessing bifunctional therapeutic properties, or having a prolonged serum half-life.

“Fab fragment” as used herein is comprised of one light chain and the CHI and variable regions of one heavy chain. The heavy chain of a Fab molecule cannot form a disulfide bond with another heavy chain molecule.

“Fc” region as used herein contains two heavy chain fragments comprising the C_{H1} and C_{H2} domains of an antibody. The two heavy chain fragments are held together by two or more disulfide bonds and by hydrophobic interactions of the CH3 domains.

“Fab′ fragment” contains one light chain and a portion of one heavy chain that contains the V_{H} domain and the C_{H1} domain and also the region between the C_{H1} and C_{H2} domains, such that an interchain disulfide bond can be formed between the two heavy chains of two Fab′ fragments to form a F(ab′)₂ molecule.

“F(ab′)₂ fragment” contains two light chains and two heavy chains containing a portion of the constant region between the C_{H1} and C_{H2} domains, such that an interchain disulfide bond is formed between the two heavy chains. A F(ab′)₂ fragment thus is composed of two Fab′ fragments that are held together by a disulfide bond between the two heavy chains.

“Fv region” comprises the variable regions from both the heavy and light chains, but lacks the constant regions.

“Single-chain antibodies” are Fv molecules in which the heavy and light chain variable regions have been connected by a flexible linker to form a single polypeptide chain, which forms an antigen-binding region. Single chain antibodies are discussed in detail in International Patent Application Publication No. WO 88/01649 and U.S. Pat. Nos. 4,946,778 and 5,260,203, the disclosures of which are incorporated by reference.

“Domain antibody” as used herein is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

“Bivalent antibody” as used herein comprises two antigen binding sites. In some instances, the two binding sites have the same antigen specificities. However, bivalent antibodies may be bispecific (see below).

“Multispecific antibody” as used herein is one that targets more than one antigen or epitope.

“Bispecific,” “dual-specific” or “bifunctional” antibody as used herein is a hybrid antibody having two different antigen binding sites. Bispecific antibodies are a species of multispecific antibody and may be produced by a variety of methods including, but not limited to, fusion of hybridomas or linking of Fab′ fragments. See, e.g., Songsivilai & Lachmann (1990) Clin. Exp. Immunol. 79:315-321; Kostelny et al. (1992) J. Immunol. 148:1547-1553. The two binding sites of a bispecific antibody will bind to two different epitopes, which may reside on the same or different protein targets.

The terms “immunogen” and “antigen” are used interchangeably herein and mean any compound (including polypeptides) to which a cellular and/or humoral immune response can be directed. In particular embodiments, an immunogen or antigen can induce a protective immune response against the effects of flavivirus infection.

“Effective amount” as used herein refers to an amount of a vector, nucleic acid, epitope, polypeptide, cell, particle, VLP, composition or formulation of the invention that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation.

The term “immunogenic amount” or “effective immunizing dose,” as used herein, unless otherwise indicated, means an amount or dose sufficient to induce an immune response (which can optionally be a protective response) in the treated subject that is greater than the inherent immunity of non-immunized subjects. An immunogenic amount or effective immunizing dose in any particular context can be routinely determined using methods known in the art.

The terms “vaccine,” “vaccination” and “immunization” are well-understood in the art, and are used interchangeably herein. For example, the terms vaccine, vaccination or immunization can be understood to be a process or composition that increases a subject's immune reaction to an immunogen (e.g., by providing an active immune response), and therefore its ability to resist, overcome and/or recover from infection (i.e., a protective immune response).

By the terms “treat,” “treating” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay in the progression of the disease or disorder. In representative embodiments, the terms “treat,” “treating” or “treatment of” (and grammatical variations thereof) refer to a reduction in the severity of viremia and/or a delay in the progression of viremia, with or without other signs of clinical disease.

A “treatment effective” amount as used herein is an amount that is sufficient to treat (as defined herein) the subject. Those skilled in the art will appreciate that the therapeutic effects need not be complete or curative, as long as some benefit is provided to the subject.

The term “prevent,” “preventing” or “prevention of” (and grammatical variations thereof) refer to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. In representative embodiments, the terms “prevent,” “preventing” or “prevention of” (and grammatical variations thereof) refer to prevention and/or delay of the onset and/or progression of viremia in the subject, with or without other signs of clinical disease. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

A “prevention effective” amount as used herein is an amount that is sufficient to prevent (as defined herein) the disease, disorder and/or clinical symptom in the subject. Those skilled in the art will appreciate that the level of prevention need not be complete, as long as some benefit is provided to the subject.

The efficacy of treating and/or preventing flavivirus infection by the methods of the present invention can be determined by detecting a clinical improvement as indicated by a change in the subject's symptoms and/or clinical parameters (e.g., viremia), as would be well known to one of skill in the art.

Unless indicated otherwise, the terms “protect,” “protecting,” “protection” and “protective” (and grammatical variations thereof) encompass both methods of preventing and treating flavivirus infection in a subject, whether against one or multiple strains, genotypes or serotypes of a flavivirus such as dengue virus.

The terms “protective” immune response or “protective” immunity as used herein indicates that the immune response confers some benefit to the subject in that it prevents or reduces the incidence and/or severity and/or duration of disease or any other manifestation of infection. For example, in representative embodiments, a protective immune response or protective immunity results in reduced viremia, whether or not accompanied by clinical disease. Alternatively, a protective immune response or protective immunity may be useful in the therapeutic treatment of existing disease.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” (Herscowitz, Immunophysiology: Cell Function and Cellular Interactions in Antibody Formation, in IMMUNOLOGY: BASIC PROCESSES 117 (Joseph A. Bellanti ed., 1985)). Alternatively stated, an active immune response is mounted by the host after exposure to immunogens by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “subject” of the invention includes any animal susceptible to flavivirus infection. Such a subject is generally a mammalian subject (e.g., a laboratory animal such as a rat, mouse, guinea pig, rabbit, primates, etc.), a farm or commercial animal (e.g., a cow, horse, goat, donkey, sheep, etc.), or a domestic animal (e.g., cat, dog, ferret, etc.). In particular embodiments, the subject is a primate subject, a non-human primate subject (e.g., a chimpanzee, baboon, monkey, gorilla, etc.) or a human. Subjects of the invention can be a subject known or believed to be at risk of infection by flavivirus. Alternatively, a subject according to the invention can also include a subject not previously known or suspected to be infected by flavivirus or in need of treatment for flavivirus infection.

Subjects may be treated for any purpose, such as for eliciting a protective immune response, for eliciting a neutralizing response, and/or for eliciting the production of antibodies in that subject, which antibodies can be collected and used for other purposes such as research or diagnostic purposes or for administering to other subjects to produce passive immunity therein, etc.

Subjects include males and/or females of any age, including neonates, juvenile, mature and geriatric subjects. With respect to human subjects, in representative embodiments, the subject can be an infant (e.g., less than about 12 months, 10 months, 9 months, 8 months, 7 months, 6 months, or younger), a toddler (e.g., at least about 12, 18 or 24 months and/or less than about 36, 30 or 24 months), or a child (e.g., at least about 1, 2, 3, 4 or 5 years of age and/or less than about 14, 12, 10, 8, 7, 6, 5, or 4 years of age). In embodiments of the invention, the subject is a human subject that is from about 0 to 3, 4, 5, 6, 9, 12, 15, 18, 24, 30, 36, 48 or 60 months of age, from about 3 to 6, 9, 12, 15, 18, 24, 30, 36, 48 or 60 months of age, from about 6 to 9, 12, 15, 18, 24, 30, 36, 48 or 60 months of age, from about 9 to 12, 15, 18, 24, 30, 36, 48 or 60 months of age, from about 12 to 18, 24, 36, 48 or 60 months of age, from about 18 to 24, 30, 36, 48 or 60 months of age, or from about 24 to 30, 36, 48 or 60 months of age.

In embodiments of the invention, the subject has maternal antibodies to a flavivirus of this invention.

A “subject in need” of the methods of the invention can be a subject known to be, or suspected of being, infected with, or at risk of being infected with, a flavivirus of this invention. A subject of this invention can include a woman of child-bearing age, a pregnant woman, a sex partner, a fetus, etc.

Pharmaceutical formulations (e.g., immunogenic formulation) comprising the flavivirus epitopes, polypeptides, and/or other compositions of the invention and a pharmaceutically acceptable carrier are also provided, and can be formulated for administration in a pharmaceutical carrier in accordance with known techniques. See, e.g., Remington, The Science And Practice of Pharmacy (latest edition). In the manufacture of a pharmaceutical composition according to embodiments of the present invention, the composition of the invention is typically admixed with inter alia, a pharmaceutically acceptable carrier. By “pharmaceutically acceptable carrier” is meant a carrier that is compatible with other ingredients in the pharmaceutical composition and that is not harmful or deleterious to the subject. The carrier may be a solid or a liquid, or both, and is preferably formulated with the composition of the invention as a unit-dose formulation, for example, a tablet, which may contain from about 0.01 or 0.5% to about 95% or 99% by weight of the composition. The pharmaceutical compositions are prepared by any of the well-known techniques of pharmacy including, but not limited to, admixing the components, optionally including one or more accessory ingredients. In certain embodiments, the pharmaceutically acceptable carrier is sterile and would be deemed suitable for administration into human subjects according to regulatory guidelines for pharmaceutical compositions comprising the carrier.

Furthermore, a “pharmaceutically acceptable” component such as a salt, carrier, excipient or diluent of a composition according to the present invention is a component that (i) is compatible with the other ingredients of the composition in that it can be combined with the compositions of the present invention without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include any of the standard pharmaceutical carriers such as phosphate buffered saline solutions, water, emulsions such as oil/water emulsion, microemulsions and various types of wetting agents.

In some embodiments, the compositions of the invention can further comprise one or more than one adjuvant. The adjuvants of the present invention can be in the form of an amino acid sequence, and/or in the form or a nucleic acid encoding an adjuvant. When in the form of a nucleic acid, the adjuvant can be a component of a nucleic acid encoding the polypeptide(s) or fragment(s) or epitope(s) and/or a separate component of the composition comprising the nucleic acid encoding the polypeptide(s) or fragment(s) or epitope(s) of the invention. According to the present invention, the adjuvant can also be an amino acid sequence that is a peptide, a protein fragment or a whole protein that functions as an adjuvant, and/or the adjuvant can be a nucleic acid encoding a peptide, protein fragment or whole protein that functions as an adjuvant. As used herein, “adjuvant” describes a substance, which can be any immunomodulating substance capable of being combined with a composition of the invention to enhance, improve or otherwise modulate an immune response in a subject.

In further embodiments, the adjuvant can be, but is not limited to, an immunostimulatory cytokine (including, but not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include without limitation MF 59, LT-K63, LT-R72 (Pal et al., Vaccine 24(6):766-75 (2005)), QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl. lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153, or a less reactogenic composition where the QS21 is quenched with cholesterol as disclosed in PCT publication number WO 96/33739. A particularly potent adjuvant formulation involving QS21 3D-MPL & tocopherol in an oil in water emulsion is described in PCT publication number WO 95/17210. In addition, the nucleic acid compositions of the invention can include an adjuvant by comprising a nucleotide sequence encoding the antigen and a nucleotide sequence that provides an adjuvant function, such as CpG sequences. Such CpG sequences, or motifs, are well known in the art. In embodiments of the invention, the adjuvant comprises an alphavirus adjuvant as described, for example in U.S. Pat. No. 7,862,829.

An adjuvant for use with the present invention, such as, for example, an immunostimulatory cytokine, can be administered before, concurrent with, and/or within a few hours, several hours, and/or 1, 2, 3, 4, 5, 6, 7, 8, 9, and/or 10 days before and/or after the administration of a composition of the invention to a subject.

Furthermore, any combination of adjuvants, such as immunostimulatory cytokines, can be co-administered to the subject before, after and/or concurrent with the administration of an immunogenic composition of the invention. For example, combinations of immunostimulatory cytokines, can consist of two or more immunostimulatory cytokines, such as GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules. The effectiveness of an adjuvant or combination of adjuvants can be determined by measuring the immune response produced in response to administration of a composition of this invention to a subject with and without the adjuvant or combination of adjuvants, using standard procedures, as described herein and as known in the art.

Boosting dosages can further be administered over a time course of days, weeks, months or years. In chronic infection, initial high doses followed by boosting doses may be advantageous.

The pharmaceutical formulations of the invention can optionally comprise other medicinal agents, pharmaceutical agents, stabilizing agents, buffers, carriers, diluents, salts, tonicity adjusting agents, wetting agents, and the like, for example, sodium acetate, sodium lactate, sodium chloride, potassium chloride, calcium chloride, sorbitan monolaurate, triethanolamine oleate, etc.

For injection, the carrier will typically be a liquid. For other methods of administration, the carrier may be either solid or liquid. For inhalation administration, the carrier will be respirable, and is typically in a solid or liquid particulate form.

The compositions of the invention can be formulated for administration in a pharmaceutical carrier in accordance with known techniques. See, e.g., Remington, The Science and Practice of Pharmacy (9^{th} Ed. 1995). In the manufacture of a pharmaceutical composition according to the invention, the VLPs are typically admixed with, inter alia, an acceptable carrier. The carrier can be a solid or a liquid, or both, and is optionally formulated with the compound as a unit-dose formulation, for example, a tablet. A variety of pharmaceutically acceptable aqueous carriers can be used, e.g., water, buffered water, 0.9% saline, 0.3% glycine, hyaluronic acid, pyrogen-free water, pyrogen-free phosphate-buffered saline solution, bacteriostatic water, or Cremophor EL[R] (BASF, Parsippany, N.J.), and the like. These compositions can be sterilized by conventional techniques. The formulations of the invention can be prepared by any of the well-known techniques of pharmacy.

The pharmaceutical formulations can be packaged for use as is, or lyophilized, the lyophilized preparation generally being combined with a sterile aqueous solution prior to administration. The compositions can further be packaged in unit/dose or multi-dose containers, for example, in sealed ampoules and vials.

The pharmaceutical formulations can be formulated for administration by any method known in the art according to conventional techniques of pharmacy. For example, the compositions can be formulated to be administered intranasally, by inhalation (e.g., oral inhalation), orally, buccally (e.g., sublingually), rectally, vaginally, topically, intrathecally, intraocularly, transdermally, by parenteral administration (e.g., intramuscular [e.g., skeletal muscle], intravenous, subcutaneous, intradermal, intrapleural, intracerebral and intra-arterial, intrathecal), or topically (e.g., to both skin and mucosal surfaces, including airway surfaces).

For intranasal or inhalation administration, the pharmaceutical formulation can be formulated as an aerosol (this term including both liquid and dry powder aerosols). For example, the pharmaceutical formulation can be provided in a finely divided form along with a surfactant and propellant. Typical percentages of the composition are 0.01-20% by weight, preferably 1-10%. The surfactant is generally nontoxic and soluble in the propellant.

Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1-20% by weight of the composition, preferably 0.25-5%. The balance of the composition is ordinarily propellant. A carrier can also be included, if desired, as with lecithin for intranasal delivery. Aerosols of liquid particles can be produced by any suitable means, such as with a pressure-driven aerosol nebulizer or an ultrasonic nebulizer, as is known to those of skill in the art. See, e.g., U.S. Pat. No. 4,501,729. Aerosols of solid particles can likewise be produced with any solid particulate medicament aerosol generator, by techniques known in the pharmaceutical art. Intranasal administration can also be by droplet administration to a nasal surface.

Injectable formulations can be prepared in conventional forms, either as liquid solutions or suspensions, solid forms suitable for solution or suspension in liquid prior to injection, or as emulsions. Alternatively, one can administer the pharmaceutical formulations in a local rather than systemic manner, for example, in a depot or sustained-release formulation.

Extemporaneous injection solutions and suspensions can be prepared from sterile powders, granules and tablets of the kind previously described. For example, an injectable, stable, sterile formulation of the invention in a unit dosage form in a sealed container can be provided. The formulation can be provided in the form of a lyophilizate, which can be reconstituted with a suitable pharmaceutically acceptable carrier to form a liquid composition suitable for injection into a subject. The unit dosage form can be from about 1 μg to about 10 grams of the formulation. When the formulation is substantially water-insoluble, a sufficient amount of emulsifying agent, which is pharmaceutically acceptable, can be included in sufficient quantity to emulsify the formulation in an aqueous carrier. One such useful emulsifying agent is phosphatidyl choline.

Pharmaceutical formulations suitable for oral administration can be presented in discrete units, such as capsules, cachets, lozenges, or tables, as a powder or granules; as a solution or a suspension in an aqueous or non-aqueous liquid; or as an oil-in-water or water-in-oil emulsion. Oral delivery can be performed by complexing a compound(s) of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the protein(s) and a suitable carrier (which may contain one or more accessory ingredients as noted above). In general, the pharmaceutical formulations are prepared by uniformly and intimately admixing the compound(s) with a liquid or finely divided solid carrier, or both, and then, if necessary, shaping the resulting mixture. For example, a tablet can be prepared by compressing or molding a powder or granules, optionally with one or more accessory ingredients. Compressed tablets are prepared by compressing, in a suitable machine, the formulation in a free-flowing form, such as a powder or granules optionally mixed with a binder, lubricant, inert diluent, and/or surface active/dispersing agent(s). Molded tablets are made by molding, in a suitable machine, the powdered protein moistened with an inert liquid binder.

Pharmaceutical formulations suitable for buccal (sub-lingual) administration include lozenges comprising the compound(s) in a flavored base, usually sucrose and acacia or tragacanth; and pastilles in an inert base such as gelatin and glycerin or sucrose and acacia.

Pharmaceutical formulations suitable for parenteral administration can comprise sterile aqueous and non-aqueous injection solutions, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of nonaqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations suitable for rectal administration are optionally presented as unit dose suppositories. These can be prepared by admixing the active agent with one or more conventional solid carriers, such as for example, cocoa butter and then shaping the resulting mixture.

Pharmaceutical formulations suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical formulation of the present invention with a lipophilic reagent (e.g., DMSO) that is capable of passing into the skin.

Pharmaceutical formulations suitable for transdermal administration can be in the form of discrete patches adapted to remain in intimate contact with the epidermis of the subject for a prolonged period of time. Formulations suitable for transdermal administration can also be delivered by iontophoresis (see, for example, Pharmaceutical Research 3:318 (1986)) and typically take the form of a buffered aqueous solution of the compound(s). Suitable formulations can comprise citrate or bis\tris buffer (pH 6) or ethanol/water and can contain from 0.1 to 0.2M active ingredient.

Further, the composition of this invention can be formulated as a liposomal formulation. The lipid layer employed can be of any conventional composition and can either contain cholesterol or can be cholesterol-free. The liposomes that are produced can be reduced in size, for example, through the use of standard sonication and homogenization techniques.

The liposomal formulations can be lyophilized to produce a lyophilizate which can be reconstituted with a pharmaceutically acceptable carrier, such as water, to regenerate a liposomal suspension.

The immunogenic formulations of the invention can optionally be sterile, and can further be provided in a closed pathogen-impermeable container.

The pharmaceutical compositions that are provided can be administered for prophylactic and/or therapeutic treatments. An “effective amount” refers generally to an amount that is a sufficient, but non-toxic, amount of the active ingredient (i.e., an anti-Canine IgE antibody or immunologically functional fragment thereof) to achieve the desired effect, which is a reduction or elimination in the severity and/or frequency of symptoms and/or improvement or remediation of damage. A “therapeutically effective amount” refers to an amount that is sufficient to remedy a disease state or symptoms, or otherwise prevent, hinder, retard or reverse the progression of a disease or any other undesirable symptom. A “prophylactically effective amount” refers to an amount that is effective to prevent, hinder or retard the onset of a disease state or symptom.

In general, toxicity and therapeutic efficacy of the antibody or fragment or immunogenic composition and/or vaccine of this invention can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

The data obtained from cell culture and/or animal studies can be used in formulating a range of dosages for subjects for treatment. The dosage of the active ingredient typically lines within a range of circulating concentrations that include the ED₅₀ with little or no toxicity. The dosage can vary within this range depending upon the dosage form employed and the route of administration utilized.

The effective amount of a pharmaceutical composition of this invention to be employed therapeutically or prophylactically will depend, for example, upon the therapeutic context and objectives. One skilled in the art will appreciate that the appropriate dosage levels for treatment, according to certain embodiments, will thus vary depending, in part, upon the molecule delivered, the indication for which the anti-composition is being used, the route of administration, and the size (body weight, body surface or organ size) and/or condition (the age and general health) of the subject. A clinician may titer the dosage and modify the route of administration to obtain the optimal therapeutic effect. Typical dosages can range from about 10 or 100 ug/Kg, or 500 or 1 mg/Kg, up to about 50 or 100 mg/Kg subject body weight, or more.

In some embodiments of the invention, the dosage of a protein (e.g., a composition comprising a polypeptide of this invention or a polypeptide linked to a carrier such as a nanoparticle) can be in a range of about 10° to about 10⁴ micrograms, +/− adjuvant.

The dosing frequency will depend upon the pharmacokinetic parameters of the composition being administered. For example, a clinician will administer the composition until a dosage is reached that achieves the desired effect. The composition may therefore be administered as a single dose, or as two or more doses (which may or may not contain the same amount of the desired molecule) over time, or as a continuous infusion via an implantation device or catheter. Treatment may be continuous over time or intermittent. Further refinement of the appropriate dosage is routinely made by those of ordinary skill in the art and is within the ambit of tasks routinely performed by them. Appropriate dosages may be ascertained through use of appropriate dose-response data.

As used herein, the term “antibody” includes intact immunoglobulin molecules as well as fragments thereof that are capable of binding the epitopic determinant of an antigen (i.e., antigenic determinant).

A neutralizing antibody is an antibody that reacts with an infectious agent (usually a virus) and destroys or inhibits its infectivity and virulence. Neutralization can, in some embodiments, be demonstrated by means of mixing serum with the suspension of infectious agent, and then injecting the mixture into animals or cell cultures that are susceptible to the agent in question. In some embodiments, a neutralizing antibody can be administered to a subject that is infected with the infectious agent (e.g., a virus) to treat the subject by neutralizing the infectious agent and alleviating the deleterious effects of the invention.

Antibodies that bind the polypeptides of this invention are prepared using intact polypeptides or fragments as the immunizing antigen. The polypeptide or fragment used to immunize a subject can be derived from enzymatic cleavage, recombinant expression, isolation from biological materials, synthesis, etc., and can be conjugated to a carrier protein, if desired. Commonly used carriers that are chemically coupled to peptides and proteins for the production of antibody include, but are not limited to, bovine serum albumin, thyroglobulin and keyhole limpet hemocyanin. The coupled peptide or protein can then used to immunize an animal (e.g., a mouse, rat, or rabbit). The polypeptide or peptide antigens can also be administered with an adjuvant, as described herein and as otherwise known in the art.

An antibody of this invention can be any type of immunoglobulin, including IgG, IgM, IgA, IgD, and/or IgE. The antibody can be monoclonal or polyclonal and can be of any species of origin, including, for example, mouse, rat, rabbit, horse, goat, sheep or human, or can be a chimeric or humanized antibody (e.g., Walker et al., Molec. Immunol. 26:403-11 (1989)). The antibodies can be recombinant monoclonal antibodies produced according to the methods disclosed in U.S. Pat. No. 4,474,893 or U.S. Pat. No. 4,816,567. The antibodies can also be chemically constructed according to methods disclosed in U.S. Pat. No. 4,676,980. The antibody can further be a single chain antibody (e.g., scFv) or bispecific antibody.

Antibody fragments included within the scope of the present invention include, for example, Fab, F(ab′)2, and Fc fragments, and the corresponding fragments obtained from antibodies other than IgG. Such fragments can be produced by known techniques. For example, F(ab′)2 fragments can be produced by pepsin digestion of the antibody molecule, and Fab fragments can be generated by reducing the disulfide bridges of the F(ab′)2 fragments. Alternatively, Fab expression libraries can be constructed to allow rapid and easy identification of monoclonal Fab fragments with the desired specificity (Huse et al., (1989) Science 254:1275-1281). Antibodies can also be obtained by phage display techniques known in the art or by immunizing a heterologous host with a cell containing an epitope of interest.

The polypeptide, fragment or antigenic epitope that is used as an immunogen can be modified or administered in an adjuvant in order to increase antigenicity. Methods of increasing the antigenicity of a protein or peptide are well known in the art and include, but are not limited to, coupling the antigen with a heterologous protein (such as globulin or β-galactosidase) or through the inclusion of an adjuvant during immunization.

For example, for the production of antibodies, various hosts including goats, rabbits, rats, mice, humans, and others, can be immunized by injection with the polypeptides and/or fragments of this invention, with or without a carrier protein. Additionally, various adjuvants may be used to increase the immunological response. Such adjuvants include, but are not limited to, Freund's complete and incomplete adjuvants, mineral gels such as aluminum hydroxide, and surface-active substances such as lysolecithin, pluronic polyols, polyanions, peptides, oil emulsions, keyhole limpet hemocyanin, and dinitrophenol. Among adjuvants used in humans, BCG (bacilli Calmette-Guerin) and Corynebacterium parvum are especially preferable.

Monoclonal antibodies can be produced in a hybridoma cell line according to the technique of Kohler and Milstein (Nature 265:495-97 (1975)). Other techniques for the production of monoclonal antibodies include, but are not limited to, the human B-cell hybridoma technique, and the EBV-hybridoma technique (Kozbor et al. 1985. J. Immunol. Methods 81:31-42; Cote et al. 1983. Proc. Natl. Acad. Sci. 80:2026-2030; Cole et al. 1984. Mol. Cell Biol. 62:109-120).

For example, to produce monoclonal antibodies, a solution containing the appropriate antigen can be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in a bacterial cell such as E. coli by recombinant techniques known to those skilled in the art (e.g., Huse. Science 246:1275-81 (1989)). Any one of a number of methods well known in the art can be used to identify the hybridoma cell, which produces an antibody with the desired characteristics. These include screening the hybridomas by ELISA assay, Western blot analysis, or radioimmunoassay. Hybridomas secreting the desired antibodies are cloned and the class and subclass are identified using standard procedures known in the art.

For polyclonal antibodies, antibody-containing serum is isolated from the immunized animal and is screened for the presence of antibodies with the desired specificity using any of the well known procedures as described herein.

The present invention further provides antibodies of this invention in detectably labeled form. Antibodies can be detectably labeled through the use of radioisotopes, affinity labels (such as biotin, avidin, etc.), enzymatic labels (such as horseradish peroxidase, alkaline phosphatase, etc.) fluorescence labels (such as FITC or rhodamine, etc.), paramagnetic atoms, gold beads, etc. Such labeling procedures are well-known in the art. The labeled antibodies of the present invention can be used for in vitro, in vivo, and in situ assays to identify a polypeptide and/or fragment of this invention in a sample.

In some embodiments, the present invention further provides the above-described antibodies immobilized on a solid support (e.g., beads, plates, slides or wells formed from materials such as latex or polystyrene). Examples of such solid supports include plastics such as polycarbonate, complex carbohydrates such as agarose and sepharose, acrylic resins and such as polyacrylamide and latex beads. Techniques for coupling antibodies to such solid supports are well known in the art (Weir et al., Handbook of Experimental Immunology 4th Ed., Blackwell Scientific Publications, Oxford, England, Chapter 10 (1986)). Antibodies can likewise be conjugated to detectable groups such as radiolabels (e.g., ³⁵S, ¹²⁵I, ¹³¹I), enzyme labels (e.g., horseradish peroxidase, alkaline phosphatase), and fluorescence labels (e.g., fluorescein) in accordance with known techniques. Determination of the formation of an antibody/antigen complex in the methods of this invention can be by detection of, for example, precipitation, agglutination, flocculation, radioactivity, color development or change, fluorescence, luminescence, etc., as is well known in the art.

In addition, techniques developed for the production of chimeric antibodies or humanized antibodies by splicing mouse antibody genes to human antibody genes to obtain a molecule with appropriate antigen specificity and biological activity can be used (Morrison et al. 1984. Proc. Natl. Acad. Sci. 81:6851-6855; Neuberger et al. 1984. Nature 312:604-608; Takeda et al. 1985. Nature 314:452-454). Alternatively, techniques described for the production of single chain antibodies can be adapted, using methods known in the art, to produce single chain antibodies specific for the polypeptides and fragments of this invention. Antibodies with related specificity, but of distinct idiotypic composition, can be generated by chain shuffling from random combinatorial immunoglobin libraries (Burton 1991. Proc. Natl. Acad. Sci. 88:11120-3).

Various immunoassays can be used for screening to identify antibodies having the desired specificity for the proteins and peptides of this invention. Numerous protocols for competitive binding or immunoradiometric assays using either polyclonal or monoclonal antibodies with established specificity are well known in the art. Such immunoassays typically involve the measurement of complex formation between an antigen and its specific antibody (e.g., antigen/antibody complex formation). For example, a two-site, monoclonal-based immunoassay utilizing monoclonal antibodies reactive to two non-interfering epitopes on the proteins or peptides of this invention can be used, as well as a competitive binding assay.

In certain embodiments, the fragments and/or polypeptides of this invention can be fused with a “carrier” protein or peptide to produce a fusion protein. Such fusion can be carried out, for example, by linking a nucleic acid of this invention in frame with a nucleic acid encoding a carrier protein or fragment thereof of this invention and expressing the linked nucleotide sequence to produce the fusion protein. For example, the carrier protein or peptide can be fused to a polypeptide and/or fragment of this invention to increase the stability thereof (e.g., decrease the turnover rate) in the cell and/or subject. Exemplary carrier proteins include, but are not limited to, glutathione-S-transferase or maltose-binding protein. The carrier protein or peptide can alternatively be a reporter protein. For example, the fusion protein can comprise a polypeptide and/or fragment of this invention and a reporter protein or peptide (e.g., green fluorescence protein (GFP), β-glucoronidase, β-galactosidase, luciferase, and the like) for easy detection of transformed cells and transgene expression. Any suitable carrier protein and/or nucleic acid encoding the carrier protein, as is well known in the art can be used to produce a fusion protein of this invention.

A variety of protocols for detecting the presence of and/or measuring the amount of polypeptides, fragments and/or peptides of this invention in a sample, using polyclonal and/or monoclonal antibodies specific for the polypeptide, fragment and/or peptide are known in the art. Examples of such protocols include, but are not limited to, enzyme immunoassays (EIA), agglutination assays, immunoblots (Western blot; dot/slot blot, etc.), radioimmunoassays (MA), immunodiffusion assays, chemiluminescence assays, antibody library screens, expression arrays, enzyme-linked immunosorbent assays (ELISA), radioimmunoassays (RIA), immunoprecipitation, Western blotting, competitive binding assays, immunofluorescence, immunohistochemical staining precipitation/flocculation assays and fluorescence-activated cell sorting (FACS). These and other assays are described, among other places, in Hampton et al. (Serological Methods, a Laboratory Manual, APS Press, St Paul, Minn. (1990)) and Maddox et al. (J. Exp. Med. 158:1211-1216 (1993)).

Furthermore, a number of assays for identification, detection and/or amplification of nucleic acid sequences are well known in the art. For example, various protocols can be employed in the methods of this invention to amplify nucleic acid. As used herein, the term “oligonucleotide-directed amplification procedure” refers to template-dependent processes that result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term “template dependent process” refers to nucleic acid synthesis of a RNA or a DNA molecule wherein the sequence of the newly synthesized strand of nucleic acid is dictated by the well-known rules of complementary base pairing. Typically, vector mediated methodologies involve the introduction of the nucleic acid fragment into a DNA or RNA vector, the clonal amplification of the vector, and the recovery of the amplified nucleic acid fragment. Examples of such methodologies are provided in U.S. Pat. No. 4,237,224 (incorporated herein by reference in its entirety). Nucleic acids, used as a template for amplification methods can be isolated from cells according to standard methodologies (Sambrook et al., 1989). The nucleic acid can be genomic DNA or fractionated or whole cell RNA. Where RNA is used, it may be desired to convert the RNA to a complementary DNA. In one embodiment, the RNA can be whole cell RNA and is used directly as the template for amplification.

Kits that include an antibody or immunologically functional fragment or immunogenic composition and/or other composition as described herein are also provided. Some kits include such an antibody, fragment, immunogen, vaccine and/or composition in a container (e.g., vial or ampule), and may also include instructions for use of the antibody or fragment, immunogenic composition and/or vaccine in the various methods disclosed above. The antibody, fragment, immunogenic fragment, vaccine or composition can be in various forms, including, for instance, as part of a solution or as a solid (e.g., lyophilized powder). The instructions may include a description of how to prepare (e.g., dissolve or resuspend) the antibody or fragment or immunogenic composition and/or vaccine in an appropriate fluid and/or how to administer the antibody or fragment or immunogenic composition and/or fragment for the treatment and/or prevention of the disorders and/or conditions described.

The kits may also include various other components, such as buffers, salts, complexing metal ions and other agents described above in the section on pharmaceutical compositions. These components may be included with the antibody or fragment or immunogenic composition and/or vaccine or may be in separate containers. The kits may also include other therapeutic agents for administration with the antibody or fragment or immunogenic composition and/or vaccine. Examples of such agents include, but are not limited to, agents to treat the disorders or conditions as described herein.

The present invention is more particularly described in the following examples, which are intended as illustrative only since numerous modifications and variations therein will be apparent to those skilled in the art.

### EXAMPLES

### Example 1. Dengue Virus Envelope Dimer Epitope (EDE) Monoclonal Antibodies Isolated from Dengue Patients are Protective Against Zika Virus

Here we report on the ability of human monoclonal antibodies and immune sera derived from dengue patients to neutralize contemporary epidemic ZIKV strains. We demonstrate that a class of human monoclonal antibodies isolated from DENV patients neutralizes ZIKV in cell culture and is protective in a lethal murine model. We also tested a large panel of convalescent immune sera from humans exposed to primary and repeat DENV infection. Although ZIKV is most closely related DENV compared to other human pathogenic flaviviruses, most DENV immune sera (73%) failed to neutralize ZIKV, while others had low (EC₅₀<100; 18%) or moderate to high levels (EC₅₀>100; 9%) of cross-neutralizing antibodies. Our results establish that ZIKV and DENV share epitopes that are targeted by neutralizing, protective human antibodies. The availability of potently neutralizing human monoclonal antibodies provides an immunotherapeutic approach to control life-threatening ZIKV infection and also points to the possibility of repurposing DENV vaccines to induce cross-protective immunity to ZIKV.

ZIKV is an emerging arbovirus that has been associated with severe neurological birth defects and fetal loss in pregnant women and Guillian Barré syndrome in adults. Currently, there is no vaccine or therapeutic for ZIKV. The identification of a class of antibodies (EDE1) that potently neutralizes ZIKV in addition to all four DENV serotypes points to a potential immunotherapeutic to combat ZIKV. This is especially salient given the precedence of antibody therapy to treat pregnant women infected with other viruses associated with microcephaly, such as cytomegalovirus and rubella virus. Furthermore, the identification of a functionally conserved epitope between ZIKV and DENV raises the possibility that a vaccine may be able to elicit neutralizing antibodies against both viruses.

ZIKV is an arbovirus in the Flaviviridae family, which includes important human pathogens such as Japanese encephalitis virus (JEV), West Nile virus (WNV), yellow fever virus (YFV), and dengue viruses 1-4 (DENV 1-4). Flaviviruses are traditionally classified as neurovirulent (WNV, JEV) or hemorrhagic (DENV, YFV). ZIKV infection has historically been characterized by self-limiting febrile illness including mild fever, rash, arthralgia, and conjunctivitis and was not considered to be a pathogen of major public health concern. However, ZIKV caused a large outbreak in Micronesia in 2007, and then throughout Polynesia and the Pacific Islands in 2013-14. In 2015, the first ZIKV outbreak in the Americas was reported in Brazil, where there was no previous evidence of circulation. Since then, 46 countries have reported novel outbreaks and ongoing transmission. Following the onset of the 2015 outbreak, several groups have identified an association between ZIKV infection and fetal malformations including spontaneous abortion, intrauterine growth restriction caused by placental insufficiency, blindness, and a causative link has been associated with microcephaly. The World Health Organization has also reported an increase in Guillian Barré syndrome and meningoencephalitis associated with ZIKV. The underlying molecular mechanisms driving these severe outcomes remain largely unknown.

The emergence of ZIKV overlaps geographically with DENV-endemic regions, and ZIKV shares approximately 60% sequence identity with DENV. Moreover, multiple dengue vaccine candidates are in phase II and III clinical trials, including a tetravalent vaccine that is currently approved for use in regions where ZIKV is emerging. Thus, a significant portion of the population in ZIKV outbreak areas has DENV reactive antibodies, which has complicated ZIKV diagnostics due to cross-reactivity. Given the extent to which DENV antibodies are present in the population, is it important to evaluate the possibility of cross-protective neutralizing epitopes that could protect against ZIKV infection. By screening a panel of monoclonal antibodies (mAbs), we found that a class of dengue serotype cross-neutralizing mAbs isolated from dengue patients, known as the envelope dimer epitope 1 (EDE1) mAbs, neutralize ZIKV in cell culture and protect from disease in a murine model. A few convalescent immune sera from dengue patients also cross-neutralized ZIKV, further demonstrating the presence conserved epitopes between ZIKV and DENV recognized by human neutralizing antibodies.

Neutralization of ZIKV by Human, Non-Human Primate, and Mouse mAbs.

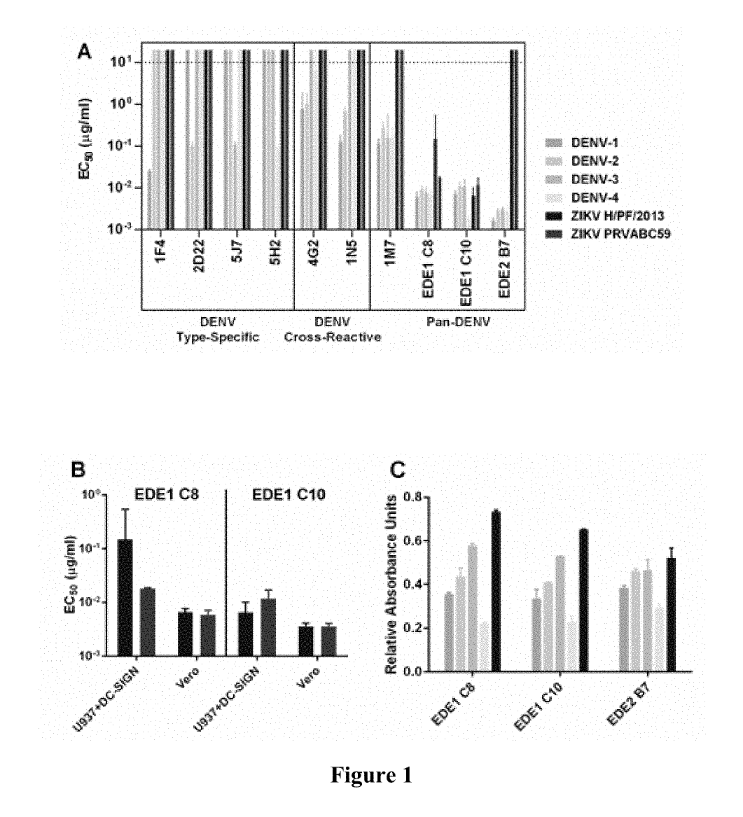

To better understand antibody cross-reactivity and functionality between DENV and ZIKV, we tested a large panel of well-characterized human and mouse mAbs for binding and neutralization of two strains of ZIKV; a French Polynesian 2013 strain representing the Asiastic lineage (H/PF/2013) and a strain circulating in the Americas in 2015 (PRVABC59). As expected, human and non-human primate type-specific mAbs that strongly neutralize DENV-1 (1F4), DENV-2 (2D22), DENV-3 (5J7), and DENV-4 (5H2) did not neutralize ZIKV (FIG. 1, Panel A). DENV cross-reactive mAbs that weakly or moderately neutralized two or more DENV serotypes (4G2, 1N5, and 1M7) also failed to neutralize ZIKV. In stark contrast, the potent DENV cross-neutralizing mAbs EDE1 C8 and EDE1 C10 strongly neutralized ZIKV infection of human monocytic cells expressing DC-SIGN. ZIKV neutralization by EDE1 C8 and ECE1 C10 was confirmed in Vero cells (FIG. 1, Panel B). EDE1 C8 and C10 neutralization of the high passage 1947 Ugandan isolate ZIKV MR766 was also confirmed in Vero cells, with EC₅₀ values of 8.9×10⁻⁴ and 3.4×10⁻⁴ μg/ml, respectively. Interestingly, EDE2 B7, which strongly neutralized all four DENV serotypes, bound but did not neutralize ZIKV (FIG. 1, Panel C). Of note, the contact residues of EDE2 B7 are all part of the EDE1 epitope except for residues 153-157, which were too disordered to resolve structurally when bound to DENV-2 (Table 1).

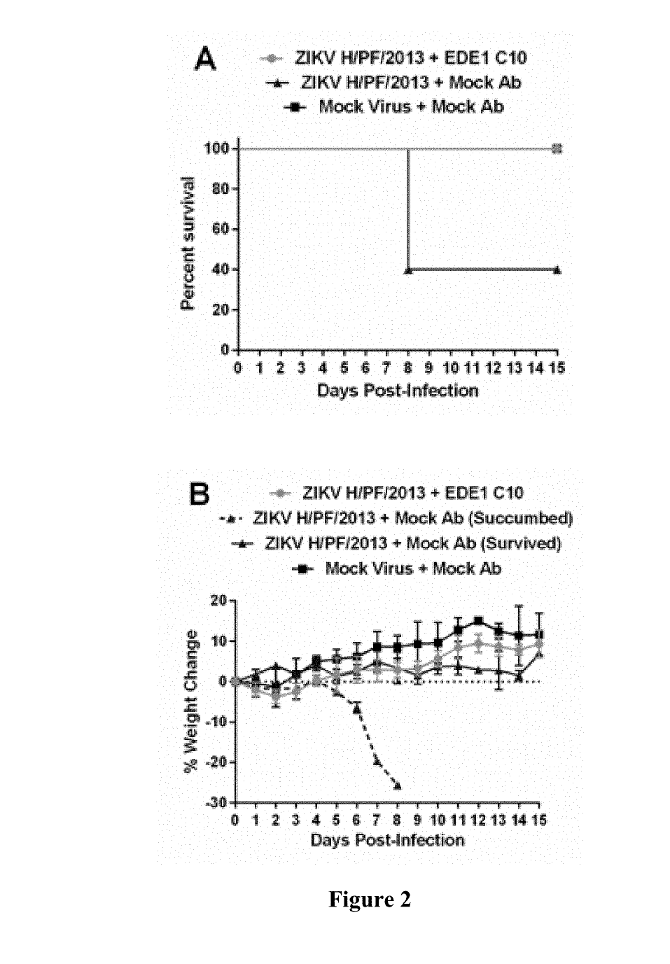

To determine if EDE mAbs protect against ZIKV in vivo, a study was performed in type I/II interferon receptor knockout mice, which develop ZIKV-induced morbidity and mortality. The mice were treated with either 10 μg EDE1 C10 or PBS at one day pre-infection and again at nine days post-infection, and challenged with 10² focus forming units of ZIKV H/PF/2013 or PBS in the footpad. The PBS-treated mice experienced 60% mortality following challenge (FIG. 2, Panel A), while the EDE1 C10-protected mice all survived (p<0.05). The EDE1 C10 treated, infected mice largely exhibited no signs of illness, their weight gain is more when compared to mice that were infected with ZIKV and mock antibody, but less than the mice that were not infected (FIG. 2, Panel B).

Neutralization of ZIKV by Convalescent Dengue Immune Sera.

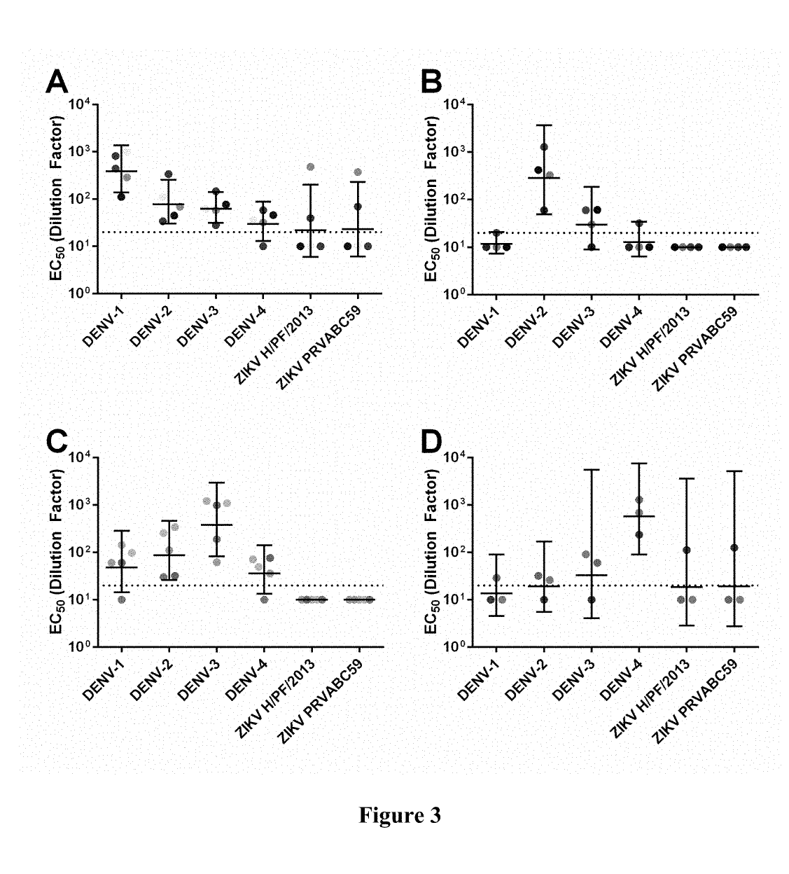

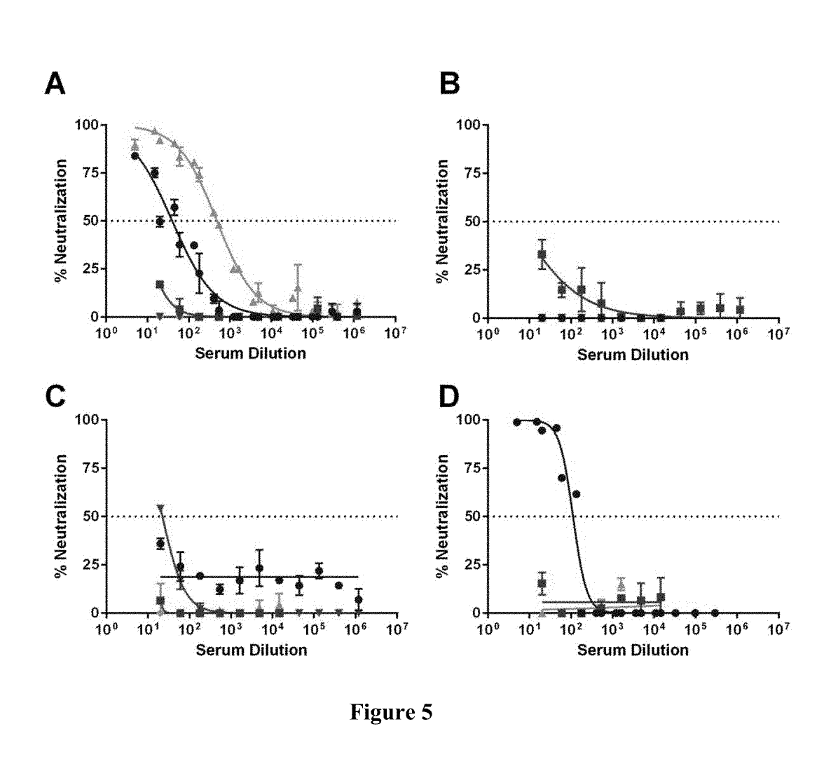

People exposed to dengue and other flavivirus infections develop antibodies that change in magnitude and quality over time. The ZIKV cross neutralizing and protective EDE1 C8 and C10 mAbs were derived from plasmablasts collected from individuals a few days after recovery from DENV infections. We tested whether convalescent immune sera collected from DENV patients several years after primary or secondary infection contained antibodies that cross-neutralized ZIKV. We tested a panel of 17 serum samples with neutralization profiles consistent with previous exposure to primary DENV-1 (n=5), DENV-2 (n=4), DENV-3 (n=5) and DENV-4 (n=3) infections for cross-neutralization of ZIKV. (Table 2) Most of the primary sera failed to cross-neutralize ZIKV. In fact, with primary DENV immune sera, we observed lower levels of ZIKV cross-neutralization compared to DENV cross neutralization (FIG. 3 and FIG. 5). Notable exceptions to this trend were two primary DENV-1 immune sera and one primary DENV-4 immune serum that contained moderate to high levels of ZIKV-neutralizing antibodies.

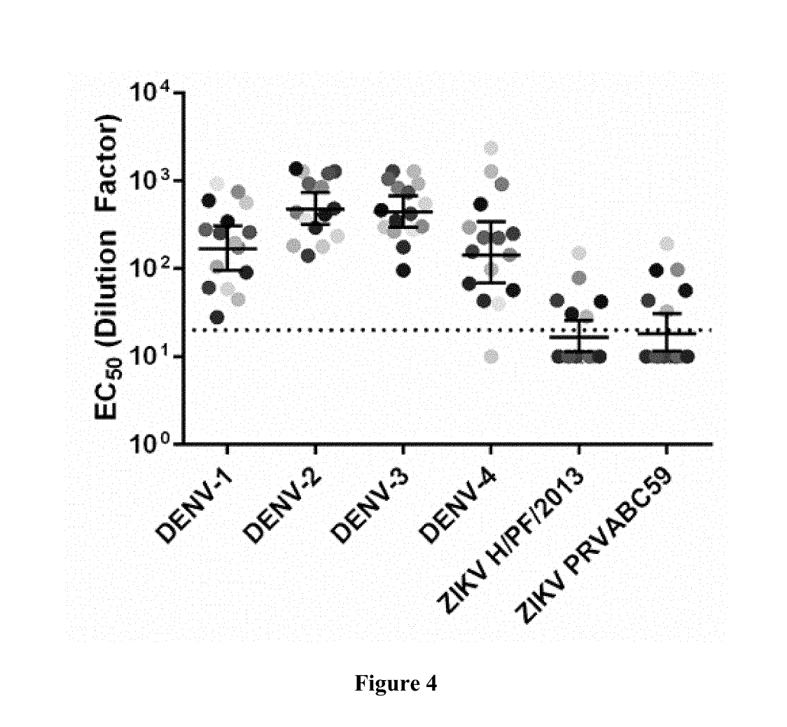

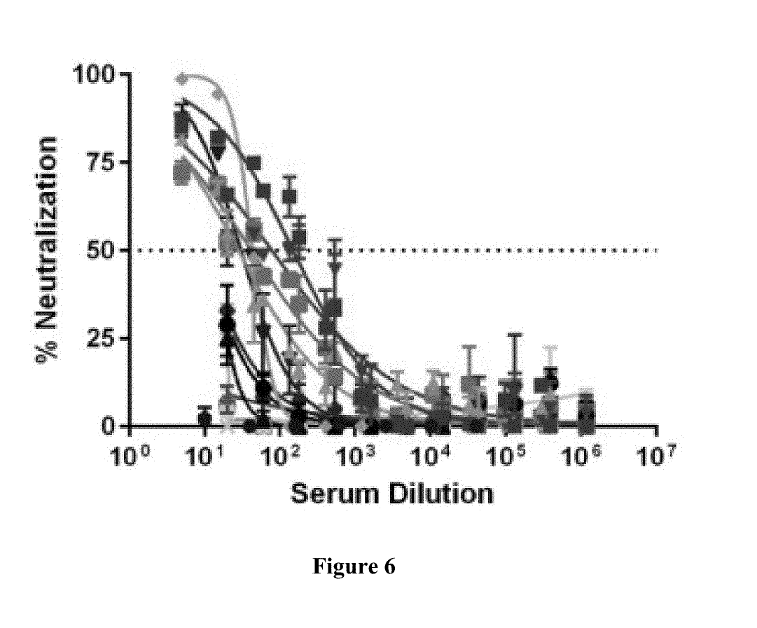

A hallmark of secondary DENV infections is the induction of dengue serotype cross-neutralizing antibodies which reduce risk of disease from subsequent DENV infections. We tested whether convalescent sera from people exposed to secondary DENV infections years previously also cross-neutralized ZIKV. All of the secondary sera samples tested neutralized DENV-1, DENV-2, and DENV-3, and 15 of 16 samples neutralized DENV-4 (FIG. 4 and FIG. 6). There were no statistically significant differences between the DENV serotypes. In contrast, secondary DENV immune sera usually had low or undetectable levels of ZIKV cross-neutralizing antibodies. Six of the 16 sera (38%) had measurable neutralization titers against ZIKV. Five of these six individuals had modest ZIKV neutralization (EC₅₀ between 1:20 and 1:100), and only one individual had strong ZIKV neutralization (EC₅₀>1:100). Overall, secondary DENV immune sera poorly neutralized ZIKC as compared to cross-neutralization phenotypes noted among other DENV serotypes, and only one individual (6%) strongly cross-neutralized ZIKV

Antigenic Cartography.

The fact that convalescent DENV immune human sera displayed low cross neutralization of ZIKV suggests that ZIKV is antigenically distantly related to DENV. To examine the antigenic relationships between ZIKV and DENV, we used antigenic cartography to calculate the Euclidean distances between sera and metric multidimensional scaling was used to render the data in three dimensions. Cartography supports the hypothesis that ZIKV is antigenically more distant from DENV-1-4 than each DENV serotype is to each other. Moreover, ZIKV-neutralizing sera did not have universally higher DENV-neutralizing titers than ZIKV non-neutralizing sera. Indeed, cartography suggests that neutralization titers of primary and secondary sera across all four DENV strains does not predict cross-neutralization outcomes with ZIKV, suggesting that these cross-neutralizing antibodies represent a rare subset of anti-DENV antibodies that develop in a subset of individuals within a population.

Because ZIKV co-circulates with other flaviviruses, especially the four DENV serotypes, an understanding of the antigenic relationships between ZIKV and other flaviviruses and how these interactions modulate ZIKV replication, disease and transmission is imperative. Among the pathogenic human flaviviruses, ZIKV is most closely related to DENV and the goal of this study was to identify any shared epitopes between DENV and ZIKV targeted by cross protective human antibodies. Primary DENV infections induce serotype specific neutralizing and protective antibody responses, whereas repeat DENV infections lead to the induction serotype cross-neutralizing and cross protective responses. We assessed the long-term immunological cross-reactivity of DENV sera with ZIKV using panels of mAbs and immune sera from people exposed to DENV. Neutralization assays with multiple type-specific and cross-reactive mAbs identified a single set of mAbs in our panel that could neutralize ZIKV and protect against lethal infection in vivo: EDE1 C8 and EDE1 C10. The EDE1 antibodies potently neutralized a French Polynesian 2013 strain representing the Asiastic lineage (H/PF/2013) and, importantly, a strain circulating in the Americas in 2015 (PRVABC59). Indeed, the dose of EDE1 C10 administered to protect in vivo (two doses of 10 μg) is far less than the 500 μg required for the fusion loop-targeting mouse mAb 2A10G6 (32). Thus, it seems likely that these EDE1 mAbs will prove efficacious against multiple ZIKV strains in vivo. Consonant with this hypothesis, an alignment of the EDE1 contact residues on DENV as previously identified by x-ray crystallography and ZIKV reveals considerable conservations among contact residues between all four DENV serotypes and ZIKV, readily explaining the cross-neutralization phenotypes noted in our studies.

The EDE2 B7 mAb did not neutralize ZIKV despite significant epitope overlap with the EDE1 antibodies. EDE2 B7 is reported to be sensitive to the glycan at position 153 of the DENV envelope protein; ZIKV has a glycan at position 154, but the amino acid insertion in the glycan loop may alter the presentation of the glycan. Moreover, EDE1 antibodies reach further into domains I and III, providing additional structural framework for robust binding that may not be as strongly impacted by the insertion in the glycan loop. These data suggest that the EDE1 epitope may be critical to eliciting antibodies that protect against both DENV and ZIKV, and that efforts to develop vaccines and therapeutics should emphasize this population of antibodies.

Some, but not all, DENV primary and secondary immune sera is capable of cross-neutralizing ZIKV. The limited cross-neutralization of ZIKV by DENV primary sera is likely attributed to the mostly type specific long-term response that follows a single DENV infection. After secondary infection, DENV-elicited antibody responses are thought to maintain the type-specific response while simultaneously generating more broadly neutralizing antibodies that typically protect from further DENV infection with any serotype (31). Surprisingly, we observed no cross neutralization of ZIKV in many individuals who had broadly cross-neutralizing antibodies to three or more DENV serotypes. We conclude that, despite the close phylogenetic relationship of DENV and ZIKV, durable long-lived antibody immune responses that broadly cross-neutralize DENV serotypes are usually not effective against ZIKV. What we did observe were clear cases of ZIKV cross-neutralization in a minority of subjects with DENV type-specific or cross-neutralizing antibody responses. The molecular basis of why some dengue-immune individuals cross-neutralize ZIKV is currently not known. Possible explanations for cross-neutralization include previous exposure to both DENV and ZIKV or the presence of EDE1 or related antibody classes in a subset of individuals. We propose that EDE1-like antibodies are, at least in part, responsible for cross neutralizing activity in immune sera.

Gamma globulin treatment of pregnant women infected with rubella virus is associated with a reduction in harmful outcomes in the fetus. Similar therapies have had mixed success in preventing cytomegalovirus-driven birth defects, and immunotherapeutic human monoclonal antibody clinical trials are still ongoing. Thus, it is reasonable to assume that human monoclonal antibody therapy may be a viable treatment option to protect the developing fetus in pregnant women infected with ZIKV. Although additional therapeutic studies are needed, the identification of an epitope that neutralizes ZIKV in vitro and in vivo represents a significant first step toward preventing ZIKV-driven fetal malformation and loss. Furthermore, the fact that the same antibodies targeting EDE1 are able to strongly neutralize both DENV and ZIKV is highly desirable, as diagnostic tests cannot always rapidly and reliably differentiate between the two infections. The strongly cross-neutralizing phenotypes of EDE1 C8 and EDE1 C10 should reduce the likelihood that a DENV patient who has been misdiagnosed with a ZIKV infection would experience disease enhancement after treatment with an EDE1 therapeutic antibody. In some individuals, the EDE1-like antibody may also be elicited by the existing tetravalent dengue vaccines already in late-stage clinical trials or available on the market.

Cells and Viruses.

All viruses were propagated in C6/36 Aedes albopictus cells as previously described. C6/36 cells were grown in minimal essential medium (Gibco, Grand Island, N.Y.) at 32° C. Vero-81 cells were grown in Dulbecco's modified Eagle's medium (Gibco, Grand Island, N.Y.) and U937+DC-SIGN cells were maintained in RPMI-1640 (Gibco, Grand Island, N.Y.) at 37° C. All media were supplemented with 10% (Vero-81) or 5% (C6/36 and U937+DC-SIGN) fetal bovine serum (HyClone, Logan, Utah), 0.1 mM nonessential amino acids (Gibco, Grand Island, N.Y.), 100 U/ml penicillin and 100 mg/ml streptomycin (Gibco, Grand Island, N.Y.). U937+DC-SIGN media was additionally supplemented with 2 mM GlutaMAX (Gibco, Grand Island, N.Y.), 10 mM HEPES (Cellgro, Manassas, Va.), and 2-mecaptoethanol (Sigma, St Louis, Mo.). All cells were incubated in the presence of 5% CO₂.

ZIKV H/PF/2013 viral stocks and ZIKV PRVABC59 viral stocks were obtained. DENV strains used in the polyclonal neutralization panel (DENV-1 WestPac74, DENV-2 S-16803, DENV-3 CH-53489, and DENV-4 TVP-376) were obtained from natural isolates maintained in the laboratory of Dr. Aravinda M. de Silva. DENV strains used in the monoclonal antibody panel (DENV-1 WestPac74, DENV-2 S-16803, DENV-3 UNC3001, and DENV-4 SriLanka 92 Å) were obtained from infectious clones in the laboratory of Dr. Ralph S. Baric.

Serum and Antibodies.

Deidentified human DENV immune sera and plasma were collected from individuals with naturally acquired DENV infections confirmed via serology. All donations were collected in compliance with the Institutional Review Board of the University of North Carolina at Chapel Hill (protocol 08-0895). Deidentified human immune sera previously collected from Pediatric Dengue Vaccine Initiative was also used. Monoclonal antibodies were purified from hybridomas (1M7 and 1N5) or synthetically generated by Lake Pharma (Belmont, Calif.) from published sequences (1F4, 2D22, 5J7, 5H2, 4G2, EDE1 C8, EDE1 C10, and EDE2 B7).

Monoclonal antibodies (mAb) C10, C8, and B7 were synthesized in transfected human HEK293 cells from cloned plasmids (Lake Pharma, Belmont Calif.). Briefly, heavy and light chain variable region sequences (Rouvinski et al. 2015) were cloned into plasmids containing the human IgG1 heavy and the human lambda 2 or kappa light chain constant regions where appropriate. HEK293T cells were transiently co-transfected with both the heavy chain and light chain plasmids, and soluble antibody was collected and protein A purified. The antibody was resuspended in a buffer containing 200 mM HEPES, 100 mM NaCl, 50 mM NaOAc, pH 7.0.

In Vitro Neutralization.

Human sera or monoclonal antibodies were serially diluted 3-fold and mixed with sufficient virus to cause 15% infection in U937+DC-SIGN cells. Dilution media contained reduced (2%) fetal bovine serum and was supplemented with 2 mM CaCl₂ and MgCl₂. The virus:antibody mixtures were incubated for 45 minutes in a 96-well plate at 37° C. Following this incubation, 5×10⁴ cells were added and the infection was allowed to proceed for 2 hours at 37° C. The volume of media in each well was increased to 200 μl, and the cells were returned to 37° C. for a total of 24 hours. After 24 hours, the cells were fixed in paraformaldehyde, permeablized, blocked with normal mouse serum in permeabilization buffer, and stained with AlexaFluor 488-conjugated (Molecular Probes, Eugene, Oreg.) 4G2 antibody. Unbound antibody was washed off, and cells were resuspended in Hank's Buffered Salt Solution (Gibco, Grand Island, N.Y.) supplemented with 2% fetal bovine serum. Assays were performed twice and in duplicate. Samples were read on a Guava easyCyte 5HT flow cytometer (Millipore) as previously described by our group (43).

Neutralization in Vero-81 cells was assessed by serially diluting the monoclonal antibodies 10-fold and mixing with approximately 150 focus forming units of virus. Dilution media contained reduced (2%) fetal bovine serum. The virus:antibody mixtures were incubated for 1 hour in a 96-well plate at 37° C., then transferred to a monolayer of Vero-81 cells in a 96-well plate. Following a further 1 hour incubation at 37° C., the monolayers were overlaid with OptiMEM (Gibco, Grand Island, N.Y.) containing 2% fetal bovine serum and 1% w/v carboxymethylcellulose (Sigma, St Louis, Mo.). Infected plates were incubated for two days at 37° C. with 5% CO₂, at which time they were fixed with paraformaldehyde, permeablized, blocked with normal goat serum (Sigma, St Louis, Mo.) in permeabilization buffer, and stained with 4G2 primary antibody followed by secondary HRP-conjugated anti-mouse IgG (KPL, Gaithersburg, Md.), washed again, and developed with TrueBlue peroxidase substrate (KPL, Gaithersburg, Md.).

Binding Assays.

High binding Microlon 600 96-well plates (VWR, Radnor, Pa.) were coated with 100 ng of 4G2 and 2H2 antibody in 0.1M carbonate buffer, pH 9.6, overnight at 4° C. Unbound antibody was rinsed with wash buffer (TBS+0.2% Tween), and wells were coated with blocking buffer (TBS+0.05% Tween) for 1 hour at 37° C. Virus was diluted in blocking buffer at a concentration sufficient to result in approximately equal reactivity with a human cross-reactive control serum and was added to the plate for one hour at 37° C. Unbound virus was rinsed in wash buffer and 1 μg of each primary antibody (or the control serum) diluted in blocking buffer was added to the plate for one hour at 37° C. Unbound primary antibody was rinsed in wash buffer and alkaline phosphatase-conjugated goat anti-human IgG antibody (Sigma, St Louis, Mo.) at 1:2,500 in blocking buffer was added to the plate for one hour at 37° C. Unbound secondary antibody was rinsed in wash buffer, and the plate was developed with SIGMAFAST p-nitrophenyl phosphate tablets (Sigma, St Louis, Mo.) and signal was read at 405 nm.

Animal Studies.

Cohorts of five (virus only and virus with antibody) or two (mock) five-week-old type I/II interferon receptor knockout mice (Ifnar^{-/-} and Ifngr^{-/-}) on a C57BL/6 backbone were used in a single experiment. On days −1 and 9 post-infection, mice received either PBS (mock and virus only) or 10 ug EDE1 C10 antibody (virus with antibody) in a 100 ul dose delivered intraperitoneally. On day 0, mice received either PBS (mock) or 100 FFU ZIKV H/PF/2013 (virus only and virus with antibody) in a 10 ul dose delivered subcutaneously in the hind left footpad. Mice were monitored daily for 14 days post-infection for weight loss and signs of illness. Mice were humanely euthanized if they became moribund and counted as deceased for that day. All work was performed in adherence to the NIH Guide for the Care and Use of Laboratory Animals.

Antigenic Cartography. Antigenic cartography was performed using the EC₅₀ values generated from the neutralization assays with DENV-1, 2, 3, and 4 in U937+DC-SIGN cells. The data was normalized. Euclidean distances between sera were calculated, and metric multidimensional scaling was used to render the data in three dimensions. All calculations and images were generated in R Studio version 0.99.467 (RStudio Inc, Boston, Mass.). Movie files were rendered using Adobe Photoshop software (Adobe, San Jose, Calif.).

Statistical Analysis.

When analyzing neutralization assays, antibody and serum concentrations were log₁₀ transformed. Next, the EC₅₀ values were calculated using the sigmoidal dose response (variable slope) equation of Prism 6 (GraphPad software, La Jolla, Calif.). Reported values were required to have: at least 5,000 recorded events per data point (in the case of the U937+DC-SIGN assay), a R squared value of greater than 0.75, a hill slope value of at least 0.7 for monoclonal antibodies and 0.5 for sera, and an EC₅₀ value within the range of the assay. Variation between groups was measured by one-way ANOVA with a Bonferroni post-hoc test. P-values of less than 0.05 were considered statistically significant. Absorbance signals for each virus group in the binding assay were multiplied or divided such that the signal for that virus against the common control serum was set to 1. Each assay was run singly with technical duplicates. Means and standard deviations were calculated in Prism 6 (GraphPad software, La Jolla, Calif.). Survival rates in the animal experiment were analyzed using the Log-rank (Mantel-Cox) test in Prism 6 for Windows (GraphPad software, La Jolla, Calif.). The virus with antibody cohort was compared to the virus only cohort.

### Example 2. Neutralization Mechanism of a Highly Potent Antibody Against Zika Virus

Zika virus (ZIKV) is a member of the flavivirus genus that includes dengue virus (DENV) and West Nile virus (WNV). ZIKV cryoEM structures show its surface proteins (envelope (E) and membrane (M) proteins) are organized similar to DENV except with a tighter packing, making the virus more thermally stable.

The virus surface consists of 180 copies of E protein arranged in icosahedral symmetry with 60 asymmetric units. In each asymmetric unit, there are three individual E proteins—molecules A, B and C. The E proteins exist as dimers; three dimers lie parallel to each other forming a raft containing two asymmetric units. There are in total 30 rafts arranged in a herringbone pattern on the virus surface.

An E protein contains three domains—DI, DII and DIII. It is known for other flaviviruses that DIII contains the receptor binding site and plays an important role in fusion of the virus with the endosomal membrane during cell entry. The tip of DII contains a fusion loop that interacts with the endosomal membrane. DI is the central domain linking DII and DIII together. The DI-DII hinge is highly flexible allowing DII to expose its fusion loop during the fusion event. The DI-DIII hinge was thought to be more rigid but it was observed to change in conformation in the post-fusion E protein trimeric structure. The fusion event is hypothesized to occur in this sequence: (1) virus E protein binds to cell receptors, (2) it is endocytosed, (3) the low pH environment of the endosome causes the E proteins to flip up exposing their fusion loops, allowing them to interact with the endosomal membrane, (4) the E proteins rearrange to trimeric structures, (5) the DIIIs of the E protein trimers change in conformation twisting the trimers leading to the fusion of viral membrane with the endosomal membrane, before the release of the viral genome into cell cytosol.

The recent explosion of the number of ZIKV cases, together with the association of ZIKV with the development of microcephaly in fetuses and Guillian-Barré syndrome in adults, ignite a pressing need for the development of therapeutics. Currently there are no published human monoclonal antibodies (HMAb) generated against ZIKV. To hasten the process of therapeutics development, DENV HMAbs were rescreened for those that cross-neutralize ZIKV. One group of antibodies has recently been shown to be highly neutralizing to ZIKV—the envelope dimer epitope binding antibodies. Of these HMAbs, C10 is one of the most potent plaque reduction neutralization test (PRNT₅₀=0.024 μgml⁻¹), as demonstrated recently in ZIKV infected cell culture and mouse model. In addition, it can prevent antibody dependent enhancement (ADE) of ZIKV infection in myeloid cells induced by dengue human sera. In this ADE model, the myeloid cells are mostly resistant to direct ZIKV infection, suggesting that its specific receptor is lacking. When sub-neutralizing concentrations of dengue human serum was added to ZIKV, cell infection was enhanced. This is because antibodies, which are attached to ZIKV, bind to the Fc receptor on myeloid cells thus bypassing the need for ZIKV to directly interact with its specific receptor. When HMAb C10 is added to this mixture, it neutralizes the ADE effect. Since HMAb C10 is also an antibody that would likely facilitate attachment to Fc receptor on myeloid cells, it likely neutralizes the virus at a post-attachment step of infection. We investigated the ability of Fab C10 to prevent virus surface protein rearrangement during fusion. We observed Fab C10 is able to lock the entire virus surface at pH6.5, and at pH5.0, the E protein raft thereby preventing structural rearrangement necessary for fusion.

Effect of Fab C10 on ZIKV Particles at Different pHs.

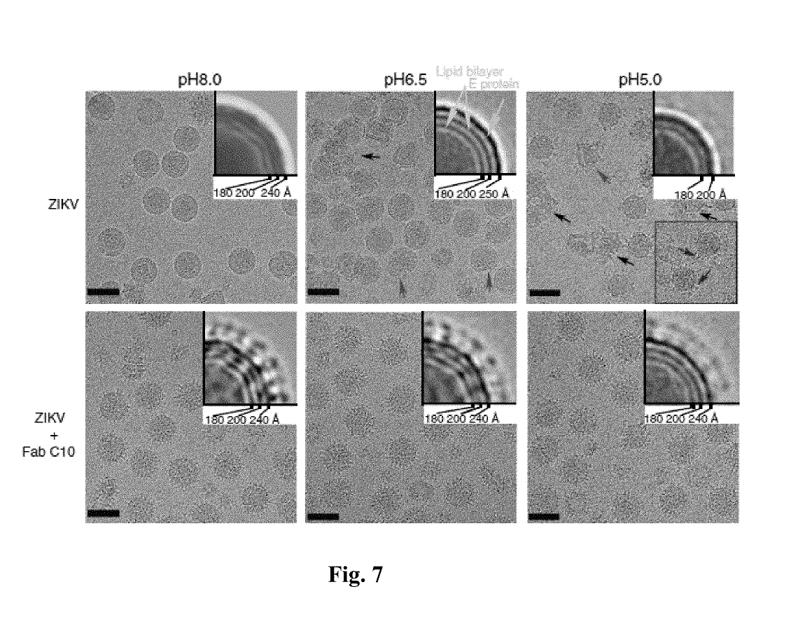

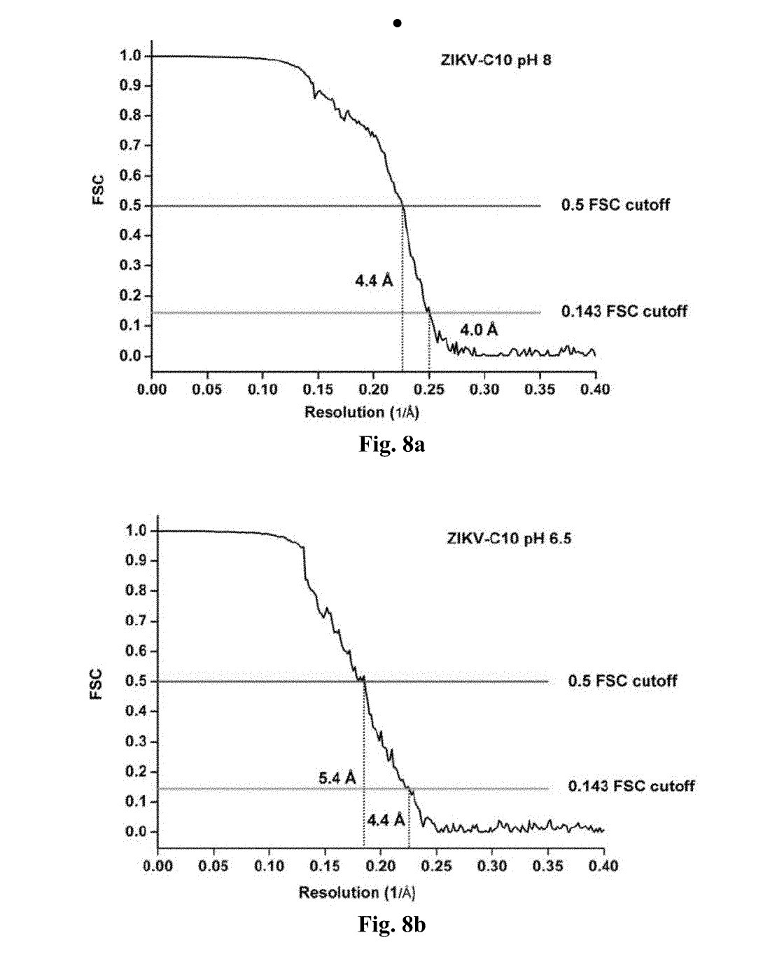

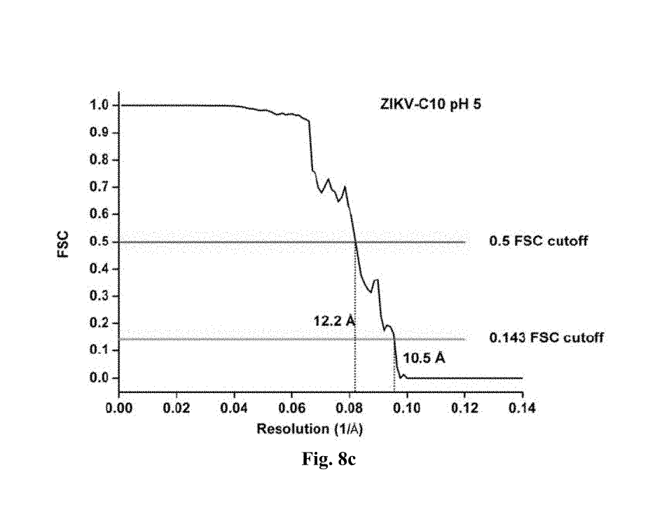

We solved the cryoEM structures of Fab C10 complexed with ZIKV at pH8.0, pH6.5 and pH5.0 mimicking the extracellular, early and late endosomal conditions, respectively, and compared them to the cryoEM maps of the uncomplexed ZIKV controls at pH8.0, pH6.5 (FIG. 8) and the two-dimensional (2D)-class average of pH5.0 particles (FIG. 7).

Micrographs of the uncomplexed ZIKV control at pH8.0 sample show mostly smooth surfaced spherical particles (FIG. 7). In the pH6.5 control sample, some virus particles aggregated, others become deformed, but there are also spherical particles present. 2D class average of the pH6.5 spherical particles (FIG. 7 inset), as well as its low resolution cryoEM map (FIGS. 8a and 8b), show the outer E protein layer has moved to a slightly larger radius compared to the pH8.0 control virus. This suggests that the E protein layer has loosened. Micrographs of the pH5.0 control sample (FIG. 7) show aggregation of some particles, while others appear to be smaller in diameter with hair-like densities protruding from the virus surface. The 2D class average of these small particles (FIG. 7 inset) showed the absence of the E protein compact layer, which was present in the pH8.0 and pH6.5 control samples. This suggests the E proteins are likely ‘flopping’ on the virus surface. The ZIKV controls demonstrate some of the structural transformation stages of the virus particles during fusion, from the compact structure at pH8.0 to a slightly expanded structure at pH6.5 and finally to the E proteins loosening and extending out from the virus lipid membrane at pH5.0.

Micrographs of the ZIKV-C10 complexes at all pH conditions show spiky looking particles, due to the Fab molecules bound to virus surface (FIG. 7). The 2D class average of the pH6.5 complex particles shows the E protein layer to remain at the same radius as the pH8.0 control (FIG. 7 inset), unlike its pH6.5 ZIKV control. The 2D class average of the pH5.0 complex particles in contrast to its pH5.0 control shows the E protein layer is still present (FIG. 7 inset).

CryoEM Structures of ZIKV-C10 at Different pHs.

The cryoEM structures of ZIKV-C10 complex at pH8.0, pH6.5 and pH5.0 are determined to 4.0, 4.4 and 12 Å resolution, respectively. In each of these structures, there are 180 copies of Fab C10 bound to the virus surface. The pH8.0 and pH6.5 complex structures are very similar to each other and their cryoEM maps correlate to 4.5 Å resolution at FSC 0.143 cutoff. Therefore, only the higher resolution pH8.0 complex structure will be described. A comparison of the E proteins of the pH8.0 complex structure with that of the previously solved uncomplexed ZIKV shows that they are largely the same. Only molecule A of the E protein in the asymmetric unit on the pH8.0 complex structure shows clear densities for the ‘150 glycan loop’. The glycan loop changed in conformation when compared to the uncomplexed virus, likely due to its interaction with the Fab molecule (Table 37). This glycan loop on ZIKV is five residues longer than in DENV. The previously solved crystal structure of DENV-C10 did not show densities corresponding to the glycan loop; therefore, it is not known if this region interacts directly with the Fab. However, mutational studies indicate that the residue 153 glycosylation site on DENV is not important for HMAb C10 binding.

In the 4.0 Å resolution pH8.0 complex cryoEM map, the likely interacting residues that form the epitope were identified, by using a cutoff of 5 Å distance (hydrogen bonds/electrostatic interaction: 4 Å and hydrophobic interactions: 5 Å) between side chains of the Fab and E proteins (Table 37). We also presented the epitope identified with a cutoff of 8 Å distance between the Ca chains of the Fab and E proteins. Each end of the E protein dimer has a Fab molecule attached. The Fabs bind across the E proteins at the intra-dimer interface. The Fab bound near the five-fold vertex end of the A-C′ dimer also likely interacts with residues from the adjacent E protein at the inter-raft interface, whereas the Fab molecule at the other end is also involved in inter-dimer interactions within the raft. The epitopes recognized by the Fab molecules that bind to B-B′ dimer also span across the inter-dimer E protein interfaces. The ability of Fab C10 to bind E proteins at the intra-dimer interface together with the virus quaternary structure-dependent sites—the inter-dimer and inter-raft interfaces, suggests that the entire E protein layer is locked. This is consistent with the cryoEM structure and the 2D class average (FIG. 7 inset) of the ZIKV-C10 complex at pH6.5 showing the E protein layer remains at a similar radius as the uncomplexed ZIKV 018.0 control, unlike its pH6.5 control.

The ZIKV-C10 complex intra-dimer epitope is located on the DII (at and around the fusion loop) on one E protein and on DIII and DI of the other E protein in the dimer. A plot of electrostatic charges of interacting residues on the E protein intra-dimer epitope and the Fab paratope showed complementary charges. A comparison with the previously published crystal structure of DENV recombinant E protein dimer complexed with Fab C10 shows their epitopes largely overlap. The conserved residues between ZIKV and DENV C10 intra-dimer epitope mainly cluster on DII near the fusion loop. Although a comparison of the C10 intra-dimer epitope to the crystal structures of other EDE antibodies, Fab C8 and A11 complexed with ZIKV recombinant E protein shows overlapping epitopes, the C10 intra-dimer epitope spans a wider area covering larger parts of DI and DIII. Furthermore, our cryoEM structure also shows the interactions of Fab C10 with other virus quaternary structure-dependent epitope at the inter-dimer or inter-raft interfaces which are not observed in the crystal structures. These interactions result in the E proteins on virus surface being locked together and could be critical for its neutralization mechanism.

A comparison of the cryoEM maps of pH5.0 complex to the pH8.0 complex shows that the E protein layer has moved to a larger radius, whereas the radii of the lipid bilayer membranes are similar. The pH5.0 complex cryoEM map was interpreted by fitting separately the Fab:A-C′ dimer and Fab:B-B′ dimer structures from the pH8.0 complex structure into their respective densities. Comparison of the E proteins in an asymmetric unit of the pH5.0 and pH8.0 complex by superimposing their molecules A shows a slight shift (3.5 Å) between the B′ molecule with respect to the A-C′ dimer. This motion has only slightly changed the distance of the interacting residues on the Fab with that on the E proteins at the inter-dimer interface (Table 38) suggesting the Fab retains its binding capability across this interface at pH5.0 and thus the E proteins within the raft are locked by Fab C10.

Comparing the pH8.0 and the pH5.0 complex structures shows that the maximum radial movement of the E protein outwards is at one end of the A-C′ dimer near the fivefold vertex (˜15 Å). This suggests the membrane associated stem regions of the E protein need not be fully extended (up to ˜65 Å in length) for this movement. In sharp contrast, a previous study describing a very low resolution cryoEM map of a DIII-binding Fab E16:WNV complex at pH6.0 showed the E protein layer moved radially outwards by ˜60 Å, even though the E protein density was not interpretable. Our pH5.0 complex structure here shows a smaller radial expansion of the E protein layer and therefore may be an even earlier event of fusion process involving the dissociation of the E protein layer from the lipid membrane. Another low resolution cryoEM structure of antibody E104 complexed with DENV was shown to inhibit another stage of the fusion process, possibly the ‘open trimeric E protein conformation’. This is likely a step prior to the formation of the closed trimeric E protein structure.

Although the E protein raft structure stays mostly intact in the ZIKV-C10 pH5.0 complex structure, the inter-raft interactions are disrupted, even though the Fab C10 in the pH8.0 complex structure forms inter-raft interactions at two sites. A calculation of the E protein electrostatic charges at pH8.0, 6.5 and 5.0 at the intra-dimer, inter-dimer and inter-raft interfaces shows the residues becoming increasingly positively charged with decreasing pH. This suggests that at pH5.0, the E proteins may have the tendencies to repel each other, consistent with the 2D class average of the uncomplexed ZIKV particles at pH5.0, showing that the compact E protein layer is disrupted. This raises a question of why the Fab inter-raft interaction in the pH5.0 complex structure is disrupted, whereas the inter-dimer interaction remains intact. We speculate that the two Fab molecules at the inter-raft interface identified in the pH8.0 complex structure may not form strong enough contacts to resist the large surface area of electrostatic repelling force at this interface. On the other hand, at the inter-dimer interface, the two Fabs in this region could still hold the dimers together, as the surface area of repelling force is much smaller.

All antibodies can cause ADE at some concentrations. HMAb C10, similar to other potent antibodies, causes ADE at a much narrower range of concentrations compared to the other weakly neutralizing antibodies. To increase the safety of HMAb C10 as a therapeutic antibody, its ability to cause ADE could be eliminated, by mutating its Fc region (LALA mutants) abolishing its interaction with the myeloid cells Fc receptors. Flavivirus such as DENV has been shown to be able to use different receptors to gain entry into different cell types. ZIKV may also behave the same, as it has been shown to bind to DC-SIGN and also TAM receptors. Since different parts of the E proteins interact with different receptors, it is unlikely that any single type of antibody could inhibit virus attachment to all cell types. In addition, ZIKV may also enter by ADE caused by pre-existing DENV antibodies in individuals, thus completely bypassing the need for virus to attach to a specific receptor for infection. However, regardless of how the virus gets into the cell, fusion is a vital step for productive infection. This emphasizes the potential of HMAb C10 as a therapeutic agent, since it can prevent structural rearrangements necessary for virus-endosomal membrane fusion.

Neutralization Test of HMAb C10 to Zika Virus.

The neutralization activity of the HMAb C10 on Zika virus strain H/PF/2013 was determined by PRNT. Two-fold serially diluted HMAb C10 starting at 0.5 μgml⁻¹ were incubated with equal volumes of virus at 37° C. for 0.5 h. One hundred microliters of each mixtures were then layered on BHK-21 cells in a 24-well plate and incubated at 37° C. for 1.5 h. The infected cells were washed with phosphate-buffered saline, overlaid with carboxyl-methyl cellulose and incubated at 37° C. for 5 days. Cells were fixed and stained, and the plaques were counted. Percentage neutralization was determined from the comparison of the number of plaques in specific antibody dilutions to the control (without antibody). PRNT₅₀ is the concentration of the antibody that causes 50% reduction in plaque numbers.

Virus Sample Preparation.

Aedes albopictus C6/36 cells (ATCC) were grown in RPMI 1640 media supplemented with 2% fetal bovine serum at 29° C. At about 80% confluency, the cells were inoculated with ZIKV strain H/PF/2013 at a multiplicity of infection of 0.5 and incubated at 29° C. for 4 days. The virus-containing media was clarified by centrifugation at 12,000 g for 1 h. Virus was precipitated overnight from the supernatant using 8% (w/v) polyethylene glycol 8000 in NTE buffer (10 mM Tris-HCl pH8.0, 120 mM NaCl and 1 mM EDTA) and the suspension was centrifuged at 14,334 g for 1 h. The resulting pellet containing the virus was resuspended in NTE buffer and then purified through a 24% (w/v) sucrose cushion followed by a linear 10-30% w/v potassium tartrate gradient. The virus band, visualized by its light scattering ability, was extracted, buffer exchanged into NTE buffer and concentrated using a concentrator with 100-kDa molecular weight cut-off filter. All steps of the purification procedure were done at 4° C. The concentrations and purity of the E protein were estimated with Coomassie blue-stained SDS-PAGE using different known concentrations of bovine serum albumin solution as standards.

Monoclonal Antibody C10 Production.

Monoclonal antibody (mAb) C10 was synthesized in transfected human cells from cloned plasmids (Lake Pharma, Belmont, Calif., USA). Briefly, previously published heavy and light chain variable region sequences were cloned into plasmids containing the human IgG1 heavy and the human lambda 2 light chain constant regions. HEK293T cells (ATCC) were transiently co-transfected with both the heavy chain and light chain plasmids, and soluble antibody was collected and protein A purified. The antibody was resuspended in a buffer containing 200 mM HEPES, 100 mM NaCl, 50 mM NaOAc, pH7.0.

Preparation of the C10 Fab Fragments.

The Fab regions of C10 IgG were produced by papain digestion. Briefly, the whole IgG (8 mg ml⁻¹) was incubated overnight with immobilized papain (Thermo Scientific) at 37° C. After digestion, the Fab fragment was purified with anion exchange chromatography (resource Q, GE Healthcare) and gel filtration (Superdex 200 increase 10/300 GL, GE Healthcare) on an AKTA purifier system.

CryoEM Sample Preparation.

The Fab C10 was mixed with ZIKV at a molar ratio of 1.5 Fab to every E protein. The mixture was incubated for 30 min at 37° C. followed by ˜1 h on ice, and then applied to a cryoEM grid (pre-cooled to 4° C.) for 10 s prior to adjusting the pH. The final pH of the virus was reached by addition of a volume ratio of 1.5 μl of 50 mM MES buffer at respective pH (pH5.0 or pH6.5) to every 1 μl of the virus-Fab mixture. The pH-adjusted samples were left on the grid for another 15 s. The grid was then blotted with filter paper and flash frozen in liquid ethane by using the Vitrobot Mark IV plunger (FEI, the Netherlands). The corresponding controls (ZIKV without Fab) for each pH were prepared similarly.

Cryoelectron Microscopy and Image Processing.

The images of the frozen ZIKV complexes were taken with the FEI Titan Krios electron microscope, equipped with 300 kV field emission gun, at nominal magnification of 47,000 for pH5.0 Fab C10 ZIKV complex, and 59,000 for pH6.5 and pH8.0 complex samples. A 4096×4096 FEI Falcon II direct electron detector was used to record the images.

Leginon was used to carry out the data collection. Images for pH8.0 and pH6.5 complexes were collected in movie mode, with total exposure of 1.6 s and total dose 38 e⁻ Å⁻² for pH8.0, total exposure of 1.05 s and total dose of 43 e⁻ Å⁻² for pH6.5 complex. The pH5.0 complex was collected at single image mode, with the dose of 20 e⁻ Å⁻². The frames from each ‘movie’ were aligned using MotionCorr to produce full dose images used for particle selection and orientation search, and images from the first several frames amounting to the dose of about 18 e⁻ Å⁻² to use in 3D reconstruction. The images were taken at underfocus in 0.5˜2.5 mm range. The astigmatic defocus parameters were estimated with Gctf and accounted for in orientation search and 3D reconstruction procedures in MPSA and Relion. In total, 3,257, 2,540 and 2,865 micrographs were collected for Zika-C10 complex at pH8.0, pH6.5 and pH5.0, respectively. The virus-Fab particles were picked with automatic selection tool Gautomatch, run through 2D classification in Relion to produce 2D class averages, broken and classes containing nonviral particles and broken particles were removed, 49,100, 45,867 and 23,810 individual particles in the Fab complex samples which were incubated at pH8.0, pH6.5 and pH5.0, respectively, were selected for further processing. The 3D reconstruction of the pH8.0 and pH6.5 complex structure was done with MPSA, whereas Relion was used for the pH5.0 complex structure. Uncomplexed ZIKV (EMDB ID EMD-8139) was used as the starting model. The gold standard protocol for structure refinement was used for all complexes. The 3D reconstruction procedure produced the complex structures with resolutions of 4.0, 4.4 and 12 Å, for pH8.0, pH6.5 and pH5.0, respectively—using the Fourier shell correlation cutoff of 0.143 for the pH8.0 and pH6.5 cryoEM maps and 0.5 for the pH5.0 cryoEM map.

Protein Structure Building.

The pH8.0 and pH6.5 ZIKV-C10 structures were interpreted by fitting in the uncomplexed ZIKV (PDB ID 5IZ7) and Fab C10 (PDB ID 4UT9) first as rigid bodies in Chimera and then finer adjustment were made by using the program Coot. The 12 Å resolution cryoEM map of the ZIKVC10 complex at pH5.0 was interpreted by first fitting in the entire E-protein raft complex with Fab molecules at pH8 structure by using the ‘fit-in-map’ function in Chimera. The individual Fabs complexed with A-C′ and also Fab complexed with B-B′ dimers within a raft were kept as separate rigid bodies groups and their fit into the density were independently optimized by using the ‘fit-in-map’ function in Chimera.

Electrostatic Potential Calculations.

Electrostatic potentials of protein surfaces were calculated using Adaptive Poisson-Boltzmann Solver (APBS) and PDB2PQR31 packages. The structures of uncomplexed ZIKV (PDB ID 5IZ7) and the ZIKV complexed with Fab C10 were processed with the PDB2PQR web server (nbcr-222.ucsd.edu/pdb2pqr_2.0.0/) to prepare the PDB files for APBS. A PARSE force field was applied and PROPKA (v3.0) was used to assign pKa values. APBS was then used to calculate the electrostatic properties of the protein surface.

Data Availability.

The cryoEM maps and the atomic models of the ZIKV-C10 complex at pH8.0, 6.5 and 5.0 have been deposited in the Electron Microscopy Data Bank (EMD) and the Protein Data Bank (PDB) under the accession codes EMD-9575, EMD-9573, EMD-9574 and 5H37, 5H30, 5H32, respectively. The data that support the findings of this study are available from the corresponding authors on request.

### Example 3. Summary of EDE Null Mutants

EDE antibodies were first described as a population of antibodies found in some dengue virus (DENV)-infected individuals, which were highly cross-reactive against all four DENV serotypes. Two overlapping by slightly different populations were defined as EDE1 and EDE2.

EDE1 antibodies, but not necessarily EDE2 antibodies, are able to neutralize Zika virus (ZIKV) and protect against lethal ZIXV challenge in a murine model.

Rationale for the construction and necessity of EDE Null viruses: Because EDE antibodies are cross-reactive, they are difficult to track. Our group has had success with transplanting epitopes between flaviviruses. These “gain” viruses are being used to track the prevalence of type-specific quaternary epitopes and to develop improved vaccines. Tamana bat virus (TABV) is a no-known-vector flavivirus (it does not replication in invertebrate cells) that is genetically and antigenically distinct from flaviviruses of public health interest (such as DENV and ZIKV).

By transplanting the TABV version of the EDE epitope into an otherwise EDE-reactive backbone, we can track the loss of antibody binding and neutralization in an EDE-specific manner. The ‘Core and EDE1-specific’ and ‘Core’ constructs can be used to track the overall EDE response. This information can be combined with the ‘EDE1-Specific’ construct to differentiate an EDE1-like response from an EDE2-like response. Structural information regarding the amino acid substitutions that were introduced to produce these constructs is provided in Tables 39-44. Nucleotide sequences of the EDE null mutants of this invention are provided as SEQ ID NOS:90-95 in the Sequence Listing.

The foregoing is illustrative of the present invention, and is not to be construed as limiting thereof. The invention is defined by the following claims, with equivalents of the claims to be included therein.

All publications, patent applications, patents, accession numbers and other references cited herein are incorporated by reference in their entireties for the teachings relevant to the sentence and/or paragraph in which the reference is presented.

SEQUENCES

AMINO ACID SEQUENCE OF DENV1 (GENBANK ACCESSION NO. U88535.1) (SEQ ID
NO: 1)
MNNQRKKTGRPSFNMLKRARNRVSTVSQLAKRFSKGLLSGQGPM
KLVMAFIAFLRFLAIPPTAGILARWGSFKKNGAIKVLRGFKKEISNMLNIMNRRKRSV
TMLLMLLPTALAFHLTTRGGEPHMIVSKQERGKSLLFKTSAGVNMCTLIAMDLGELCE
DTMTYKCPRITETEPDDVDCWCNATETWVTYGTCSQTGEHRRDKRSVALAPHVGLGLE
TRTETWMSSEGAWKQIQKVETWALRHPGFTVIALFLAHAIGTSITQKGIIFILLMLVT
PSMAMRCVGIGNRDFVEGLSGATWVDVVLEHGSCVTTMAKDKPTLDIELLKTEVTNPA
VLRKLCIEAKISNTTTDSRCPTQGEATLVEEQDTNFVCRRTFVDRGWGNGCGLFGKGS
LITCAKFKCVTKLEGKIVQYENLKYSVIVTVHTGDQHQVGNETTEHGTTATITPQAPT
SEIQLTDYGALTLDCSPRTGLDFNEMVLLTMEKKSWLVHKQWFLDLPLPWTSGASTSQ
ETWNRQDLLVTFKTAHAKKQEVVVLGSQEGAMHTALTGATEIQTSGTTTIFAGHLKCR
LKMDKLTLKGMSYVMCTGSFKLEKEVAETQHGTVLVQVKYEGTDAPCKIPFSSQDEKG
VTQNGRLITANPIVTDKEKPVNIEAEPPFGESYIVVGAGEKALKLSWFKKGSSIGKMF
EATARGARRMAILGDTAWDFGSIGGVFTSVGKLIHQIFGTAYGVLFSGVSWTMKIGIG
ILLTWLGLNSRSTSLSMTCIAVGMVTLYLGVMVQADSGCVINWKGRELKCGSGIFVTN
EVHTWTEQYKFQADSPKRLSAAIGKAWEEGVCGIRSATRLENIMWKQISNELNHILLE
NDMKFTVVVGDVSGILAQGKKMIRPQPMEHKYSWKSWGKAKIIGADVQNTTFIIDGPN
TPECPDNQRAWNIWEVEDYGFGIFTTNIWLKLRDSYTQVCDHRLMSAAIKDSKAVHAD
MGYWIESEKNETWKLARASFIEVKTCIWPKSHTLWSNGVLESEMIIPKIYGGPISQHN
YRPGYFTQTAGPWHLGKLELDFDLCEGTTVVVDEHCGNRGPSLRTTTVTGKTIHEWCC
RSCTLPPLRFKGEDGCWYGMEIRPVKEKEENLVKSMVSAGSGEVDSFSLGLLCISIMI
EEVMRSRWSRKMLMTGTLAVFLLLTMGQLTWNDLIRLCIMVGANASDKMGMGTTYLAL
MATFRMRPMFAVGLLFRRLTSREVLLLTVGLSLVASVELPNSLEELGDGLAMGIMMLK
LLTDFQSHQLWATLLSLTFVKTTFSLHYAWKTMAMILSIVSLFPLCLSTTSQKTTWLP
VLLGSLGCKPLTMFLITENKIWGRKSWPLNEGIMAVGIVSILLSSLLKNDVPLAGPLI
AGGMLIACYVISGSSADLSLEKAAEVSWEEEAEHSGASHNILVEVQDDGTMKIKDEER
DDTLTILLKATLLAISGVYPMSIPATLFVWYFWQKKKQRSGVLWDTPSPPEVERAVLD
DGIYRILQRGLLGRSQVGVGVFQEGVFHTMWHVTRGAVLMYQGKRLEPSWASVKKDLI
SYGGGWRFQGSWNAGEEVQVIAVEPGKNPKNVQTAPGTFKTPEGEVGAIALDFKPGTS
GSPIVNREGKIVGLYGNGVVTTSGTYVSAIAQAKASQEGPLPEIEDEVFRKRNLTIMD
LHPGSGKTRRYLPAIVREAIRRNVRTLVLAPTRVVASEMAEALKGMPIRYQTTAVKSE
HTGKEIVDLMCHATFTMRLLSPVRVPNYNMIIMDEAHFTDPASIAARGYISTRVGMGE
AAAIFMTATPPGSVEAFPQSNAVIQDEERDIPERSWNSGYDWITDFPGKTVWFVPSIK
SGNDIANCLRKNGKRVVQLSRKTFDTEYQKTKNNDWDYVVTTDISEMGANFRADRVID
PRRCLKPVILKDGPERVILAGPMPVTVASAAQRRGRIGRNQNKEGDQYIYMGQPLNND
EDHAHWTEAKMLLDNINTPEGIIPALFEPEREKSAAIDGEYRLRGEARKTFVELMRRG
DLPVWLSYKVASEGFQYSDRRWCFDGERNNQVLEENMDVEIWTKEGERKKLRPRWLDA
RTYSDPLALREFKEFAAGRRSVSGDLILEIGKLPQHLTQRAQNALDNLVMLHNSEQGG
KAYRHAMEELPDTIETLMLLALIAVLTGGVTLFFLSGRGLGKTSIGLLCVIASSALLW
MASVEPHWIAASIILEFFLMVLLIPEPDRQRTPQDNQLAYVVIGLLFMILTAAANEMG
LLETTKKDLGIGHAAAENHHHAAMLDVDLHPASAWTLYAVATTIITPMMRHTIENTTA
NISLTAIANQAAILMGLDKGWPISKMDIGVPLLALGCYSQVNPLTLTAAVFMLVAHYA
IIGPGLQAKATREAQKRTAAGIMKNPTVDGIVAIDLDPVVYDAKFEKQLGQIMLLILC
TSQILLMRTTWALCESITLATGPLTTLWEGSPGKFWNTTIAVSMANIFRGSYLAGAGL
AFSLMKSLGGGRRGTGAQGETLGEKWKRQLNQLSKSEFNTYKRSGIIEVDRSEAKEGL
KRGEPTKHAVSRGTAKLRWFVERNLVKPEGKVIDLGCGRGGWSYYCAGLKKVTEVKGY
TKGGPGHEEPIPMATYGWNLVKLYSGKDVFFTPPEKCDTLLCDIGESSPNPTIEEGRT
LRVLKMVEPWLRGNQFCIKILNPYMPSVVETLEQMQRKHGGMLVRNPLSRNSTHEMYW
VSCGTGNIVSAVNMTSRMLLNRFTMAHRKPTYERDVDLGAGTRHVAVEPEVANLDIIG
QRIENIKNGHKSTWHYDEDNPYKTWAYHGSYEVKPSGSASSMVNGVVRLLTKPWDVIP
MVTQIAMTDTTPFGQQRVFKEKVDTRTPKAKRGTAQIMEVTARWLWGFLSRNKKPRIC
TREEFTRKVRSNAAIGAVFVDENQWNSAKEAVEDERFWDLVHRERELHKQGKCATCVY
NMMGKREKKLGEFGKAKGSRAIWYMWLGARFLEFEALGFMNEDHWFSRENSLSGVEGE
GLHKLGYILRDISKIPGGNMYADDTAGWDTRITEDDLQNEAKITDIMEPEHALLATSI
FKLTYQNKVVRVQRPAKNGTVMDVISRRDQRGSGQVGTYGLNTFTNMEAQLIRQMESE
GIFSPSELETPNLAERVLDWLKKHGTERLKRMAISGDDCVVKPIDDRFATALTALNDM
GKVRKDIPQWEPSKGWNDWQQVPFCSHHFHQLIMKDGREIVVPCRNQDELVGRARVSQ
GAGWSLRETACLGKSYAQMWQLMYFHRRDLRLAANAICSAVPVDWVPTSRTTWSIHAH
HQWMTTEDMLSVWNRVWIEENPWMEDKTHVSSWEDVPYLGKREDRWCGSLIGLTARAT
WATNIQVAINQVRRLIGNENYLDFMTSMKRFKNESDPEGALW
AMINO ACID SEQUENCE OF DENV2 GENBANK ACCESSION NO. GU289914.1 (SEQ ID NO: 2)
MNNQRKKARNTPFNMLKRERNRVSTVQQLTKRFSLGMLQGRGPL
KLFMALVAFLRFLTIPPTAGILKRWGTIKKSKAINVLRGFRKEIGRMLNILNRRRRSA
GMIIMLIPTVMAFELTTRNGEPHMIVSRQEKGKSLLFKTEDGVNMCTLMAMDLGELCE
DTITYNCPLLRQNEPEDIDCWCNSTSTWVTYGTCTTTGEHRREKRSVALVPHVGMGLE
TRTETWMSSEGAWKHAQRIETWILRHPGFTIMAAILAYTIGTTHFQRALIFILLTAVA
PSMTMRCIGISNRDFVEGVSGGSWVDIVLEHGSCVTTMAKNKPTLDFELIKTEAKQPA
TLRKYCIEAKLTNTTTESRCPTQGEPSLNEEQDKRFVCKHSMVDRGWGNGCGLFGKGG
IVTCAMFTCKKNMEGKVVQPENLEYTIVVTPHSGEEHAVGNDTGKHGKEIKVTPQSSI
TEAELTGYGTVTMECSPRTGLDFNEMVLLQMENKAWLVHRQWFLDLPLPWLPGADTQG
SNWIQKETLVTFKNPHAKKQDVVVLGSQEGAMHTALTGATEIQMSSGNLLFTGHLKCR
LRMDKLQLKGMSYSMCTGKFKVVKEIAETQHGTIVIRVQYEGDGSPCKIPFEIMDLEK
RHVLGRLITVNPIVTEKDSPVNIEAEPPFGDSYIIIGVDPGQLKLNWFKKGSSIGQMF
ETTMRGAKRMAILGDTAWDFGSLGGVFTSIGKALHQVFGAIYGAAFSGVSWTMKILIG
VIITWIGMNSRSTSLSVSLVLVGIVTLYLGVMVQADSGCVVSWKNKELKCGSGIFITD
NVHTWTEQYKFQPESPSKLASAIQKAQEEGICGIRSVTRLENLMWKQITPELNHILAE
NEVKLTIMTGDIKGIMQAGKRSLRPQPTELKYSWKTWGKAKMLSTESHNQTFLIDGPE
TAECPNTNRAWNSLEVEDYGFGVFTTNIWLKLKEKQDAFCDSKLMSAAIKDNRAVHAD
MGYWIESALNDTWKIEKASFIEVKNCHWPKSHTLWSNGVLESEMIIPKNLAGPVSQHN
YRPGYHTQIAGPWHLGKLEMDFDFCDGTTVVVTEDCGNRGPSLRTTTASGKLITEWCC
RSCTLPPLRYRGEDGCWYGMEIRPLKEKEENLVNSLVTAGHGQVDNFSLGVLGMALFL
EEMLRTRVGTKHAILLVAVSFVTLITGNMSFRDLGRVVVMVGATMTDDIGMGVTYLAL
LAAFKVRPTFAAGLLLRKLTSKELMMTTIGIVLLSQSTIPETILELTDALALGMMVLK
MVRNMEKYQLAVTIMAILCVPNAVILQNAWKVSCTILAVVSVSPLLLTSSQQKTDWIP
LALTIKGLNPTAIFLTTLSRTSKKRSWPLNEAIMAVGMVSILASSLLKNDIPMTGPLV
AGGLLTVCYVLTGRSADLELERAADVKWEDQAEISGSSPILSITISEDGSMSIKNEEE
EQTLTILIRTGLLVISGLFPVSIPITAAAWYLWEVKKQRAGVLWDVPSPPPMGKAELE
DGAYRIKQKGILGYSQIGAGVYKEGTFHTMWHVTRGAVLMHKGKRIEPSWADVKKDLI
SYGGGWKLEGEWKEGEEVQVLALEPGKNPRAVQTKPGLFRTNAGTIGAVSLDFSPGTS
GSPIIDKKGKVVGLYGNGVVTRSGAYVSAIAQTEKSIEDNPEIEDDIFRKRRLTIMDL
HPGAGKTKRYLPAIVREAIKRGLRTLILAPTRVVAAEMEEALRGLPIRYQTPAIRAEH
TGREIVDLMCHATFTMRLLSPVRVPNYNLIIMDEAHFTDPASIAARGYISTRVEMGEA
AGIFMTATPPGSRDPFPQSNAPIIDEEREIPERSWNSGHEWVTDFKGKTVWFVPSIKA
GNDIAACLRKNGKKVIQLSRKTFDSEYVKTRTNDWDFVVTTDISEMGANFKAERVIDP
RRCMKPVILTDGEERVILAGPMPVTHSSAAQRRGRIGRNPKNENDQYIYMGEPLENDE
DCAHWKEAKMLLDNINTPEGIIPSMFEPEREKVDAIDGEYRLRGEARKTFVDLMRRGD
LPVWLAYKVAAEGINYADRRWCFDGIKNNQILEENVEVEIWTKEGERKKLKPRWLDAR
IYSDPLALKEFKEFAAGRKSLTLNLITEMGRLPTFMTQKARDALDNLAVLHTAEAGGR
AYNHALSELPETLETLLLLTLLATVTGGIFLFLMSGRGIGKMTLGMCCIITASILLWY
AQIQPHWIAASIILEFFLIVLLIPEPEKQRTPQDNQLTYVVIAILTVVAATMANEMGF
LEKTKKDLGLGSIATQQPESNILDIDLRPASAWTLYAVATTFVTPMLRHSIENSSVNV
SLTAIANQATVLMGLGKGWPLSKMDIGVPLLAIGCYSQVNPITLTAALLLLVAHYAII
GPGLQAKATREAQKRAAAGIMKNPTVDGITVIDLDPIPYDPKFEKQLGQVMLLVLCVT
QVLMMRTTWALCEALTLATGPISTLWEGNPGRFWNTTIAVSMANIFRGSYLAGAGLLF
SIMKNTTNTRRGTGNIGETLGEKWKSRLNALGKSEFQIYKKSGIQEVDRTLAKEGIKR
GETDHHAVSRGSAKLRWFVERNMVTPEGKVVDLGCGRGGWSYYCGGLKNVREVKGLTK
GGPGHEEPIPMSTYGWNLVRLQSGVDVFFIPPEKCDTLLCDIGESSPNPTVEAGRTLR
VLNLVENWLNNNTQFCIKVLNPYMPSVIEKMETLQRKYGGALVRNPLSRNSTHEMYWV
SNASGNIVSSVNMISRMLINRFTMRHKKATYEPDVDLGSGTRNIGIESEIPNLDIIGK
RIEKIKQEHETSWHYDQDHPYKTWAYHGSYETKQTGSASSMVNGVVRLLTKPWDVVPM
VTQMAMTDTTPFGQQRVFKEKVDTRTQEPKEGTKKLMKITAEWLWKELGKKKTPRMCT
REEFTRKVRSNAALGAIFTDENKWKSAREAVEDSRFWELVDKERNLHLEGKCETCVYN
MMGKREKKLGEFGKAKGSRAIWYMWLGARFLEFEALGELNEDHWFSRENSLSGVEGEG
LHKLGYILRDVSKKEGGAMYADDTAGWDTRITLEDLKNEEMVTNHMEGEHKKLAEAIF
KLTYQNKVVRVQRPTPRGTVMDIISRRDQRGSGQVGTYGLNTFTNMEAQLIRQMEGEG
VFKNIQHLTVTEETAVQNWLARVGRERLSRMAISGDDCVVKPLDDRFASALTALNDMG
KIRKDIQQWEPSRGWNDWTQVPFCSHHFHELIMKDGRVLVVPCRNQDELIGRARISQG
AGWSLRETACLGKSYAQMWSLMYFHRRDLRLAANAICSAVPSHWVPTSRTTWSIHAKH
EWMTTEDMLTVWNRVWIQENPWMEDKTPVESWEEIPYLGKREDQWCGSLIGLTSRATW
AKNIQAAINQVRSLIGNEEYTDYMPSMKRFRREEEEAGVLW
AMINO ACID SEQUENCE OF DENV3 GENBANK ACCESSION NO. JQ411814.1 (SEQ
ID NO: 3)
MNNQRKKTGKPSINMLKRVRNRVSTGPQLAKRFSKGLLNGQGPM
KLVMAFIAFLRFLAIPPTAGVLARWGTFKKSGAIKVLKGFKKEISNMLSIINKRKKTS
LCLMMILPAALAFHLTSRDGEPRMIVGKNERGKSLLFKTASGINMCTLIAMDLGEMCD
DTVTYKCPHITEVEPEDIDCWCNLTSTWVTYGTCNQAGEHRRDKRSVALAPHVGMGLD
TRTQTWMSAEGAWRQVEKVETWALRHPGFTILALFLAHYIGTSLTQKVVIFILLMLVT
PSMTMRCVGIGNRDFVEGLSGATWVDVVLEHGGCVTTMAKNKPTLDIELQKTEATQLA
TLRKLCIEGKITNITTDSRCPTQGEAVLPEEQDQNYVCKHTYVDRGWGNGCGLFGKGS
LVTCAKFQCLEPIEGKVVQYENLKYTVIITVHTGDQHQVGNETQGVTAEITPQASTTE
AILPEYGTLGLECSPRTGLDFNEMILLTMKNKAWMVHRQWFFDLPLPWTSGATTETPT
WNRKELLVTFKNAHAKKQEVVVLGSQEGAMHTALTGATEIQNSGGTSIFAGHLKCRLK
MDKLELKGMSYAMCTNTFVLKKEVSETQHGTILIKVEYKGEDAPCKIPFSTEDGQGKA
HNGRLITANPVVTKKEEPVNIEAEPPFGESNIVIGIGDNALKINWYKKGSSIGKMFEA
TARGARRMAILGDTAWDFGSVGGVLNSLGKMVHQIFGSAYTALFSGVSWVMKIGIGVL
LTWIGLNSKNTSMSFSCTATGIITLYLGAVVQADMGCVINWKGKELKCGSGIFVTNEV
HTWTEQYKFQADSPKRLATAIAGAWENGVCGIRSTTRMENLLWKQIANELNYILWENN
IKLTVVVGDTIGVLEQGKRTLTPQPMELKYSWKTWGKAKIVTAETQNSSFIIDGPNTP
ECPSASRAWNVWEVEDYGFGVFTTNIWLKLRDVYTQLCDHRLMSAAVKDERAVHADMG
YWIESQKNGSWKLEKASLIEVKTCTWPKSHTLWSNGVLESDMIIPKSLAGPISQHNYR
PGYHTQTAGPWHLGKLELDFNYCEGTTVVITENCGTRGPSLRTTTVSGKLIHEWCCRS
CTLPPLRYMGEDGCWYGMEIRPISEKEENMVKSLVSAGSGKVDNFTMGVLCLAILFEE
VMRGKFGKKHMIAGVLFTFVLLLSGQITWRDMAHTLIMIGSNASDRMGMGVTYLALIA
TFKIQPFLALGFFLRKLTSRENLLLGVGLAMATTLQLPEDIEQMANGIALGLMALKLI
TQFETYQLWTALVSLTCSNTILTLTVAWRTATLILAGVSLLPVCQSSSMRKTDWLPMT
VAAMGVPPLPLFIFSLKDTLKRRSWPLNEGVMAVGLVSILASSLLRNDVPMAGPLVAG
GLLIACYVITGTSADLTVEKAADVTWEEEAEQTGVSHNLMITVDDDGTMRIKDDETEN
ILTVLLKTALLIVSGIFPYSIPATLLVWHTWQKQTQRSGVLWDVPSPPETQKAELEEG
VYRIKQQGILGKTQVGVGVQKEGVFHTMWHVTRGAVLTHNGKRLEPNWASVKKDLISY
GGGWRLSAQWQKGEEVQVIAVEPGKNPKNFQTMPGTFQTTTGEIGAIALDFKPGTSGS
PIINREGKVVGLYGNGVVTKNGGYVSGIAQTNAEPDGPTPELEEEMFKKRNLTIMDLH
PGSGKTRKYLPAIVREAIKRRLRTLILAPTRVVAAEMEEALKGLPIRYQTTATKSEHT
GREIVDLMCHATFTMRLLSPVRVPNYNLIIMDEAHFTDPASIAARGYISTRVGMGEAA
AIFMTATPPGTADAFPQSNAPIQDEERDIPERSWNSGNEWITDFAGKTVWFVPSIKAG
NDIANCLRKNGKKVIQLSRKTFDTEYQKTKLNDWDFVVTTDISEMGANFKADRVIDPR
RCLKPVILTDGPERVILAGPMPVTAASAAQRRGRVGRNPQKENDQYIFTGQPLNNDED
HAHWTEAKMLLDNINTPEGIIPALFEPEREKSAAIDGEYRLKGESRKTFVELMRRGDL
PVWLAHKVASEGIKYTDRKWCFDGQRNNQILEENMDVEIWTKEGEKKKLRPRWLDART
YSDPLALKEFKDFAAGRKSIALDLVTEIGRVPSHLAHRTRNALDNLVMLHTSEHGGRA
YRHAVEELPETMETLLLLGLMILLTGGAMLFLISGKGIGKTSIGLICVIASSGMLWMA
EIPLQWIASAIVLEFFMMVLLIPEPEKQRTPQDNQLAYVVIGILTLAAIIAANEMGLL
ETTKRDLGMSKEPGVVSPTSYLDVDLHPASAWTLYAVATTVITPMLRHTIENSTANVS
LAAIANQAVVLMGLDKGWPISKMDLGVPLLALGCYSQVNPLTLTAAVLLLITHYAIIG
PGLQAKATREAQKRTAAGIMKNPTVDGIMTIDLDPVIYDSKFEKQLGQVMLLVLCAVQ
LLLMRTSWALCEALTLATGPITTLWEGSPGKFWNTTIAVSMANIFRGSYLAGAGLAFS
IMKSVGTGKRGTGSQGETLGEKWKKKLNQLSRKEFDLYKKSGITEVDRTEAKEGLKRG
EITHHAVSRGSAKLQWFVERNMVIPEGRVIDLGCGRGGWSYYCAGLKKVTEVRGYTKG
GPGHEEPVPMSTYGWNIVKLMSGKDVFYLPPEKCDTLLCDIGESSPSPTVEESRTIRV
LKMVEPWLKNNQFCIKVLNPYMPTVIEHLERLQRKHGGMLVRNPLSRNSTHEMYWISN
GTGNIVSSVNMVSRLLLNRFTMTHRRPTIEKDVDLGAGTRHVNAEPETPNMDVIGERI
KRIKEEHNSTWHYDDENPYKTWAYHGSYEVKATGSASSMINGVVKLLTKPWDVVPMVT
QMAMTDTTPFGQQRVFKEKVDTRTPRPMPGTRKVMGITAEWLWRTLGRNKRPRLCTRE
EFTKKVRTNAAMGAVFTEENQWDSAKAAVEDEEFWKLVDRERELHKLGKCGSCVYNMM
GKREKKLGEFGKAKGSRAIWYMWLGARYLEFEALGFLNEDHWFSRDNSYSGVEGEGLH
KLGYILRDISKIPGGAMYADDTAGWDTRITEDDLHNEEKITQQMDPEHRQLANAIFKL
TYQNKVVKVQRPTPTGTVMDIISRKDQRGSGQVGTYGLNTFTNMEVQLVRQMEGEGVL
SKADLENPHLPEKKITQWLETKGVERLKRMAISGDDCVVKPIDDRFANALLALNDMGK
VRKDIPQWQPSKGWHDWQQVPFCSHHFHELIMKDGRKLVVPCRPQDELIGRARISQGA
GWSLRETACLGKAYAQMWSLMYFHRRDLRLASNAICSAVPVHWVPTSRTTWSIHAHHQ
WMTTEDMLTVWNRVWIEDNPWMEDKTPVTTWENVPYLGKREDQWCGSLIGLTSRATWA
QNIPTAIQQVRSLIGNEEFLDYMPSMKRFRKEEESEGAIW
AMINO ACID SEQUENCE OF DENV4 GENBANK ACCESSION NO. KJ160504.1 (SEQ
ID NO: 4)
MNQRKKVVRPPFNMLKRERNRVSTPQGLVKRFSTGLFSGKGPLR
MVLAFITFLRVLSIPPTAGILKRWGQLKKNKAIKILIGFRKEIGRMLNILNGRKRSTI
TLLCLIPTVMAFHLSTRDGEPLMIVAKHERGRPLLFKTTEGINKCTLIAMDLGEMCED
TVTYKCPLLVNTEPEDIDCWCNLTSTWVMYGTCTQSGERRREKRSVALTPHSGMGLET
RAETWMSSEGAWKHAQRVESWILRNPGFALLAGFMAYMIGQTGIQRTVFFVLMMLVAP
SYGMRCVGVGNRDFVEGVSGGAWVDLVLEHGGCVTTMAQGKPTLDFELTKTTAKEVAL
LRTYCIEASISNITTATRCPTQGEPYLKEEQDQQYICRRDVVDRGWGNGCGLFGKGGV
VTCAKFSCSGKITGNLVQIENLEYTVVVTVHNGDTHAVGNDTSNHGVTATITPRSPSV
EVKLPDYGELTLDCEPRSGIDFNEMILMKMKKKTWLVHKQWFLDLPLPWTAGADTSEV
HWNYKERMVTFKVPHAKRQDVTVLGSQEGAMHSALAGATEVDSGDGNHMFAGHLKCKV
RMEKLRIKGMSYTMCSGKFSIDKEMAETQHGTTVVKVKYEGAGAPCKVPIEIRDVNKE
KVVGRVISSTPLAENTNSVTNIELEPPFGDSYIVIGVGNSALTLHWFRKGSSIGKMFE
STYRGAKRMAILGETAWDFGSVGGLFTSLGKAVHQVFGSVYTTMFGGVSWMIRILIGF
LVLWIGTNSRNTSMAMTCIAVGGITLFLGFTVQADMGCVVSWSGRELKCGSGIFVVDN
VHTWTEQYKFQPESPARLASAILNAHKDGVCGIRSTTRLENVMWKQITNELNYVLWEG
GHDLTVVAGDVKGVLTKGKRALTPPVSDLKYSWKTWGKAKIFTPEARNSTFLIDGPDT
SECPNERRAWNSLEVEDYGFGMFTTNIWMKFREGSSEVCDHRLMSAAIKDQKAVHADM
GYWIESSKNQTWQIEKASLIEVKTCLWPKTHTLWSNGVLESQMLIPKSYAGPFSQHNY
RQGYATQTVGPWHLGKLEIDFGECPGTTVTIQEDCDHRGPSLRTTTASGKLVTQWCCR
SCTMPPLRFLGEDGCWYGMEIRPLSEKEENMVKSQVTAGQGTSETFSMGLLCLTLFVE
ECLRRRVTRKHMILVVVITLCAIILGGLTWMDLLRALIMLGDTMSGRIGGQIHLAIMA
VFKMSPGYVLGVFLRKLTSRETALMVIGMAMTTVLSIPHDLMELIDGISLGLILLKIV
TQFDNTQVGTLALSLTFIRSTMPLVMAWRTIMAVLFVVTLIPLCRTSCLQKQSHWVEI
TALILGAQALPVYLMTLMKGASRRSWPLNEGIMAVGLVSLLGSALLKNDVPLAGPMVA
GGLLLAAYVMSGSSADLSLEKAANVQWDEMADITGSSPIIEVKQDEDGSFSIRDVEET
NMITLLVKLALITVSGLYPLAIPITMTLWYMWQVKTQRSGALWDVPSPAATQKAALSE
GVYRIMQRGLFGKTQVGVGIHMEGVFHTMWHVTRGSVICHETGRLEPSWADVRNDMIS
YGGGWRLGDKWDKEEDVQVLAIEPGKNPKHVQTKPGLFKTLTGEIGAVTLDFKPGTSG
SPIINRKGKVIGLYGNGVVTKSGDYVSAITQAERIGEPDYEVDEDIFRKKRLTIMDLH
PGAGKTKRILPSIVREALKRRLRTLILAPTRVVAAEMEEALRGLPIRYQTPAVKSEHT
GREIVDLMCHATFTTRLLSSTRVPNYNLIVMDEAHFTDPSSVAARGYISTRVEMGEAA
AIFMTATPPGATDPFPQSNSPIEDIEREIPERSWNTGFDWITDYQGKTVWFVPSIKAG
NDIANCLRKSGKKVIQLSRKTFGTEYPKTKLTDWDFVVTTDISEMGANFRAGRVIDPR
RCLKPVILTDGPERVILAGPIPVTPASAAQRRGRVGRNPAQEDDQYVFSGDPLKNDED
HAHWTEAKMLLDNIYTPEGIIPTLFGPEREKTQAIDGEFRLRGEQRKTFVELMRRGDL
PVWLSYKVASAGISYKDREWCFTGERNNQILEENMEVEIWTREGEKKKLRPRWLDARV
YADPMALKDFKEFASGRKSITLDILTEIASLPAYLSSRAKLALDNIVMLHTTERGGRA
YQHALNELPESLETLMLVALLGAMTAGIFLFFMQGKGIGKLSMGLITIAVASGLLWIA
EIQPQWIAASIILEFFLMVLLIPEPEKQRTPQDNQLIYVILTVLTIIGLIAANEMGLI
EKTKTDFGFYQVKTETTILDVDLRPASAWTLYAVATTVLTPMLRHTIENTSANLSLAA
IANQAAVLMGLGKGWPLHRMDLGVPLLAMGCYSQVNPTTLTASLVMLFVHYAIIGPGL
QAKATREAQKRTAAGIMKNPTVDGITVIDLEPISYDPKFEKQLGQVMLLVLCAGQLLL
MRTTWAFCEVLTLATGPILTLWEGNPGRFWNTTIAVSTANIFRGSYLAGAGLAFSLIK
NAQTPRRGTGTTGETLGEKWKRQLNSLDRKEFEEYKRSGILEVDRTEAKSALKDGSKI
KHAVSRGSSKIRWIVERGMVKPKGKVVDLGCGRGGWSYYMATLKNVTEVKGYTKGGPG
HEEPIPMATYGWNLVKLHSGVDVFYKPTEQVDTLLCDIGESSSNPTIEEGRTLRVLKM
VEPWLSSKPEFCIKVLNPYMPTVIEELEKLQRKHGGNLVRCPLSRNSTHEMYWVSGAS
GNIVSSVNTTSKMLLNRFTTRHRKPTYEKDVDLGAGTRSVSTETEKPDMTIIGRRLQR
LQEEHKETWHYDQENPYRTWAYHGSYEAPSTGSASSMVNGVVKLLTKPWDVIPMVTQL
AMTDTTPFGQQRVFKEKVDTRTPQPKPGTRMVMTTTANWLWALLGKKKNPRLCTREEF
ISKVRSNAAIGAVFQEEQGWTSASEAVNDSRFWELVDKERALHQEGKCESCVYNMMGK
REKKLGEFGRAKGSRAIWYMWLGARFLEFEALGFLNEDHWFGRENSWSGVEGEGLHRL
GYILEEIDKKDGDLMYADDTAGWDTRITEDDLQNEELITEQMAPHHKILAKAIFKLTY
QNKVVKVLRPTPRGAVMDIISRKDQRGSGQVGTYGLNTFTNMEVQLIRQMEAEGVITQ
DDMQNPKGLKERVEKWLRECGVDRLKRMAISGDDCVVKPLDERFGTSLLFLNDMGKVR
KDIPQWEPSKGWKNWQEVPFCSHHFHKIFMKDGRSLVVPCRNQDELIGRARISQGAGW
SLRETACLGKAYAQMWSLMYFHRRDLRLASMAICSAVPTEWFPTSRTTWSIHAHHQWM
TTEDMLKVWNRVWIEDNPNMTDKTPVHSWEDIPYLGKREDLWCGSLIGLSSRATWAKN
IHTAITQVRNLIGKEEYVDYMPVMKRYSAPSESEGVL
AMINO ACID SEQUENCE OF ZIKA VIRUS GENBANK ACCESSION NO. KJ776791.2 (SEQ ID
NO: 5)
MKNPKKKSGGFRIVNMLKRGVARVSPFGGLKRLPAGLLLGHGPI
RMVLAILAFLRFTAIKPSLGLINRWGSVGKKEAMEIIKKFKKDLAAMLRIINARKEKK
RRGADTSVGIVGLLLTTAMAAEVTRRGSAYYMYLDRNDAGEAISFPTTLGMNKCYIQI
MDLGHMCDATMSYECPMLDEGVEPDDVDCWCNTTSTWVVYGTCHHKKGEARRSRRAVT
LPSHSTRKLQTRSQTWLESREYTKHLIRVENWIFRNPGFALAAAAIAWLLGSSTSQKV
IYLVMILLIAPAYSIRCIGVSNRDFVEGMSGGTWVDVVLEHGGCVTVMAQDKPTVDIE
LVTTTVSNMAEVRSYCYEASISDMASDSRCPTQGEAYLDKQSDTQYVCKRTLVDRGWG
NGCGLFGKGSLVTCAKFACSKKMTGKSIQPENLEYRIMLSVHGSQHSGMIVNDTGHET
DENRAKVEITPNSPRAEATLGGFGSLGLDCEPRTGLDFSDLYYLTMNNKHWLVHKEWF
HDIPLPWHAGADTGTPHWNNKEALVEFKDAHAKRQTVVVLGSQEGAVHTALAGALEAE
MDGAKGRLSSGHLKCRLKMDKLRLKGVSYSLCTAAFTFTKIPAETLHGTVTVEVQYAG
TDGPCKVPAQMAVDMQTLTPVGRLITANPVITESTENSKMMLELDPPFGDSYIVIGVG
EKKITHHWHRSGSTIGKAFEATVRGAKRMAVLGDTAWDFGSVGGALNSLGKGIHQIFG
AAFKSLFGGMSWFSQILIGTLLMWLGLNTKNGSISLMCLALGGVLIFLSTAVSADVGC
SVDFSKKETRCGTGVFVYNDVEAWRDRYKYHPDSPRRLAAAVKQAWEDGICGISSVSR
MENIMWRSVEGELNAILEENGVQLTVVVGSVKNPMWRGPQRLPVPVNELPHGWKAWGK
SYFVRAAKTNNSFVVDGDTLKECPLKHRAWNSFLVEDHGFGVFHTSVWLKVREDYSLE
CDPAVIGTAVKGKEAVHSDLGYWIESEKNDTWRLKRAHLIEMKTCEWPKSHTLWTDGI
EESDLIIPKSLAGPLSHHNTREGYRTQMKGPWHSEELEIRFEECPGTKVHVEETCGTR
GPSLRSTTASGRVIEEWCCRECTMPPLSFRAKDGCWYGMEIRPRKEPESNLVRSMVTA
GSTDHMDHFSLGVLVILLMVQEGLKKRMTTKIIISTSMAVLVAMILGGFSMSDLAKLA
ILMGATFAEMNTGGDVAHLALIAAFKVRPALLVSFIFRANWTPRESMLLALASCLLQT
AISALEGDLMVLINGFALAWLAIRAMVVPRTDNITLAILAALTPLARGTLLVAWRAGL
ATCGGFMLLSLKGKGSVKKNLPFVMALGLTAVRLVDPINVVGLLLLTRSGKRSWPPSE
VLTAVGLICALAGGFAKADIEMAGPMAAVGLLIVSYVVSGKSVDMYIERAGDITWEKD
AEVTGNSPRLDVALDESGDFSLVEDDGPPMREIILKVVLMTICGMNPIAIPFAAGAWY
VYVKTGKRSGALWDVPAPKEVKKGETTDGVYRVMTRRLLGSTQVGVGVMQEGVFHTMW
HVTKGSALRSGEGRLDPYWGDVKQDLVSYCGPWKLDAAWDGHSEVQLLAVPPGERARN
IQTLPGIFKTKDGDIGAVALDYPAGTSGSPILDKCGRVIGLYGNGVVIKNGSYVSAIT
QGRREEETPVECFEPSMLKKKQLTVLDLHPGAGKTRRVLPEIVREAIKTRLRTVILAP
TRVVAAEMEEALRGLPVRYMTTAVNVTHSGTEIVDLMCHATFTSRLLQPIRVPNYNLY
IMDEAHFTDPSSIAARGYISTRVEMGEAAAIFMTATPPGTRDAFPDSNSPIMDTEVEV
PERAWSSGFDWVTDHSGKTVWFVPSVRNGNEIAACLTKAGKRVIQLSRKTFETEFQKT
KHQEWDFVVTTDISEMGANFKADRVIDSRRCLKPVILDGERVILAGPMPVTHASAAQR
RGRIGRNPNKPGDEYLYGGGCAETDEDHAHWLEARMLLDNIYLQDGLIASLYRPEADK
VAAIEGEFKLRTEQRKTFVELMKRGDLPVWLAYQVASAGITYTDRRWCFDGTTNNTIM
EDSVPAEVWTRHGEKRVLKPRWMDARVCSDHAALKSFKEFAAGKRGAAFGVMEALGTL
PGHMTERFQEAIDNLAVLMRAETGSRPYKAAAAQLPETLETIMLLGLLGTVSLGIFFV
LMRNKGIGKMGFGMVTLGASAWLMWLSEIEPARIACVLIVVFLLLVVLIPEPEKQRSP
QDNQMAIIIMVAVGLLGLITANELGWLERTKSDLSHLMGRREEGATIGFSMDIDLRPA
SAWAIYAALTTFITPAVQHAVTTSYNNYSLMAMATQAGVLFGMGKGMPFYAWDFGVPL
LMIGCYSQLTPLTLIVAIILLVAHYMYLIPGLQAAAARAAQKRTAAGIMKNPVVDGIV
VTDIDTMTLDPQVEKKMGQVLLIAVAVSSAILSRTAWGWGEAGALITAATSTLWEGSP
NKYWNSSTATSLCNIFRGSYLAGASLIYTVTRNAGLVKRRGGGTGETLGEKWKARLNQ
MSALEFYSYKKSGITEVCREEARRALKDGVATGGHAVSRGSAKLRWLVERGYLQPYGK
VIDLGCGRGGWSYYAATIRKVQEVKGYTKGGPGHEEPMLVQSYGWNIVRLKSGVDVFH
MAAEPCDTLLCDIGESSSSPEVEEARTLRVLSMVGDWLEKRPGAFCIKVLCPYTSTMM
ETLERLQRRYGGGLVRVPLSRNSTHEMYWVSGAKSNTIKSVSTTSQLLLGRMDGPRRP
VKYEEDVNLGSGTRAVVSCAEAPNMKIIGNRIERIRSEHAETWFFDENHPYRTWAYHG
SYEAPTQGSASSLINGVVRLLSKPWDVVTGVTGIAMTDTTPYGQQRVFKEKVDTRVPD
PQEGTRQVMSMVSSWLWKELGKHKRPRVCTKEEFINKVRSNAALGAIFEEEKEWKTAV
EAVNDPRFWALVDKEREHHLRGECQSCVYNMMGKREKKQGEFGKAKGSRAIWYMWLGA
RFLEFEALGFLNEDHWMGRENSGGGVEGLGLQRLGYVLEEMSRIPGGRMYADDTAGWD
TRISRFDLENEALITNQMEKGHRALALAIIKYTYQNKVVKVLRPAEKGKTVMDIISRQ
DQRGSGQVVTYALNTFTNLVVQLIRNMEAEEVLEMQDLWLLRRSEKVTNWLQSNGWDR
LKRMAVSGDDCVVKPIDDRFAHALRFLNDMGKVRKDTQEWKPSTGWDNWEEVPFCSHH
FNKLHLKDGRSIVVPCRHQDELIGRARVSPGAGWSIRETACLAKSYAQMWQLLYFHRR
DLRLMANAICSSVPVDWVPTGRTTWSIHGKGEWMTTEDMLVVWNRVWIEENDHMEDKT
PVTKWTDIPYLGKREDLWCGSLIGHRPRTTWAENIKNTVNMVRRIIGDEEKYMDYLST
QVRYLGEEGSTPGVL
AMINO ACID SEQUENCE OF TAMANA BAT VIRUS GENBANK ACCESSION NO. NC_003996 (SEQ
ID NO: 6)
MNKNKKQKPNTKMRSNPQKNKIKLVNKKLQTFFMAFFQILLAVF
KNFKVSLQLRFLEKVYRTVFKNLAIRSPKGAVAIMKWGVGYLKKRKQKRQKSSGGYEI
EAARQALYPITLGVMVIFCGYQSGNEPVCVDFNNQTNIVPYENGKVIFGNVTIVPPID
RFGLLIPTDNYDTIECVRMSPGIELSEEVCGFMGPKYDVLLRTQWGKRETEHMHRTRR
SVTETTHESSGGEHWDYSEYLTDFEHKMSKYKMPVLIILVGYLVLKTQNMALLIILVI
AQFYLADAGHCHDPEKFDIIEAGGYTKMTTFLKHDKCFMVRVPEHDVVEITVVKKTIR
PTTGCKTFLLGASVNTESRAKCPGAGSATIPKTPGDKTFCHIEHVDRGWDSGCFIFGK
GEVVTCAAVTYSKPFYAWMVDSEQITWEVEVNFGGDSNRKKTAEISMDSEYEEFEDPK
NTGSKIRLYCKTPRQEWDNYYMVARPGGSLYKVRRELINDLNLPWTDFKYPYVTTTIV
RNGESILRFKRPTMTYIPVTVAAGLERLYMDVVLQGPVLKCSGNHCTKEQWSGNSETE
LPTITEDVGTLKCEIDYSGLKVRVTNVALCTHTSTIVSGPLVTNDIVMIVLRGTGSDT
TECKLKVEGKSASSNNWITNYCAPHSRSMSVKPNEQTTVKMTCPNGIIDLRFNGKTTT
QVEVKYSRTMKLVSSMMASFKDVTENVMIHESGFETFKKVAKMLTTPVVGFFKSILGL
ASNWYILIFALLLVYVGVNKQGPVGNIMLALGLICLLTYYFEVVAADKYVLMVDTDDM
SLSVGKAHHIEWDWKITMPLIPSLPYQVTLIGLDPKYGITIYRVDSQEEYTNLAKVLS
EKYDLVRKITGWYGNSWIKVPCRVIASDAALNLTADCEKAISENSKVAVLTSEHTPIV
CRDCIEKFVKLSLDKEEQGAFRNHKHYNFQNLTREECVIDGNWTYSSAIEVYSKYELE
NEDEITTTQGGGYGTITIGEKTWKYVEVKFGPISVKNTILFQGVLDSVVPTKRKWVNM
TNWACNFFKKENQVNRGHILAKSLGGPAAGYNCMIQLVKENNEDEDIEKSALDSALKQ
STSYRREIKPKNIKAIYYTQKVDERNIMCGWNGKDSVNVVKASKMNKDNIQDLITKNL
NVLMVIDNNDESSIRALIGDKMLFCEANWVDCNTHDYELSFWTSALITTKSRSYEHKP
CKIQPKDEIFEDADSFFSDEKVVLTTAGISRDNVNFYWLFTFFLGWILFEWAVIVDLG
SIFLYNIKKRWTVLLIIPTVFQLMVNGIITSIVSITVFFCGSMKNTLVTATDIVEGIN
LGHWKRTAMVTLMVSGFFKNEFLSLLMILNFTLNWTHFVPILMCLPHFLGLPNPWIWL
GVLVLALIGLINGKLELDFQNKLVPLPQKKFETQLQLGKVYWTASGIEVEIREEIRMA
KYDYPLLALYLTLVFSASLIHWSFGITTLMLGGSLFNLRDKSKGLIPMPELEEVHDPP
EKLKDGFYSISVAGLCWKYQIGIGYVHNGTLTTQYHVTCGRDLWWKGKSYSPVEENVV
SDYACYFGPWSFPKLQEGSEFTIMMRWKNIALKTMGKITGNYYAIPTEKIPKGQSGTP
IFQEINGTLKPVALAGNSIVFGGESVQVGENPSFAPHLDGKIKQLLNTRGCWEEFVLK
CGAGKTRGVIKDTIQHATRMGVKVLVLVPTRVVANEAYNVLKDVGTVGIQTSTYRNDV
GLMHTIMCHSTFARRIMRKGKRILRNWQLIIVDESHFCNPETLALHNLFMRDLRSPTP
TYSVMYLTATGYTSYDSNSNFFVHDHPVASKNDFLEIIKQNKEKKIVYFVASGPEANE
IAGKLRAEIQNIIIVTRQTFNNTYEAAKTMEQGLILTTNISEMGANYNADIVMDPLEE
IIPVLGEEGVQLEKTVCTRASKIQRRGRVGRSKPGDYYYLAVNEGQINQQMHHQQDIV
WAEAEMCSNLMGEIWEPENFQGSEPPAPINYYLTPQKLATIVNKTEMENGWTYYTSYH
LTDDYGNSIPYNVTDPQLHYTPNHCPKCTGLHFPVWDERNHDMLVSCGRDLDNNDVNT
RTRQNVGVIQPLVQRVFSGIRMQSSGEGCELFSILFGVQCYGWMTYFDMPASWIALAH
QALPPQWRETITPMKLLFILFGFMLKVVYDKIMVNKHLVYNYAPYKGHSQMLMFLTVA
STMLQNEALKIMSYPIWQILFVVFITVIVVETVCGITQREKSTGEVTQLVTIGAVIEI
IYYILADGEILTLIKKLIKVKIQQIENNYEVPQQPIEDTRIRTWSLFDRPPVYLYFTL
HPVIIQLARSTSVKALIQFVDNVKLRKTPVNMPQPTVYFIVAVFISIFNFYYYSFPHL
VASIIPSILLYCVIYCAHYTDIFKRFVDTNHELGNSNNQHIPYSDPAYSEIYRVLVIV
IEITYLLVYKTLTLDSVLVVLHMTSPWIRGPIGEGLNNYGVVILAMSMKHLMIPQSIV
AITAVVLDHLKTTQRFRSSIFTTQILNRMVEHYQDLEWRGRQYKFTLNKLNKHDFLQF
KGENVYELENKDDFPSRGFAKMDWMIKSTLFQPRGFVVDGGCGAGGFSARMCFEKPVA
KILGYTIGKDKHHTPDLNGKKVYKKFNWEIGDLFKKRIRCDTFVMDIGESHPNQEIEA
IGDLKRLNWFKENVDCKNWCIKIMVPTHEQVLHALPKGSTLIRNPWSRNSTLEMYCVP
GSNEPGRMVSNVIKVLNQRINRTSWQGSIMKKLYYPEDEEQVGSTKVNYIIEKEKLQN
FTLQEERRVFNHWKCLGSVEASRKGSSGQNYNSIITAITSGLRKVVPSIEHWMLTSTK
PEDTFSTFLRKVDQPPQPLGNYKSEFQEKFKWVQQWYKSKNKHGRILTNEEVISKFKS
DGALGFRCQHYGSIQEALENPNFFKDLEAEEINHLHGKCTRGVFDVIGKKEKKDLYGK
PKGSRLIMYLDLVERFLEHKYLGFLNSDHWCHPENLPSGVSAVSPYEYPRMIVEKCKI
DPRSDLMENWVIQDDTAGWDTRLHDDVLECEQSFLCDFAESEEHIKHILRIYKNYRNP
MIKLTDDSGTRDLILIGKGQRCSGTVVTYSMNTITNTVVQMMRMQEVLELSNEECLHK
MMVSGDDCLLVLKPEEAIKVSKSLKFINSTGFIRKDVPRHVPSPVVKDWKNISFCSHG
IAKGRMQNGEYLWTLGKNEAEIIGKAQLQIGAFGDEINEQSQAKAMALYLLLTFPMRR
DIRLIAKAIMACCQEGLVPMGKVKEPLIWGEPWLDSMDIVNIINKIYGTNFYTLEEVP
YVRHSLDMERGSTIHTNERSNWKKFLLEKMVPDLSRRNNTAPNMNWLVRNGNRGNLLV
ZIKV/DENV 5J7 Swap (SEQ ID NO: 7)
MKNPKKKSGGFRIVNMLKRGVARVSPFGGLKRLPAGLLLGHGPIRMVLAILAFLRFT
AIKPSLGLINRWGSVGKKEAMEIIKKFKKDLAAMLRIINARKEKKRRGADTSVGIVGL
LLTTAMAAEVTRRGSAYYMYLDRNDAGEAISFPTTLGMNKCYIQIMDLGHMCDAT
MSYECPMLDEGVEPDDVDCWCNTTSTWVVYGTCHHKKGEARRSRRAVTLPSHSTR
KLQTRSQTWLESREYTKHLIRVENWIFRNPGFALAAAAIAWLLGSSTSQKVIYLVMIL
LIAPAYSIRCIGVSNRDFVEGMSGGTWVDVVLEHGGCVTVMAQDKPTVDIELVTTEA
TQLATLRKLCIEGSISDMASDSRCPTQGEAYLDKQSDTQYVCKRTLVDRGWGNGCG
LFGKGSLVTCAKFACLEPIEGKVVQYENLEYRIMLSVHGSQHSGMIVNDTGHETDEN
RAKVEITPNSPRAEATLGGFGSLGLDCEPRTGLDFSEMIILTMKNKAWMVHRQWFFD
LPLPWTSGATTETPTWNNKEALVEFKDAHAKRQTVVVLGSQEGAVHTALAGALEIQ
MNSGGTSIFAGHLKCRLKMDKLRLKGVSYSLCTAAFTFTKIPAETLHGTVTVEVQYA
GTDGPCKVPAQMAVDMQTLTPVGRLITANPVITESTENSKMMLELDPPFGDSYIVIG
VGEKKITHHWHRSGSTIGKAFEATVRGAKRMAVLGDTAWDFGSVGGALNSLGKGI
HQIFGAAFKSLFGGMSWFSQILIGTLLMWLGLNTKNGSISLMCLALGGVLIFLSTAVS
ADVGCSVDFSKKETRCGTGVFVYNDVEAWRDRYKYHPDSPRRLAAAVKQAWEDGI
CGISSVSRMENIMWRSVEGELNAILEENGVQLTVVVGSVKNPMWRGPQRLPVPVNE
LPHGWKAWGKSYFVRAAKTNNSFVVDGDTLKECPLKHRAWNSFLVEDHGFGVFHT
SVWLKVREDYSLECDPAVIGTAVKGKEAVHSDLGYWIESEKNDTWRLKRAHLIEMK
TCEWPKSHTLWTDGIEESDLIIPKSLAGPLSHHNTREGYRTQMKGPWHSEELEIRFEE
CPGTKVHVEETCGTRGPSLRSTTASGRVIEEWCCRECTMPPLSFRAKDGCWYGMEIR
PRKEPESNLVRSMVTAGSTDHMDHFSLGVLVILLMVQEGLKKRMTTKIIISTSMAVL
VAMILGGFSMSDLAKLAILMGATFAEMNTGGDVAHLALIAAFKVRPALLVSFIFRAN
WTPRESMLLALASCLLQTAISALEGDLMVLINGFALAWLAIRAMVVPRTDNITLAIL
AALTPLARGTLLVAWRAGLATCGGFMLLSLKGKGSVKKNLPFVMALGLTAVRLVD
PINVVGLLLLTRSGKRSWPPSEVLTAVGLICALAGGFAKADIEMAGPMAAVGLLIVS
YVVSGKSVDMYIERAGDITWEKDAEVTGNSPRLDVALDESGDFSLVEDDGPPMREII
LKVVLMTICGMNPIAIPFAAGAWYVYVKTGKRSGALWDVPAPKEVKKGETTDGVY
RVMTRRLLGSTQVGVGVMQEGVFHTMWHVTKGSALRSGEGRLDPYWGDVKQDLV
SYCGPWKLDAAWDGHSEVQLLAVPPGERARNIQTLPGIFKTKDGDIGAVALDYPAG
TSGSPILDKCGRVIGLYGNGVVIKNGSYVSAITQGRREEETPVECFEPSMLKKKQLTV
LDLHPGAGKTRRVLPEIVREAIKTRLRTVILAPTRVVAAEMEEALRGLPVRYMTTAV
NVTHSGTEIVDLMCHATFTSRLLQPIRVPNYNLYIMDEAHFTDPSSIAARGYISTRVE
MGEAAAIFMTATPPGTRDAFPDSNSPIMDTEVEVPERAWSSGFDWVTDHSGKTVWF
VPSVRNGNEIAACLTKAGKRVIQLSRKTFETEFQKTKHQEWDFVVTTDISEMGANFK
ADRVIDSRRCLKPVILDGERVILAGPMPVTHASAAQRRGRIGRNPNKPGDEYLYGGG
CAETDEDHAHWLEARMLLDNIYLQDGLIASLYRPEADKVAAIEGEFKLRTEQRKTFV
ELMKRGDLPVWLAYQVASAGITYTDRRWCFDGTTNNTIMEDSVPAEVWTRHGEKR
VLKPRWMDARVCSDHAALKSFKEFAAGKRGAAFGVMEALGTLPGHMTERFQEAID
NLAVLMRAETGSRPYKAAAAQLPETLETIMLLGLLGTVSLGIFFVLMRNKGIGKMGF
GMVTLGASAWLMWLSEIEPARIACVLIVVFLLLVVLIPEPEKQRSPQDNQMAIIIMVA
VGLLGLITANELGWLERTKSDLSHLMGRREEGATIGFSMDIDLRPASAWAIYAALTT
FITPAVQHAVTTSYNNYSLMAMATQAGVLFGMGKGMPFYAWDFGVPLLMIGCYSQ
LTPLTLIVAIILLVAHYMYLIPGLQAAAARAAQKRTAAGIMKNPVVDGIVVTDIDTMT
IDPQVEKKMGQVLLIAVAVSSAILSRTAWGWGEAGALITAATSTLWEGSPNKYWNS
STATSLCNIFRGSYLAGASLIYTVTRNAGLVKRRGGGTGETLGEKWKARLNQMSAL
EFYSYKKSGITEVCREEARRALKDGVATGGHAVSRGSAKLRWLVERGYLQPYGKVI
DLGCGRGGWSYYAATIRKVQEVKGYTKGGPGHEEPMLVQSYGWNIVRLKSGVDVF
HMAAEPCDTLLCDIGESSSSPEVEEARTLRVLSMVGDWLEKRPGAFCIKVLCPYTST
MMETLERLQRRYGGGLVRVPLSRNSTHEMYWVSGAKSNTIKSVSTTSQLLLGRMDG
PRRPVKYEEDVNLGSGTRAVVSCAEAPNMKIIGNRIERIRSEHAETWFFDENHPYRT
WAYHGSYEAPTQGSASSLINGVVRLLSKPWDVVTGVTGIAMTDTTPYGQQRVFKEK
VDTRVPDPQEGTRQVMSMVSSWLWKELGKHKRPRVCTKEEFINKVRSNAALGAIFE
EEKEWKTAVEAVNDPRFWALVDKEREHHLRGECQSCVYNMMGKREKKQGEFGKA
KGSRAIWYMWLGARFLEFEALGFLNEDHWMGRENSGGGVEGLGLQRLGYVLEEMS
RIPGGRMYADDTAGWDTRISRFDLENEALITNQMEKGHRALALAIIKYTYQNKVVK
VLRPAEKGKTVMDIISRQDQRGSGQVVTYALNTFTNLVVQLIRNMEAEEVLEMQDL
WLLRRSEKVTNWLQSNGWDRLKRMAVSGDDCVVKPIDDRFAHALRFLNDMGKVR
KDTQEWKPSTGWDNWEEVPFCSHHFNKLHLKDGRSLVVPCRHQDELIGRARVSPGA
GWSIRETACLAKSYAQMWQLLYFHRRDLRLMANAICSSVPVDWVPTGRTTWSIHG
KGEWMTTEDMLVVWNRVWIEENDHMEDKTPVTKWTDIPYLGKREDLWCGSLIGH
RPRTTWAENIKNTVNMVRRIIGDEEKYMDYLSTQVRYLGEEGSTPGVL
DENV/ZIKV 5J7 Swap (SEQ ID NO: 8)
MNNQRKKTGKPSINMLKRVRNRVSTGPQLAKRFSKGLLNGQGPMKLVMAFIAFLRF
LAIPPTAGVLARWGTFKKSGAIKVLKGFKKEISNMLSIINKRKKTSLCLMMILPAALA
FHLTSRDGEPRMIVGKNERGKSLLFKTASGINMCTLIAMDLGEMCDDTVTYKCPHIT
EVEPEDIDCWCNLTSTWVTYGTCNQAGEHRRDKRSVALAPHVGMGLDTRTQTWMS
AEGAWRQVEKVETWALRHPGFTILALFLAHYIGTSLTQKVVIFILLMLVTPSMTMRC
VGIGNRDFVEGLSGATWVDVVLEHGGCVTTMAKNKPTLDIELQKTTVSNMAEVRS
YCYEAKITNITTDSRCPTQGEAVLPEEQDQNYVCKHTYVDRGWGNGCGLFGKGSLV
TCAKFQCSKKMTGKSIQPENLKYTVIITVHTGDQHQVGNETQGVTAEITPQASTTEAI
LPEYGTLGLECSPRTGLDFNDLYLLTMNNKHWLVHKEWFHDIPLPWHAGADTGTPH
WNRKELLVTFKNAHAKKQEVVVLGSQEGAMHTALTGATEAEMDGAKGRLSSGHL
KCRLKMDKLELKGMSYAMCTNTFVLKKEVSETQHGTILIKVEYKGEDAPCKIPFSTE
DGQGKAHNGRLITANPVVTKKEEPVNIEAEPPFGESNIVIGIGDNALKINWYKKGSSI
GKMFEATARGARRMAILGDTAWDFGSVGGVLNSLGKMVHQIFGSAYTALFSGVSW
VMKIGIGVLLTWIGLNSKNTSMSFSCIAIGIITLYLGAVVQADMGCVINWKGKELKCG
SGIFVTNEVHTWTEQYKFQADSPKRLATAIAGAWENGVCGIRSTTRMENLLWKQIA
NELNYILWENNIKLTVVVGDTIGVLEQGKRTLTPQPMELKYSWKTWGKAKIVTAET
QNSSFIIDGPNTPECPSASRAWNVWEVEDYGFGVFTTNIWLKLRDVYTQLCDHRLMS
AAVKDERAVHADMGYWIESQKNGSWKLEKASLIEVKTCTWPKSHTLWSNGVLESD
MIIPKSLAGPISQHNYRPGYHTQTAGPWHLGKLELDFNYCEGTTVVITENCGTRGPSL
RTTTVSGKLIHEWCCRSCTLPPLRYMGEDGCWYGMEIRPISEKEENMVKSLVSAGSG
KVDNFTMGVLCLAILFEEVMRGKFGKKHMIAGVLFTFVLLLSGQITWRDMAHTLIMI
GSNASDRMGMGVTYLALIATFKIQPFLALGFFLRKLTSRENLLLGVGLAMATTLQLP
EDIEQMANGIALGLMALKLITQFETYQLWTALVSLTCSNTILTLTVAWRTATLILAGV
SLLPVCQSSSMRKTDWLPMTVAAMGVPPLPLFIFSLKDTLKRRSWPLNEGVMAVGL
VSILASSURNDVPMAGPLVAGGLLIACYVITGTSADLTVEKAADVTWEEEAEQTGV
SHNILMITVDDDGTMRIKDDETENILTVLLKTALLIVSGIFPYSIPATLLVWHTWQKQT
QRSGVLWDVPSPPETQKAELEEGVYRIKQQGILGKTQVGVGVQKEGVFHTMWHVT
RGAVLTHNGKRLEPNWASVKKDLISYGGGWRLSAQWQKGEEVQVIAVEPGKNPKN
FQTMPGTFQTTTGEIGAIALDFKPGTSGSPIINREGKVVGLYGNGVVTKNGGYVSGIA
QTNAEPDGPTPELEEEMFKKRNLTIMDLHPGSGKTRKYLPAIVREAIKRRLRTLILAPT
RVVAAEMEEALKGLPIRYQTTATKSEHTGREIVDLMCHATFTMRLLSPVRVPNYNLII
MDEAHFTDPASIAARGYISTRVGMGEAAAIFMTATPPGTADAFPQSNAPIQDEERDIP
ERSWNSGNEWITDFAGKTVWFVPSIKAGNDIANCLRKNGKKVIQLSRKTFDTEYQKT
KLNDWDFVVTTDISEMGANFKADRVIDPRRCLKPVILTDGPERVILAGPMPVTAASA
AQRRGRVGRNPQKENDQYIFTGQPLNNDEDHAHWTEAKMLLDNINTPEGIIPALFEP
EREKSAAIDGEYRLKGESRKTFVELMRRGDLPVWLAHKVASEGIKYTDRKWCFDGQ
RNNQILEENMDVEIWTKEGEKKKLRPRWLDARTYSDPLALKEFKDFAAGRKSIALD
LVTEIGRVPSHLAHRTRNALDNLVMLHTSEHGGRAYRHAVEELPETMETLLLLGLMI
LLTGGAMLFLISGKGIGKTSIGLICVIASSGMLWMAEIPLQWIASAIVLEFFMMVLLIP
EPEKQRTPQDNQLAYVVIGILTLAAIIAANEMGLLETTKRDLGMSKEPGVVSPTSYLD
VDLHPASAWTLYAVATTVITPMLRHTIENSTANVSLAAIANQAVVLMGLDKGWPIS
KMDLGVPLLALGCYSQVNPLTLTAAVLLLITHYAIIGPGLQAKATREAQKRTAAGIM
KNPTVDGIMTIDLDPVIYDSKFEKQLGQVMLLVLCAVQLLLMRTSWALCEALTLAT
GPITTLWEGSPGKFWNTTIAVSMANIFRGSYLAGAGLAFSIMKSVGTGKRGTGSQGE
TLGEKWKKKLNQLSRKEFDLYKKSGITEVDRTEAKEGLKRGEITHHAVSRGSAKLQ
WFVERNMVIPEGRVIDLGCGRGGWSYYCAGLKKVTEVRGYTKGGPGHEEPVPMST
YGWNIVKLMSGICDVFYLPPEKCDTLLCDIGESSPSPTVEESRTIRVLKMVEPWLKNN
QFCIKVLNPYMPTVIEHLERLQRKHGGMLVRNPLSRNSTHEMYWISNGTGNIVSSVN
MVSRLLLNRFTMTHRRPTIEKDVDLGAGTRHVNAEPETPNMDVIGERIKRIKEEHNS
TWHYDDENPYKTWAYHGSYEVKATGSASSMINGVVKLLTKPWDVVPMVTQMAM
TDTTPFGQQRVFKEKVDTRTPRPMPGTRKVMGITAEWLWRTLGRNKRPRLCTREEF
TKKVRTNAAMGAVFTEENQWDSAKAAVEDEEFWKLVDRERELHKLGKCGSCVYN
MMGKREKKLGEFGKAKGSRAIWYMWLGARYLEFEALGFLNEDHWFSRDNSYSGV
EGEGLHKLGYILRDISKIPGGAMYADDTAGWDTRITEDDLHNEEKITQQMDPEHRQL
ANAIFKLTYQNKVVKVQRPTPTGTVMDIISRKDQRGSGQVGTYGLNTFTNMEVQLV
RQMEGEGVLSKADLENPHLPEKKITQWLETKGVERLKRMAISGDDCVVKPIDDRFA
NALLALNDMGKVRKDIPQWQPSKGWHDWQQVPFCSHHFHELIMKDGRKLVVPCRP
QDELIGRARISQGAGWSLRETACLGKAYAQMWSLMYFHRRDLRLASNAICSAVPVH
WVPTSRTTWSIHAHHQWMTTEDMLTVWNRVWIEDNPWMEDKTPVTTWENVPYLG
KREDQWCGSLIGLTSRATWAQNIPTAIQQVRSLIGNEEFLDYMPSMKRFRKEEESEG
AIW
DENV/ZIKV EDIII Swap (SEQ ID NO: 9)
MNQRKKVVRPPFNMLKRERNRVSTPQGLVKRFSTGLFSGKGPLRMVLAFITFLRVLS
IPPTAGILKRWGQLKKNKAIKILIGFRKEIGRMLNILNGRKRSTITLLCLIPTVMAFHLS
TRDGEPLMIVAKHERGRPLLFKTTEGINKCTLIAMDLGEMCEDTVTYKCPLLVNTEP
EDIDCWCNLTSTWVMYGTCTQSGERRREKRSVALTPHSGMGLETRAETWMSSEGA
WKHAQRVESWILRNPGFALLAGFMAYMIGQTGIQRTVFFVLMMLVAPSYGMRCVG
VGNRDFVEGVSGGAWVDLVLEHGGCVTTMAQGKPTLDFELTKTTAKEVALLRTYC
IEASISNITTATRCPTQGEPYLKEEQDQQYICRRDVVDRGWGNGCGLFGKGGVVTCA
KFSCSGKITGNLVQIENLEYTVVVTVHNGDTHAVGNDTSNHGVTATITPRSPSVEVK
LPDYGELTLDCEPRSGIDFNEMILMKMKKKTWLVHKQWFLDLPLPWTAGADTSEVH
WNYKERMVTFKVPHAKRQDVTVLGSQEGAMHSALAGATEVDSGDGNHMFAGHLK
CKVRMEKLRIKGVSYSLCTAAFTFTKIPAETLHGTVTVEVQYAGTDGPCKVPAQMA
VDMQTLTPVGRLITANPVITESTENSKMMLELDPPFGDSYIVIGVGEKKITHHWHRSG
STIGKMFESTYRGAKRMAILGETAWDFGSVGGLFTSLGKAVHQVFGSVYTTMFGGV
SWMIRILIGFLVLWIGTNSRNTSMAMTCIAVGGITLFLGFTVQADMGCVVSWSGREL
KCGSGIFVVDNVHTWTEQYKFQPESPARLASAILNAHKDGVCGIRSTTRLENVMWK
QITNELNYVLWEGGHDLTVVAGDVKGVLTKGKRALTPPVSDLKYSWKTWGKAKIF
TPEARNSTFLIDGPDTSECPNERRAWNSLEVEDYGFGMFTTNIWMKFREGSSEVCDH
RLMSAAIKDQKAVHADMGYWIESSKNQTWQIEKASLIEVKTCLWPKTHTLWSNGV
LESQMLIPKSYAGPFSQHNYRQGYATQTVGPWHLGKLEIDFGECPGTTVTIQEDCDH
RGPSLRTTTASGKLVTQWCCRSCTMPPLRFLGEDGCWYGMEIRPLSEKEENMVKSQ
VTAGQGTSETFSMGLLCLTLFVEECLRRRVTRIGIMILVVVITLCAIILGGLTWMDLLR
ALIMLGDTMSGRIGGQIHLAIMAVFKMSPGYVLGVFLRKLTSRETALMVIGMAMTT
VLSIPHDLMELIDGISLGLILLKIVTQFDNTQVGTLALSLTFIRSTMPLVMAWRTIMAV
LFVVTLIPLCRTSCLQKQSHWVEITALILGAQALPVYLMTLMKGASRRSWPLNEGIM
AVGLVSLLGSALLKNDVPLAGPMVAGGLLLAAYVMSGSSADLSLEKAANVQWDE
MADITGSSPIIEVKQDEDGSFSIRDVEETNMITLLVKLALITVSGLYPLAIPITMTLWY
MWQVKTQRSGALWDVPSPAATQKAALSEGVYRIMQRGLFGKTQVGVGIHMEGVF
HTMWHVTRGSVICHETGRLEPSWADVRNDMISYGGGWRLGDKWDKEEDVQVLAIE
PGKNPKHVQTKPGLFKTLTGEIGAVTLDFKPGTSGSPIINRKGKVIGLYGNGVVTKSG
DYVSAITQAERIGEPDYEVDEDIFRKKRLTIMDLHPGAGKTKRILPSIVREALKRRLRT
LILAPTRVVAAEMEEALRGLPIRYQTPAVKSEHTGREIVDLMCHATFTTRLLSSTRVP
NYNLIVMDEAHFTDPSSVAARGYISTRVEMGEAAAIFMTATPPGATDPFPQSNSPIEDI
EREIPERSWNTGFDWITDYQGKTVWFVPSIKAGNDIANCLRKSGKKVIQLSRKTFGTE
YPKTKLTDWDFVVTTDISEMGANFRAGRVIDPRRCLKPVILTDGPERVILAGPIPVTP
ASAAQRRGRVGRNPAQEDDQYVFSGDPLKNDEDHAHWTEAKMLLDNIYTPEGIIPT
LFGPEREKTQAIDGEFRLRGEQRKTFVELMRRGDLPVWLSYKVASAGISYKDREWCF
TGERNNQILEENMEVEIWTREGEKKKLRPRWLDARVYADPMALKDFKEFASGRKSI
TLDILTEIASLPAYLSSRAKLALDNIVMLHTTERGGRAYQHALNELPESLETLMLVAL
LGAMTAGIFLFFMQGKGIGKLSMGLITIAVASGLLWIAEIQPQWIAASIILEFFLMVLLI
PEPEKQRTPQDNQUYVILTVLTIIGLIAANEMGLIEKTKTDFGFYQVKTETTILDVDLR
PASAWTLYAVATTVLTPMLRHTIENTSANLSLAAIANQAAVLMGLGKGWPLHRMD
LGVPLLAMGCYSQVNPTTLTASLVMLFVHYAIIGPGLQAKATREAQKRTAAGIMKN
PTVDGITVIDLEPISYDPKFEKQLGQVMLLVLCAGQLLLMRTTWAFCEVLTLATGPIL
TLWEGNPGRFWNTTIAVSTANIFRGSYLAGAGLAFSLIKNAQTPRRGTGTTGETLGE
KWKRQLNSLDRKEFEEYKRSGILEVDRTEAKSALKDGSKIKHAVSRGSSKIRWIVER
GMVKPKGKVVDLGCGRGGWSYYMATLKNVTEVKGYTKGGPGHEEPIPMATYGWN
LVKLHSGVDVFYKPTEQVDTLLCDIGESSSNPTIEEGRTLRVLKMVEPWLSSKPEFCI
KVLNPYMPTVIEELEKLQRKHGGNLVRCPLSRNSTHEMYWVSGASGNIVSSVNTTSK
MLLNRFTTRHRKPTYEKDVDLGAGTRSVSTETEKPDMTIIGRRLQRLQEEHKETWHY
DQENPYRTWAYHGSYEAPSTGSASSMVNGVVKLLTKPWDVIPMVTQLAMTDTTPF
GQQRVFKEKVDTRTPQPKPGTRMVMTTTANWLWALLGKKKNPRLCTREEFISKVRS
NAAIGAVFQEEQGWTSASEAVNDSRFWELVDKERALHQEGKCESCVYNMMGKREK
KLGEFGRAKGSRAIWYMWLGARFLEFEALGFLNEDHWFGRENSWSGVEGEGLHRL
GYILEEIDKKDGDLMYADDTAGWDTRITEDDLQNEELITEQMAPHHKILAKAIFKLT
YQNKVVKVLRPTPRGAVMDIISRKDQRGSGQVGTYGLNTFTNMEVQLIRQMEAEGV
ITQDDMQNPKGLKERVEKWLRECGVDRLKRMAISGDDCVVKPLDERFGTSLLFLND
MGKVRKDIPQWEPSKGWKNWQEVPFCSHHFHKIFMKDGRSLVVPCRNQDELIGRA
RISQGAGWSLRETACLGKAYAQMWSLMYFHRRDLRLASMAICSAVPTEWFPTSRTT
WSIHAHHQWMTTEDMLKVWNRVWIEDNPNMTDKTPVHSWEDIPYLGKREDLWCG
SLIGLSSRATWAKNIHTAITQVRNLIGKEEYVDYMPVMKRYSAPSESEGVL
DENV/ZIKV Z20 Swap (SEQ ID NO: 10)
MNQRKKVVRPPFNMLKRERNRVSTPQGLVKRFSTGLFSGKGPLRMVLAFITFLRVLS
IPPTAGILKRWGQLKKNKAIKILIGFRKEIGRMLNILNGRKRSTITLLCLIPTVMAFHLS
TRDGEPLMIVAKHERGRPLLFKTTEGINKCTLIAMDLGEMCEDTVTYKCPLLVNTEP
EDIDCWCNLTSTWVMYGTCTQSGERRREKRSVALTPHSGMGLETRAETWMSSEGA
WKHAQRVESWILRNPGFALLAGFMAYMIGQTGIQRTVFFVLMMLVAPSYGMRCVG
VGNRDFVEGVSGGAWVDLVLEHGGCVTTMAQGKPTLDFELTKTTAKEVALLRTYC
IEASISDMATATRCPTQGEPYLKKEQDQQYICRRDVVDRGWGNGCGLFGKGGVVTC
AKFACSGKITGNLVQIENLEYTVVVTVHNGDTHAVGNDTSNHGVTATITPRSPSVEV
KLPDYGELTLDCEPRSGIDFNEMILMKMKNKTWLVHKQWFLDLPLPWTAGADTGT
VHWNYKERMVTFKVPHAKRQDVTVLGSQEGAMHSALAGATEVEMDGDGNHMFA
GHLKCKVRMEKLRIKGMSYTMCSGKFSIDKEMAETQHGTTVVKVKYEGAGAPCKV
PIEIRDVNKEKVVGRVISSTPLAENTNSVTNIELEPPFGDSYIVIGVGNSALTLHWFRK
GSSIGKMFESTYRGAKRMAILGETAWDFGSVGGLFTSLGKAVHQVFGSVYTTMFGG
VSWMIRILIGELVLWIGTNSRNTSMAMTCIAVGGITLFLGFTVQADMGCVVSWSGRE
LKCGSGIFVVDNVHTWTEQYKFQPESPARLASAILNAHKDGVCGIRSTTRLENVMW
KQITNELNYVLWEGGHDLTVVAGDVKGVLTKGKRALTPPVSDLKYSWKTWGKAKI
FTPEARNSTFLIDGPDTSECPNERRAWNSLEVEDYGFGMFTTNIWMKFREGSSEVCD
HRLMSAAIKDQKAVHADMGYWIESSKNQTWQIEKASLIEVKTCLWPKTHTLWSNG
VLESQMLIPKSYAGPFSQHNYRQGYATQTVGPWHLGKLEIDFGECPGTTVTIQEDCD
HRGPSLRTTTASGKLVTQWCCRSCTMPPLRFLGEDGCWYGMEIRPLSEKEENMVKS
QVTAGQGTSETFSMGLLCLTLFVEECLRRRVTRICHMILVVVITLCAIILGGLTWMDL
LRALIMLGDTMSGRIGGQIHLAIMAVFKMSPGYVLGVFLRKLTSRETALMVIGMAM
TTVLSIPHDLMELIDGISLGLILLKIVTQFDNTQVGTLALSLTFIRSTMPLVMAWRTIM
AVLFVVTLIPLCRTSCLQKQSHWVEITALILGAQALPVYLMTLMKGASRRSWPLNEG
IMAVGLVSLLGSALLKNDVPLAGPMVAGGLLLAAYVMSGSSADLSLEKAANVQWD
EMADITGSSPIIEVKQDEDGSFSIRDVEETNMITLLVKLALITVSGLYPLAIPITMTLWY
MWQVKTQRSGALWDVPSPAATQKAALSEGVYRIMQRGLFGKTQVGVGIHMEGVF
HTMWHVTRGSVICHETGRLEPSWADVRNDMISYGGGWRLGDKWDKEEDVQVLAIE
PGKNPKHVQTKPGLFKTLTGEIGAVTLDFKPGTSGSPIINRKGKVIGLYGNGVVTKSG
DYVSAITQAERIGEPDYEVDEDIFRKKRLTIMDLHPGAGKTKRILPSIVREALKRRLRT
LILAPTRVVAAEMEEALRGLPIRYQTPAVKSEHTGREIVDLMCHATFTTRLLSSTRVP
NYNLIVMDEAHFTDPSSVAARGYISTRVEMGEAAAIFMTATPPGATDPFPQSNSPIEDI
EREIPERSWNTGFDWITDYQGKTVWFVPSIKAGNDIANCLRKSGKKVIQLSRKTFGTE
YPKTKLTDWDFVVTTDISEMGANFRAGRVIDPRRCLKPVILTDGPERVILAGPIPVTP
ASAAQRRGRVGRNPAQEDDQYVFSGDPLKNDEDHAHWTEAKMLLDNIYTPEGIIPT
LFGPEREKTQAIDGEFRLRGEQRKTFVELMRRGDLPVWLSYKVASAGISYKDREWCF
TGERNNQILEENMEVEIWTREGEKKKLRPRWLDARVYADPMALKDFKEFASGRKSI
TLDILTEIASLPAYLSSRAKLALDNIVMLHTTERGGRAYQHALNELPESLETLMLVAL
LGAMTAGIFLFFMQGKGIGKLSMGLITIAVASGLLWIAEIQPQWIAASIILEFFLMVLLI
PEPEKQRTPQDNQUYVILTVLTIIGLIAANEMGLIEKTKTINGFYQVKTETTILDVDLR
PASAWTLYAVATTVLTPMLRHTIENTSANLSLAAIANQAAVLMGLGKGWPLHRMD
LGVPLLAMGCYSQVNPTTLTASLVMLFVHYAIIGPGLQAKATREAQKRIAAG1MKN
PTVDGITVIDLEPISYDPKFEKQLGQVMLLVLCAGQLLLMRTTWAFCEVLTLATGPIL
TLWEGNPGRFWNTTIAVSTANIFRGSYLAGAGLAFSLIKNAQTPRRGTGTTGETLGE
KWKRQLNSLDRKEFEEYKRSGILEVDRTEAKSALKDGSKIKHAVSRGSSKIRWIVER
GMVKPKGKVVDLGCGRGGWSYYMATLKNVTEVKGYTKGGPGHEEPIPMATYGWN
LVKLHSGVDVFYKPTEQVDTLLCDIGESSSNPTIEEGRTLRVLKMVEPWLSSKPEFCI
KVLNPYMPTVIEELEKLQRKHGGNLVRCPLSRNSTHEMYWVSGASGNIVSSVNTTSK
MLLNRFTTRHRKPTYEKDVDLGAGTRSVSTETEKPDMTIIGRRLQRLQEEHKETWHY
DQENPYRTWAYHGSYEAPSTGSASSMVNGVVKLLTKPWDVIPMVTQLAMTDTTPF
GQQRVFKEKVDTRTPQPKPGTRMVMTTTANWLWALLGKKKNPRLCTREEFISKVRS
NAAIGAVFQEEQGWTSASEAVNDSRFWELVDKERALHQEGKCESCVYNMMGKREK
KLGEFGRAKGSRAIWYMWLGARFLEFEALGFLNEDHWFGRENSWSGVEGEGLHRL
GYILEEIDKKDGDLMYADDTAGWDTRITEDDLQNEELITEQMAPHHKILAKAIFKLT
YQNKVVKVLRPTPRGAVMDIISRKDQRGSGQVGTYGLNTFTNMEVQLIRQMEAEGV
ITQDDMQNPKGLKERVEKWLRECGVDRLKRMAISGDDCVVKPLDERFGTSLLFLND
MGKVRKDIPQWEPSKGWKNWQEVPFCSHHFHKIFMKDGRSLVVPCRNQDELIGRA
RISQGAGWSLRETACLGKAYAQMWSLMYHIRRDLRLASMAICSAVPTEWETTSRTT
WSIHAHHQWMTTEDMLKVWNRVWIEDNPNMTDKTPVHSWEDIPYLGKREDLWCG
SLIGLSSRATWAKNIHTAITQVRNLIGKEEYVDYMPVMKRYSAPSESEGVL
DENV/ZIKV Z3L1 Swap (SEQ ID NO: 11)
MNQRKKVVRPPFNMLKRERNRVSTPQGLVKRFSTGLFSGKGPLRMVLAFITFLRVLS
IPPTAGILKRWGQLKKNKAIKILIGFRKEIGRMLNILNGRKRSTITLLCLIPTVMAFHLS
TRDGEPLMIVAKHERGRPLLFKTTEGINKCTLIAMDLGEMCEDTVTYKCPLLVNTEP
EDIDCWCNLTSTWVMYGTCTQSGERRREKRSVALTPHSGMGLETRAETWMSSEGA
WKHAQRVESWILRNPGFALLAGFMAYMIGQTGIQRTVFFVLMMLVAPSYGMRCVG
VGNRDFVEGVSGGAWVDLVLEHGGCVTTMAQGKPTLDFELVTTTAKNVALLRTYC
IEASISNITTATRCPTQGEPYLKEEQDQQYICRRDVVDRGWGNGCGLFGKGGVVTCA
KFSCSGKITGNLVQIENLEYRVMVTVHNGDTHATGHETSNHGVKAEITPRSPSVEVK
LPDYGELTLDCEPRSGIDFNEMILMKMKNKTWLVHKQWFLDLPLPWTAGADTSEVH
WNYKERMVTFKVPHAKRQDVTVLGSQEGAMHSALAGATEVEMDGAKNRMFAGH
LKCKVRMEKLRIKGMSYTMCSGKFSIDKEMAETQHGTTVVKVKYEGAGAPCKVPIE
IRDVNKEKVVGRVISSTPLAENTNSVTNIELEPPFGDSYIVIGVGNSALTLHWFRKGSS
IGKMFESTYRGAKRMAILGETAWDFGSVGGLFTSLGKAVHQVFGSVYTTMFGGVS
WMIRILIGFLVLWIGTNSRNTSMAMTCIAVGGITLFLGFIVQADMGCVVSWSGRELK
CGSGIFVVDNVHTWTEQYKFQPESPARLASAILNAHKDGVCGIRSTTRLENVMWKQI
TNELNYVLWEGGHDLTVVAGDVKGVLTKGKRALTPPVSDLKYSWKTWGKAKIFTP
EARNSTFLIDGPDTSECPNERRAWNSLEVEDYGFGMFTTNIWMKFREGSSEVCDHRL
MSAAIKDQKAVHADMGYWIESSKNQTWQIEKASLIEVKTCLWPKTHTLWSNGVLES
QMLIPKSYAGPFSQHNYRQGYATQTVGPWHLGKLEIDFGECPGTTVTIQEDCDHRGP
SLRTTTASGKLVTQWCCRSCTMPPLRFLGEDGCWYGMEIRPLSEKEENMVKSQVTA
GQGTSETFSMGLLCLTLFVEECLRRRVTRKHMILVVVITLCAIILGGLTWMDLLRALI
MLGDTMSGRIGGQIHLAIMAVFKMSPGYVLGVFLRKLTSRETALMVIGMAMTTVLSI
PHDLMELIDGISLGLILLKIVTQFDNTQVGTLALSLTFIRSTMPLVMAWRTIMAVLFV
VTLIPLCRTSCLQKQSHWVEITALILGAQALPVYLMTLMKGASRRSWPLNEGIMAVG
LVSLLGSALLKNDVPLAGPMVAGGLLLAAYVMSGSSADLSLEKAANVQWDEMADI
TGSSPIIEVKQDEDGSFSIRDVEETNMITLLVKLALITVSGLYPLAIPITMTLWYMWQV
KTQRSGALWDVPSPAATQKAALSEGVYRIMQRGLFGKTQVGVGIHMEGVFHTMWH
VTRGSVICHETGRLEPSWADVRNDMISYGGGWRLGDKWDKEEDVQVLAIEPGKNP
KHVQTKPGLFKTLTGEIGAVTLDFKPGTSGSPIINRKGKVIGLYGNGVVTKSGDYVSA
ITQAERIGEPDYEVDEDIFRKKRLTIMDLHPGAGKTKRILPSIVREALKRRLRTLILAPT
RVVAAEMEEALRGLPIRYQTPAVKSEHTGREIVDLMCHATFTTRLLSSTRVPNYNLIV
MDEAHFTDPSSVAARGYISTRVEMGEAAAIFMTATPPGATDPFPQSNSPIEDIEREIPE
RSWNTGFDWITDYQGKTVWFVPSIKAGNDIANCLRKSGKKVIQLSRKTFGTEYPKTK
LTDWDFVVTTDISEMGANFRAGRVIDPRRCLKPVILTDGPERVILAGPIPVTPASAAQ
RRGRVGRNPAQEDDQYVFSGDPLKNDEDHAHWTEAKMLLDNIYTPEGIIPTLFGPER
EKTQAIDGEFRLRGEQRKTFVELMRRGDLPVWLSYKVASAGISYKDREWCFTGERN
NQILEENMEVEIWTREGEKKKLRPRWLDARVYADPMALKDFKEFASGRKSITLDILT
EIASLPAYLSSRAKLALDNIVMLHTTERGGRAYQHALNELPESLETLMLVALLGAMT
AGIFLFFMQGKGIGKLSMGLITIAVASGLLWIAEIQPQWIAASIILEFFLMVLLIPEPEK
QRTPQDNQLIYVILTVLTIIGLIAANEMGLIEKTKTDFGFYQVKTETTILDVDLRPASA
WTLYAVATTVLTPMLRHTIENTSANLSLAAIANQAAVLMGLGKGWPLHRMDLGVP
LLAMGCYSQVNPTTLTASLVMLFVHYAIIGPGLQAKATREAQKRTAAGIMKNPTVD
GITVIDLEPISYDPKFEKQLGQVMLLVLCAGQLLLMRTTWAFCEVLTLATGPILTLWE
GNPGRFWNTTIAVSTANIFRGSYLAGAGLAFSLIKNAQTPRRGTGTTGETLGEKWKR
QLNSLDRKEFEEYKRSGILEVDRTEAKSALKDGSKIKHAVSRGSSKIRWIVERGMVK
PKGKVVDLGCGRGGWSYYMATLKNVTEVKGYTKGGPGHEEPIPMATYGWNLVKL
HSGVDVFYKPTEQVDTLLCDIGESSSNPTIEEGRTLRVLKMVEPWLSSKPEFCIKVLN
PYMPTVIEELEKLQRKHGGNLVRCPLSRNSTHEMYWVSGASGNIVSSVNTTSKMLL
NRFTTRHRKPTYEKDVDLGAGTRSVSTETEKPDMTIIGRRLQRLQEEHKETWHYDQE
NPYRTWAYHGSYEAPSTGSASSMVNGVVKLLTKPWDVIPMVTQLAMTDTTPFGQQ
RVFKEKVDTRTPQPKPGTRMVMTTTANWLWALLGKKKNPRLCTREEFISKVRSNAA
IGAVFQEEQGWTSASEAVNDSRFWELVDKERALHQEGKCESCVYNMMGKREKKLG
EFGRAKGSRAIWYMWLGARFLEFEALGFLNEDHWFGRENSWSGVEGEGLHRLGYIL
EEIDKKDGDLMYADDTAGWDTRITEDDLQNEELITEQMAPHHKILAKAIFKLTYQNK
VVKVLRPTPRGAVMDIISRKDQRGSGQVGTYGLNTFTNMEVQLIRQMEAEGVITQD
DMQNPKGLKERVEKWLRECGVDRLKRMAISGDDCVVKPLDERFGTSLLFLNDMGK
VRKDIPQWEPSKGWKNWQEVPFCSHHFHKIFMKDGRSLVVPCRNQDELIGRARISQ
GAGWSLRETACLGKAYAQMWSLMYFHRRDLRLASMAICSAVPTEWFPTSRTTWSI
HAHHQWMTTEDMLKVWNRVWIEDNPNMTDKTPVHSWEDIPYLGKREDLWCGSLI
GLSSRATWAKNIHTAITQVRNLIGKEEYVDYMPVMKRYSAPSESEGVL
DENV/ZIKV Z20/Z3L1 Double Swap (SEQ ID NO: 12)
MNQRKKVVRPPFNMLKRERNRVSTPQGLVKRFSTGLFSGKGPLRMVLAFITFLRVLS
IPPTAGILKRWGQLKKNKAIKILIGFRKEIGRMLNILNGRKRSTITLLCLIPTVMAFHLS
TRDGEPLMIVAKHERGRPLLFKTTEGINKCTLIAMDLGEMCEDTVTYKCPLLVNTEP
EDIDCWCNLTSTWVMYGTCTQSGERRREKRSVALTPHSGMGLETRAETWMSSEGA
WKHAQRVESWILRNPGFALLAGFMAYMIGQTGIQRTVFFVLMMLVAPSYGMRCVG
VGNRDFVEGVSGGAWVDLVLEHGGCVTTMAQGKPTLDFELVTTTAKNVALLRTYC
IEASISDMATATRCPTQGEPYLKKEQDQQYICRRDVVDRGWGNGCGLFGKGGVVTC
AKFACSGKITGNLVQIENLEYRVMVTVHNGDTHATGHETSNHGVKAEITPRSPSVEV
KLPDYGELTLDCEPRSGIDFNEMILMKMKNKTWLVHKQWFLDLPLPWTAGADTGT
VHWNYKERMVTFKVPHAKRQDVTVLGSQEGAMHSALAGATEVEMDGAKNRMFA
GHLKCKVRMEKLRIKGMSYTMCSGKFSIDKEMAETQHGTTVVKVKYEGAGAPCKV
PIEIRDVNKEKVVGRVISSTPLAENTNSVTNIELEPPFGDSYIVIGVGNSALTLHWFRK
GSSIGKMFESTYRGAKRMAILGETAWDFGSVGGLFTSLGKAVHQVFGSVYTTMFGG
VSWMIRILIGFLVLWIGTNSRNTSMAMTCIAVGGITLFLGFTVQADMGCVVSWSGRE
LKCGSGIFVVDNVHTWTEQYKFQPESPARLASAILNAHKDGVCGIRSTTRLENVMW
KQITNELNYVLWEGGHDLTVVAGDVKGVLTKGKRALTPPVSDLKYSWKTWGKAKI
FTPEARNSTFLIDGPDTSECPNERRAWNSLEVEDYGFGMFTTNIWMKFREGSSEVCD
HRLMSAAIKDQKAVHADMGYWIESSKNQTWQIEKASLIEVKTCLWPKTHTLWSNG
VLESQMLIPKSYAGPFSQHNYRQGYATQTVGPWHLGKLEIDFGECPGTTVTIQEDCD
HRGPSLRTTTASGKLVTQWCCRSCTMPPLRFLGEDGCWYGMEIRPLSEKEENMVKS
QVTAGQGTSETFSMGLLCLTLFVEECLRRRVTRKHMILVVVITLCAIILGGLTWMDL
LRALIMLGDTMSGRIGGQIHLAIMAVFKMSPGYVLGVFLRKLTSRETALMVIGMAM
TTVLSIPHDLMELIDGISLGLILLKIVTQFDNTQVGTLALSLTFIRSTMPLVMAWRTIM
AVLFVVTLIPLCRTSCLQKQSHWVEITALILGAQALPVYLMTLMKGASRRSWPLNEG
IMAVGLVSLLGSALLKNDVPLAGPMVAGGLLLAAYVMSGSSADLSLEKAANVQWD
EMADITGSSPIIEVKQDEDGSFSIRDVEETNMITLLVKLALITVSGLYPLAIPITMTLWY
MWQVKTQRSGALWDVPSPAATQKAALSEGVYRIMQRGLFGKTQVGVGIHMEGVF
HTMWHVTRGSVICHETGRLEPSWADVRNDMISYGGGWRLGDKWDKEEDVQVLAIE
PGKNPKHVQTKPGLFKTLTGEIGAVTLDFKPGTSGSPIINRKGKVIGLYGNGVVTKSG
DYVSAITQAERIGEPDYEVDEDIFRKKRLTIMDLHPGAGKTKRILPSIVREALKRRLRT
LILAPTRVVAAEMEEALRGLPIRYQTPAVKSEHTGREIVDLMCHATFTTRLLSSTRVP
NYNLIVMDEAHFTDPSSVAARGYISTRVEMGEAAAIFMTATPPGATDPFPQSNSPIEDI
EREIPERSWNTGFDWITDYQGKTVWFVPSIKAGNDIANCLRKSGKKVIQLSRKTFGTE
YPKTKLTDWDFVVTTDISEMGANFRAGRVIDPRRCLKPVILTDGPERVILAGPIPVTP
ASAAQRRGRVGRNPAQEDDQYVFSGDPLKNDEDHAHWTEAKMLLDNIYTPEGIIPT
LFGPEREKTQAIDGEFRLRGEQRKTFVELMRRGDLPVWLSYKVASAGISYKDREWCF
TGERNNQILEENMEVEIWTREGEKKKLRPRWLDARVYADPMALKDFKEFASGRKSI
TLDILTEIASLPAYLSSRAKLALDNIVMLHTTERGGRAYQHALNELPESLETLMLVAL
LGAMTAGIFLFFMQGKGIGKLSMGLITIAVASGLLWIAEIQPQWIAASIILEFFLMVLLI
PEPEKQRTPQDNQLIYVILTVLTIIGLIAANEMGLIEKTKTDFGFYQVKTETTILDVDLR
PASAWTLYAVATTVLTPMLRHTIENTSANLSLAAIANQAAVLMGLGKGWPLHRMD
LGVPLLAMGCYSQVNPTTLTASLVMLFVHYAIIGPGLQAKATREAQKRTAAGIMKN
PTVDGITVIDLEPISYDPKFEKQLGQVMLLVLCAGQLLLMRTTWAFCEVLTLATGPIL
TLWEGNPGRFWNTTIAVSTANIFRGSYLAGAGLAFSLIKNAQTPRRGTGTTGETLGE
KWKRQLNSLDRKEFEEYKRSGILEVDRTEAKSALKDGSKIKHAVSRGSSKIRWIVER
GMVKPKGKVVDLGCGRGGWSYYMATLKNVTEVKGYTKGGPGHEEPIPMATYGWN
LVKLHSGVDVFYKPTEQVDTLLCDIGESSSNPTIEEGRTLRVLKMVEPWLSSKPEFCI
KVLNPYMPTVIEELEKLQRKHGGNLVRCPLSRNSTHEMYWVSGASGNIVSSVNTTSK
MLLNRFTTRHRKPTYEKDVDLGAGTRSVSTETEKPDMTIIGRRLQRLQEEHKETWHY
DQENPYRTWAYHGSYEAPSTGSASSMVNGVVKLLTKPWDVIPMVTQLAMTDTTPF
GQQRVFKEKVDTRTPQPKPGTRMVMTTTANWLWALLGKKKNPRLCTREEFISKVRS
NAAIGAVFQEEQGWTSASEAVNDSRFWELVDKERALHQEGKCESCVYNMMGKREK
KLGEFGRAKGSRAIWYMWLGARFLEFEALGFLNEDHWFGRENSWSGVEGEGLHRL
GYILEEIDKKDGDLMYADDTAGWDTRITEDDLQNEELITEQMAPHHKILAKAIFKLT
YQNKVVKVLRPTPRGAVMDIISRKDQRGSGQVGTYGLNTFTNMEVQLIRQMEAEGV
ITQDDMQNPKGLKERVEKWLRECGVDRLKRMAISGDDCVVKPLDERFGTSLLFLND
MGKVRKDIPQWEPSKGWKNWQEVPFCSHHFHKIFMKDGRSLVVPCRNQDELIGRA
RISQGAGWSLRETACLGKAYAQMWSLMYFHRRDLRLASMAICSAVPTEWFPTSRTT
WSIHAHHQWMTTEDMLKVWNRVWIEDNPNMTDKTPVHSWEDIPYLGKREDLWCG
SLIGLSSRATWAKNIHTAITQVRNLIGKEEYVDYMPVMKRYSAPSESEGVL
Flaviviral E Glycoprotein Epitope Swaps
ZikV Backbone with DENV1 “5J7” Homologous epitope (SEQ ID NO: 13)
IRCIGVSNRDFVEGMSGGTWVDVVLEHGGCVTVMAQDKPTVDIELVTTEVTNPAVL
RKLCIEASISDMASDSRCPTQGEAYLDKQSDTQYVCKRTLVDRGWGNGCGLFGKGS
LVTCAKFACVTKLEGKIVQYENLEYRIMLSVHGSQHSGMIVNDTGHETDENRAKVEI
TPNSPRAEATLGGFGSLGLDCEPRTGLDFSEMVLLTMENKSWLVHKQWFLDLPLPW
TSGASTSTPTWNNKEALVEFKDAHAKRQTVVVLGSQEGAVHTALAGALEIQTSGTT
TIFAGHLKCRLKMDKLRLKGVSYSLCTAAFTFTKIPAETLHGTVTVEVQYAGTDGPC
KVPAQMAVDMQTLTPVGRLITANPVITESTENSKMMLELDPPFGDSYIVIGVGEKKIT
HHWHRSG
ZikV Backbone with DENV2 “5J7” Homologous epitope (SEQ ID NO: 14)
IRCIGVSNRDFVEGMSGGTWVDVVLEHGGCVTVMAQDKPTVDIELVTTEAKQPATL
RKYCIEASISDMASDSRCPTQGEAYLDKQSDTQYVCKRTLVDRGWGNGCGLFGKGS
LVTCAKFACKKNMEGKVVQPENLEYRIMLSVHGSQHSGMIVNDTGHETDENRAKV
EITPNSPRAEATLGGFGSLGLDCEPRTGLDFSEMVLLTMENKAWLVHRQWFLDLPLP
WLPGADTQTPNWNNKEALVEFKDAHAKRQTVVVLGSQEGAVEITALAGALEIQMSS
GNLLFTGHLKCRLKMDKLRLKGVSYSLCTAAFTFTKIPAETLHGTVTVEVQYAGTD
GPCKVPAQMAVDMQTLTPVGRLITANPVITESTENSKMMLELDPPFGDSYIVIGVGE
KKITHHWHRSG
ZikV Backbone with DENV3 5J7 epitope (SEQ ID NO: 15)
IRCIGVSNRDFVEGMSGGTWVDVVLEHGGCVTVMAQDKPTVDIELVTTEATQLATL
RKLCIEGSISDMASDSRCPTQGEAYLDKQSDTQYVCKRTLVDRGWGNGCGLFGKGS
LVTCAKFACLEPIEGKVVQYENLEYRIMLSVHGSQHSGMIVNDTGHETDENRAKVEI
TPNSPRAEATLGGFGSLGLDCEPRTGLDFSEMIILTMKNKAWMVHRQWFFDLPLPW
TSGATTETPTWNNKEALVEFKDAHAKRQTVVVLGSQEGAVHTALAGALEIQNSGGT
SIFAGHLKCRLKMDKLRLKGVSYSLCTAAFTFTKIPAETLHGTVTVEVQYAGTDGPC
KVPAQMAVDMQTLTPVGRLITANPVITESTENSKMMLELDPPFGDSYIVIGVGEKKIT
HHWHRSG
ZikV Backbone with DENV4 “5J7” Homologous epitope (SEQ ID NO: 16)
IRCIGVSNRDFVEGMSGGTWVDVVLEHGGCVTVMAQDKPTVDIELVTTTAKEVALL
RTYCIEASISDMASDSRCPTQGEAYLDKQSDTQYVCKRTLVDRGWGNGCGLFGKGS
LVTCAKFACSGKITGNLVQIENLEYRIMLSVHGSQHSGMIVNDTGHETDENRAKVEI
TPNSPRAEATLGGFGSLGLDCEPRTGLDFSEMILMKMKKKTWLVHKQWFLDLPLPW
TAGADTSEVHWNNKEALVEFKDAHAKRQTVVVLGSQEGAVHTALAGALEVDSGD
GNHMFAGHLKCRLKMDKLRLKGVSYSLCTAAFTFTKIPAETLHGTVTVEVQYAGTD
GPCKVPAQMAVDMQTLTPVGRLITANPVITESTENSKMMLELDPPFGDSYIVIGVGE
KKITHHWHRSG
DENV1 Backbone with 5J7 Homologous ZikV epitope (SEQ ID NO: 17)
MRCVGIGNRDFVEGLSGATWVDVVLEHGSCVTTMAKDKPTLDIELLKTTVSNMAE
VRSYCYEAKISNTTTDSRCPTQGEATLVEEQDTNFVCRRTFVDRGWGNGCGLFGKG
SLITCAKFKCSKKMTGKSIQPENLKYSVIVTVHTGDQHQVGNETTEHGTTATITPQAP
TSEIQLTDYGALTLDCSPRTGLDFNDLYLLTMNNKHWLVHKEWFHDIPLPWHAGAD
TGTPHWNRQDLLVTFKTAHAKKQEVVVLGSQEGAMHTALTGATEAEMDGAKGRL
SSGHLKCRLKMDKLTLKGMSYVMCTGSFKLEKEVAETQHGTVLVQVKYEGTDAPC
KIPFSSQDEKGVTQNGRLITANPIVTDKEKPVNIEAEPPFGESYIVVGAGEKALKLSWF
KK
DENV2 Backbone with 5J7 Homologous ZikV epitope (SEQ ID NO: 18)
MRCIGISNRDFVEGVSGGSWVDIVLEHGSCVTTMAKNKPTLDFELIKTTVSNMAEVR
SYCYEAKLTNTTTESRCPTQGEPSLNEEQDKRFVCKHSMVDRGWGNGCGLFGKGGI
VTCAMFTCSKKMTGKSIQPENLEYTIVVTPHSGEEHAVGNDTGKHGKEIKVTPQSSIT
EAELTGYGTVTMECSPRTGLDFNDLYLLTMNNKHWLVHKEWFHDIPLPWHAGADT
GTPHWIQKETLVTFKNPHAKKQDVVVLGSQEGAMHTALTGATEAEMDGAKGRLSS
GHLKCRLRMDKLQLKGMSYSMCTGKFKVVKEIAETQHGTIVIRVQYEGDGSPCKIPF
EIMDLEKRHVLGRLITVNPIVTEKDSPVNIEAEPPFGDSYIIIGVDPGQLKLNWFKK
DENV3 Backbone with 5J7 Homologous ZikV epitope (SEQ ID NO: 19)
MRCVGIGNRDFVEGLSGATWVDVVLEHGGCVTTMAKNKPTLDIELQKTTVSNMAE
VRSYCYEAKITNITTDSRCPTQGEAVLPEEQDQNYVCKHTYVDRGWGNGCGLFGKG
SLVTCAKFQCSKKMTGKSIQPENLKYTVIITVHTGDQHQVGNETQGVTAEITPQASTT
EAILPEYGTLGLECSPRTGLDFNDLYLLTMNNKHWLVHKEWFHDIPLPWHAGADTG
TPHWNRKELLVTFKNAHAKKQEVVVLGSQEGAMHTALTGATEAEMDGAKGRLSS
GHLKCRLKMDKLELKGMSYAMCTNTFVLKKEVSETQHGTILIKVEYKGEDAPCKIPF
STEDGQGKAHNGRLITANPVVTKKEEPVNIEARREPPFGESNIVIGIGDNALKINWYK
K
DENV4 Backbone with 5J7 Homologous ZikV epitope (SEQ ID NO: 20)
MRCVGVGNRDFVEGVSGGAWVDLVLEHGGCVTTMAQGKPTLDFELTKTTVSNMA
EVRSYCYEASISNITTATRCPTQGEPYLKEEQDQQYICRRDVVDRGWGNGCGLFGKG
GVVTCAKFSCSKKMTGKSIQPENLEYTVVVTVHNGDTHAVGNDTSNHGVTATITPR
SPSVEVKLPDYGELTLDCEPRSGIDFNDLYLLTMNNKHWLVHKEWFHDIPLPWHAG
ADTGTPHWNYKERMVTFKVPHAKRQDVTVLGSQEGAMHSALAGATEAEMDGAKG
RLSSGHLKCKVRMEKLRIKGMSYTMCSGKFSIDKEMAETQHGTTVVKVKYEGAGA
PCKVPIEIRDVNKEKVVGRVISSTPLAENTNSVTNIELEPPFGDSYIVIGVGNSALTLH
WFRK
DENV1 Backbone with EDIII from ZikV (SEQ ID NO: 21)
MRCVGIGNRDFVEGLSGATWVDVVLEHGSCVTTMAKDKPTLDIELLKTEVTNPAVL
RKLCIEAKISNTTTDSRCPTQGEATLVEEQDTNFVCRRTFVDRGWGNGCGLFGKGSLI
TCAKFKCVTKLEGKIVQYENLKYSVIVTVHTGDQHQVGNETTEHGTTATITPQAPTS
EIQLTDYGALTLDCSPRTGLDFNEMVLLTMEKKSWLVHKQWFLDLPLPWTSGASTS
QETWNRQDLLVTFKTAHAKKQEVVVLGSQEGAMHTALTGATEIQTSGTTTIFAGHL
KCRLKMDKLTLKGMSYSLCTAAFTFTKIPAETLHGTVTVEVQYAGTDGPCKVPAQM
AVDMQTLTPVGRLITANPVITESTENSKMMLELDPPFGDSYIVIGVGEKKITHHWHRS
G
DENV2 Backbone with EDIII from ZikV (SEQ ID NO: 22)
MRCIGISNRDFVEGVSGGSWVDIVLEHGSCVTTMAKNKPTLDFELIKTEAKQPATLR
KYCIEAKLTNTTTESRCPTQGEPSLNEEQDKRFVCKHSMVDRGWGNGCGLFGKGGI
VTCAMFTCKKNMEGKVVQPENLEYTIVVTPHSGEEHAVGNDTGKHGKEIKVTPQSSI
TEAELTGYGTVTMECSPRTGLDFNEMVLLQMENKAWLVHRQWFLDLPLPWLPGAD
TQGSNWIQKETLVTFKNPHAKKQDVVVLGSQEGAMHTALTGATEIQMSSGNLLFTG
HLKCRLRMDKLQLKGMSYSLCTAAFTFTKIPAETLHGTVTVEVQYAGTDGPCKVPA
QMAVDMQTLTPVGRLITANPVITESTENSKMMLELDPPFGDSYIVIGVGEKKITHHW
HRSG
DENV3 Backbone with EDIII from ZikV (SEQ ID NO: 23)
MRCVGIGNRDFVEGLSGATWVDVVLEHGGCVTTMAKNKPTLDIELQKTEATQLATL
RKLCIEGKITNITTDSRCPTQGEAVLPEEQDQNYVCKHTYVDRGWGNGCGLFGKGSL
VTCAKFQCLEPIEGKVVQYENLKYTVIITVHTGDQHQVGNETQGVTAEITPQASTTE
AILPEYGTLGLECSPRTGLDFNEMILLTMKNKAWMVHRQWFFDLPLPWTSGATTETP
TWNRKELLVTFKNAHAKKQEVVVLGSQEGAMHTALTGATEIQNSGGTSIFAGHLKC
RLKMDKLELKGMSYSLCTAAFTFTKIPAETLHGTVTVEVQYAGTDGPCKVPAQMAV
DMQTLTPVGRLITANPVITESTENSKMMLELDPPFGDSYIVIGVGEKKITHHWHRSG
DENV4 Backbone with EDIII from ZikV (version 1) (SEQ ID NO: 24)
MRCVGVGNRDFVEGVSGGAWVDLVLEHGGCVTTMAQGKPTLDFELTKTTAKEVA
LLRTYCIEASISNITTATRCPTQGEPYLKEEQDQQYICRRDVVDRGWGNGCGLFGKG
GVVTCAKFSCSGKITGNLVQIENLEYTVVVTVHNGDTHAVGNDTSNHGVTATITPRS
PSVEVKLPDYGELTLDCEPRSGIDFNEMILMKMKKKTWLVHKQWFLDLPLPWTAGA
DTSEVHWNYKERMVTFKVPHAKRQDVTVLGSQEGAMHSALAGATEVDSGDGNHM
FAGHLKCKVRMEKLRIKGVSYSLCTAAFTFTKIPAETLHGTVTVEVQYAGTDGPCKV
PAQMAVDMQTLTPVGRLITANPVITESTENSKMMLELDPPFGDSYIVIGVGEKKITHH
WHRSG
DENV4 Backbone with EDIII from ZikV (version 2) (SEQ ID NO: 97)
MRCVGVGNRDFVEGVSGGAWVDLVLEHGGCVTTMAQGKPTLDFELTKTTAKEVA
LLRTYCIEASISNITTATRCPTQGEPYLKEEQDQQYICRRDVVDRGWGNGCGLFGKG
GVVTCAKFSCSGKITGNLVQIENLEYTVVVTVHNGDTHAVGNDTSNHGVTATITPRS
PSVEVKLPDYGELTLDCEPRSGIDFNEMILMKMKKKTWLVHKQWFLDLPLPWTAGA
DTSEVHWNYKERMVTFKVPHAKRQDVTVLGSQEGAMHSALAGATEVDSGDGNHM
FAGHLKCKVRMEKLRIKGVSYSLCTAAFTFTKIPAETLHGTVTVEVQYAGTDGPCKV
PAQMAVDMQTLTPVGRLITANPVITESTENSKMMLELDPPFGDSYIVIGVGEKKITHH
WHRSG
DENV1 Backbone with ZIKV Z20 Epitope (SEQ ID NO: 25)
MRCVGIGNRDFVEGLSGATWVDVVLEHGSCVTTMAKDKPTLDIELLKTEVTNPAVL
RKLCIEAKISDMATDSRCPTQGEATLVKEQDTNFVCRRTFVDRGWGNGCGLFGKGS
LITCAKFACVTKLEGKIVQYENLKYSVIVTVHTGDQHQVGNETTEHGTTATITPQAPT
SEIQLTDYGALTLDCSPRTGLDFNEMVLLTMENKSWLVHKQWFLDLPLPWTSGAST
GTETWNRQDLLVTFKTAHAKKQEVVVLGSQEGAMHTALTGATEIEMDSGTTTIFAG
HLKCRLKMDKLTLKGMSYVMCTGSFKLEKEVAETQHGTVLVQVKYEGTDAPCKIP
FSSQDEKGVTQNGRLITANPIVTDKEKPVNIEAEPPFGESYIVVGAGEKALKLSWFKK
DENV2 Backbone with ZIKV Z20 Epitope (SEQ ID NO: 26)
MRCIGISNRDFVEGVSGGSWVDIVLEHGSCVTTMAKNKPTLDFELIKTEAKQPATLR
KYCIEAKLTDMATESRCPTQGEPSLNKEQDKRFVCKHSMVDRGWGNGCGLFGKGGI
VTCAMFACKKNMEGKVVQPENLEYTIVVTPHSGEEHAVGNDTGKHGKEIKVTPQSS
ITEAELTGYGTVTMECSPRTGLDFNEMVLLQMENKAWLVHRQWFLDLPLPWLPGA
DTGTSNWIQKETLVTFKNPHAKKQDVVVLGSQEGAMHTALTGATEIEMDSSGNLLF
TGHLKCRLRMDKLQLKGMSYSMCTGKFKVVKEIAETQHGTIVIRVQYEGDGSPCKIP
FEIMDLEKRHVLGRLITVNPIVTEKDSPVNIEAEPPFGDSYIIIGVDPGQLKLNWFKK
DENV3 Backbone with ZIKV Z20 Epitope (SEQ ID NO: 27)
MRCVGIGNRDFVEGLSGATWVDVVLEHGGCVTTMAKNKPTLDIELQKTEATQLATL
RKLCIEGKITDMATDSRCPTQGEAVLPKEQDQNYVCKHTYVDRGWGNGCGLFGKG
SLVTCAKFACLEPIEGKVVQYENLKYTVIITVHTGDQHQVGNETQGVTAEITPQASTT
EAILPEYGTLGLECSPRTGLDFNEMILLTMKNKAWMVHRQWFFDLPLPWTSGATTG
TPTWNRKELLVTFKNAHAKKQEVVVLGSQEGAMHTALTGATEIEMDSGGTSIFAGH
LKCRLKMDKLELKGMSYAMCTNTFVLKKEVSETQHGTILIKVEYKGEDAPCKIPFST
EDGQGKAHNGRLITANPVVTKKEEPVNIEAEPPFGESNIVIGIGDNALKINWYKK
DENV4 Backbone with ZIKV Z20 Epitope (version 1) (SEQ ID NO: 28)
MRCVGVGNRDFVEGVSGGAWVDLVLEHGGCVTTMAQGKPTLDFELTKTTAKEVA
LLRTYCIEASISDMATATRCPTQGEPYLKKEQDQQYICRRDVVDRGWGNGCGLFGK
GGVVTCAKFACSGKITGNLVQIENLEYTVVVTVHNGDTHAVGNDTSNHGVTATITP
RSPSVEVKLPDYGELTLDCEPRSGIDFNEMILMKMKNKTWLVHKQWFLDLPLPWTA
GADTGTVHWNYKERMVTFKVPHAKRQDVTVLGSQEGAMHSALAGATEVEMDGD
GNHMFAGHLKCKVRMEKLRIKGMSYTMCSGKFSIDKEMAETQHGTTVVKVKYEGA
GAPCKVPIEIRDVNKEKVVGRVISSTPLAENTNSVTNIELEPPFGDSYIVIGVGNSALTL
HWFRK
DENV4 Backbone with ZIKV Z20 Epitope (version 2) (SEQ ID NO: 96)
MRCVGVGNRDFVEGVSGGAWVDLVLEHGGCVTTMAQGKPTLDFELTKTTAKEVA
LLRTYCIEASISDMATATRCPTQGEPYLKKEQDQQYICRRDVVDRGWGNGCGLFGK
GGVVTCAKFACSGKITGNLVQIENLEYTVVVTVHNGDTHAVGNDTSNHGVTATITP
RSPSVEVKLPDYGELTLDCEPRSGIDFNEMILMKMKNKTWLVHKQWFLDLPLPWTA
GADTGTVHWNYKERMVTFKVPHAKRQDVTVLGSQEGAMHSALAGATEVEMDGD
GNHMFAGHLKCKVRMEKLRIKGMSYTMCSGKFSIDKEMAETQ
DENV1 Backbone with ZIKV Z3L1 Epitope (SEQ ID NO: 29)
MRCVGIGNRDFVEGLSGATWVDVVLEHGSCVTTMAKDKPTLDIELVTTEVTNPAVL
RKLCIEAKISNTTTDSRCPTQGEATLVEEQDTNFVCRRTFVDRGWGNGCGLFGKGSLI
TCAKFKCVTKLEGKIVQYENLKYRVMVTVHTGDQHQTGHETTEHGTKAEITPQAPT
SEIQLTDYGALTLDCSPRTGLDFNEMVLLTMENKSWLVHKQWFLDLPLPWTSGAST
SQETWNRQDLLVTFKTAHAKKQEVVVLGSQEGAMHTALTGATEIEMDSAKTRIFAG
HLKCRLKMDKLTLKGMSYVMCTGSFKLEKEVAETQHGTVLVQVKYEGTDAPCKIP
FSSQDEKGVTQNGRLITANPIVTDKEKPVNIEAEPPFGESYIVVGAGEKALKLSWFKK
DENV2 Backbone with ZIKV Z3L1 Epitope (SEQ ID NO: 30)
MRCIGISNRDFVEGVSGGSWVDIVLEHGSCVTTMAKNKPTLDFELVTTEAKNPATLR
KYCIEAKLTNTTTESRCPTQGEPSLNEEQDKRFVCKHSMVDRGWGNGCGLFGKGGI
VTCAMFTCKKNMEGKVVQPENLEYRIMVTPHSGEEHATGHETGKHGKKIEVTPQSSI
TEAELTGYGTVTMECSPRTGLDFNEMVLLQMENKAWLVHRQWFLDLPLPWLPGAD
TQGSNWIQKETLVTFKNPHAKKQDVVVLGSQEGAMHTALTGATEIEMDSAKNRLFT
GHLKCRLRMDKLQLKGMSYSMCTGKFKVVKEIAETQHGTIVIRVQYEGDGSPCKIPF
EIMDLEKRHVLGRLITVNPIVTEKDSPVNIEAEPPFGDSYHIGVDPGQLKLNWFKK
DENV3 Backbone with ZIKV Z3L1 Epitope (SEQ ID NO: 31)
MRCVGIGNRDFVEGLSGATWVDVVLEHGGCVTTMAKNKPTLDIELVTTEATNLATL
RKLCIEGKITNITTDSRCPTQGEAVLPEEQDQNYVCKHTYVDRGWGNGCGLFGKGSL
VTCAKFQCLEPIEGKVVQYENLKYRVMITVHTGDQHQTGHETQGVKAEITPQASTTE
AILPEYGTLGLECSPRTGLDFNEMILLTMKNKAWMVHRQWFFDLPLPWTSGATTETP
TWNRKELLVTFKNAHAKKQEVVVLGSQEGAMHTALTGATEIEMDSAKTRIFAGHL
KCRLKMDKLELKGMSYAMCTNTFVLKKEVSETQHGTILIKVEYKGEDAPCKIPFSTE
DGQGKAHNGRLITANPVVTKKEEPVNIEAEPPFGESNIVIGIGDNALKINWYKK
DENV4 Backbone with ZIKV Z3L1 Epitope (SEQ ID NO: 32)
MRCVGVGNRDFVEGVSGGAWVDLVLEHGGCVTTMAQGKPTLDFELVTTTAKNVA
LLRTYCIEASISNITTATRCPTQGEPYLKEEQDQQYICRRDVVDRGWGNGCGLFGKG
GVVTCAKFSCSGKITGNLVQIENLEYRVMVTVHNGDTHATGHETSNHGVKAEITPRS
PSVEVKLPDYGELTLDCEPRSGIDFNEMILMKMKNKTWLVHKQWFLDLPLPWTAGA
DTSEVHWNYKERMVTFKVPHAKRQDVTVLGSQEGAMHSALAGATEVEMDGAKNR
MFAGHLKCKVRMEKLRIKGMSYTMCSGKFSIDKEMAETQHGTTVVKVKYEGAGAP
CKVPIEIRDVNKEKVVGRVISSTPLAENTNSVTNIELEPPFGDSYIVIGVGNSALTLHW
FRK
DENV1 Backbone with ZIKV Z20 and Z3L1 Epitopes (SEQ ID NO: 33)
MRCVGIGNRDFVEGLSGATWVDVVLEHGSCVTTMAKDKPTLDIELVTTEVTNPAVL
RKLCIEAKISDMATDSRCPTQGEATLVKEQDTNFVCRRTFVDRGWGNGCGLFGKGS
LITCAKFACVTKLEGKIVQYENLKYRVMVTVHTGDQHQTGHETTEHGTKAEITPQAP
TSEIQLTDYGALTLDCSPRTGLDFNEMVLLTMENKSWLVHKQWFLDLPLPWTSGAS
TGTETWNRQDLLVTFKTAHAKKQEVVVLGSQEGAMHTALTGATEIEMDSAKTRIFA
GHLKCRLKMDKLTLKGMSYVMCTGSFKLEKEVAETQHGTVLVQVKYEGTDAPCKI
PFSSQDEKGVTQNGRLITANPIVTDKEKPVNIEAEPPFGESYIVVGAGEKALKLSWFK
K
DENV2 Backbone with ZIKV Z20 and Z3L1 Epitopes (SEQ ID NO: 34)
MRCIGISNRDFVEGVSGGSWVDIVLEHGSCVTTMAKNKPTLDFELVTTEAKNPATLR
KYCIEAKLTDMATESRCPTQGEPSLNKEQDKRFVCKHSMVDRGWGNGCGLFGKGGI
VTCAMFACKKNMEGKVVQPENLEYRIMVTPHSGEEHATGHETGKHGKKIEVTPQSS
ITEAELTGYGTVTMECSPRTGLDFNEMVLLQMENKAWLVHRQWFLDLPLPWLPGA
DTGTSNWIQKETLVTFKNPHAKKQDVVVLGSQEGAMHTALTGATEIEMDSAKNRLF
TGHLKCRLRMDKLQLKGMSYSMCTGKFKVVKEIAETQHGTIVIRVQYEGDGSPCKIP
FEIMDLEKRHVLGRLITVNPIVTEKDSPVNIEAEPPFGDSYIIIGVDPGQLKLNWFKK
DENV3 Backbone with ZIKV Z20 and Z3L1 Epitopes (SEQ ID NO: 35)
MRCVGIGNRDFVEGLSGATWVDVVLEHGGCVTTMAKNKPTLDIELVTTEATNLATL
RKLCIEGKITDMATDSRCPTQGEAVLPKEQDQNYVCKHTYVDRGWGNGCGLFGKG
SLVTCAKFACLEPIEGKVVQYENLKYRVMITVHTGDQHQTGHETQGVKAEITPQAST
TEAILPEYGTLGLECSPRTGLDFNEMILLTMKNKAWMVHRQWFFDLPLPWTSGATT
GTPTWNRKELLVTFKNAHAKKQEVVVLGSQEGAMHTALTGATEIEMDSAKTRIFAG
HLKCRLKMDKLELKGMSYAMCTNTFVLKKEVSETQHGTILIKVEYKGEDAPCKIPFS
TEDGQGKAHNGRLITANPVVTKKEEPVNIEAEPPFGESNIVIGIGDNALKINWYKK
DENV4 Backbone with ZIKV Z20 and Z3L1 Epitopes (SEQ ID NO: 36)
MRCVGVGNRDFVEGVSGGAWVDLVLEHGGCVTTMAQGKPTLDFELVTTTAKNVA
LLRTYCIEASISDMATATRCPTQGEPYLKKEQDQQYICRRDVVDRGWGNGCGLFGK
GGVVTCAKFACSGKITGNLVQIENLEYRVMVTVHNGDTHATGHETSNHGVKAEITP
RSPSVEVKLPDYGELTLDCEPRSGIDFNEMILMKMKNKTWLVHKQWFLDLPLPWTA
GADTGTVHWNYKERMVTFKVPHAKRQDVTVLGSQEGAMHSALAGATEVEMDGA
KNRMFAGHLKCKVRMEKLRIKGMSYTMCSGKFSIDKEMAETQHGTTVVKVKYEGA
GAPCKVPIEIRDVNKEKVVGRVISSTPLAENTNSVTNIELEPPFGDSYIVIGVGNSALTL
HWFRK
ZIKV Z20 Epitope Ablation by DENV1 (SEQ ID NO: 37)
IRCIGVSNRDFVEGMSGGTWVDVVLEHGGCVTVMAQDKPTVDIELVTTTVSNMAEV
RSYCYEASISNTTSDSRCPTQGEAYLDEQSDTQYVCKRTLVDRGWGNGCGLFGKGS
LVTCAKFKCSKKMTGKSIQPENLEYRIMLSVHGSQHSGMIVNDTGHETDENRAKVEI
TPNSPRAEATLGGFGSLGLDCEPRTGLDFSDLYYLTMNKKHWLVHKEWFHDIPLPW
HAGADTSQPHWNNKEALVEFKDAHAKRQTVVVLGSQEGAVHTALAGALEAQTDG
AKGRLSSGHLKCRLKMDKLRLKGVSYSLCTAAFTFTKIPAETLHGTVTVEVQYAGT
DGPCKVPAQMAVDMQTLTPVGRLITANPVITESTENSKMMLELDPPFGDSYIVIGVG
EKKITHHWHRSG
ZIKV Z20 Epitope Ablation by DENV2 (SEQ ID NO: 38)
IRCIGVSNRDFVEGMSGGTWVDVVLEHGGCVTVMAQDKPTVDIELVTTTVSNMAEV
RSYCYEASISNTTSDSRCPTQGEAYLDEQSDTQYVCKRTLVDRGWGNGCGLFGKGS
LVTCAKFTCSKKMTGKSIQPENLEYRIMLSVHGSQHSGMIVNDTGHETDENRAKVEI
TPNSPRAEATLGGFGSLGLDCEPRTGLDFSDLYYLTMNNKHWLVHKEWFHDIPLPW
HAGADTQGPHWNNKEALVEFKDAHAKRQTVVVLGSQEGAVHTALAGALEAQMDG
AKGRLSSGHLKCRLKMDKLRLKGVSYSLCTAAFTFTKIPAETLHGTVTVEVQYAGT
DGPCKVPAQMAVDMQTLTPVGRLITANPVITESTENSKMMLELDPPFGDSYIVIGVG
EKKITHHWHRSG
ZIKV Z20 Epitope Ablation by DENV3 (SEQ ID NO: 39)
IRCIGVSNRDFVEGMSGGTWVDVVLEHGGCVTVMAQDKPTVDIELVTTTVSNMAEV
RSYCYEASISNITSDSRCPTQGEAYLDEQSDTQYVCKRTLVDRGWGNGCGLFGKGSL
VTCAKFQCSKKMTGKSIQPENLEYRIMLSVHGSQHSGMIVNDTGHETDENRAKVEIT
PNSPRAEATLGGFGSLGLDCEPRTGLDFSDLYYLTMNNKHWLVHKEWFHDIPLPWH
AGADTETPHWNNKEALVEFKDAHAKRQTVVVLGSQEGAVHTALAGALEAQNDGA
KGRLSSGHLKCRLKMDKLRLKGVSYSLCTAAFTFTKIPAETLHGTVTVEVQYAGTD
GPCKVPAQMAVDMQTLTPVGRLITANPVITESTENSKMMLELDPPFGDSYIVIGVGE
KKITHHWHRSG
ZIKV Z20 Epitope Ablation by DENV4 (SEQ ID NO: 40)
IRCIGVSNRDFVEGMSGGTWVDVVLEHGGCVTVMAQDKPTVDIELVTTTVSNMAEV
RSYCYEASISNITSDSRCPTQGEAYLDEQSDTQYVCKRTLVDRGWGNGCGLFGKGSL
VTCAKFSCSKKMTGKSIQPENLEYRIMLSVHGSQHSGMIVNDTGHETDENRAKVEIT
PNSPRAEATLGGFGSLGLDCEPRTGLDFSDLYYLTMNKKHWLVHKEWFHDIPLPWH
AGADTSEPHWNNKEALVEFKDAHAKRQTVVVLGSQEGAVHTALAGALEADSDGA
KGRLSSGHLKCRLKMDKLRLKGVSYSLCTAAFTFTKIPAETLHGTVTVEVQYAGTD
GPCKVPAQMAVDMQTLTPVGRLITANPVITESTENSKMMLELDPPFGDSYIVIGVGE
KKITHHWHRSG
ZIKV Z3L1 Epitope Ablation by DENV1 (SEQ ID NO: 41)
IRCIGVSNRDFVEGMSGGTWVDVVLEHGGCVTVMAQDKPTVDIELLKTTVSNMAEV
RSYCYEASISDMASDSRCPTQGEAYLDKQSDTQYVCKRTLVDRGWGNGCGLFGKGS
LVTCAKFACSKKMTGKSIQPENLEYSIILSVHGSQHSGMIVNDVGNETDENRATVTIT
PNSPRAEATLGGFGSLGLDCEPRTGLDFSDLYYLTMNKKHWLVHKEWFHDIPLPWH
AGADTGTPHWNNKEALVEFKDAHAKRQTVVVLGSQEGAVHTALAGALEAQTDGG
TGTLSSGHLKCRLKMDKLRLKGVSYSLCTAAFTFTKIPAETLHGTVTVEVQYAGTDG
PCKVPAQMAVDMQTLTPVGRLITANPVITESTENSKMMLELDPPFGDSYIVIGVGEK
KITHHWHRSG
ZIKV Z3L1 Epitope Ablation by DENV2 (SEQ ID NO: 42)
IRCIGVSNRDFVEGMSGGTWVDVVLEHGGCVTVMAQDKPTVDIELIKTTVSQMAEV
RSYCYEASISDMASDSRCPTQGEAYLDKQSDTQYVCKRTLVDRGWGNGCGLFGKGS
LVTCAKFACSKKMTGKSIQPENLEYTIVLSVHGSQHSGMIVNDVGNDTDENRAEVKI
TPNSPRAEATLGGFGSLGLDCEPRTGLDFSDLYYLTMNNKHWLVHKEWFHDIPLPW
HAGADTGTPHWNNKEALVEFKDAHAKRQTVVVLGSQEGAVHTALAGALEAQMDG
SGGLLSSGHLKCRLKMDKLRLKGVSYSLCTAAFTFTKIPAETLHGTVTVEVQYAGTD
GPCKVPAQMAVDMQTLTPVGRLITANPVITESTENSKMMLELDPPFGDSYIVIGVGE
KKITHHWHRSG
ZIKV Z3L1 Epitope Ablation by DENV3 (SEQ ID NO: 43)
IRCIGVSNRDFVEGMSGGTWVDVVLEHGGCVTVMAQDKPTVDIELQKTTVSQMAE
VRSYCYEASISDMASDSRCPTQGEAYLDKQSDTQYVCKRTLVDRGWGNGCGLFGK
GSLVTCAKFACSKKMTGKSIQPENLEYTIILSVHGSQHSGMIVNDVGNETDENRATV
EITPNSPRAEATLGGFGSLGLDCEPRTGLDFSDLYYLTMNNKHWLVHKEWFHDIPLP
WHAGADTGTPHWNNKEALVEFKDAHAKRQTVVVLGSQEGAVHTALAGALEAQND
GGGGSLSSGHLKCRLKMDKLRLKGVSYSLCTAAFTFTKIPAETLHGTVTVEVQYAGT
DGPCKVPAQMAVDMQTLTPVGRLITANPVITESTENSKMMLELDPPFGDSYIVIGVG
EKKITHHWHRSG
ZIKV Z3L1 Epitope Ablation by DENV4 (SEQ ID NO: 44)
IRCIGVSNRDFVEGMSGGTWVDVVLEHGGCVTVMAQDKPTVDIELTKTTVSEMAEV
RSYCYEASISDMASDSRCPTQGEAYLDKQSDTQYVCKRTLVDRGWGNGCGLFGKGS
LVTCAKFACSKKMTGKSIQPENLEYTIVLSVHGSQHSGMIVNDVGNDTDENRATVTI
TPNSPRAEATLGGFGSLGLDCEPRTGLDFSDLYYLTMNKKHWLVHKEWFHDIPLPW
HAGADTGTPHWNNKEALVEFKDAHAKRQTVVVLGSQEGAVHTALAGALEADSDG
DGGHLSSGHLKCRLKMDKLRLKGVSYSLCTAAFTFTKIPAETLHGTVTVEVQYAGT
DGPCKVPAQMAVDMQTLTPVGRLITANPVITESTENSKMMLELDPPFGDSYIVIGVG
EKKITHHWHRSG
ZIKV Z20 and Z3L1 Epitope Ablation by DENV1 (SEQ ID NO: 45)
IRCIGVSNRDFVEGMSGGTWVDVVLEHGGCVTVMAQDKPTVDIELLKTTVSNMAEV
RSYCYEASISNTTSDSRCPTQGEAYLDEQSDTQYVCKRTLVDRGWGNGCGLFGKGS
LVTCAKFKCSKICMTGKSIQPENLEYSIILSVHGSQHSGMIVNDVGNETDENRATVTIT
PNSPRAEATLGGFGSLGLDCEPRTGLDFSDLYYLTMNKKHWLVHKEWFHDIPLPWH
AGADTSQPHWNNKEALVEFKDAHAKRQTVVVLGSQEGAVHTALAGALEAQTDGG
TGTLSSGHLKCRLKMDKLRLKGVSYSLCTAAFTFTKIPAETLHGTVTVEVQYAGTDG
PCKVPAQMAVDMQTLTPVGRLITANPVITESTENSKMMLELDPPFGDSYIVIGVGEK
KITHHWHRSG
ZIKV Z20 and Z3L1 Epitope Ablation by DENV2 (SEQ ID NO: 46)
IRCIGVSNRDFVEGMSGGTWVDVVLEHGGCVTVMAQDKPTVDIELIKTTVSQMAEV
RSYCYEASISNTTSDSRCPTQGEAYLDEQSDTQYVCKRTLVDRGWGNGCGLFGKGS
LVTCAKFTCSKKMTGKSIQPENLEYTIVLSVHGSQHSGMIVNDVGNDTDENRAEVKI
TPNSPRAEATLGGFGSLGLDCEPRTGLDFSDLYYLTMNNKHWLVHKEWFHDIPLPW
HAGADTQTPHWNNKEALVEFKDAHAKRQTVVVLGSQEGAVHTALAGALEAQMDG
SGGLLSSGHLKCRLKMDKLRLKGVSYSLCTAAFTFTKIPAETLHGTVTVEVQYAGTD
GPCKVPAQMAVDMQTLTPVGRLITANPVITESTENSKMMLELDPPFGDSYIVIGVGE
KKITHHWHRSG
ZIKV Z20 and Z3L1 Epitope Ablation by DENV3 (SEQ ID NO: 47)
IRCIGVSNRDFVEGMSGGTWVDVVLEHGGCVTVMAQDKPTVDIELQKTTVSQMAE
VRSYCYEASISNTSDSRCPTQGEAYLDEQSDTQYVCKRTLVDRGWGNGCGLFGKGS
LVTCAKFQCSKKMTGKSIQPENLEYTIILSVHGSQHSGMIVNDVGNETDENRATVEIT
PNSPRAEATLGGFGSLGLDCEPRTGLDFSDLYYLTMNNKHWLVHKEWFHDIPLPWH
AGADTETPHWNNKEALVEFKDAHAKRQTVVVLGSQEGAVHTALAGALEAQNDGG
GGSLSSGHLKCRLKMDKLRLKGVSYSLCTAAFTFTKIPAETLHGTVTVEVQYAGTDG
PCKVPAQMAVDMQTLTPVGRLITANPVITESTENSKMMLELDPPFGDSYIVIGVGEK
KITHHWHRSG
ZIKV Z20 and Z3L1 Epitope Ablation by DENV4 (SEQ ID NO: 48)
IRCIGVSNRDFVEGMSGGTWVDVVLEHGGCVTVMAQDKPTVDIELTKTTVSEMAEV
RSYCYEASISNITSDSRCPTQGEAYLDEQSDTQYVCKRTLVDRGWGNGCGLFGKGSL
VTCAKFSCSKKMTGKSIQPENLEYTIVLSVHGSQHSGMIVNDVGNDTDENRATVTIT
PNSPRAEATLGGFGSLGLDCEPRTGLDFSDLYYLTMNKKHWLVHKEWFHDIPLPWH
AGADTSEPHWNNKEALVEFKDAHAKRQTVVVLGSQEGAVHTALAGALEADSDGD
GGHLSSGHLKCRLKMDKLRLKGVSYSLCTAAFTFTKIPAETLHGTVTVEVQYAGTD
GPCKVPAQMAVDMQTLTPVGRLITANPVITESTENSKMMLELDPPFGDSYIVIGVGE
KKITHHWHRSG
ZIKV E Glycoprotein WT (SEQ ID NO: 49)
IRCIGVSNRDFVEGMSGGTWVDVVLEHGGCVTVMAQDKPTVDIELVTTTVSNMAEV
RSYCYEASISDMASDSRCPTQGEAYLDKQSDTQYVCKRTLVDRGWGNGCGLFGKGS
LVTCAKFACSKKMTGKSIQPENLEYRIMLSVHGSQHSGMIVNDTGHETDENRAKVEI
TPNSPRAEATLGGFGSLGLDCEPRTGLDFSDLYYLTMNNKHWLVHKEWFHDIPLPW
HAGADTGTPHWNNKEALVEFKDAHAKRQTVVVLGSQEGAVHTALAGALEAEMDG
AKGRLSSGHLKCRLKMDKLRLKGVSYSLCTAAFTFTKIPAETLHGTVTVEVQYAGT
DGPCKVPAQMAVDMQTLTPVGRLITANPVITESTENSKMMLELDPPFGDSYIVIGVG
EKKITHHWHRSG
ZIKV EDE1 Null Mutant- Core (SEQ ID NO: 50)
IRCIGVSNRDFVEGMSGGTWVDVVLEHGGCVTVMAQDKPTVDIELVTTTVSNMAEV
RSYCYEASISDMESRAKCPTQGEAYLDKQSDTQYVCKRTLVDRGWDSGCGLFGKGS
LVTCAKFACSKKMTGKSIQPENLEYRIMLSVHGSQHSGMIVNDTGHETDENRAKVEI
TPNSPRAEATLGGFGSLGLDCEPRTGLDFSDLYYLTMNNKHWLVHKEWFHDIPLPW
HAGADTGTPHWNNKEALVEFKDAHATYIPVVVLGSQEGAVHTALAGALEAEMDGA
KGRLSSGHLKCRLKMDKLRLKGVSYSLCTAAFTFTKIPAETLHGTVTVEVQYAGTD
GPCKVPAQMAVDMQTLTPVGRLITANPVITESTENSKMMLELDPPFGDSYIVIGVGE
KKITHHWHRSG
ZIKV EDE1 Null Mutant- Core and Specific (SEQ ID NO: 51)
IHCIGVSNRDFVEGMSGGTWVDVVLEHDGCVTVMAQDKPTVDITVVTTTVSNMAE
VRSYCYEASISDMESRAKCPTAGEAYIDKQSDTQYVCKRTLVDRGWDSGCFLFGKG
SLVTCAKFACSKKMTGKSIQPENLEYRIMLSVHGSQNSGKIVNDTGHETDEKRAKVE
ITPNSPRAEATLGGFGSLGLDCEPRTGLDFSDLYYLTMNNKHWLVHKEWFHDIPLPW
HAGADTGTPHWNNKEALVEFKDAHATYIPVVVLGSQEGAVHTALAGALEAKMDG
AKGRLSSGHLKCRLKMDKLRLKGVSYSLCTAAFTFTSGPAETLHGTVTVLVGYAGT
DGPCKVPAQMAVDMQTLTPVGRLITANPVITESTENSKMMLELDPPFGDSYIVIGVG
EKKITHHWHRSG
ZIKV EDE1 Null Mutant- Specific (SEQ ID NO: 52)
IHCIGVSNRDFVEGMSGGTWVDVVLEHDGCVTVMAQDKPTVDITVVTTTVSNMAE
VRSYCYEASTSDMASDSRCPTAGEAYIDKQSDTQYVCKRTLVDRGWGNGCFLFGKG
SLVTCAKFACSKKMTGKSIQPENLEYRIMLSVHGSQNSGKIVNDTGHETDEKRAKVE
ITPNSPRAEATLGGFGSLGLDCEPRTGLDFSDLYYLTMNNKHWLVHKEWFHDIPLPW
HAGADTGTPHWNNKEALVEFKDAHAKRQTVVVLGSQEGAVHTALAGALEAKMDG
AKGRLSSGHLKCRLKMDKLRLKGVSYSLCTAAFTFTSGPAETLHGTVTVLVGYAGT
DGPCKVPAQMAVDMQTLTPVGRLITANPVITESTENSKMMLELDPPFGDSYIVIGVG
EKKITHHWHRSG
DENV1 E Protein WT (SEQ ID NO: 53)
MRCVGIGNRDFVEGLSGATWVDVVLEHGSCVTTMAKDKPTLDIELLKTEVTNPAVL
RKLCIEAKISNTTTDSRCPTQGEATLVEEQDTNFVCRRTFVDRGWGNGCGLFGKGSLI
TCAKFKCVTKLEGKIVQYENLKYSVIVTVHTGDQHQVGNETTEHGTTATITPQAPTS
EIQLTDYGALTLDCSPRTGLDFNEMVLLTMEKKSWLVHKQWFLDLPLPWTSGASTS
QETWNRQDLLVTFKTAHAKKQEVVVLGSQEGAMHTALTGATEIQTSGTTTIFAGHL
KCRLKMDKLTLKGMSYVMCTGSFKLEKEVAETQHGTVLVQVKYEGTDAPCKIPFSS
QDEKGVTQNGRLITANPIVTDKEKPVNIEAEPPFGESYIVVGAGEKALKLSWFKK
DENV1 EDE1 Null Mutant- Core (SEQ ID NO: 54)
MRCVGIGNRDFVEGLSGATWVDVVLEHGSCVTTMAKDKPTLDIELLKTEVTNPAVL
RKLCIEAKISNTETRAKCPTQGEATLVEEQDTNFVCRRTFVDRGWDSGCGLFGKGSLI
TCAKFKCVTKLEGKIVQYENLKYSVIVTVHTGDQHQVGNETTEHGTIATITPQAPTSE
IQLTDYGALTLDCSPRTGLDFNEMVLLTMKKKSWLVHKQWFLDLPLPWTSGASTSQ
ETWNRQDLLVTFKTAHATYIPVVVLGSQEGAMHTALTGATEIQTSGTTTIFAGHLKC
RLKMDKLTLKGMSYVMCTGSFKLEKEVAETQHGTVLVQVKYEGTDAPCKIPFSSQD
EKGVTQNGRLITANPIVTDKEKPVNIEAEPPFGESYIVVGAGEKALKLSWFKK
DENV1 EDE1 Null Mutant- Core and Specific (SEQ ID NO: 55)
MHCVGIGNRDFVEGLSGATWVDVVLEHDSCVTTMAKDKPTLDITVLKTEVTNPAVL
RKLCIEAKISNTETRAKCPTAGEATIVEEQDTNFVCRRTFVDRGWDSGCFLFGKGSLI
TCAKFKCVTKLEGKIVQYENLKYSVIVTVHTGDNHKVGNETTEKGTIATITPQAPTSE
IQLTDYGALTLDCSPRTGLDFNEMVLLTMKKKSWLVHKQWFLDLPLPWTSGASTSQ
ETWNRQDLLVTFKTAHATYIPVVVLGSQEGAMHTALTGATEIKTSGTTTIFAGHLKC
RLKMDKLTLKGMSYVMCTGSFKLESGVAETQHGTVLVLVGYEGTDAPCKIPFSSQD
EKGVTQNGRLITANPIVTDKEKPVNIEAEPPFGESYIVVGAGEKALKLSWFKK
DENV1 EDE1 Null Mutant- Specific (SEQ ID NO: 56)
MHCVGIGNRDFVEGLSGATWVDVVLEHDSCVTTMAKDKPTLDITVLKTEVTNPAVL
RKLCIEAKISNTTTDSRCPTAGEATIVEEQDTNFVCRRTFVDRGWGNGCFLFGKGSLI
TCAKGKCVTKLEGKIVQYENLKYSVIVTVHTGDNHKVGNETTEKGTIATITPQAPTSE
IQLTDYGALTLDCSPRTGLDFNEMVLLTMKKKSWLVHKQWFLDLPLPWTSGASTSQ
ETWNRQDLLVTFKTAHAKKQEVVVLGSQEGAMHTALTGATEIKTSGTTTIFAGHLK
CRLKMDKLTLKGMSYVMCTGSFKLESGVAETQHGTVLVLVGYEGTDAPCKIPFSSQ
DEKGVTQNGRLITANPIVTDKEKPVNIEAEPPFGESYIVVGAGEKALKLSWFKK
DENV2 EDE1 Null Mutant- Core (SEQ ID NO: 57)
MRCIGISNRDFVEGVSGGSWVDIVLEHGSCVTTMAKNKPTLDFELIKTEAKQPATLR
KYCIEAKLTNTETRAKCPTQGEPSLNEEQDKRFVCKHSMVDRGWDSGCGLFGKGGI
VTCAMFTCKKNMEGKVVQPENLEYTIVVTPHSGEEHAVGNDTGKHGKIIKVTPQSSI
TEAELTGYGTVTMECSPRTGLDFNEMVLLQMKNKAWLVHRQWFLDLPLPWLPGAD
TQGSNWIQKETLVTFKNPHATYIPVVVLGSQEGAMHTALTGATEIQMSSGNLLFTGH
LKCRLRMDKLQLKGMSYSMCTGKFKVVKEIAETQHGTIVIRVQYEGDGSPCKIPFEI
MDLEKRHVLGRLITVNPIVTEKDSPVNIEAEPPFGDSYIIIGVDPGQLKLNWFKK
DENV2 EDE1 Null Mutant- Core and Specific (SEQ ID NO: 58)
MHCIGISNRDFVEGVSGGSWVDIVLEHDSCVTTMAKNKPTLDFTVIKTEAKQPATLR
KYCIEAKLTNTETRAKCPTAGEPSINEEQDKRFVCKEISMVDRGWDSGCFLFGKGGIV
TCAMFTCKKNMEGKVVQPENLEYTIVVTPHSGENHKVGNDTGKKGKIIKVTPQSSIT
EAELTGYGTVTMECSPRTGLDFNEMVLLQMKNKAWLVHRQWFLDLPLPWLPGADT
QGSNWIQKETLVTFKNPHATYIPVVVLGSQEGAMHTALTGATEIKMSSGNLLFTGHL
KCRLRMDKLQLKGMSYSMCTGKFKVVSGIAETQHGTIVILVGYEGDGSPCKIPFEIM
DLEKRHVLGRLITVNPIVTEKDSPVNIEAEPPFGDSYIIIGVDPGQLKLNWFKK
DENV2 EDE1 Null Mutant- Specific (SEQ ID NO: 59)
MHCIGISNRDFVEGVSGGSWVDIVLEHDSCVTTMAKNKPTLDFTVIKTEAKQPATLR
KYCIEAKLTNTTTESRCPTAGEPSINEEQDKRFVCKHSMVDRGWGNGCFLFGKGGIV
TCAMFTCKKNMEGKVVQPENLEYTIVVTPHSGENHKVGNDTGKKGKIIKVTPQSSIT
EAELTGYGTVTMECSPRTGLDFNEMVLLQMKNKAWLVHRQWFLDLPLPWLPGADT
QGSNWIQKETLVTFKNPHAKKQDVVVLGSQEGAMHTALTGATEIKMSSGNLLFTGH
LKCRLRMDKLQLKGMSYSMCTGKFKVVSGIAETQHGTIVILVGYEGDGSPCKIPFEI
MDLEKRHVLGRLITVNPIVTEKDSPVNIEAEPPFGDSYIIIGVDPGQLKLNWFKK
DENV3 EDE1 Null Mutant- Core (SEQ ID NO: 60)
MRCVGIGNRDFVEGLSGATWVDVVLEHGGCVTTMAKNKPTLDIELQKTEATQLATL
RKLCIEGKITNIETRAKCPTQGEAVLPEEQDQNYVCKHTYVDRGWDSGCGLFGKGSL
VTCAKFQCLEPIEGKVVQYENLKYTVIITVHTGDQHQVGNETQGVIAEITPQASTTEA
ILPEYGTLGLECSPRTGLDFNEMILLTMKNKAWMVHRQWFFDLPLPWTSGATTETPT
WNRKELLVTFKNAHATYIPVVVLGSQEGAMHTALTGATEIQNSGGTSIFAGHLKCRL
KMDKLELKGMSYAMCTNTFVLKKEVSETQHGTILIKVEYKGEDAPCKIPFSTEDGQG
KAHNGRLITANPVVTKKEEPVNIEAEPPFGESNIVIGIGDNALKINWYKK
DENV3 EDE1 Null Mutant- Core and Specific (SEQ ID NO: 61)
MHCVGIGNRDFVEGLSGATWVDVVLEHDGCVTTMAKNKPTLDITVQKTEATQLAT
LRKLCIEGKITNIETRAKCPTAGEAVIPEEQDQNYVCKHTYVDRGWDSGCFLFGKGS
LVTCAKFQCLEPIEGKVVQYENLKYTVIITVHTGDNHKVGNETKGVIAEITPQASTTE
AILPEYGTLGLECSPRTGLDFNEMILLTMKNKAWMVHRQWFFDLPLPWTSGATTETP
TWNRKELLVTFKNAHATYIPVVVLGSQEGAMHTALTGATEIKNSGGTSIFAGHLKCR
LKMDKLELKGMSYAMCTNTFVLKSGVSETQHGTILILVGYKGEDAPCKIPFSTEDGQ
GKAHNGRLITANPVVTKKEEPVNIEAEPPFGESNIVIGIGDNALKINWYKK
DENV3 EDE1 Null Mutant- Specific (SEQ ID NO: 62)
MHCVGIGNRDFVEGLSGATWVDVVLEHDGCVTTMAKNKPTLDITVQKTEATQLAT
LRKLCIEGKITNITTDSRCPTAGEAVIPEEQDQNYVCICHTYVDRGWGNGCFLFGKGS
LVTCAKFQCLEPIEGKVVQYENLKYTVIITVHTGDNHKVGNETKGVIAEITPQASTTE
AILPEYGTLGLECSPRTGLDFNEMILLTMKNKAWMVHRQWFFDLPLPWTSGATTETP
TWNRKELLVTFKNAHAKKQEVVVLGSQEGAMHTALTGATEIKNSGGTSIFAGHLKC
RLKMDKLELKGMSYAMCTNTFVLKSGVSETQHGTILILVGYKGEDAPCKIPFSTEDG
QGKAHNGRLITANPVVTKKEEPVNIEAEPPFGESNIVIGIGDNALKINWYKK
DENV4 EDE1 Null Mutant- Core (SEQ ID NO: 63)
MRCVGVGNRDFVEGVSGGAWVDLVLEHGGCVTTMAQGKPTLDFELTKTTAKEVA
LLRTYCIEASISNIETRAKCPTQGEPYLKEEQDQQYICRRDVVDRGWDSGCGLFGKG
GVVTCAKFSCSGKITGNLVQIENLEYTVVVTVHNGDTHAVGNDTSNHGVIATITPRS
PSVEVKLPDYGELTLDCEPRSGIDFNEMILMKMKKKTWLVHKQWFLDLPLPWTAGA
DTSEVHWNYKERMVTFKVPHATYIPVTVLGSQEGAMHSALAGATEVDSGDGNHMF
AGHLKCKVRMEKLRIKGMSYTMCSGKFSIDKEMAETQHGTTVVKVKYEGAGAPCK
VPIEIRDVNKEKVVGRVISSTPLAENTNSVTNIELEPPFGDSYIVIGVGNSALTLHWFR
K
DENV4 EDE1 Null Mutant- Core and Specific (SEQ ID NO: 64)
MHCVGVGNRDFVEGVSGGAWVDLVLEHDGCVTTMAQGKPTLDFTVTKTTAKEVA
LLRTYCIEASISNIETRAKCPTAGEPYIKEEQDQQYICRRDVVDRGWDSGCFLFGKGG
VVTCAKFSCSGKITGNLVQIENLEYTVVVTVHNGDNHKVGNDTSNKGVIATITPRSPS
VEVKLPDYGELTLDCEPRSGIDFNEMILMKMKKKTWLVHKQWFLDLPLPWTAGAD
TSEVHWNYKERMVTFKVPHATYIPVTVLGSQEGAMHSALAGATEVKSGDGNHMFA
GHLKCKVRMEKLRIKGMSYTMCSGKFSIDSGMAETQHGTTVVLVGYEGAGAPCKV
PIEIRDVNKEKVVGRVISSTPLAENTNSVTNIELEPPFGDSYIVIGVGNSALTLHWFRK
DENV4 EDE1 Null Mutant- Specific (SEQ ID NO: 65)
MHCVGVGNRDFVEGVSGGAWVDLVLEHDGCVTTMAQGKPTLDFTVTKTTAKEVA
LLRTYCIEASISNITTATRCPTAGEPYIKEEQDQQYICRRDVVDRGWGNGCFLFGKGG
VVTCAKFSCSGKITGNLVQIENLEYTVVVTVHNGDNHKVGNDTSNKGVIATITPRSPS
VEVKLPDYGELTLDCEPRSGIDFNEMILMKMKKKTWLVHKQWFLDLPLPWTAGAD
TSEVHWNYKERMVTFKVPHAKRQDVTVLGSQEGAMHSALAGATEVKSGDGNHMF
AGHLKCKVRMEKLRIKGMSYTMCSGKFSIDSGMAETQHGTTVVLVGYEGAGAPCK
VPIEIRDVNKEKVVGRVISSTPLAENTNSVTNIELEPPFGDSYIVIGVGNSALTLHWFR
K
Zika and Dengue EDE1 Epitope Swaps (C10 and C8)
ZIKV with DENV1 C10 Epitope (SEQ ID NO: 66)
IRCIGVSNRDFVEGMSGGTWVDVVLEHGGCVTVMAQDKPTVDIELLKTTVSNMAV
VRSYCYEASISNTTTDSRCPTQGEATLVEQSDTQYVCKRTLVDRGWGNGCGLFGKG
SLVTCAKFACSKKMTGKSIQPENLEYSIILSVHGSQQSGMIVNQTGHETDENGAKVEI
TPNSPRAEATLTDFGALGLDCEPRTGLDFSDLYYLTMNNKHWLVHKEWFHDIPLPW
HAGASTGTPHWNNKEALVEFKDAHAKKQEVVVLGSQEGAVHTALAGALEAQTSG
AKGTLSSGHLKCRLKMDKLTLKGVSYSLCTAAFTFEKIPAETLHGTVTVQVKYAGT
DGPCKVPAQMAVDMQTLTPVGRLITANPVITESEENKPMMLELDPPFGDSYIVIGVG
EKKITHHWHRSG
ZIKV with DENV2 C10 Epitope (SEQ ID NO: 67)
IRCIGVSNRDFVEGMSGGTWVDVVLEHGGCVTVMAQDKPTVDIELIKTTVSQMATV
RSYCYEASISNTTTESRCPTQGEASLNEQSDTQYVCKRTLVDRGWGNGCGLFGKGSI
VTCAKFACSKKMTGKSIQPENLEYTIVLSVHGSQESGMIVNATGHETDENGAKVEIT
PNSPRAEATLTGFGTLGLDCEPRTGLDFSDLYYLTMNNKHWLVHKEWFHDIPLPWH
AGADTGTPHWNNKEALVEFKDAHAKKQDVVVLGSQEGAVHTALAGALEAQMSSA
KGLLSSGHLKCRLKMDKLQLKGVSYSLCTAAFTFVKIPAETLHGTVTVRVQYAGTD
GPCKVPAQMAVDMQTLTPVGRLITANPVITESDENSPMMLELDPPFGDSYIVIGVGE
KKITHHWHRSG
ZIKV with DENV3 C10 Epitope (SEQ ID NO: 68)
IRCIGVSNRDFVEGMSGGTWVDVVLEHGGCVTVMAQDKPTVDIELQKTTVSQMAT
VRSYCYEASISNITTDSRCPTQGEAVLPEQSDTQYVCKRTLVDRGWGNGCGLFGKGS
LVTCAKFACSKKMTGKSIQPENLEYTIILSVHGSQQSGMIVNQTGHETDENGAKVEIT
PNSPRAEATLPEFGTLGLDCEPRTGLDFSDLYYLTMNNKHWLVHKEWFHDIPLPWH
AGATTGTPHWNNKEALVEFKDAHAKKQEVVVLGSQEGAVHTALAGALEAQNSGA
KGSLSSGHLKCRLKMDKLELKGVSYSLCTAAFTFKKIPAETLHGTVTVKVEYAGTD
GPCKVPAQMAVDMQTLTPVGRLITANPVITESEENEPMMLELDPPFGDSYIVIGVGE
KKITHHWHRSG
ZIKV with DENV4 C10 Epitope (SEQ ID NO: 69)
IRCIGVSNRDFVEGMSGGTWVDVVLEHGGCVTVMAQDKPTVDIELTKTTVSEMALV
RSYCYEASISNITTATRCPTQGEAYLKEQSDTQYVCKRTLVDRGWGNGCGLFGKGS
VVTCAKFACSKKMTGKSIQPENLEYTIVLSVHGSQTSGMIVNATGHETDENGAKVEI
TPNSPRAEATLPDFGELGLDCEPRTGLDFSDLYYLTMNNKHWLVHKEWFHDIPLPW
HAGADTGTPHWNNKEALVEFKDAHAKRQDVVVLGSQEGAVHTALAGALEADSGD
AKGHLSSGHLKCRLKMDKLRLKGVSYSLCTAAFTFDKIPAETLHGTVTVKVKYAGT
DGPCKVPAQMAVDMQTLTPVGRLITANPVITESNENSVMMLELDPPFGDSYIVIGVG
EKKITHHWHRSG
ZIKV with DENV1 C10 Epitope- Non-neurotropic (SEQ ID NO: 70)
IRCIGVSNRDFVEGMSGGTWVDVVLEHGGCVTVMAQDKPTVDIELLKTTVSNMAV
VRSYCYEASISNTTTDSRCPTQGEATLVEQSDTQYVCKRTLVDRGWGNGCGLFGKG
SLVTCAKFACSKKMTGKSIQPENLEYSIILSVHGSQQNQTGHETDENGAKVEITPNSP
RAEATLTDFGALGLDCEPRTGLDFSDLYYLTMNNKHWLVHKEWFHDIPLPWHAGA
STGTPHWNNKEALVEFKDAHAKKQEVVVLGSQEGAVHTALAGALEAQTSGAKGTL
SSGHLKCRLKMDKLTLKGVSYSLCTAAFTFEKIPAETLHGTVTVQVKYAGTDGPCK
VPAQMAVDMQTLTPVGRLITANPVITESEENKPMMLELDPPFGDSYIVIGVGEKKITH
HWHRSG
ZIKV with DENV2 C10 Epitope- Non-neurotropic (SEQ ID NO: 71)
IRCIGVSNRDFVEGMSGGTWVDVVLEHGGCVTVMAQDKPTVDIELIKTTVSQMATV
RSYCYEASISNTTTESRCPTQGEASLNEQSDTQYVCKRTLVDRGWGNGCGLFGKGSI
VTCAKFACSKKMTGKSIQPENLEYTIVLSVHGSQENATGHETDENGAKVEITPNSPR
AEATLTGFGTLGLDCEPRTGLDFSDLYYLTMNNKHWLVHKEWFHDIPLPWHAGAD
TGTPHWNNKEALVEFKDAHAKKQDVVVLGSQEGAVHTALAGALEAQMSSAKGLL
SSGHLKCRLKMDKLQLKGVSYSLCTAAFTFVKIPAETLHGTVTVRVQYAGTDGPCK
VPAQMAVDMQTLTPVGRLITANPVITESDENSPMMLELDPPFGDSYIVIGVGEKKITH
HWHRSG
ZIKV with DENV3 C10 Epitope- Non-neurotropic (SEQ ID NO: 72)
IRCIGVSNRDFVEGMSGGTWVDVVLEHGGCVTVMAQDICPTVDIELQKTTVSQMAT
VRSYCYEASISNITTDSRCPTQGEAVLPEQSDTQYVCKRTLVDRGWGNGCGLFGKGS
LVTCAKFACSKKMTGKSIQPENLEYTIILSVHGSQQNQTGHETDENGAKVEITPNSPR
AEATLPEFGTLGLDCEPRTGLDFSDLYYLTMNNKHWLVHKEWFHDIPLPWHAGATT
GTPHWNNKEALVEFKDAHAKKQEVVVLGSQEGAVHTALAGALEAQNSGAKGSLSS
GHLKCRLKMDKLELKGVSYSLCTAAFTFKKIPAETLHGTVTVKVEYAGTDGPCKVP
AQMAVDMQTLTPVGRLITANPVITESEENEPMMLELDPPFGDSYIVIGVGEKKITHH
WHRSG
ZIKV with DENV4 C10 Epitope- Non-neurotropic (SEQ ID NO: 73)
IRCIGVSNRDFVEGMSGGTWVDVVLEHGGCVTVMAQDKPTVDIELTKTTVSEMALV
RSYCYEASISNITTATRCPTQGEAYLKEQSDTQYVCKRTLVDRGWGNGCGLFGKGS
VVTCAKFACSKKMTGKSIQPENLEYTIVLSVHGSQTNATGHETDENGAKVEITPNSP
RAEATLPDFGELGLDCEPRTGLDFSDLYYLTMNNKHWLVHKEWFHDIPLPWHAGA
DTGTPHWNNKEALVEFKDAHAKRQDVVVLGSQEGAVHTALAGALEADSGDAKGH
LSSGHLKCRLKMDKLRLKGVSYSLCTAAFTFDKIPAETLHGTVTVKVKYAGTDGPC
KVPAQMAVDMQTLTPVGRLITANPVITESNENSVMMLELDPPFGDSYIVIGVGEKKIT
HHWHRSG
ZIKV with DENV1 C8 Epitope (SEQ ID NO: 74)
IRCIGVSNRDFVEGMSGGTWVDVVLEHGGCVTVMAQDKPTVDIELVTTTVSNMAEV
RSYCYEASISNTTTDSRCPTQGEAYLVEQSDTQYVCKRTLVDRGWGNGCGLFGKGS
LVTCAKFACSKKMTGKSIQPENLEYRIMLSVHGSQQSGMIVNDTGHETDEHRAKVEI
TPNSPRAEATLGGFGSLGLDCEPRTGLDFSDLYYLTMNNKHWLVHKEWFHDIPLPW
HAGADTGTPHWNNKEALVEFKDAHAKKQEVVVLGSQEGAVHTALAGALEAEMSG
AKGRLSSGHLKCRLKMDKLRLKGVSYSLCTAAFTFEKEPAETLHGTVTVQVKYAGT
DGPCKVPAQMAVDMQTLTPVGRLITANPVITESTENSKMMLELDPPFGDSYIVIGVG
EKKITHHWHRSG
ZIKV with DENV2 C8 Epitope (SEQ ID NO: 75)
IRCIGVSNRDFVEGMSGGTWVDVVLEHGGCVTVMAQDKPTVDIELVTTTVSNMAEV
RSYCYEASISNTTTESRCPTQGEAYLNEQSDTQYVCKRTLVDRGWGNGCGLFGKGSI
VTCAKFACSKKMTGKSIQPENLEYRIMLSVHGSQESGMIVNDTGHETDEHRAKVEIT
PNSPRAEATLGGFGSLGLDCEPRTGLDFSDLYYLTMNNKHWLVHKEWFHDIPLPWH
AGADTGTPHWNNKEALVEFKDAHAKKQDVVVLGSQEGAVHTALAGALEAEMSSA
KGRLSSGHLKCRLKMDKLRLKGVSYSLCTAAFTFVKEPAETLHGTVTVRVQYAGTD
GPCKVPAQMAVDMQTLTPVGRLITANPVITESTENSKMMLELDPPFGDSYIVIGVGE
KKITHHWHRSG
ZIKV with DENV3 C8 Epitope (SEQ ID NO: 76)
IRCIGVSNRDFVEGMSGGTWVDVVLEHGGCVTVMAQDKPTVDIELVTTTVSNMAEV
RSYCYEASISNITTDSRCPTQGEAYLPEQSDTQYVCKRTLVDRGWGNGCGLFGKGSL
VTCAKFACSKKMTGKSIQPENLEYRIMLSVHGSQQSGMIVNDTGHETDEQRAKVEIT
PNSPRAEATLGGFGSLGLDCEPRTGLDFSDLYYLTMNNKHWLVHKEWFHDIPLPWH
AGADTGTPHWNNKEALVEFKDAHAKKQEVVVLGSQEGAVHTALAGALEAEMSGA
KGRLSSGHLKCRLKMDKLRLKGVSYSLCTAAFTFKKEPAETLHGTVTVKVEYAGTD
GPCKVPAQMAVDMQTLTPVGRLITANPVITESTENSKMMLELDPPFGDSYIVIGVGE
KKITHHWHRSG
ZIKV with DENV4 C8 Epitope (SEQ ID NO: 77)
IRCIGVSNRDFVEGMSGGTWVDVVLEHGGCVTVMAQDKPTVDIELVTTTVSNMAEV
RSYCYEASISNITTATRCPTQGEAYLKEQSDTQYVCKRTLVDRGWGNGCGLFGKGS
VVTCAKFACSKKMTGKSIQPENLEYRIMLSVHGSQTSGMIVNDTGHETDEHRAKVEI
TPNSPRAEATLGGFGSLGLDCEPRTGLDFSDLYYLTMNNKHWLVHKEWFHDIPLPW
HAGADTGTPHWNNKEALVEFKDAHAKRQDVVVLGSQEGAVHTALAGALEAEMGD
AKGRLSSGHLKCRLKMDKLRLKGVSYSLCTAAFTFDKEPAETLHGTVTVKVKYAGT
DGPCKVPAQMAVDMQTLTPVGRLITANPVITESTENSKMMLELDPPFGDSYIVIGVG
EKKITHHWHRSG
DENV1 E glycoprotein with ZIKV C10 (SEQ ID NO: 78)
MRCVGIGNRDFVEGLSGATWVDVVLEHGSCVTTMAKDKPTLDIELVTTEVTNPAEL
RICLCIEAKISDMASDSRCPTQGEAYLDKEQDTNFVCRRTFVDRGWGNGCGLFGKGS
LITCAKFKCVTKLEGKIVQYENLKYRVMVTVHTGDHHDVGNETTEHRTTATITPQAP
TSEIQLGGYGSLTLDCSPRTGLDFNEMVLLTMEKKSWLVHKQWFLDLPLPWTSGAD
TSQETWNRQDLLVTFKTAHAKRQTVVVLGSQEGAMHTALTGATEIEMDGTTRIFAG
HLKCRLKMDKLRLKGMSYVMCTGSFKLTKEVAETQHGTVLVEVQYEGTDAPCKIPF
SSQDEKGVTQNGRLITANPIVTDKTSKVNIEAEPPFGESYIVVGAGEKALKLSWFKK
DENV2 E glycoprotein with ZIKV C10 (SEQ ID NO: 79)
MRCIGISNRDFVEGVSGGSWVDIVLEHGSCVTTMAKNKPTLDFELVTTEAKNPAELR
KYCIEAKLTDMASDSRCPTQGEPYLDKEQDKRFVCKHSMVDRGWGNGCGLFGKGG
LVTCAMFTCKKNMEGKVVQPENLEYRIMVTPHSGEHHDVGNDTGKHRKEIKVTPQS
SITEAELGGYGSVTMECSPRTGLDFNEMVLLQMENKAWLVHRQWFLDLPLPWLPG
ADTQGSNWIQKETLVTFKNPHAKRQTVVVLGSQEGAMHTALTGATEIEMDGGNRLF
TGHLKCRLRMDKLRLKGMSYSMCTGKFKVTKEIAETQHGTIVIEVQYEGDGSPCKIP
FEIMDLEKRHVLGRLITVNPIVTEKTSKVNIEAEPPFGDSYIIIGVDPGQLKLNWFKK
DENV3 E glycoprotein with ZIKV C10 (SEQ ID NO: 80)
MRCVGIGNRDFVEGLSGATWVDVVLEHGGCVTTMAKNKPTLDIELVTTEATNLAEL
RKLCIEGKITDMASDSRCPTQGEAYLDKEQDQNYVCKHTYVDRGWGNGCGLFGKG
SLVTCAKFQCLEPIEGKVVQYENLKYRVMITVHTGDHHDVGNETQRVTAEITPQAST
TEAILGGYGSLGLECSPRTGLDFNEMILLTMKNKAWMVHRQWFFDLPLPWTSGADT
ETPTWNRKELLVTFKNAHAKRQTVVVLGSQEGAMHTALTGATEIEMDGGTRIFAGH
LKCRLKMDKLRLKGMSYAMCTNTFVLTKEVSETQHGTILIEVQYKGEDAPCKIPFST
EDGQGKAHNGRLITANPVVTKKTSKVNIEAEPPFGESNIVIGIGDNALKINWYKK
DENV4 E glycoprotein with ZIKV C10 (SEQ ID NO: 81)
MRCVGVGNRDFVEGVSGGAWVDLVLEHGGCVTTMAQGKPTLDFELVTTTAKNVA
ELRTYCIEASISDMASDSRCPTQGEPYLDKEQDQQYICRRDVVDRGWGNGCGLFGK
GGLVTCAKFSCSGKITGNLVQIENLEYRVMVTVHNGDHHDVGNDTSNHRVTATITP
RSPSVEVKLGGYGSLTLDCEPRSGIDFNEMILMKMKKKTWLVHKQWFLDLPLPWTA
GADTSEVHWNYKERMVTFKVPHAKRQTVTVLGSQEGAMHSALAGATEVEMDGGN
RMFAGHLKCKVRMEKLRIKGMSYTMCSGKFSITKEMAETQHGTTVVEVQYEGAGA
PCKVPIEIRDVNKEKVVGRVISSTPLAENTTSKTNIELEPPFGDSYIVIGVGNSALTLH
WFRK
DENV1 E glycoprotein with ZIKV C10- Neurotropic insertions (SEQ ID NO: 82)
MRCVGIGNRDFVEGLSGATWVDVVLEHGSCVTTMAKDKPTLDIELVTTEVTNPAEL
RKLCIEAKISDMASDSRCPTQGEAYLDKEQDTNFVCRRTFVDRGWGNGCGLFGKGS
LITCAKFKCVTKLEGKIVQYENLKYRVMVTVHTGDHSGMIVHDVGNETTEHRTTATI
TPQAPTSEIQLGGYGSLTLDCSPRTGLDFNEMVLLTMEKKSWLVHKQWFLDLPLPW
TSGADTSQETWNRQDLLVTFKTAHAKRQTVVVLGSQEGAMHTALTGATEIEMDGT
TGRIFAGHLKCRLKMDKLRLKGMSYVMCTGSFKLTKEVAETQHGTVLVEVQYEGT
DAPCKIPFSSQDEKGVTQNGRLITANPIVTDKTENSKVNIEAEPPFGESYIVVGAGEKA
LKLSWFKK
DENV2 E glycoprotein with ZIKV C10- Neurotropic insertions (SEQ ID NO: 83)
MRCIGISNRDFVEGVSGGSWVDIVLEHGSCVTTMAKNKPTLDFELVTTEAKNPAELR
KYCIEAKLTDMASDSRCPTQGEPYLDKEQDKRFVCKHSMVDRGWGNGCGLFGKGG
LVTCAMFTCKKNMEGKVVQPENLEYRIMVTPHSGEHSGMIVHDVGNDTGKHRKEI
KVTPQSSITEAELGGYGSVTMECSPRTGLDFNEMVLLQMENKAWLVHRQWFLDLPL
PWLPGADTQGSNWIQKETLVTFKNPHAKRQTVVVLGSQEGAMHTALTGATEIEMD
GGNGRLFTGHLKCRLRMDKLRLKGMSYSMCTGKFKVTKEIAETQHGTIVIEVQYEG
DGSPCKIPFEIMDLEKRHVLGRLITVNPIVTEKTENSKVNIEAEPPFGDSYIIIGVDPGQ
LKLNWFKK
DENV3 E glycoprotein with ZIKV C10- Neurotropic insertions (SEQ ID NO: 84)
MRCVGIGNRDFVEGLSGATWVDVVLEHGGCVTTMAKNKPTLDIELVTTEATNLAEL
RKLCIEGKITDMASDSRCPTQGEAYLDKEQDQNYVCKHTYVDRGWGNGCGLFGKG
SLVTCAKFQCLEPIEGKVVQYENLKYRVMITVHTGDHSGMIVHDVGNETQRVTAEIT
PQASTTEAILGGYGSLGLECSPRTGLDFNEMILLTMKNKAWMVHRQWFFDLPLPWT
SGADTETPTWNRKELLVTFKNAHAKRQTVVVLGSQEGAMHTALTGATEIEMDGGT
GRIFAGHLKCRLKMDKLRLKGMSYAMCTNTFVLTKEVSETQHGTILIEVQYKGEDA
PCKIPFSTEDGQGKAHNGRLITANPVVTKKTENSKVNIEAEPPFGESNIVIGIGDNALKI
NWYKK
DENV4 E glycoprotein with ZIKV C10- Neurotropic insertions (SEQ ID NO: 85)
MRCVGVGNRDFVEGVSGGAWVDLVLEHGGCVTTMAQGKPTLDFELVTTTAKNVA
ELRTYCIEASISDMASDSRCPTQGEPYLDKEQDQQYICRRDVVDRGWGNGCGLFGK
GGLVTCAKFSCSGKITGNLVQIENLEYRVMVTVHNGDHSGMIVHDVGNDTSNHRVT
ATITPRSPSVEVKLGGYGSLTLDCEPRSGIDFNEMILMKMKKKTWLVHKQWFLDLPL
PWTAGADTSEVHWNYKERMVTFKVPHAKRQTVTVLGSQEGAMHSALAGATEVEM
DGGNGRMFAGHLKCKVRMEKLRIKGMSYTMCSGKFSITKEMAETQHGTTVVEVQY
EGAGAPCKVPIEIRDVNKEKVVGRVISSTPLAENTTENSKTNIELEPPFGDSYIVIGVG
NSALTLHWFRK
DENV1 E glycoprotein with ZIKV C8 (SEQ ID NO: 86)
MRCVGIGNRDFVEGLSGATWVDVVLEHGSCVTTMAKDKPTLDIELLKTEVTNPAVL
RKLCIEAKISDMASDSRCPTQGEATLDKEQDTNFVCRRTFVDRGWGNGCGLFGKGS
LITCAKFKCVTKLEGKIVQYENLKYSVIVTVHTGDHHQVGNETTENGTTATITPQAPT
SEIQLTDYGALTLDCSPRTGLDFNEMVLLTMEKKSWLVHKQWFLDLPLPWTSGAST
SQETWNRQDLLVTFKTAHAKRQTVVVLGSQEGAMHTALTGATEIQTDGTTTIFAGH
LKCRLKMDKLTLKGMSYVMCTGSFKLTKIVAETQHGTVLVEVQYEGTDAPCKIPFS
SQDEKGVTQNGRLITANPIVTDKEKPVNIEAEPPFGESYIVVGAGEKALKLSWFKK
DENV2 E glycoprotein with ZIKV C8 (SEQ ID NO: 87)
MRCIGISNRDFVEGVSGGSWVDIVLEHGSCVTTMAKNKPTLDFELIKTEAKQPATLR
KYCIEAKLTDMASDSRCPTQGEPSLDKEQDKRFVCKHSMVDRGWGNGCGLFGKGG
LVTCAMFTCKKNMEGKVVQPENLEYTIVVTPHSGEHHAVGNDTGKNGKEIKVTPQS
SITEAELTGYGTVTMECSPRTGLDFNEMVLLQMENKAWLVHRQWFLDLPLPWLPGA
DTQGSNWIQKETLVTFKNPHAKRQTVVVLGSQEGAMHTALTGATEIQMDGGNLLFT
GHLKCRLRMDKLQLKGMSYSMCTGKFKVTKIIAETQHGTIVIEVQYEGDGSPCKIPF
EIMDLEKRHVLGRLITVNPIVTEKDSPVNIEAEPPFGDSYIIIGVDPGQLKLNWFKK
DENV3 E glycoprotein with ZIKV C8 (SEQ ID NO: 88)
MRCVGIGNRDFVEGLSGATWVDVVLEHGGCVTTMAKNKPTLDIELQKTEATQLATL
RKLCIEGKITDMASDSRCPTQGEAYLDKEQDQNYVCKHTYVDRGWGNGCGLFGKG
SLVTCAKFQCLEPIEGKVVQYENLKYTVIITVHTGDHHQVGNETNGVTAEITPQASTT
EAILPEYGTLGLECSPRTGLDFNEMILLTMKNKAWMVHRQWFFDLPLPWTSGATTE
TPTWNRKELLVTFKNAHAKRQTVVVLGSQEGAMHTALTGATEIQNDGGTSIFAGHL
KCRLKMDKLELKGMSYAMCTNTFVLTKEVSETQHGTILIEVQYKGEDAPCKIPFSTE
DGQGKAHNGRLITANPVVTKKEEPVNIEAEPPFGESNIVIGIGDNALKINWYKK
DENV4 E glycoprotein with ZIKV C8 (SEQ ID NO: 89)
MRCVGVGNRDFVEGVSGGAWVDLVLEHGGCVTTMAQGKPTLDFELTKTTAKEVA
LLRTYCIEASISDMASDSRCPTQGEPYLDKEQDQQYICRRDVVDRGWGNGCGLFGK
GGLVTCAKFSCSGKITGNLVQIENLEYTVVVTVHNGDHHAVGNDTSNNGVTATITPR
SPSVEVKLPDYGELTLDCEPRSGIDFNEMILMKMKKKTWLVHKQWFLDLPLPWTAG
ADTSEVHWNYKERMVTFKVPHAKRQTVTVLGSQEGAMHSALAGATEVDSDGGNH
MFAGHLKCKVRMEKLRIKGMSYTMCSGKFSITKIMAETQHGTTVVEVQYEGAGAPC
KVPIEIRDVNKEKVVGRVISSTPLAENTNSVTNIELEPPFGDSYIVIGVGNSALTLHWF
RK

TABLE 1
Amino Acid Diversity Among DENV and ZIKV Strains in EDE1 and EDE2
Contact Residues

| | N-Linked | | | | | ZIKV | ZIKV | EDE1 | EDE1 | EDE2 |
|---|---|---|---|---|---|---|---|---|---|---|
| Position | Glycosylation | DENV-1 | DENV-2 | DENV-3 | DENV-4 | H/PF/2013 | PRVABC59 | C8 | C10 | B7 |
| 2 | | R | R | R | R | R | R | | Yes | |
| 27 | | H | H | H | H | H | H | | Yes | |
| 28 | | G | G | G | G | G | G | | Yes | |
| 44 | | E | E | E | E | E | E | | Yes | |
| 45 | | L | L | L | L | L | L | | Yes | |
| 46 | | L | I | Q | T | V | V | | Yes | |
| 47 | | K | K | K | K | T | T | | Yes | |
| 67 | Yes (DENV | N | N | N | N | D | D | Yes | Yes | Yes |
| | Only) | | | | | | | | | |
| 68 | | T | T | I | I | M | M | Yes | Yes | Yes |
| 69 | | T | T | T | T | A | A | Yes | Yes | Yes |
| 70 | | T | T | T | T | S | S | Yes | Yes | Yes |
| 71 | | D | E | D | A | D | D | Yes | Yes | Yes |
| 72 | | S | S | S | T | S | S | Yes | Yes | Yes |
| 73 | | R | R | R | R | R | R | Yes | Yes | Yes |
| 74 | | C | C | C | C | C | C | Yes | Yes | Yes |
| 77 | | Q | Q | Q | Q | Q | Q | Yes | Yes | |
| 81 | | T | S | V | Y | Y | Y | | Yes | |
| 82 | | L | L | L | L | L | L | | Yes | |
| 83 | | V | N | P | K | D | D | Yes | Yes | Yes |
| 84 | | E | E | E | E | K | K | Yes | Yes | |
| 97 | | V | V | V | V | V | V | Yes | Yes | Yes |
| 98 | | D | D | D | D | D | D | Yes | Yes | Yes |
| 99 | | R | R | R | R | R | R | Yes | Yes | Yes |
| 101 | | W | W | W | W | W | W | Yes | Yes | Yes |
| 102 | | G | G | G | G | G | G | Yes | Yes | Yes |
| 103 | | N | N | N | N | N | N | Yes | Yes | Yes |
| 104 | | G | G | G | G | G | G | Yes | Yes | Yes |
| 105 | | C | C | C | C | C | C | Yes | Yes | |
| 106 | | G | G | G | G | G | G | Yes | Yes | |
| 113 | | L | I | L | V | L | L | Yes | Yes | Yes |
| 115 | | T | T | T | T | T | T | | Yes | |
| 148 | | Q | E | Q | T | H | H | Yes | | |
| 151 | | Q | A | Q | A | M | M | ? | ? | |
| 152 | | V | V | V | V | I | I | ? | ? | |
| 153 | | G | G | G | G | V | V | ? | ? | Yes |
| 154 | Yes (DENV & | N | N | N | N | N | N | ? | ? | Yes |
| | ZIKV) | | | | | | | | | |
| 155 | | E | D | E | D | D | D | ? | ? | Yes |
| 156 | | T | T | T | T | T | T | ? | ? | Yes |
| 157 | | T | G | — | S | G | G | ? | ? | Yes |
| 163 | | H | H | Q | H | N | N | Yes | | |
| 251 | | K | K | K | K | K | K | Yes | Yes | Yes |
| 252 | | K | K | K | R | R | R | Yes | Yes | Yes |
| 253 | | Q | Q | Q | Q | Q | Q | Yes | Yes | Yes |
| 254 | | E | D | E | D | T | T | Yes | Yes | Yes |
| 276 | | Q | Q | Q | D | E | E | | Yes | |
| 278 | | S | S | S | G | D | D | Yes | Yes | |
| 279 | | G | S | G | D | G | G | Yes | Yes | |
| 315 | | E | V | K | D | T | T | Yes | Yes | |
| 316 | | K | K | K | K | K | K | Yes | Yes | |
| 317 | | E | E | E | E | I | I | Yes | | |
| 329 | | Q | R | K | K | E | E | Yes | Yes | |
| 331 | | K | Q | E | K | Q | Q | | Yes | |
| 371 | | E | D | E | N | N | N | Yes | Yes | |

TABLE 2
Characteristics of Human Plasma and Sera Used in Neutralization Assays

| Serum Classification | Serum ID | Location | Year of Infection | Year of Collection |
|---|---|---|---|---|
| Primary DENV-1 | DT145 | South America | 2005 | Spring 2016 |
| Primary DENV-1 | DT147 | South America | Fall 2015 | Spring 2016 |
| Primary DENV-1 | DT153 | South America | 2010 | End 2014 |
| Primary DENV-1 | SS05/262 | Asia | Unknown | Unknown |
| Primary DENV-1 | SS99/1230 | Asia | Unknown | Unknown |
| Primary DENV-2 | DT001 | Asia | 1996 | Spring 2016 |
| Primary DENV-2 | DT155 | South America | 2000 | Spring 2015 |
| Primary DENV-2 | SS08/90 | Asia | Unknown | Unknown |
| Primary DENV-2 | SS08/91 | Asia | Unknown | Unknown |
| Primary DENV-3 | DT115 | Asia | Unknown | Winter 2009 |
| Primary DENV-3 | DT118 | South America | 2009 | Fall 2010 |
| Primary DENV-3 | DT125 | South America | 2007 | Spring 2012 |
| Primary DENV-3 | DT133 | South America | 2002 | Summer 2012 |
| Primary DENV-3 | SS06/297 | Asia | Unknown | Unknown |
| Primary DENV-4 | GS0239 | Unknown | Unknown | Unknown |
| Primary DENV-4 | SS06/105 | Asia | Unknown | Unknown |
| Primary DENV-4 | SS06/302 | Asia | Unknown | Unknown |
| Secondary DENV | DT000 | Asia | <1982 | Spring 2016 |
| Secondary DENV | DT112 | South America | 2001 | Spring 2009 |
| Secondary DENV | DT116 | Asia | Unknown | Fall 2009 |
| Secondary DENV | DT141 | South America | 2006 | Fall 2014 |
| Secondary DENV | DT146 | Asia | 2013 | Fall 2014 |
| Secondary DENV | DT160 | South America | 2015 | Summer 2015 |
| Secondary DENV | DV003 | Asia | Unknown | Unknown |
| Secondary DENV | DT144 | Asia | Unknown | Unknown |
| Secondary DENV | SS06/123 | Asia | Unknown | Unknown |
| Secondary DENV | SS06/124 | Asia | Unknown | Unknown |
| Secondary DENV | SS06/125 | Asia | Unknown | Unknown |
| Secondary DENV | SS09/157 | Asia | Unknown | Unknown |
| Secondary DENV | SS09/159 | Asia | Unknown | Unknown |
| Secondary DENV | SS09/165 | Asia | Unknown | Unknown |
| Secondary DENV | SS09/250 | Asia | Unknown | Unknown |
| Secondary DENV | SS09/251 | Asia | Unknown | Unknown |

TABLE 3

| | Abbreviation | |
|---|---|---|
| Amino Acid Residue | Three-Letter Code | One-Letter Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE 4

| | |
|---|---|
| Modified Amino Acid Residue | Abbreviation |

Amino Acid Residue Derivatives

| | |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,2′-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | alle |
| Methionine sulfoxide | MSO |
| N-Methylglycine, sarcosine | MeGly |
| N-Methylisoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-Cl) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

TABLE 5
ZIKV EDE1 null

| | Residue # | | | | | | | | | | | |
|---|---|---|---|---|---|---|---|---|---|---|---|---|
| | 2 | 28 | 44 | 45 | 69 | 71 | 72 | 73 | 77 | 82 | 102 | 103 |
| Zika WT | R | G | E | L | A | D | S | R | Q | L | G | N |
| Core | R | G | E | L | E | R | A | K | Q | L | D | S |
| Core & | H | D | T | V | E | R | A | K | A | I | D | S |
| Specific | | | | | | | | | | | | |
| Specific | H | D | T | V | A | D | S | R | A | I | G | N |

| | Residue # | | | | | | | | | | | | |
|---|---|---|---|---|---|---|---|---|---|---|---|---|---|
| | 106 | 148 | 151 | 163 | 251 | 252 | 253 | 254 | 276 | 316 | 317 | 329 | 331 |
| Zika WT | G | H | M | N | K | R | Q | T | E | K | I | E | Q |
| Core | G | H | M | N | T | Y | I | P | E | K | I | E | Q |
| Core & | F | N | K | K | T | Y | I | P | K | S | G | L | G |
| Specific | | | | | | | | | | | | | |
| Specific | F | N | K | K | K | R | Q | T | K | S | G | L | G |

TABLE 6
DENV1 EDE1 null

| | Residue # | | | | | | | | | | | | |
|---|---|---|---|---|---|---|---|---|---|---|---|---|---|
| | 2 | 28 | 44 | 45 | 69 | 71 | 72 | 73 | 77 | 82 | 102 | 103 | 106 |
| DENV1 WT | R | G | E | L | T | D | S | R | Q | L | G | N | G |
| Core | R | G | E | L | E | R | A | K | Q | L | D | S | G |
| Core & | H | D | T | V | E | R | A | K | A | I | D | S | F |
| Specific | | | | | | | | | | | | | |
| Specific | H | D | T | V | T | D | S | R | A | I | G | N | F |

| | Residue # | | | | | | | | | | | | | |
|---|---|---|---|---|---|---|---|---|---|---|---|---|---|---|
| | 148 | 150 | 158 | 161 | 202 | 246 | 247 | 248 | 249 | 271 | 310 | 311 | 323 | 325 |
| DENV1 WT | Q | Q | H | T | E | K | K | Q | E | Q | K | E | Q | K |
| Core | Q | Q | H | I | K | T | Y | I | P | Q | K | E | Q | K |
| Core & | N | K | K | I | K | T | Y | I | P | K | S | G | L | G |
| Specific | | | | | | | | | | | | | | |
| Specific | N | K | K | I | K | K | K | Q | E | K | S | G | L | G |

TABLE 7
DENV2 EDE1 null

| | Residue # | | | | | | | | | | | | |
|---|---|---|---|---|---|---|---|---|---|---|---|---|---|
| | 2 | 28 | 44 | 45 | 69 | 71 | 72 | 73 | 77 | 82 | 102 | 103 | 106 |
| DENV2 WT | R | G | E | L | T | E | S | R | Q | L | G | N | G |
| Core | R | G | E | L | E | R | A | K | Q | L | D | S | G |
| Core & | H | D | T | V | E | R | A | K | A | I | D | S | F |
| Specific | | | | | | | | | | | | | |
| Specific | H | D | T | V | T | E | S | R | A | I | G | N | F |

| | Residue # | | | | | | | | | | | | | |
|---|---|---|---|---|---|---|---|---|---|---|---|---|---|---|
| | 148 | 150 | 158 | 161 | 202 | 246 | 247 | 248 | 249 | 271 | 310 | 311 | 323 | 325 |
| DENV2 WT | E | A | H | E | E | K | K | Q | D | Q | K | E | R | Q |
| Core | E | A | H | I | K | T | Y | I | P | Q | K | E | R | Q |
| Core & | N | K | K | I | K | T | Y | I | P | K | S | G | L | G |
| Specific | | | | | | | | | | | | | | |
| Specific | N | K | K | I | K | K | K | Q | D | K | S | G | L | G |

TABLE 8
DENV3 EDE1 null

| | Residue # | | | | | | | | | | | | |
|---|---|---|---|---|---|---|---|---|---|---|---|---|---|
| | 2 | 28 | 44 | 45 | 69 | 71 | 72 | 73 | 77 | 82 | 102 | 103 | 106 |
| DENV3 WT | R | G | E | L | T | D | S | R | Q | L | G | N | G |
| Core | R | G | E | L | E | R | A | K | Q | L | D | S | G |
| Core & | H | D | T | V | E | R | A | K | A | I | D | S | F |
| Specific | | | | | | | | | | | | | |
| Specific | H | D | T | V | T | D | S | R | A | I | G | N | F |
| | Residue # | | | | | | | | | | | | |
| | 148 | 150 | 156 | 159 | 244 | 245 | 246 | 247 | 269 | 308 | 309 | 321 | 323 |
| DENV3 WT | Q | Q | Q | T | K | K | Q | E | Q | K | E | K | E |
| Core | Q | Q | Q | I | T | Y | I | P | Q | K | E | K | E |
| Core & | N | K | K | I | T | Y | I | P | K | S | G | L | G |
| Specific | | | | | | | | | | | | | |
| Specific | N | K | K | I | K | K | Q | E | K | S | G | L | G |

TABLE 9
DENV4 EDE1 null

| | Residue # | | | | | | | | | | | | |
|---|---|---|---|---|---|---|---|---|---|---|---|---|---|
| | 2 | 28 | 44 | 45 | 69 | 71 | 72 | 73 | 77 | 82 | 102 | 103 | 106 |
| DENV4 WT | R | G | E | L | T | A | T | R | Q | L | G | N | G |
| Core | R | G | E | L | E | R | A | K | Q | L | D | S | G |
| Core & | H | D | T | V | E | R | A | K | A | I | D | S | F |
| Specific | | | | | | | | | | | | | |
| Specific | H | D | T | V | T | A | T | R | A | I | G | N | F |
| | Residue # | | | | | | | | | | | | |
| | 148 | 150 | 158 | 161 | 246 | 247 | 248 | 249 | 271 | 310 | 311 | 323 | 325 |
| DENV4 WT | T | A | H | T | K | R | Q | D | D | K | E | K | K |
| Core | T | A | H | I | T | Y | I | P | D | K | E | K | K |
| Core & | N | K | K | I | T | Y | I | P | K | S | G | L | G |
| Specific | | | | | | | | | | | | | |
| Specific | N | K | K | I | K | R | Q | D | K | S | G | L | G |

TABLE 10
Zika C10

| | Residue #* | | | | | | | | | | | | |
|---|---|---|---|---|---|---|---|---|---|---|---|---|---|
| | 46 | 47 | 52 | 55 | 67 | 68 | 69 | 70 | 71 | 72 | 73 | 74 | 81 |
| Zika | V | T | N | E | D | M | A | S | D | S | R | C | Y |
| DENV1 | L | K | N | V | N | T | T | T | D | S | R | C | T |
| DENV2 | I | K | Q | T | N | T | T | T | E | S | R | C | S |
| DENV3 | Q | K | Q | T | N | I | T | T | D | S | R | C | V |
| DENV4 | T | K | E | L | N | I | T | T | A | T | R | C | Y |

| | Residue #* | | | | | | | | | | | | |
|---|---|---|---|---|---|---|---|---|---|---|---|---|---|
| | 82 | 83 | 84 | 113 | 138 | 140 | 148 | 149 | 150 | 151 | 152 | 153 | 155 |
| Zika | L | D | K | L | R | M | H | S | G | M | I | V | D |
| DENV1 | L | V | E | L | S | I | Q | | | | | | Q |
| DENV2 | L | N | E | I | T | V | E | | | | | | A |
| DENV3 | L | P | E | L | T | I | Q | | | | | | Q |
| DENV4 | L | K | E | V | T | V | T | | | | | | A |

| | Residue #* | | | | | | | | | | | | |
|---|---|---|---|---|---|---|---|---|---|---|---|---|---|
| | 164 | 180 | 181 | 182 | 184 | 185 | 229 | 230 | 249 | 251 | 252 | 253 | 254 |
| Zika | R | L | G | G | G | S | A | D | H | K | R | Q | T |
| DENV1 | G | L | T | D | G | A | A | S | H | K | K | Q | E |
| DENV2 | G | L | T | G | G | T | A | D | H | K | K | Q | D |
| DENV3 | G | L | P | E | G | T | A | T | H | K | K | Q | E |
| DENV4 | G | L | P | D | G | E | A | D | H | K | R | Q | D |

| | Residue #* | | | | | | | | | | | | | | |
|---|---|---|---|---|---|---|---|---|---|---|---|---|---|---|---|
| | 276 | 277 | 278 | 279 | 283 | 299 | 315 | 316 | 329 | 331 | 369 | 370 | 371 | 372 | 373 |
| Zika | E | M | D | G | R | R | T | K | E | Q | T | E | N | S | K |
| DENV1 | Q | T | S | G | T | T | E | K | Q | K | E | | | K | P |
| DENV2 | Q | M | S | S | L | Q | V | K | R | Q | D | | | S | P |
| DENV3 | Q | N | S | G | S | E | K | K | K | E | E | | | E | P |
| DENV4 | D | S | G | D | H | R | D | K | K | K | N | | | S | V |

*Relative to ZIKV WT E

TABLE 11
Zika C8

| | Residue #* | | | | | | | | | |
|---|---|---|---|---|---|---|---|---|---|---|
| | 67 | 68 | 69 | 70 | 71 | 72 | 73 | 74 | 83 | 84 |
| Zika | D | M | A | S | D | S | R | C | D | K |
| DENV1 | N | T | T | T | D | S | R | C | V | E |
| DENV2 | N | T | T | T | E | S | R | C | N | E |
| DENV3 | N | I | T | T | D | S | R | C | P | E |
| DENV4 | N | I | T | T | A | T | R | C | K | E |
| | Residue #* | | | | | | | | | |
| | 113 | 148 | 163 | 252 | 254 | 278 | 279 | 315 | 317 | 329 |
| Zika | L | H | N | R | T | D | G | T | I | E |
| DENV1 | L | Q | H | K | E | S | G | E | E | Q |
| DENV2 | I | E | H | K | D | S | S | V | E | R |
| DENV3 | L | Q | Q | K | E | S | G | K | E | K |
| DENV4 | V | T | H | R | D | G | D | D | E | K |

*Relative to ZIKV WT E

TABLE 12
DENV1 C10

| | Residue #* | | | | | | | | | | | | |
|---|---|---|---|---|---|---|---|---|---|---|---|---|---|
| | 46 | 47 | 52 | 55 | 67 | 68 | 69 | 70 | 71 | 72 | 73 | 74 | 81 |
| Zika | V | T | N | E | D | M | A | S | D | S | R | C | Y |
| DENV1 | L | K | N | V | N | T | T | T | D | S | R | C | T |
| DENV2 | I | K | Q | T | N | T | T | T | E | S | R | C | S |
| DENV3 | Q | K | Q | T | N | I | T | T | D | S | R | C | V |
| DENV4 | T | K | E | L | N | I | T | T | A | T | R | C | Y |
| | Residue #* | | | | | | | | | | | | |
| | 82 | 83 | 84 | 113 | 138 | 140 | 148 | | | | | | 150 |
| Zika | L | D | K | L | R | M | H | S | G | M | I | V | D |
| DENV1 | L | V | E | L | S | I | Q | | | | | | Q |
| DENV2 | L | N | E | I | T | V | E | | | | | | A |
| DENV3 | L | P | E | L | T | I | Q | | | | | | Q |
| DENV4 | L | K | E | V | T | V | T | | | | | | A |
| | Residue #* | | | | | | | | | | | | |
| | 159 | 175 | 176 | 177 | 179 | 180 | 224 | 225 | 244 | 246 | 247 | 248 | 249 |
| Zika | R | L | G | G | G | S | A | D | H | K | R | Q | T |
| DENV1 | G | L | T | D | G | A | A | S | H | K | K | Q | E |
| DENV2 | G | L | T | G | G | T | A | D | H | K | K | Q | D |
| DENV3 | G | L | P | E | G | T | A | T | H | K | K | Q | E |
| DENV4 | G | L | P | D | G | E | A | D | H | K | R | Q | D |
| | Residue #* | | | | | | | | | | | | |
| | 271 | 272 | 273 | 274 | 278 | 293 | 309 | 310 | 323 | 325 | 362 | 363 | 364 |
| Zika | E | M | D | G | R | R | T | K | E | Q | T | S | K |
| DENV1 | Q | T | S | G | T | T | E | K | Q | K | E | K | P |
| DENV2 | Q | M | S | S | L | Q | V | K | R | Q | D | S | P |
| DENV3 | Q | N | S | G | S | E | K | K | K | E | E | E | P |
| DENV4 | D | S | G | D | H | R | D | K | K | K | N | S | V |

*Relative to DENV1 WT E

### *Relative to DENV1 WT E

TABLE 13
DENV1 C8

Residue #*

| | 67 | 68 | 69 | 70 | 71 | 72 | 73 | 74 | 83 | 84 |
|---|---|---|---|---|---|---|---|---|---|---|
| Zika | D | M | A | S | D | S | R | C | D | K |
| DENV1 | N | T | T | T | D | S | R | C | V | E |
| DENV2 | N | T | T | T | E | S | R | C | N | E |
| DENV3 | N | I | T | T | D | S | R | C | P | E |
| DENV4 | N | I | T | T | A | T | R | C | K | E |

| | Residue #* | | | | | | | | | |
|---|---|---|---|---|---|---|---|---|---|---|
| | 113 | 148 | 158 | 247 | 249 | 273 | 274 | 309 | 311 | 323 |
| Zika | L | H | N | R | T | D | G | T | I | E |
| DENV1 | L | Q | H | K | E | S | G | E | E | Q |
| DENV2 | I | E | H | K | D | S | S | V | E | R |
| DENV3 | L | Q | Q | K | E | S | G | K | E | K |
| DENV4 | V | T | H | R | D | G | D | D | E | K |

*Relative to DENV1 WT E

TABLE 14
DENV2 C10

| | Residue #* | | | | | | | | | | | | |
|---|---|---|---|---|---|---|---|---|---|---|---|---|---|
| | 46 | 47 | 52 | 55 | 67 | 68 | 69 | 70 | 71 | 72 | 73 | 74 | 81 |
| Zika | V | T | N | E | D | M | A | S | D | S | R | C | Y |
| DENV1 | L | K | N | V | N | T | T | T | D | S | R | C | T |
| DENV2 | I | K | Q | T | N | T | T | T | E | S | R | C | S |
| DENV3 | Q | K | Q | T | N | I | T | T | D | S | R | C | V |
| DENV4 | T | K | E | L | N | I | T | T | A | T | R | C | Y |
| | Residue #* | | | | | | | | | | | | |
| | 82 | 83 | 84 | 113 | 138 | 140 | 148 | | | | | | 150 |
| Zika | L | D | K | L | R | M | H | S | G | M | I | V | D |
| DENV1 | L | V | E | L | S | I | Q | | | | | | Q |
| DENV2 | L | N | E | I | T | V | E | | | | | | A |
| DENV3 | L | P | E | L | T | I | Q | | | | | | Q |
| DENV4 | L | K | E | V | T | V | T | | | | | | A |
| | Residue #* | | | | | | | | | | | | |
| | 159 | 175 | 176 | 177 | 179 | 180 | 224 | 225 | 244 | 246 | 247 | 248 | 249 |
| Zika | R | L | G | G | G | S | A | D | H | K | R | Q | T |
| DENV1 | G | L | T | D | G | A | A | S | H | K | K | Q | E |
| DENV2 | G | L | T | G | G | T | A | D | H | K | K | Q | D |
| DENV3 | G | L | P | E | G | T | A | T | H | K | K | Q | E |
| DENV4 | G | L | P | D | G | E | A | D | H | K | R | Q | D |
| | Residue #* | | | | | | | | | | | | |
| | 271 | 272 | 273 | 274 | 278 | 293 | 309 | 310 | 323 | 325 | 362 | 363 | 364 |
| Zika | E | M | D | G | R | R | T | K | E | Q | T | S | K |
| DENV1 | Q | T | S | G | T | T | E | K | Q | K | E | K | P |
| DENV2 | Q | M | S | S | L | Q | V | K | R | Q | D | S | P |
| DENV3 | Q | N | S | G | S | E | K | K | K | E | E | E | P |
| DENV4 | D | S | G | D | H | R | D | K | K | K | N | S | V |

*Relative to DENV2 WT E

TABLE 15
DENV2 C8

Residue #*

| | 67 | 68 | 69 | 70 | 71 | 72 | 73 | 74 | 83 | 84 |
|---|---|---|---|---|---|---|---|---|---|---|
| Zika | D | M | A | S | D | S | R | C | D | K |
| DENV1 | N | T | T | T | D | S | R | C | V | E |
| DENV2 | N | T | T | T | E | S | R | C | N | E |
| DENV3 | N | I | T | T | D | S | R | C | P | E |
| DENV4 | N | I | T | T | A | T | R | C | K | E |

| | Residue #* | | | | | | | | | |
|---|---|---|---|---|---|---|---|---|---|---|
| | 113 | 148 | 158 | 247 | 249 | 273 | 274 | 309 | 311 | 323 |
| Zika | L | H | N | R | T | D | G | T | I | E |
| DENV1 | L | Q | H | K | E | S | G | E | E | Q |
| DENV2 | I | E | H | K | D | S | S | V | E | R |
| DENV3 | L | Q | Q | K | E | S | G | K | E | K |
| DENV4 | V | T | H | R | D | G | D | D | E | K |

*Relative to DENV2 WT E

TABLE 16
DENV3 C10

| | Residue #* | | | | | | | | | | | | |
|---|---|---|---|---|---|---|---|---|---|---|---|---|---|
| | 46 | 47 | 52 | 55 | 67 | 68 | 69 | 70 | 71 | 72 | 73 | 74 | 81 |
| Zika | V | T | N | E | D | M | A | S | D | S | R | C | Y |
| DENV1 | L | K | N | V | N | T | T | T | D | S | R | C | T |
| DENV2 | I | K | Q | T | N | T | T | T | E | S | R | C | S |
| DENV3 | Q | K | Q | T | N | I | T | T | D | S | R | C | V |
| DENV4 | T | K | E | L | N | I | T | T | A | T | R | C | Y |
| | Residue #* | | | | | | | | | | | | |
| | 82 | 83 | 84 | 113 | 138 | 140 | 148 | | | | | | 150 |
| Zika | L | D | K | L | R | M | H | S | G | M | I | V | D |
| DENV1 | L | V | E | L | S | I | Q | | | | | | Q |
| DENV2 | L | N | E | I | T | V | E | | | | | | A |
| DENV3 | L | P | E | L | T | I | Q | | | | | | Q |
| DENV4 | L | K | E | V | T | V | T | | | | | | A |
| | Residue #* | | | | | | | | | | | | |
| | 159 | 173 | 174 | 175 | 177 | 178 | 222 | 223 | 242 | 244 | 245 | 246 | 247 |
| Zika | R | L | G | G | G | S | A | D | H | K | R | Q | T |
| DENV1 | G | L | T | D | G | A | A | S | H | K | K | Q | E |
| DENV2 | G | L | T | G | G | T | A | D | H | K | K | Q | D |
| DENV3 | G | L | P | E | G | T | A | T | H | K | K | Q | E |
| DENV4 | G | L | P | D | G | E | A | D | H | K | R | Q | D |
| | Residue #* | | | | | | | | | | | | |
| | 269 | 270 | 271 | 272 | 276 | 291 | 307 | 308 | 321 | 323 | 360 | 361 | 362 |
| Zika | E | M | D | G | R | R | T | K | E | Q | T | S | K |
| DENV1 | Q | T | S | G | T | T | E | K | Q | K | E | K | P |
| DENV2 | Q | M | S | S | L | Q | V | K | R | Q | D | S | P |
| DENV3 | Q | N | S | G | S | E | K | K | K | E | E | E | P |
| DENV4 | D | S | G | D | H | R | D | K | K | K | N | S | V |

*Relative to DENV3 WT E

TABLE 17
DENV3 C8

Residue #*

| | 67 | 68 | 69 | 70 | 71 | 72 | 73 | 74 | 83 | 84 |
|---|---|---|---|---|---|---|---|---|---|---|
| Zika | D | M | A | S | D | S | R | C | D | K |
| DENV1 | N | T | T | T | D | S | R | C | V | E |
| DENV2 | N | T | T | T | E | S | R | C | N | E |
| DENV3 | N | I | T | T | D | S | R | C | P | E |
| DENV4 | N | I | T | T | A | T | R | C | K | E |

| | Residue #* | | | | | | | | | |
|---|---|---|---|---|---|---|---|---|---|---|
| | 113 | 148 | 158 | 245 | 247 | 271 | 272 | 307 | 309 | 321 |
| Zika | L | H | N | R | T | D | G | T | I | E |
| DENV1 | L | Q | H | K | E | S | G | E | E | Q |
| DENV2 | I | E | H | K | D | S | S | V | E | R |
| DENV3 | L | Q | Q | K | E | S | G | K | E | K |
| DENV4 | V | T | H | R | D | G | D | D | E | K |

*Relative to DENV3 WT E

TABLE 18
DENV4 C10

| | Residue #* | | | | | | | | | | | | |
|---|---|---|---|---|---|---|---|---|---|---|---|---|---|
| | 46 | 47 | 52 | 55 | 67 | 68 | 69 | 70 | 71 | 72 | 73 | 74 | 81 |
| Zika | V | T | N | E | D | M | A | S | D | S | R | C | Y |
| DENV1 | L | K | N | V | N | T | T | T | D | S | R | C | T |
| DENV2 | I | K | Q | T | N | T | T | T | E | S | R | C | S |
| DENV3 | Q | K | Q | T | N | I | T | T | D | S | R | C | V |
| DENV4 | T | K | E | L | N | I | T | T | A | T | R | C | Y |
| | Residue #* | | | | | | | | | | | | |
| | 82 | 83 | 84 | 113 | 138 | 140 | 148 | | | | | | 150 |
| Zika | L | D | K | L | R | M | H | S | G | M | I | V | D |
| DENV1 | L | V | E | L | S | I | Q | | | | | | Q |
| DENV2 | L | N | E | I | T | V | E | | | | | | A |
| DENV3 | L | P | E | L | T | I | Q | | | | | | Q |
| DENV4 | L | K | E | V | T | V | T | | | | | | A |
| | Residue #* | | | | | | | | | | | | |
| | 159 | 175 | 176 | 177 | 179 | 180 | 224 | 225 | 244 | 246 | 247 | 248 | 249 |
| Zika | R | L | G | G | G | S | A | D | H | K | R | Q | T |
| DENV1 | G | L | T | D | G | A | A | S | H | K | K | Q | E |
| DENV2 | G | L | T | G | G | T | A | D | H | K | K | Q | D |
| DENV3 | G | L | P | E | G | T | A | T | H | K | K | Q | E |
| DENV4 | G | L | P | D | G | E | A | D | H | K | R | Q | D |
| | Residue #* | | | | | | | | | | | | |
| | 271 | 272 | 273 | 274 | 278 | 293 | 309 | 310 | 323 | 325 | 362 | 363 | 364 |
| Zika | E | M | D | G | R | R | T | K | E | Q | T | S | K |
| DENV1 | Q | T | S | G | T | T | E | K | Q | K | E | K | P |
| DENV2 | Q | M | S | S | L | Q | V | K | R | Q | D | S | P |
| DENV3 | Q | N | S | G | S | E | K | K | K | E | E | E | P |
| DENV4 | D | S | G | D | H | R | D | K | K | K | N | S | V |

*Relative to DENV4 WT E

TABLE 19
DENV4 C8

| | Residue #* | | | | | | | | | |
|---|---|---|---|---|---|---|---|---|---|---|
| | 67 | 68 | 69 | 70 | 71 | 72 | 73 | 74 | 83 | 84 |
| Zika | D | M | A | S | D | S | R | C | D | K |
| DENV1 | N | T | T | T | D | S | R | C | V | E |
| DENV2 | N | T | T | T | E | S | R | C | N | E |
| DENV3 | N | I | T | T | D | S | R | C | P | E |
| DENV4 | N | I | T | T | A | T | R | C | K | E |
| | Residue #* | | | | | | | | | |
| | 113 | 148 | 158 | 247 | 249 | 273 | 274 | 309 | 311 | 323 |
| Zika | L | H | N | R | T | D | G | T | I | E |
| DENV1 | L | Q | H | K | E | S | G | E | E | Q |
| DENV2 | I | E | H | K | D | S | S | V | E | R |
| DENV3 | L | Q | Q | K | E | S | G | K | E | K |
| DENV4 | V | T | H | R | D | G | D | D | E | K |

*Relative to DENV4 WT E

TABLE 20
ZIKV-DENV 5J7 Swap

| | Residue #* | | | | | | | | | | | | |
|---|---|---|---|---|---|---|---|---|---|---|---|---|---|
| | 49 | 50 | 51 | 52 | 53 | 54 | 55 | 56 | 57 | 58 | 59 | 60 | 61 |
| DENV1 | E | V | T | N | P | A | V | L | R | K | L | C | I |
| DENV2 | E | A | K | Q | P | A | T | L | R | K | Y | C | I |
| DENV3 | E | A | T | Q | L | A | T | L | R | K | L | C | I |
| DENV4 | T | A | K | E | V | A | L | L | R | T | Y | C | I |
| Zika WT | T | V | S | N | M | A | E | V | R | S | Y | C | Y |
| | Residue #* | | | | | | | | | | | | |
| | 62 | 63 | 122 | 123 | 124 | 125 | 126 | 129 | 130 | 132 | 200 | 201 | 202 |
| DENV1 | E | A | V | T | K | L | E | I | V | Y | E | M | V |
| DENV2 | E | A | K | K | N | M | E | V | V | P | E | M | V |
| DENV3 | E | G | L | E | P | I | E | V | V | Y | E | M | I |
| DENV4 | E | A | S | G | K | I | T | L | V | I | E | M | I |
| Zika WT | E | A | S | K | K | M | T | S | I | P | D | L | Y |
| | Residue #* | | | | | | | | | | | | |
| | 203 | 207 | 210 | 212 | 215 | 216 | 219 | 221 | 226 | 227 | 230 | 232 | 235 |
| DENV1 | L | E | S | L | K | Q | L | L | T | S | S | S | T |
| DENV2 | L | E | A | L | R | Q | L | L | L | P | D | Q | N |
| DENV3 | L | K | A | M | R | Q | F | L | T | S | T | E | T |
| DENV4 | L | K | T | L | K | Q | L | L | T | A | D | S | H |
| Zika WT | Y | N | H | L | K | E | H | I | H | A | D | G | H |

| | Residue #* | | | | | | | | | |
|---|---|---|---|---|---|---|---|---|---|---|
| | 275 | 276 | 278 | 279 | 280 | 281 | 283 | 284 | 285 | 286 |
| DENV1 | I | Q | S | G | T | T | T | I | F | A |
| DENV2 | I | Q | S | S | G | N | L | L | F | T |
| DENV3 | I | Q | S | G | G | T | S | I | F | A |
| DENV4 | V | D | G | D | G | N | H | M | F | A |
| Zika WT | A | E | D | G | A | K | R | L | S | S |

*Relative to ZIKV E WT

TABLE 21
ZIKV-DENV1 5J7 Swap

| | Residue #* | | | | | | | | | | | | |
|---|---|---|---|---|---|---|---|---|---|---|---|---|---|
| | 49 | 50 | 51 | 52 | 53 | 54 | 55 | 56 | 57 | 58 | 59 | 60 | 61 |
| DENV1 5J7 | E | V | T | N | P | A | V | L | R | K | L | C | I |
| DENV2 5J7 | E | A | K | Q | P | A | T | L | R | K | Y | C | I |
| DENV3 5J7 | E | A | T | Q | L | A | T | L | R | K | L | C | I |
| DENV4 5J7 | T | A | K | E | V | A | L | L | R | T | Y | C | I |
| Zika WT | T | V | S | N | M | A | E | V | R | S | Y | C | Y |
| | Residue #* | | | | | | | | | | | | |
| | 62 | 63 | 122 | 123 | 124 | 125 | 126 | 129 | 130 | 132 | 195 | 196 | 197 |
| DENV1 5J7 | E | A | V | T | K | L | E | I | V | Y | E | M | V |
| DENV2 5J7 | E | A | K | K | N | M | E | V | V | P | E | M | V |
| DENV3 5J7 | E | G | L | E | P | I | E | V | V | Y | E | M | I |
| DENV4 5J7 | E | A | S | G | K | I | T | L | V | I | E | M | I |
| Zika WT | E | A | S | K | K | M | T | S | I | P | D | L | Y |
| | Residue #* | | | | | | | | | | | | |
| | 198 | 202 | 205 | 207 | 210 | 211 | 214 | 216 | 221 | 222 | 225 | 227 | 230 |
| DENV1 5J7 | L | E | S | L | K | Q | L | L | T | S | S | S | T |
| DENV2 5J7 | L | E | A | L | R | Q | L | L | L | P | D | Q | N |
| DENV3 5J7 | L | K | A | M | R | Q | F | L | T | S | T | E | T |
| DENV4 5J7 | L | K | T | L | K | Q | L | L | T | A | D | S | H |
| Zika WT | Y | N | H | L | K | E | H | I | H | A | D | G | H |

| | Residue #* | | | | | | | | | |
|---|---|---|---|---|---|---|---|---|---|---|
| | 270 | 271 | 273 | 274 | 275 | 276 | 278 | 279 | 280 | 281 |
| DENV1 5J7 | I | Q | S | G | T | T | T | I | F | A |
| DENV2 5J7 | I | Q | S | S | G | N | L | L | F | T |
| DENV3 5J7 | I | Q | S | G | G | T | S | I | F | A |
| DENV4 5J7 | V | D | G | D | G | N | H | M | F | A |
| Zika WT | A | E | D | G | A | K | R | L | S | S |

*Relative to DENV1 E WT

TABLE 22
ZIKV-DENV2 5J7 Swap

| | Residue #* | | | | | | | | | | | | |
|---|---|---|---|---|---|---|---|---|---|---|---|---|---|
| | 49 | 50 | 51 | 52 | 53 | 54 | 55 | 56 | 57 | 58 | 59 | 60 | 61 |
| DENV1 5J7 | E | V | T | N | P | A | V | L | R | K | L | C | I |
| DENV2 5J7 | E | A | K | Q | P | A | T | L | R | K | Y | C | I |
| DENV3 5J7 | E | A | T | Q | L | A | T | L | R | K | L | C | I |
| DENV4 5J7 | T | A | K | E | V | A | L | L | R | T | Y | C | I |
| Zika WT | T | V | S | N | M | A | E | V | R | S | Y | C | Y |
| | Residue #* | | | | | | | | | | | | |
| | 62 | 63 | 122 | 123 | 124 | 125 | 126 | 129 | 130 | 132 | 195 | 196 | 197 |
| DENV1 5J7 | E | A | V | T | K | L | E | I | V | Y | E | M | V |
| DENV2 5J7 | E | A | K | K | N | M | E | V | V | P | E | M | V |
| DENV3 5J7 | E | G | L | E | P | I | E | V | V | Y | E | M | I |
| DENV4 5J7 | E | A | S | G | K | I | T | L | V | I | E | M | I |
| Zika WT | E | A | S | K | K | M | T | S | I | P | D | L | Y |
| | Residue #* | | | | | | | | | | | | |
| | 198 | 202 | 205 | 207 | 210 | 211 | 214 | 216 | 221 | 222 | 225 | 227 | 230 |
| DENV1 5J7 | L | E | S | L | K | Q | L | L | T | S | S | S | T |
| DENV2 5J7 | L | E | A | L | R | Q | L | L | L | P | D | Q | N |
| DENV3 5J7 | L | K | A | M | R | Q | F | L | T | S | T | E | T |
| DENV4 5J7 | L | K | T | L | K | Q | L | L | T | A | D | S | H |
| Zika WT | Y | N | H | L | K | E | H | I | H | A | D | G | H |

| | Residue #* | | | | | | | | | |
|---|---|---|---|---|---|---|---|---|---|---|
| | 270 | 271 | 273 | 274 | 275 | 276 | 278 | 279 | 280 | 281 |
| DENV1 5J7 | I | Q | S | G | T | T | T | I | F | A |
| DENV2 5J7 | I | Q | S | S | G | N | L | L | F | T |
| DENV3 5J7 | I | Q | S | G | G | T | S | I | F | A |
| DENV4 5J7 | V | D | G | D | G | N | H | M | F | A |
| Zika WT | A | E | D | G | A | K | R | L | S | S |

*Relative to DENV2 E WT

TABLE 23
ZIKV-DENV3 5J7 Swap

| | Residue #* | | | | | | | | | | | | |
|---|---|---|---|---|---|---|---|---|---|---|---|---|---|
| | 49 | 50 | 51 | 52 | 53 | 54 | 55 | 56 | 57 | 58 | 59 | 60 | 61 |
| DENV1 5J7 | E | V | T | N | P | A | V | L | R | K | L | C | I |
| DENV2 5J7 | E | A | K | Q | P | A | T | L | R | K | Y | C | I |
| DENV3 5J7 | E | A | T | Q | L | A | T | L | R | K | L | C | I |
| DENV4 5J7 | T | A | K | E | V | A | L | L | R | T | Y | C | I |
| Zika WT | T | V | S | N | M | A | E | V | R | S | Y | C | Y |
| | Residue #* | | | | | | | | | | | | |
| | 62 | 63 | 122 | 123 | 124 | 125 | 126 | 129 | 130 | 132 | 193 | 194 | 195 |
| DENV1 5J7 | E | A | V | T | K | L | E | I | V | Y | E | M | V |
| DENV2 5J7 | E | A | K | K | N | M | E | V | V | P | E | M | V |
| DENV3 5J7 | E | G | L | E | P | I | E | V | V | Y | E | M | I |
| DENV4 5J7 | E | A | S | G | K | I | T | L | V | I | E | M | I |
| Zika WT | E | A | S | K | K | M | T | S | I | P | D | L | Y |
| | Residue #* | | | | | | | | | | | | |
| | 196 | 200 | 203 | 205 | 208 | 209 | 212 | 214 | 219 | 220 | 223 | 225 | 228 |
| DENV1 5J7 | L | E | S | L | K | Q | L | L | T | S | S | S | T |
| DENV2 5J7 | L | E | A | L | R | Q | L | L | L | P | D | Q | N |
| DENV3 5J7 | L | K | A | M | R | Q | F | L | T | S | T | E | T |
| DENV4 5J7 | L | K | T | L | K | Q | L | L | T | A | D | S | H |
| Zika WT | Y | N | H | L | K | E | H | I | H | A | D | G | H |

| | Residue #* | | | | | | | | | |
|---|---|---|---|---|---|---|---|---|---|---|
| | 268 | 269 | 271 | 272 | 273 | 274 | 276 | 277 | 278 | 279 |
| DENV1 5J7 | I | Q | S | G | T | T | T | I | F | A |
| DENV2 5J7 | I | Q | S | S | G | N | L | L | F | T |
| DENV3 5J7 | I | Q | S | G | G | T | S | I | F | A |
| DENV4 5J7 | V | D | G | D | G | N | H | M | F | A |
| Zika WT | A | E | D | G | A | K | R | L | S | S |

*Relative to DENV3 E WT

TABLE 24
ZIKV-DENV4 5J7 Swap

| | Residue #* | | | | | | | | | | | | |
|---|---|---|---|---|---|---|---|---|---|---|---|---|---|
| | 49 | 50 | 51 | 52 | 53 | 54 | 55 | 56 | 57 | 58 | 59 | 60 | 61 |
| DENV1 5J7 | E | V | T | N | P | A | V | L | R | K | L | C | I |
| DENV2 5J7 | E | A | K | Q | P | A | T | L | R | K | Y | C | I |
| DENV3 5J7 | E | A | T | Q | L | A | T | L | R | K | L | C | I |
| DENV4 5J7 | T | A | K | E | V | A | L | L | R | T | Y | C | I |
| Zika WT | T | V | S | N | M | A | E | V | R | S | Y | C | Y |
| | Residue #* | | | | | | | | | | | | |
| | 62 | 63 | 122 | 123 | 124 | 125 | 126 | 129 | 130 | 132 | 195 | 196 | 197 |
| DENV1 5J7 | E | A | V | T | K | L | E | I | V | Y | E | M | V |
| DENV2 5J7 | E | A | K | K | N | M | E | V | V | P | E | M | V |
| DENV3 5J7 | E | G | L | E | P | I | E | V | V | Y | E | M | I |
| DENV4 5J7 | E | A | S | G | K | I | T | L | V | I | E | M | I |
| Zika WT | E | A | S | K | K | M | T | S | I | P | D | L | Y |
| | Residue #* | | | | | | | | | | | | |
| | 198 | 202 | 205 | 207 | 210 | 211 | 214 | 216 | 221 | 222 | 225 | 227 | 230 |
| DENV1 5J7 | L | E | S | L | K | Q | L | L | T | S | S | S | T |
| DENV2 5J7 | L | E | A | L | R | Q | L | L | L | P | D | Q | N |
| DENV3 5J7 | L | K | A | M | R | Q | F | L | T | S | T | E | T |
| DENV4 5J7 | L | K | T | L | K | Q | L | L | T | A | D | S | H |
| Zika WT | Y | N | H | L | K | E | H | I | H | A | D | G | H |

| | Residue #* | | | | | | | | | |
|---|---|---|---|---|---|---|---|---|---|---|
| | 270 | 271 | 273 | 274 | 275 | 276 | 278 | 279 | 280 | 281 |
| DENV1 5J7 | I | Q | S | G | T | T | T | I | F | A |
| DENV2 5J7 | I | Q | S | S | G | N | L | L | F | T |
| DENV3 5J7 | I | Q | S | G | G | T | S | I | F | A |
| DENV4 5J7 | V | D | G | D | G | N | H | M | F | A |
| Zika WT | A | E | D | G | A | K | R | L | S | S |

*Relative to DENV4 E WT

TABLE 25
DENV-ZIKV Z20 Swap

| | Residue #* | | | | | | | | | | |
|---|---|---|---|---|---|---|---|---|---|---|---|
| | 67 | 68 | 69 | 84 | 120 | 208 | 232 | 233 | 276 | 277 | 278 |
| Zika | D | M | A | K | A | N | G | T | E | M | D |
| DENV1 | N | T | T | E | K | K | S | Q | Q | T | |
| DENV2 | N | T | T | E | T | N | Q | G | Q | M | |
| DENV3 | N | I | T | E | Q | N | E | T | Q | N | |
| DENV4 | N | I | T | E | S | K | S | E | D | S | |

*Relative to ZikV WT

| | Residue #* | | | | | | | | | | |
|---|---|---|---|---|---|---|---|---|---|---|---|
| | 67 | 68 | 69 | 84 | 120 | 203 | 227 | 228 | 271 | 272 | 273 |
| Zika | D | M | A | K | A | N | G | T | E | M | D |
| DENV1 | N | T | T | E | K | K | S | Q | Q | T | |
| DENV2 | N | T | T | E | T | N | Q | G | Q | M | |
| DENV3 | N | I | T | E | Q | N | E | T | Q | N | |
| DENV4 | N | I | T | E | S | K | S | E | D | S | |

*Relative to DENV1 WT

| | Residue #* | | | | | | | | | | |
|---|---|---|---|---|---|---|---|---|---|---|---|
| | 67 | 68 | 69 | 84 | 120 | 203 | 227 | 228 | 271 | 272 | 273 |
| Zika | D | M | A | K | A | N | G | T | E | M | D |
| DENV1 | N | T | T | E | K | K | S | Q | Q | T | |
| DENV2 | N | T | T | E | T | N | Q | G | Q | M | |
| DENV3 | N | I | T | E | Q | N | E | T | Q | N | |
| DENV4 | N | I | T | E | S | K | S | E | D | S | |

*Relative to DENV2 WT

| | Residue #* | | | | | | | | | | |
|---|---|---|---|---|---|---|---|---|---|---|---|
| | 67 | 68 | 69 | 84 | 120 | 201 | 225 | 226 | 269 | 270 | 271 |
| Zika | D | M | A | K | A | N | G | T | E | M | D |
| DENV1 | N | T | T | E | K | K | S | Q | Q | T | |
| DENV2 | N | T | T | E | T | N | Q | G | Q | M | |
| DENV3 | N | I | T | E | Q | N | E | T | Q | N | |
| DENV4 | N | I | T | E | S | K | S | E | D | S | |

*Relative to DENV3 WT

| | Residue #* | | | | | | | | | | |
|---|---|---|---|---|---|---|---|---|---|---|---|
| | 67 | 68 | 69 | 84 | 120 | 203 | 227 | 228 | 271 | 272 | 273 |
| Zika | D | M | A | K | A | N | G | T | E | M | D |
| DENV1 | N | T | T | E | K | K | S | Q | Q | T | |
| DENV2 | N | T | T | E | T | N | Q | G | Q | M | |
| DENV3 | N | I | T | E | Q | N | E | T | Q | N | |
| DENV4 | N | I | T | E | S | K | S | E | D | S | |

*Relative to DENV4 WT

TABLE 26
DENV-ZIKV Z3L1 Swap

| | Residue #* | | | | | | | | |
|---|---|---|---|---|---|---|---|---|---|
| | 46 | 47 | 52 | 138 | 140 | 156 | 158 | 159 | 166 |
| Zika | V | T | N | R | M | T | H | E | K |
| DENV1 | L | K | N | S | I | V | N | E | T |
| DENV2 | I | K | Q | T | V | V | N | D | E |
| DENV3 | Q | K | Q | T | I | V | N | E | T |
| DENV4 | T | K | E | T | V | V | N | D | T |

| | Residue #* | | | | | | | |
|---|---|---|---|---|---|---|---|---|
| | 168 | 208 | 276 | 277 | 278 | 280 | 281 | 283 |
| Zika | E | N | E | M | D | A | K | R |
| DENV1 | T | K | Q | T | | G | T | T |
| DENV2 | K | N | Q | M | | S | G | L |
| DENV3 | E | N | Q | N | | G | G | S |
| DENV4 | T | K | D | S | | D | G | H |

*Relative to ZikV WT

TABLE 27
DENV1-ZIKV Z3L1 Swap

| | Residue #* | | | | | | | | |
|---|---|---|---|---|---|---|---|---|---|
| | 46 | 47 | 52 | 138 | 140 | 151 | 153 | 154 | 161 |
| Zika | V | T | N | R | M | T | H | E | K |
| DENV1 | L | K | N | S | I | V | N | E | T |
| DENV2 | I | K | Q | T | V | V | N | D | E |
| DENV3 | Q | K | Q | T | I | V | N | E | T |
| DENV4 | T | K | E | T | V | V | N | D | T |

| | Residue #* | | | | | | | |
|---|---|---|---|---|---|---|---|---|
| | 163 | 203 | 271 | 272 | 273 | 275 | 276 | 278 |
| Zika | E | N | E | M | D | A | K | R |
| DENV1 | T | K | Q | T | | G | T | T |
| DENV2 | K | N | Q | M | | S | G | L |
| DENV3 | E | N | Q | N | | G | G | S |
| DENV4 | T | K | D | S | | D | G | H |

*Relative to DENV1 WT

TABLE 28
DENV2-ZIKV Z3L1 Swap

| | Residue #* | | | | | | | | |
|---|---|---|---|---|---|---|---|---|---|
| | 46 | 47 | 52 | 138 | 140 | 151 | 153 | 154 | 161 |
| Zika | V | T | N | R | M | T | H | E | K |
| DENV1 | L | K | N | S | I | V | N | E | T |
| DENV2 | I | K | Q | T | V | V | N | D | E |
| DENV3 | Q | K | Q | T | I | V | N | E | T |
| DENV4 | T | K | E | T | V | V | N | D | T |

| | Residue #* | | | | | | | |
|---|---|---|---|---|---|---|---|---|
| | 163 | 203 | 271 | 272 | 273 | 275 | 276 | 278 |
| Zika | E | N | E | M | D | A | K | R |
| DENV1 | T | K | Q | T | | G | T | T |
| DENV2 | K | N | Q | M | | S | G | L |
| DENV3 | E | N | Q | N | | G | G | S |
| DENV4 | T | K | D | S | | D | G | H |

*Relative to DENV2 WT

TABLE 29
DENV3-ZIKV Z3L1 Swap

| | Residue #* | | | | | | | | |
|---|---|---|---|---|---|---|---|---|---|
| | 46 | 47 | 52 | 138 | 140 | 151 | 153 | 154 | 159 |
| Zika | V | T | N | R | M | T | H | E | K |
| DENV1 | L | K | N | S | I | V | N | E | T |
| DENV2 | I | K | Q | T | V | V | N | D | E |
| DENV3 | Q | K | Q | T | I | V | N | E | T |
| DENV4 | T | K | E | T | V | V | N | D | T |

| | Residue #* | | | | | | | |
|---|---|---|---|---|---|---|---|---|
| | 161 | 201 | 269 | 270 | 271 | 273 | 274 | 276 |
| Zika | E | N | E | M | D | A | K | R |
| DENV1 | T | K | Q | T | | G | T | T |
| DENV2 | K | N | Q | M | | S | G | L |
| DENV3 | E | N | Q | N | | G | G | S |
| DENV4 | T | K | D | S | | D | G | H |

*Relative to DENV3 WT

TABLE 30
DENV4-ZIKV Z3L1 Swap

| | Residue #* | | | | | | | | |
|---|---|---|---|---|---|---|---|---|---|
| | 46 | 47 | 52 | 138 | 140 | 151 | 153 | 154 | 161 |
| Zika | V | T | N | R | M | T | H | E | K |
| DENV1 | L | K | N | S | I | V | N | E | T |
| DENV2 | I | K | Q | T | V | V | N | D | E |
| DENV3 | Q | K | Q | T | I | V | N | E | T |
| DENV4 | T | K | E | T | V | V | N | D | T |

| | Residue #* | | | | | | | |
|---|---|---|---|---|---|---|---|---|
| | 163 | 203 | 271 | 272 | 273 | 275 | 276 | 278 |
| Zika | E | N | E | M | D | A | K | R |
| DENV1 | T | K | Q | T | | G | T | T |
| DENV2 | K | N | Q | M | | S | G | L |
| DENV3 | E | N | Q | N | | G | G | S |
| DENV4 | T | K | D | S | | D | G | H |

*Relative to DENV4 WT

TABLE 31
DENV-ZIKV Z20 and Z3L1 Swaps

| | Residue #* | | | | | | | | | | | |
|---|---|---|---|---|---|---|---|---|---|---|---|---|
| | 46 | 47 | 52 | 67 | 68 | 69 | 84 | 120 | 138 | 140 | 156 | 158 |
| Zika | V | T | N | D | M | A | K | A | R | M | T | H |
| DENV1 | L | K | N | N | T | T | E | K | S | I | V | N |
| DENV2 | I | K | Q | N | T | T | E | T | T | V | V | N |
| DENV3 | Q | K | Q | N | I | T | E | Q | T | I | V | N |
| DENV4 | T | K | E | N | I | T | E | S | T | V | V | N |

| | Residue #* | | | | | | | | | | | |
|---|---|---|---|---|---|---|---|---|---|---|---|---|
| | 159 | 166 | 168 | 208 | 232 | 233 | 276 | 277 | 278 | 280 | 281 | 283 |
| Zika | E | K | E | N | G | T | E | M | D | A | K | R |
| DENV1 | E | T | T | K | S | Q | Q | T | | G | T | T |
| DENV2 | D | E | K | N | Q | G | Q | M | | S | G | L |
| DENV3 | E | T | E | N | E | T | Q | N | | G | G | S |
| DENV4 | D | T | T | K | S | E | D | S | | D | G | H |

*Relative to ZikV WT

TABLE 32
DENV1-ZIKV Z20 and Z3L1 Swaps

| | Residue #* | | | | | | | | | | | |
|---|---|---|---|---|---|---|---|---|---|---|---|---|
| | 46 | 47 | 52 | 67 | 68 | 69 | 84 | 120 | 138 | 140 | 151 | 153 |
| Zika | V | T | N | D | M | A | K | A | R | M | T | H |
| DENV1 | L | K | N | N | T | T | E | K | S | I | V | N |
| DENV2 | I | K | Q | N | T | T | E | T | T | V | V | N |
| DENV3 | Q | K | Q | N | I | T | E | Q | T | I | V | N |
| DENV4 | T | K | E | N | I | T | E | S | T | V | V | N |
| | Residue #* | | | | | | | | | | | |
| | 154 | 161 | 163 | 203 | 227 | 228 | 271 | 272 | 273 | 275 | 276 | 278 |
| Zika | E | K | E | N | G | T | E | M | D | A | K | R |
| DENV1 | E | T | T | K | S | Q | Q | T | | G | T | T |
| DENV2 | D | E | K | N | Q | G | Q | M | | S | G | L |
| DENV3 | E | T | E | N | E | T | Q | N | | G | G | S |
| DENV4 | D | T | T | K | S | E | D | S | | D | G | H |

*Relative to DENV1 WT

TABLE 33
DENV2-ZIKV Z20 and Z3L1 Swaps

| | Residue #* | | | | | | | | | | | |
|---|---|---|---|---|---|---|---|---|---|---|---|---|
| | 46 | 47 | 52 | 67 | 68 | 69 | 84 | 120 | 138 | 140 | 151 | 153 |
| Zika | V | T | N | D | M | A | K | A | R | M | T | H |
| DENV1 | L | K | N | N | T | T | E | K | S | I | V | N |
| DENV2 | I | K | Q | N | T | T | E | T | T | V | V | N |
| DENV3 | Q | K | Q | N | I | T | E | Q | T | I | V | N |
| DENV4 | T | K | E | N | I | T | E | S | T | V | V | N |
| | Residue #* | | | | | | | | | | | |
| | 154 | 161 | 163 | 203 | 227 | 228 | 271 | 272 | 273 | 275 | 276 | 278 |
| Zika | E | K | E | N | G | T | E | M | D | A | K | R |
| DENV1 | E | T | T | K | S | Q | Q | T | | G | T | T |
| DENV2 | D | E | K | N | Q | G | Q | M | | S | G | L |
| DENV3 | E | T | E | N | E | T | Q | N | | G | G | S |
| DENV4 | D | T | T | K | S | E | D | S | | D | G | H |

*Relative to DENV2 WT

TABLE 34
DENV3-ZIKV Z20 and Z3L1 Swaps

| | Residue #* | | | | | | | | | | | |
|---|---|---|---|---|---|---|---|---|---|---|---|---|
| | 46 | 47 | 52 | 67 | 68 | 69 | 84 | 120 | 138 | 140 | 151 | 153 |
| Zika | V | T | N | D | M | A | K | A | R | M | T | H |
| DENV1 | L | K | N | N | T | T | E | K | S | I | V | N |
| DENV2 | I | K | Q | N | T | T | E | T | T | V | V | N |
| DENV3 | Q | K | Q | N | I | T | E | Q | T | I | V | N |
| DENV4 | T | K | E | N | I | T | E | S | T | V | V | N |
| | Residue #* | | | | | | | | | | | |
| | 154 | 159 | 161 | 201 | 225 | 226 | 269 | 270 | 271 | 273 | 274 | 276 |
| Zika | E | K | E | N | G | T | E | M | D | A | K | R |
| DENV1 | E | T | T | K | S | Q | Q | T | | G | T | T |
| DENV2 | D | E | K | N | Q | G | Q | M | | S | G | L |
| DENV3 | E | T | E | N | E | T | Q | N | | G | G | S |
| DENV4 | D | T | T | K | S | E | D | S | | D | G | H |

*Relative to DENV3 WT

TABLE 35
DENV4-ZIKV Z20 and Z3L1 Swaps

| | Residue #* | | | | | | | | | | | |
|---|---|---|---|---|---|---|---|---|---|---|---|---|
| | 46 | 47 | 52 | 67 | 68 | 69 | 84 | 120 | 138 | 140 | 151 | 153 |
| Zika | V | T | N | D | M | A | K | A | R | M | T | H |
| DENV1 | L | K | N | N | T | T | E | K | S | I | V | N |
| DENV2 | I | K | Q | N | T | T | E | T | T | V | V | N |
| DENV3 | Q | K | Q | N | I | T | E | Q | T | I | V | N |
| DENV4 | T | K | E | N | I | T | E | S | T | V | V | N |
| | Residue #* | | | | | | | | | | | |
| | 154 | 161 | 163 | 203 | 227 | 228 | 271 | 272 | 273 | 275 | 276 | 278 |
| Zika | E | K | E | N | G | T | E | M | D | A | K | R |
| DENV1 | E | T | T | K | S | Q | Q | T | | G | T | T |
| DENV2 | D | E | K | N | Q | G | Q | M | | S | G | L |
| DENV3 | E | T | E | N | E | T | Q | N | | G | G | S |
| DENV4 | D | T | T | K | S | E | D | S | | D | G | H |

*Relative to DENV4 WT

TABLE 36
Alignment of Zika-Dengue WT Sequences

| | Residue # | | | | | | | | | | | | | |
|---|---|---|---|---|---|---|---|---|---|---|---|---|---|---|
| | 1 | 2 | 3 | 4 | 5 | 6 | 7 | 8 | 9 | 10 | 11 | 12 | 13 | 14 |
| Zika WT | I | R | C | I | G | V | S | N | R | D | F | V | E | G |
| DENV1 WT | M | R | C | V | G | I | G | N | R | D | F | V | E | G |
| DENV2 WT | M | R | C | I | G | I | S | N | R | D | F | V | E | G |
| DENV3 WT | M | R | C | V | G | I | G | N | R | D | F | V | E | G |
| DENV4 WT | M | R | C | V | G | V | G | N | R | D | F | V | E | G |
| | Residue # | | | | | | | | | | | | | |
| | 15 | 16 | 17 | 18 | 19 | 20 | 21 | 22 | 23 | 24 | 25 | 26 | 27 | 28 |
| Zika WT | M | S | G | G | T | W | V | D | V | V | L | E | H | G |
| DENV1 WT | L | S | G | A | T | W | V | D | V | V | L | E | H | G |
| DENV2 WT | V | S | G | G | S | W | V | D | I | V | L | E | H | G |
| DENV3 WT | L | S | G | A | T | W | V | D | V | V | L | E | H | G |
| DENV4 WT | V | S | G | G | A | W | V | D | L | V | L | E | H | G |
| | Residue # | | | | | | | | | | | | | |
| | 29 | 30 | 31 | 32 | 33 | 34 | 35 | 36 | 37 | 38 | 39 | 40 | 41 | 42 |
| Zika WT | G | C | V | T | V | M | A | Q | D | K | P | T | V | D |
| DENV1 WT | S | C | V | T | T | M | A | K | D | K | P | T | L | D |
| DENV2 WT | S | C | V | T | T | M | A | K | N | K | P | T | L | D |
| DENV3 WT | G | C | V | T | T | M | A | K | N | K | P | T | L | D |
| DENV4 WT | G | C | V | T | T | M | A | Q | G | K | P | T | L | D |
| | Residue # | | | | | | | | | | | | | |
| | 43 | 44 | 45 | 46 | 47 | 48 | 49 | 50 | 51 | 52 | 53 | 54 | 55 | 56 |
| Zika WT | I | E | L | V | T | T | T | V | S | N | M | A | E | V |
| DENV1 WT | I | E | L | L | K | T | E | V | T | N | P | A | V | L |
| DENV2 WT | F | E | L | I | K | T | E | A | K | Q | P | A | T | L |
| DENV3 WT | I | E | L | Q | K | T | E | A | T | Q | L | A | T | L |
| DENV4 WT | F | E | L | T | K | T | T | A | K | E | V | A | L | L |
| | Residue # | | | | | | | | | | | | | |
| | 57 | 58 | 59 | 60 | 61 | 62 | 63 | 64 | 65 | 66 | 67 | 68 | 69 | 70 |
| Zika WT | R | S | Y | C | Y | E | A | S | I | S | D | M | A | S |
| DENV1 WT | R | K | L | C | I | E | A | K | I | S | N | T | T | T |
| DENV2 WT | R | K | Y | C | I | E | A | K | L | T | N | T | T | T |
| DENV3 WT | R | K | L | C | I | E | G | K | I | T | N | I | T | T |
| DENV4 WT | R | T | Y | C | I | E | A | S | I | S | N | I | T | T |
| | Residue # | | | | | | | | | | | | | |
| | 71 | 72 | 73 | 74 | 75 | 76 | 77 | 78 | 79 | 80 | 81 | 82 | 83 | 84 |
| Zika WT | D | S | R | C | P | T | Q | G | E | A | Y | L | D | K |
| DENV1 WT | D | S | R | C | P | T | Q | G | E | A | T | L | V | E |
| DENV2 WT | E | S | R | C | P | T | Q | G | E | P | S | L | N | E |
| DENV3 WT | D | S | R | C | P | T | Q | G | E | A | V | L | P | E |
| DENV4 WT | A | T | R | C | P | T | Q | G | E | P | Y | L | K | E |
| | Residue # | | | | | | | | | | | | | |
| | 85 | 86 | 87 | 88 | 89 | 90 | 91 | 92 | 93 | 94 | 95 | 96 | 97 | 98 |
| Zika WT | Q | S | D | T | Q | Y | V | C | K | R | T | L | V | D |
| DENV1 WT | E | Q | D | T | N | F | V | C | R | R | T | F | V | D |
| DENV2 WT | E | Q | D | K | R | F | V | C | K | H | S | M | V | D |
| DENV3 WT | E | Q | D | Q | N | Y | V | C | K | H | T | Y | V | D |
| DENV4 WT | E | Q | D | Q | Q | Y | I | C | R | R | D | V | V | D |
| | Residue # | | | | | | | | | | | | | |
| | 99 | 100 | 101 | 102 | 103 | 104 | 105 | 106 | 107 | 108 | 109 | 110 | 111 | 112 |
| Zika WT | R | G | W | G | N | G | C | G | L | F | G | K | G | S |
| DENV1 WT | R | G | W | G | N | G | C | G | L | F | G | K | G | S |
| DENV2 WT | R | G | W | G | N | G | C | G | L | F | G | K | G | G |
| DENV3 WT | R | G | W | G | N | G | C | G | L | F | G | K | G | S |
| DENV4 WT | R | G | W | G | N | G | C | G | L | F | G | K | G | G |
| | Residue # | | | | | | | | | | | | | |
| | 113 | 114 | 115 | 116 | 117 | 118 | 119 | 120 | 121 | 122 | 123 | 124 | 125 | 126 |
| Zika WT | L | V | T | C | A | K | F | A | C | S | K | K | M | T |
| DENV1 WT | L | I | T | C | A | K | F | K | C | V | T | K | L | E |
| DENV2 WT | I | V | T | C | A | M | F | T | C | K | K | N | M | E |
| DENV3 WT | L | V | T | C | A | K | F | Q | C | L | E | P | I | E |
| DENV4 WT | V | V | T | C | A | K | F | S | C | S | G | K | I | T |
| | Residue # | | | | | | | | | | | | | |
| | 127 | 128 | 129 | 130 | 131 | 132 | 133 | 134 | 135 | 136 | 137 | 138 | 139 | 140 |
| Zika WT | G | K | S | I | Q | P | E | N | L | E | Y | R | I | M |
| DENV1 WT | G | K | I | V | Q | Y | E | N | L | K | Y | S | V | I |
| DENV2 WT | G | K | V | V | Q | P | E | N | L | E | Y | T | I | V |
| DENV3 WT | G | K | V | V | Q | Y | E | N | L | K | Y | T | V | I |
| DENV4 WT | G | N | L | V | Q | I | E | N | L | E | Y | T | V | V |
| | Residue # | | | | | | | | | | | | | |
| | 141 | 142 | 143 | 144 | 145 | 146 | 147 | 148 | 149 | 150 | 151 | 152 | 153 | 154 |
| Zika WT | L | S | V | H | G | S | Q | H | S | G | M | I | V | N |
| DENV1 WT | V | T | V | H | T | G | D | Q | | | | | | H |
| DENV2 WT | V | T | P | H | S | G | E | E | | | | | | H |
| DENV3 WT | I | T | V | H | T | G | D | Q | | | | | | H |
| DENV4 WT | V | T | V | H | N | G | D | T | | | | | | H |
| | Residue # | | | | | | | | | | | | | |
| | 155 | 156 | 157 | 158 | 159 | 160 | 161 | 162 | 163 | 164 | 165 | 166 | 167 | 168 |
| Zika WT | D | T | G | H | E | T | D | E | N | R | A | K | V | E |
| DENV1 WT | Q | V | G | N | E | T | T | E | H | G | T | T | A | T |
| DENV2 WT | A | V | G | N | D | T | G | K | H | G | K | E | I | K |
| DENV3 WT | Q | V | G | N | E | | | T | Q | G | V | T | A | E |
| DENV4 WT | A | V | G | N | D | T | S | N | H | G | V | T | A | T |
| | Residue # | | | | | | | | | | | | | |
| | 169 | 170 | 171 | 172 | 173 | 174 | 175 | 176 | 177 | 178 | 179 | 180 | 181 | 182 |
| Zika WT | I | T | P | N | S | P | R | A | E | A | T | L | G | G |
| DENV1 WT | I | T | P | Q | A | P | T | S | E | I | Q | L | T | D |
| DENV2 WT | V | T | P | Q | S | S | I | T | E | A | E | L | T | G |
| DENV3 WT | I | T | P | Q | A | S | T | T | E | A | I | L | P | E |
| DENV4 WT | I | T | P | R | S | P | S | V | E | V | K | L | P | D |
| | Residue # | | | | | | | | | | | | | |
| | 183 | 184 | 185 | 186 | 187 | 188 | 189 | 190 | 191 | 192 | 193 | 194 | 195 | 196 |
| Zika WT | F | G | S | L | G | L | D | C | E | P | R | T | G | L |
| DENV1 WT | Y | G | A | L | T | L | D | C | S | P | R | T | G | L |
| DENV2 WT | Y | G | T | V | T | M | E | C | S | P | R | T | G | L |
| DENV3 WT | Y | G | T | L | G | L | E | C | S | P | R | T | G | L |
| DENV4 WT | Y | G | E | L | T | L | D | C | E | P | R | S | G | I |
| | Residue # | | | | | | | | | | | | | |
| | 197 | 198 | 199 | 200 | 201 | 202 | 203 | 204 | 205 | 206 | 207 | 208 | 209 | 210 |
| Zika WT | D | F | S | D | L | Y | Y | L | T | M | N | N | K | H |
| DENV1 WT | D | F | N | E | M | V | L | L | T | M | E | K | K | S |
| DENV2 WT | D | F | N | E | M | V | L | L | Q | M | E | N | K | A |
| DENV3 WT | D | F | N | E | M | I | L | L | T | M | K | N | K | A |
| DENV4 WT | D | F | N | E | M | I | L | M | K | M | K | K | K | T |
| | Residue # | | | | | | | | | | | | | |
| | 211 | 212 | 213 | 214 | 215 | 216 | 217 | 218 | 219 | 220 | 221 | 222 | 223 | 224 |
| Zika WT | W | L | V | H | K | E | W | F | H | D | I | P | L | P |
| DENV1 WT | W | L | V | H | K | Q | W | F | L | D | L | P | L | P |
| DENV2 WT | W | L | V | H | R | Q | W | F | L | D | L | P | L | P |
| DENV3 WT | W | M | V | H | R | Q | W | F | F | D | L | P | L | P |
| DENV4 WT | W | L | V | H | K | Q | W | F | L | D | L | P | L | P |
| | Residue # | | | | | | | | | | | | | |
| | 225 | 226 | 227 | 228 | 229 | 230 | 231 | 232 | 233 | 234 | 235 | 236 | 237 | 238 |
| Zika WT | W | H | A | G | A | D | T | G | T | P | H | W | N | N |
| DENV1 WT | W | T | S | G | A | S | T | S | Q | E | T | W | N | R |
| DENV2 WT | W | L | P | G | A | D | T | Q | G | S | N | W | I | Q |
| DENV3 WT | W | T | S | G | A | T | T | E | T | P | T | W | N | R |
| DENV4 WT | W | T | A | G | A | D | T | S | E | V | H | W | N | Y |
| | Residue # | | | | | | | | | | | | | |
| | 239 | 240 | 241 | 242 | 243 | 244 | 245 | 246 | 247 | 248 | 249 | 250 | 251 | 252 |
| Zika WT | K | E | A | L | V | E | F | K | D | A | H | A | K | R |
| DENV1 WT | Q | D | L | L | V | T | F | K | T | A | H | A | K | K |
| DENV2 WT | K | E | T | L | V | T | F | K | N | P | H | A | K | K |
| DENV3 WT | K | E | L | L | V | T | F | K | N | A | H | A | K | K |
| DENV4 WT | K | E | R | M | V | T | F | K | V | P | H | A | K | R |
| | Residue # | | | | | | | | | | | | | |
| | 253 | 254 | 255 | 256 | 257 | 258 | 259 | 260 | 261 | 262 | 263 | 264 | 265 | 266 |
| Zika WT | Q | T | V | V | V | L | G | S | Q | E | G | A | V | H |
| DENV1 WT | Q | E | V | V | V | L | G | S | Q | E | G | A | M | H |
| DENV2 WT | Q | D | V | V | V | L | G | S | Q | E | G | A | M | H |
| DENV3 WT | Q | E | V | V | V | L | G | S | Q | E | G | A | M | H |
| DENV4 WT | Q | D | V | T | V | L | G | S | Q | E | G | A | M | H |
| | Residue # | | | | | | | | | | | | | |
| | 267 | 268 | 269 | 270 | 271 | 272 | 273 | 274 | 275 | 276 | 277 | 278 | 279 | 280 |
| Zika WT | T | A | L | A | G | A | L | E | A | E | M | D | G | A |
| DENV1 WT | T | A | L | T | G | A | T | E | I | Q | T | S | G | T |
| DENV2 WT | T | A | L | T | G | A | T | E | I | Q | M | S | S | G |
| DENV3 WT | T | A | L | T | G | A | T | E | I | Q | N | S | G | G |
| DENV4 WT | S | A | L | A | G | A | T | E | V | D | S | G | D | G |
| | Residue # | | | | | | | | | | | | | |
| | 281 | 282 | 283 | 284 | 285 | 286 | 287 | 288 | 289 | 290 | 291 | 292 | 293 | 294 |
| Zika WT | K | G | R | L | S | S | G | H | L | K | C | R | L | K |
| DENV1 WT | T | | T | I | F | A | G | H | L | K | C | R | L | K |
| DENV2 WT | N | | L | L | F | T | G | H | L | K | C | R | L | R |
| DENV3 WT | T | | S | I | F | A | G | H | L | K | C | R | L | K |
| DENV4 WT | N | | H | M | F | A | G | H | L | K | C | K | V | R |
| | Residue # | | | | | | | | | | | | | |
| | 295 | 296 | 297 | 298 | 299 | 300 | 301 | 302 | 303 | 304 | 305 | 306 | 307 | 308 |
| Zika WT | M | D | K | L | R | L | K | G | V | S | Y | S | L | C |
| DENV1 WT | M | D | K | L | T | L | K | G | M | S | Y | V | M | C |
| DENV2 WT | M | D | K | L | Q | L | K | G | M | S | Y | S | M | C |
| DENV3 WT | M | D | K | L | E | L | K | G | M | S | Y | A | M | C |
| DENV4 WT | M | E | K | L | R | I | K | G | M | S | Y | T | M | C |
| | Residue # | | | | | | | | | | | | | |
| | 309 | 310 | 311 | 312 | 313 | 314 | 315 | 316 | 317 | 318 | 319 | 320 | 321 | 322 |
| Zika WT | T | A | A | F | T | F | T | K | I | P | A | E | T | L |
| DENV1 WT | T | G | S | F | K | L | E | K | E | V | A | E | T | Q |
| DENV2 WT | T | G | K | F | K | V | V | K | E | I | A | E | T | Q |
| DENV3 WT | T | N | T | F | V | L | K | K | E | V | S | E | T | Q |
| DENV4 WT | S | G | K | F | S | I | D | K | E | M | A | E | T | Q |
| | Residue # | | | | | | | | | | | | | |
| | 323 | 324 | 325 | 326 | 327 | 328 | 329 | 330 | 331 | 332 | 333 | 334 | 335 | 336 |
| Zika WT | H | G | T | V | T | V | E | V | Q | Y | A | G | T | D |
| DENV1 WT | H | G | T | V | L | V | Q | V | K | Y | E | G | T | D |
| DENV2 WT | H | G | T | I | V | I | R | V | Q | Y | E | G | D | G |
| DENV3 WT | H | G | T | I | L | I | K | V | E | Y | K | G | E | D |
| DENV4 WT | H | G | T | T | V | V | K | V | K | Y | E | G | A | G |
| | Residue # | | | | | | | | | | | | | |
| | 337 | 338 | 339 | 340 | 341 | 342 | 343 | 344 | 345 | 346 | 347 | 348 | 349 | 350 |
| Zika WT | G | P | C | K | V | P | A | Q | M | A | V | D | M | Q |
| DENV1 WT | A | P | C | K | I | P | F | S | S | Q | D | E | K | G |
| DENV2 WT | S | P | C | K | I | P | F | E | I | M | D | L | E | K |
| DENV3 WT | A | P | C | K | I | P | F | S | T | E | D | G | Q | G |
| DENV4 WT | A | P | C | K | V | P | I | E | I | R | D | V | N | K |
| | Residue # | | | | | | | | | | | | | |
| | 351 | 352 | 353 | 354 | 355 | 356 | 357 | 358 | 359 | 360 | 361 | 362 | 363 | 364 |
| Zika WT | T | L | T | P | V | G | R | L | I | T | A | N | P | V |
| DENV1 WT | V | T | Q | | N | G | R | L | I | T | A | N | P | I |
| DENV2 WT | R | H | V | | L | G | R | L | I | T | V | N | P | I |
| DENV3 WT | K | A | H | | N | G | R | L | I | T | A | N | P | V |
| DENV4 WT | E | K | V | | V | G | R | V | I | S | S | T | P | L |
| | Residue # | | | | | | | | | | | | | |
| | 365 | 366 | 367 | 368 | 369 | 370 | 371 | 372 | 373 | 374 | 375 | 376 | 377 | 378 |
| Zika WT | I | T | E | S | T | E | N | S | K | M | M | L | E | L |
| DENV1 WT | V | T | D | K | E | | | K | P | V | N | I | E | A |
| DENV2 WT | V | T | E | K | D | | | S | P | V | N | I | E | A |
| DENV3 WT | V | T | K | K | E | | | E | P | V | N | I | E | A |
| DENV4 WT | A | E | N | T | N | | | S | V | T | N | I | E | L |
| | Residue # | | | | | | | | | | | | | |
| | 379 | 380 | 381 | 382 | 383 | 384 | 385 | 386 | 387 | 388 | 389 | 390 | 391 | 392 |
| Zika WT | D | P | P | F | G | D | S | Y | I | V | I | G | V | G |
| DENV1 WT | E | P | P | F | G | E | S | Y | I | V | V | G | A | G |
| DENV2 WT | E | P | P | F | G | D | S | Y | I | I | I | G | V | D |
| DENV3 WT | E | P | P | F | G | E | S | N | I | V | I | G | I | G |
| DENV4 WT | E | P | P | F | G | D | S | Y | I | V | I | G | V | G |

| | Residue # | | | | | | | | | | |
|---|---|---|---|---|---|---|---|---|---|---|---|
| | 393 | 394 | 395 | 396 | 397 | 398 | 399 | 400 | 401 | 402 | 403 |
| Zika WT | E | K | K | I | T | H | H | W | H | R | S |
| DENV1 WT | E | K | A | L | K | L | S | W | F | K | K |
| DENV2 WT | P | G | Q | L | K | L | N | W | F | K | K |
| DENV3 WT | D | N | A | L | K | I | N | W | Y | K | K |
| DENV4 WT | N | S | A | L | T | L | H | W | F | R | K |

TABLE 37
List of interaction sites of the three individual Fabs C10 with the E proteins in
the asymmetric unit in the ZIKV-CH pH 8 structure.

| C10 Fab | | | | | |
|---|---|---|---|---|---|
| | | Fab residue | | | |
| | | (H—Heavy chain, | | | |
| | | L—light chain, CDR | | | |
| | | loops and frame | E protein residues | | |
| | | work regions are | Molecules A, B, C in an asymmetric unit | | |
| | Main interacting | indicated in | indicated as A, B and C, respectively | | |
| Fab molecule | E protein dimer | brackets) | Intra-dimer | Inter-dimer | Inter-raft |
| I | A-C′ (near the | H-G53 (CDR2) | A-D278 | | |
| | 5 fold) | H-G54(CDR2) | A-276, A-D278 | | |
| | | H-K58 (CDR2) | C-A69 | | |
| | | H-Q61 (CDR2) | C-D67 | | |
| | | H-Q64 (CDR2) | C-D67 | | |
| | | H-R71 (FH3) | A-D278 | | |
| | | H-K96 (CDR3) | A-D155 | | |
| | | H-D98 | A-R2 | | |
| | | (CDR3) | | | |
| | | H-D99 | A-R2, A-V46, A- | | |
| | | (CDR3) | M140 | | |
| | | H-Y100 | A-R2, A-H27, A- | | |
| | | (CDR3) | V46, A-V47 | | |
| | | H-D100B (CDR3) | C-K251, | | |
| | | H-W100D (CDR3) | C-S70, C- | | |
| | | | K251, C-R252, | | |
| | | | C-R253 | | |
| | | H-F100E | C-S72, C- | | |
| | | (CDR3) | R99, C-G102, C- | | |
| | | | N103 | | |
| | | H-P100F | C-G102 | | |
| | | (CDR3) | | | |
| | | H-L100H (CDR3) | A-S149, A- | | |
| | | | G150, A-M151 | | |
| | | H-W1001 | A-G150, A-V153 | | |
| | | (CDR3) | | | |
| | | L-S26(CDR1) | C-R73 | | A-K297 |
| | | L-G27(CDR1) | C-Q77 | | |
| | | L-G28(CDR1) | C-R73, C-C74, C- | | A-K297 |
| | | | Q77 | | |
| | | L-F29(CDR1) | C-S72, C- | | |
| | | | R73, C-C74, C- | | |
| | | | N103 | | |
| | | L-N30(CDR1) | C-G104 | | |
| | | L-Y31(CDR1) | C-G102, C-N103 | | |
| | | L-Y48(FL2) | A-G150, A- | | |
| | | | M151, A-I152 | | |
| | | L-D49(CDR2) | A-M151 | | |
| | | L-T51(CDR2) | A-M151, A- | | |
| | | | N371, A-K373 | | |
| | | L-R53(CDR2) | A-E370, A-N371 | | |
| | | L-P54(CDR2) | A-V153 | | |
| | | L-V57(FL3) | A-N371 | | |
| | | L-S59(FL3) | A-E370 | | |
| | | L-K65(FL3) | | | A-R299 |
| | | L-S66(FL3) | | | A-L180, A- |
| | | | | | G181, A- |
| | | | | | G182, A- |
| | | | | | G184, A- |
| | | | | | R299 |
| | | L-G67(FL3) | | | A-G184, A- |
| | | | | | R299 |
| | | L-N68(FL3) | | | A-S185 |
| | | L-S92(CDR3) | C-S70, C-D71, C- | | |
| | | | S72, C-R73 | | |
| | | L-R93(CDR3) | C-A69, C-S70, C- | | |
| | | | D71, C-L82, C- | | |
| | | | D83, C-K84 | | |
| | | L-G94(CDR3) | C-S70 | | |
| II | A-C′ (near the | H-K58(CDR2) | A-M68, A-A69, | | |
| | 3-fold vertex) | | A-S70 | | |
| | | H-Q61(CDR2) | A-K84 | | |
| | | H-D99 | C-R2, C-V46, C- | | |
| | | (CDR3) | R138, C-M140, | | |
| | | H-Y100(CDR3) | A-H249, A- | | |
| | | | K251, C-H27, C- | | |
| | | | E44, C-V46, C- | | |
| | | | T47, C-R283 | | |
| | | H-D100B (CDR3) | A-R252, C-E276 | | |
| | | H-W100D (CDR3) | A-M68, A- | | |
| | | | S70, A-R252, A- | | |
| | | | Q253 | | |
| | | H-F100E (CDR3) | A-S72, A-D98, | | |
| | | | A-N103, A- | | |
| | | | L113, A-R252 | | |
| | | H-P100F (CDR3) | A-W101, A-G102 | | |
| | | H-L100H (CDR3) | A-G102, A- | | |
| | | | N103, A-G104, C- | | |
| | | | S149 | | |
| | | H-W100I(CDR3) | C-S149 | | |
| | | L-S26(CDR1) | A-R73 | B′-E55 | |
| | | L-S26A (CDR1) | A-R73 | | |
| | | L-D26B (CDR1) | A-R73 | | |
| | | L-G27(CDR1) | A-Q77 | | |
| | | L-G28(CDR1) | A-C74, A-Q77, A- | | |
| | | | C104, A-C105 | | |
| | | L-F29(CDR1) | A-S72, A-R73, A- | | |
| | | | C74, A-N103, A- | | |
| | | | G104 | | |
| | | L-N30(CDR1) | A-W101, A- | | |
| | | | N103, A-G104, A- | | |
| | | | C105, A-G106, | | |
| | | | C-K316 | | |
| | | L-Y31(CDR1) | A-G102, A- | | |
| | | | N103, A-G104 | | |
| | | L-Y48(FL2) | C-H148, C-S149 | | |
| | | L-D49(CDR2) | C-K316 | | |
| | | L-T51(CDR2) | C-T315, C- | | |
| | | | N371, C-K373 | | |
| | | L-S52(CDR2) | C-H148, C- | | |
| | | | S372, C-K373 | | |
| | | L-R53(CDR2) | C-T369, C-E370, | | |
| | | | C-N371 | | |
| | | L-S59(FL3) | C-T369, C-E370 | | |
| | | L-R60(FL3) | C-E370 | | |
| | | L-F61(CDR2) | C-E370 | | |
| | | L-S66(CDR2) | | B′-N52 | |
| | | L-G67(CDR2) | | B′-N52 | |
| | | L-S92(CDR3) | A-S70, A-D71, A- | | |
| | | | S72, A-R73 | | |
| | | L-R93(CDR3) | A-S70, A-D71, A- | | |
| | | | L82, A-D83, A- | | |
| | | | K84 | | |
| | | L-G94(CDR3) | A-S70 | | |
| III | B-B′(near the | H-N54(CDR2) | B-M277, B-R283 | | |
| | 2-fold vertex) | H-K58(CDR2) | B′-M68, B′-A69, | | |
| | | | B′-S70 | | |
| | | H-Q61(CDR2) | B′-K84 | | |
| | | H-Q64(CDR2) | B′-D67, B′-K84 | | |
| | | H-K96(CDR3) | B-R164 | | |
| | | H-D98(CDR3) | B-R2, B′-K251 | | |
| | | H-D99(CDR3) | B-R2, B-E44, B- | | |
| | | | V46, B-T47, B- | | |
| | | | M140, B-R164 | | |
| | | H-Y100 (CDR3) | B-H27, B- | | |
| | | | G28, B-E44, B- | | |
| | | | V46, B-T47, C- | | |
| | | | R283 | | |
| | | H-D100B (CDR3) | B′-R252, B- | | |
| | | | E276, B-R283 | | |
| | | H-W100D (CDR3) | B′-S70, B′- | | |
| | | | T115, B′-R252, | | |
| | | | B′-Q253 | | |
| | | H-F100E (CDR3) | B′-S72, B′- | | |
| | | | R99, B′-N103, | | |
| | | | B′-L113, B′- | | |
| | | | R252 | | |
| | | H-P100F (CDR3) | B′-N103 | | |
| | | H-L100H (CDR3) | B′-G102, B-S149 | | |
| | | H-W100I(CDR3) | B-S149 | | |
| | | L-S26(CDR3) | B′-R73, B′-Q77 | A-A229, A- | |
| | | | | D230 | |
| | | L-S26A (CDR1) | B′-R73 | | |
| | | L-G28(CDR1) | B′C74,, B′- | | |
| | | | Q77, B′-C105 | | |
| | | L-F29(CDR1) | B′-S72, B′- | | |
| | | | R73, B′-C74, B′- | | |
| | | | R99, B′-N103, B′- | | |
| | | | G104, B′-C105 | | |
| | | L-N30(CDR1) | B′-G104, B′- | | |
| | | | C105, B′-G106, | | |
| | | | B-K316 | | |
| | | L-Y31(CDR1) | B′-N103, B′- | | |
| | | | G104 | | |
| | | L-Y48(FL2) | B-H148, B-S149 | | |
| | | L-D49(CDR2) | B-S149, B-K316 | | |
| | | L-T51(CDR2) | B-T315, B-E329, | | |
| | | | B-Q331, B-K373 | | |
| | | L-S52(CDR2) | B-148, B-T315, | | |
| | | | B-E329, B- | | |
| | | | Q331, B-K373 | | |
| | | L-R53(CDR2) | B-148, B-E370, | | |
| | | | B-N371, B-K373 | | |
| | | L-V57(FL3) | B-T369 | | |
| | | L-T59(FL3) | B-T369, B-E370 | | |
| | | L-S66(FL3) | | A-N52 | |
| | | L-G67(FL3) | | A-N52 | |
| | | L-S92(CDR3) | B′-S70, B′- | | |
| | | | D71, B′-S72, B′- | | |
| | | | R73 | | |
| | | L-R93(CDR3) | B′-A69, B′- | | |
| | | | S70, B′-D71, B′- | | |
| | | | S72, B′-L82, B′- | | |
| | | | D83, B′-K84 | | |
| | | L-G94(CDR3) | B′D71 | | |

TABLE 38
The distances between the interacting residues of Fab C10 and
the E proteins at two sites at the inter-dimer interfaces of
the ZIKV-C10 complex structure at pH 8 and pH 5.

| Interacting residues | | | |
|---|---|---|---|
| | ZIKV E protein | Distances between the | |
| | Molecules A, B and | Fab and E protein at | |
| C10 Fab | C are indicated as A, | the interdimer interface. | |
| H—heavy clain | B and C, | ZIKV-C10 pH8 | ZIKV-C10 pH5 |
| L—light Chain | respectively. | structure | structure |
| L-G67 | B′-N52 | 6.3 Å | 6.5 Å |
| L-S26 | B′-A54 | 7.6 Å | 8.0 Å |
| L-G28 | B′-A54 | 8.0 Å | 9.6 Å |
| H-G67 | A-N52 | 6.1 Å | 6.9 Å |
| H-S26 | A-A54 | 7.9 Å | 8.9 Å |
| H-S26 | A-A229 | 7.4 Å | 7.6 Å |

TABLE 39
Construct name: ZIKV H/PF/2013 - EDE Null - Core and
EDE1-Specific Backbone: ZIXV H/PF/2013

| Position in ZIKV | ZIKV H/PF/2013 | TABV |
|---|---|---|
| Envelope Protein | Parental AA | Transplanted AA |
| 2 | R | H |
| 28 | G | D |
| 44 | E | T |
| 45 | L | V |
| 69 | A | E |
| 71 | D | R |
| 72 | S | A |
| 73 | R | K |
| 77 | Q | A |
| 82 | L | I |
| 102 | G | D |
| 103 | N | S |
| 106 | G | F |
| 148 | H | N |
| 151 | M | K |
| 163 | N | K |
| 251 | K | T |
| 252 | R | Y |
| 253 | Q | I |
| 254 | T | P |
| 276 | E | K |
| 316 | K | S |
| 317 | I | G |
| 329 | E | L |
| 331 | Q | G |

TABLE 40
Construct name: ZIKV H/PF/2013 - EDE Null - Core Only
Backbone: ZIXV H/PF/2013

| Position in ZIKV | ZIKV H/PF/2013 | TABV |
|---|---|---|
| Envelope Protein | Parental AA | Transplanted AA |
| 69 | A | E |
| 71 | D | R |
| 72 | S | A |
| 73 | R | K |
| 102 | G | D |
| 103 | N | S |
| 251 | K | T |
| 252 | R | Y |
| 253 | Q | I |
| 254 | T | P |

TABLE 41
Construct name: ZIKV H/PF/2013 - EDE Null - EDE1-Specific
Only Backbone: ZIXV H/PF/2013

| Position in ZIKV | ZIKV H/PF/2013 | TABV |
|---|---|---|
| Envelope Protein | Parental AA | Transplanted AA |
| 2 | R | H |
| 28 | G | D |
| 44 | E | T |
| 45 | L | V |
| 77 | Q | A |
| 82 | L | I |
| 106 | G | F |
| 148 | H | N |
| 151 | M | K |
| 163 | N | K |
| 276 | E | K |
| 316 | K | S |
| 317 | I | G |
| 329 | E | L |
| 331 | Q | G |

TABLE 42
Construct name: DENV1 WestPac74 - EDE Null - Core and
EDE1-Specific Backbone: ZIXV H/PF/2013

| Position in DENV-1 | DENV-1 Westpac74 | TABV |
|---|---|---|
| Envelope Protein | Parental AA | Transplanted AA |
| 2 | R | H |
| 28 | G | D |
| 44 | E | T |
| 45 | L | V |
| 69 | T | E |
| 71 | D | R |
| 72 | S | A |
| 73 | R | K |
| 77 | Q | A |
| 82 | L | I |
| 102 | G | D |
| 103 | N | S |
| 106 | G | F |
| 148 | Q | N |
| 150 | Q | K |
| 158 | H | K |
| 246 | K | T |
| 247 | K | Y |
| 248 | Q | I |
| 249 | E | P |
| 271 | Q | K |
| 310 | K | S |
| 311 | E | G |
| 323 | Q | L |
| 325 | K | G |

TABLE 43
Construct name: DENV1 WestPac74 - EDE Null - Core Only
Backbone: ZIXV H/PF/2013

| Position in DENV-1 | DENV-1 Westpac74 | TABV |
|---|---|---|
| Envelope Protein | Parental AA | Transplanted AA |
| 69 | T | E |
| 71 | D | R |
| 72 | S | A |
| 73 | R | K |
| 102 | G | D |
| 103 | N | S |
| 246 | K | T |
| 247 | K | Y |
| 248 | Q | I |
| 249 | E | P |

TABLE 44
Construct name: DENV1 WestPac74 - EDE Null - EDE1-Specific
Only Backbone: ZIXV H/PF/2013

| Position in DENV-1 | DENV-1 Westpac74 | TABV |
|---|---|---|
| Envelope Protein | Parental AA | Transplanted AA |
| 2 | R | H |
| 28 | G | D |
| 44 | E | T |
| 45 | L | V |
| 77 | Q | A |
| 82 | L | I |
| 106 | G | F |
| 148 | Q | N |
| 150 | Q | K |
| 158 | H | K |
| 271 | Q | K |
| 310 | K | S |
| 311 | E | G |
| 323 | Q | L |
| 325 | K | G |

What is claimed is:

1. A method of protecting a fetus from the effects of Zika virus infection, comprising administering to the fetus and/or the mother of the fetus an effective amount of an EDE1 antibody and/or an epitope that binds an EDE1 antibody.

2. A method of producing an immune response to a Zika virus in a subject, comprising administering to the subject an effective amount of an epitope that binds an EDE1 antibody.

3. The method of claim 1, wherein the EDE1 antibody is selected from the group consisting of C8, C10 and any combination thereof.

4. A chimeric flavivirus E glycoprotein comprising amino acid residue substitutions that introduce a heterologous epitope that induces an antibody response to Zika virus, selected from the group consisting of:
a) Zika virus backbone with DENV1 “5J7” epitope;
b) Zika virus backbone with DENV2 “5J7” epitope;
c) Zika virus backbone with DENV3 “5J7” epitope;
d) Zika virus backbone with DENV4 “5J7” epitope;
e) a DENV1 backbone with “5J7” Zika epitope;
f) a DENV2 backbone with “5J7” Zika epitope;
g) a DENV3 backbone with “5J7” Zika epitope;
h) a DENV4 backbone with “5J7” Zika epitope;
i) a DENV1 backbone with EDIII from Zika;
j) a DENV2 backbone with EDIII from Zika;
k) a DENV3 backbone with EDIII from Zika;
l) a DENV4 backbone with EDIII from Zika;
m) a DENV1 backbone with “Z20” Zika epitope;
n) a DENV2 backbone with “Z20” Zika epitope;
o) a DENV3 backbone with “Z20” Zika epitope;
p) a DENV4 backbone with “Z20” Zika epitope;
q) a DENV1 backbone with “Z3L1” Zika epitope;
r) a DENV2 backbone with “Z3L1” Zika epitope;
s) a DENV3 backbone with “Z3L1” Zika epitope;
t) a DENV4 backbone with “Z3L1” Zika epitope;
u) a DENV1 backbone with “Z20” and “Z3L1” Zika epitopes;
v) a DENV2 backbone with “Z20” and “Z3L1” Zika epitopes;
w) a DENV3 backbone with “Z20” and “Z3L1” Zika epitopes;
x) a DENV4 backbone with “Z20” and “Z3L1” Zika epitopes;
y) a Zika virus backbone with a DENV1 C10 epitope;
z) a Zika virus backbone with a DENV2 C10 epitope;
aa) a Zika virus backbone with a DENV3 C10 epitope;
bb) a Zika virus backbone with a DENV4 C10 epitope;
cc) a Zika virus backbone with a DENV1 C10 epitope (non-neurotropic);
dd) a Z y) a Zika virus backbone with a DENV1 C10 epitope;
z) a Zika virus backbone with a DENV2 C10 epitope;
aa) a Zika virus backbone with a DENV3 C10 epitope;
bb) a Zika virus backbone with a DENV4 C10 epitope;
cc) a Zika virus backbone with a DENV1 C10 epitope (non-neurotropic);
dd) a Zika virus backbone with a DENV2 C10 epitope (non-neurotropic
ee) a Zika virus backbone with a DENV3 C10 epitope (non-neurotropic);
ff) a Zika virus backbone with a DENV4 C10 epitope (non-neurotropic);
gg) a Zika virus backbone with a DENV1 C8 epitope;
hh) a Zika virus backbone with a DENV2 C8 epitope;
ii) a Zika virus backbone with a DENV3 C8 epitope;
jj) a Zika virus backbone with a DENV4 C8 epitope;
kk) a DENV1 backbone with a ZIKV C10 epitope;
ll) a DENV2 backbone with a ZIKV C10 epitope;
mm) a DENV3 backbone with a ZIKV C10 epitope;
nn) a DENV4 backbone with a ZIKV C10 epitope;
oo) a DENV1 backbone with a ZIKV C10 epitope (neurotropic);
pp) a DENV2 backbone with a ZIKV C10 epitope (neurotropic);
qq) a DENV3 backbone with a ZIKV C10 epitope (neurotropic);
rr) a DENV4 backbone with a ZIKV C10 epitope (neurotropic);
ss) a DENV1 backbone with a ZIKV C8 epitope;
tt) a DENV2 backbone with a ZIKV C8 epitope;
uu) a DENV3 backbone with a ZIKV C8 epitope; and
vv) a DENV4 backbone with a ZIKV C8 epitope.

5. A method of treating a Zika virus infection in a subject, comprising administering to the subject an effective amount of the chimeric protein of claim 4.

6. A method of protecting a subject from the effects of Zika virus infection, comprising administering to the subject an effective amount of the chimeric protein of claim 4.

7. A method of protecting a fetus from the effects of Zika virus infection, comprising administering to the fetus and/or the mother of the fetus an effective amount of the chimeric protein of claim 4.

8. A method of reducing the complications caused by Zika virus infection in a subject, comprising administering to the subject an effective amount of the chimeric protein of claim 4.

9. A method of treating a disorder caused by Zika virus infection in a subject, comprising administering to the subject an effective amount of the chimeric protein of claim 4.

10. A method of producing an immune response to a Zika virus in a subject, comprising administering to the subject an effective amount of the chimeric protein of claim 4.

11. A method of preventing a Zika virus infection and/or preventing the effects of a Zika virus infection in a subject, comprising administering to the subject an effective amount of the chimeric protein of claim 4.

12. A chimeric flavivirus E glycoprotein comprising substitutions in the amino acid sequence that ablate an epitope that induces an immune response to Zika virus, selected from the group consisting of:
a) a Zika virus backbone with “Z20” epitope ablation by DENV1;
b) a Zika virus backbone with “Z20” epitope ablation by DENV2;
c) a Zika virus backbone with “Z20” epitope ablation by DENV3;
d) a Zika virus backbone with “Z20” epitope ablation by DENV4;
e) a Zika virus backbone with “Z3L1” epitope ablation by DENV1;
f) a Zika virus backbone with “Z3L1” epitope ablation by DENV2;
g) a Zika virus backbone with “Z3L1” epitope ablation by DENV3;
h) a Zika virus backbone with “Z3L1” epitope ablation by DENV4;
i) a Zika virus backbone with “Z20” and “Z3L1” epitope ablation by DENV1;
j) a Zika virus backbone with “Z20” and “Z3L1” epitope ablation by DENV2;
k) a Zika virus backbone with “Z20” and “Z3L1” epitope ablation by DENV3;
l) a Zika virus backbone with “Z20” and “Z3L1” epitope ablation by DENV4;
m) Zika virus backbone with EDE1 epitope ablation;
n) Zika virus backbone with core ablation;
o) Zika virus backbone with EDE1 epitope and core ablation;
p) DENV1 backbone with EDE1 epitope ablation;
q) DENV1 backbone with core ablation;
r) DENV1 backbone with EDE1 epitope and core ablation;
s) DENV2 backbone with EDE1 epitope ablation;
t) DENV2 backbone with core ablation;
u) DENV2 backbone with EDE1 epitope and core ablation;
v) DENV3 backbone with EDE1 epitope ablation;
w) DENV3 backbone with core ablation;
x) DENV3 backbone with EDE1 epitope and core ablation;
y) DENV4 backbone with EDE1 epitope ablation;
z) DENV4 backbone with core ablation; and
aa) DENV4 backbone with EDE1 epitope and core ablation.
